(19) United States
(12) Patent Application Publication
(10) Pub. No.: US 2021/0347780 A1
(43) Pub. Date: Nov. 11, 2021

(54) CDPK1 INHIBITORS, COMPOSITIONS, AND METHODS RELATED THERETO

(71) Applicants: Vyera Pharmaceuticals, LLC, New York, NY (US); Washington University, St. Louis, MO (US)

(72) Inventors: Allen T. HOPPER, Lexington, MA (US); L. David SIBLEY, University City, MO (US); James W. JANETKA, St. Louis, MO (US)

(21) Appl. No.: 17/277,603

(22) PCT Filed: Sep. 19, 2019
(86) PCT No.: PCT/US2019/051877

§ 371 Date: Mar. 18, 2021

Related U.S. Application Data
(60) Provisional application No. 62/733,361, filed on Sep. 19, 2018.

(51) Int. Cl. C07D 487/04

(52) U.S. Cl. CPC C07D 487/04

(57) ABSTRACT

The invention relates to inhibitors of calcium-dependent protein kinase 1 (CDPK1) and pharmaceutical preparations thereof. The invention further relates to methods of treatment of parasitic infections, such as T. gondii, P. falciparum, C. hominis, or C. parvum infections, using the novel inhibitors of the invention.

### CROSS-REFERENCE TO RELATED APPLICATIONS

This application claims the benefit of priority to U.S. Provisional Patent Application No. 62/733,361, filed Sep. 19, 2018, which is incorporated by reference herein in its entirety.

### BACKGROUND OF THE INVENTION

The phylum Apicomplexa contains parasites that are the causative agents for many serious human and animal diseases. Apicomplexans have complex life cycles sometimes existing in a single host and in other cases alternating between hosts. In the case of Toxoplasma gondii (T. gondii), cats transmit the disease by shedding infectious oocysts, which can contaminate food and water. Herbivorous hosts such as agricultural animals are also susceptible and in such animals infection culminates in the formation of long-lived tissue cysts that characterize chronic infections. Humans can become infected by ingesting oocysts found in contaminated water or by eating undercooked meat that contains tissue cysts. Although many strains of T. gondii are non-pathogenic, infection with some isolates is associated with severe infection in immunocompetent individuals. In some regions of the world, notably South America, toxoplasmosis can lead to severe eye disease and loss of vision.

Other human pathogens within the Apicomplexa include Plasmodium spp., (e.g. Plasmodium falciparum, Plasmodium vivax), Babesia spp. (eg. Babesia microti, Babesia bigemina), Cyclospora cayetanensis, Isospora belli, Sarcocystis neurona, and Cryptosporidium spp. (e.g., Cryptosporidium parvum or Cryptosporidium hominis), which cause malaria, toxoplasmosis, babesiosis, cyclosporiasis, isosporiasis, sarcocystosis, and cryptosporidiosis respectively. As well, there are a number of apicomplexan parasites that cause serious economic loss in agricultural animals including Eimeria spp., the causative agent of coccidiosis, and Sarcocystis spp., which causes Equine Protozoal Myeloencephalitis, as well as parasites that cause disease in companion animals such as Neospora canimum that causes neosporosis in dogs. Treatment options for all of these infections are severely limited.

Existing treatments for toxoplasmosis include administration of pyrimethamine, usually in combination with a dihydropteroate synthase (DHPS) sulfonamide inhibitor (e.g., sulfadiazine) to improve efficacy and the tetrahydrofolate folinic acid also called leucovorin to improve tolerability. Allergic reactions to sulfonamide drugs are common and therefore some patients are not able to receive the combination therapy. Pyrimethamine treatment may cause severe side-effects and toxicity, including nausea, vomiting, leukopenia, bone marrow toxicity, teratogenicity and central nervous system toxicity.

In addition, the existing treatments for toxoplasmosis do not eradicate chronic infection, which posses the major risk in immunocompromised patients. Approximately 1-2 billion people are estimated to be chronically infected worldwide. Thus, there is a need for new treatments for acute and chronic toxoplasmosis. Likewise, there is a need for new treatments for infections with Plasmodium spp., Babesia spp., Cryptosporidium spp., 10 Eimeria spp., Cydospora cayetanensis, Isospora belli, Sarcoiystis neurona, and Neospora caninum.

### SUMMARY OF THE INVENTION

In certain embodiments, the present invention relates to compounds having the structure of formula (I):

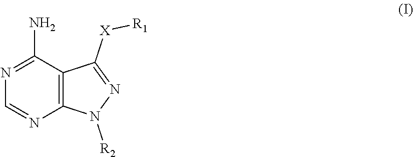

wherein:
X is R⁶ or O;
R¹ is phenyl or 5-10 membered heteroaryl;
R² is C₅₋₇ cycloalkyl, 4-7 membered heterocyclyl. C₁₋₆ alkyl, heteroaralkyl, carbocyclylalkyl, heterocyclylalkyl, C₂₋₆ alkenyl, C₄₋₆ cycloalkenyl, or H; and
R⁶ is C₁₋₆ alkylene;
   or a pharmaceutically acceptable salt thereof.

The invention further relates to pharmaceutical compositions of such compounds, as well as methods of using such compounds to treat infections (e.g., parasitic infections, such as Plasmodium spp. (e.g., Plasmodium falciparum, Plasmodium vivax), Babesia spp. (e.g., Babesia microti, Babesia bigemina). Cyclospora cayetanensis, Isospora belli, Sarcocystis neurona, and Cryptosporidium spp. (e.g., Cryptosporidium parvum or Cryptosporidium hominis), which cause malaria, toxoplasmosis, babesiosis, cyclosporiasis, isosporiasis, sarcocystosis, and cryptosporidiosis respectively).

### DETAILED DESCRIPTION OF THE INVENTION

In some aspects, the present invention relates to compounds having the structure of formula (I):

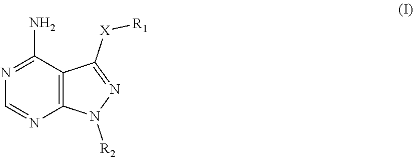

wherein:
X is R⁶ or O;
R¹ is phenyl or 5-10 membered heteroaryl;
R² is C₅₋₇ cycloalkyl, 4-7 membered heterocyclyl, C₁₋₆ alkyl, heteroaralkyl, carbocyclylalkyl, heterocyclylalkyl, C₂₋₆ alkenyl, C₄₋₆ cycloalkenyl, or H; and
R⁶ is C₁₋₆ alkylene;
   or a pharmaceutically acceptable salt thereof.

In certain embodiments, X is R⁶. In certain preferred embodiments, X is O.

In certain embodiments, R¹ is unsubstituted.

In certain embodiments, R¹ is substituted with one or more R⁵, and each R⁵ is independently selected from alkyl, such as haloalkyl, cycloalkyl, halo, hydroxyl, alkoxy, acyloxy, cyano, and amide. In some such embodiments, each R⁵ is independently selected from alkyl, C₁₋₃ haloalkyl, halo, hydroxyl, alkoxy, cyano, acyloxy, and amide. In certain embodiments, each R⁵ is independently selected from C₁₋₃ alkyl, C₁₋₃ alkoxy, C₁₋₃ haloalkyl. C₂₋₄ acyloxy, cyano, and halo. In certain embodiments, each R⁵ is independently selected from C₁₋₃ alkoxy, C₂₋₄ acyloxy, C₁₋₃ alkyl, C₁₋₃ haloalkyl, and halo. In certain embodiments, each R⁵ is independently selected from C₁₋₃ alkyl, C₁₋₃ alkoxy, trifluoromethyl, cyano, and halo. In certain embodiments, R⁵ is not chloro. In certain preferred embodiments, each R⁵ is independently selected from methyl, trifluoromethyl, cyano, chloro, methoxy, acetoxy, and fluoro. In certain preferred embodiments, each R⁵ is independently selected from methyl, trifluoromethyl, cyano, methoxy, acetoxy, and fluoro. In certain preferred embodiments, each R⁵ is independently selected from methyl, trifluoromethyl, methoxy, acetoxy, chloro, and fluoro. In certain preferred embodiments, each R⁵ is independently selected from methyl, trifluoromethyl, methoxy, acetoxy, and fluoro. In certain embodiments, each R⁵ is independently selected from methyl, trifluoromethyl, chloro, and fluoro. In certain embodiments, no R⁵ is fluoro. In certain embodiments, R⁵ is fluoro. In other embodiments, at least one R⁵ is fluoro. In certain embodiments, no R⁵ is chloro. In other embodiments, R⁵ is chloro. In other embodiments, at least one R⁵ is chloro.

In certain embodiments. R¹ is phenyl, pyridyl, or indolyl. In some such embodiments, R¹ is indolyl.

In certain embodiments, R¹ is phenyl, and is optionally substituted as described above. In certain preferred embodiments, R¹ is phenyl substituted at the meta-position with R⁵. In certain preferred embodiments, R¹ is 3-chlorophenyl, 3-cyanophenyl, or 3-methylphenyl. In certain preferred embodiments, R¹ is 3-cyanophenyl or 3-methylphenyl.

In certain embodiments. R¹ is a 6 membered heteroaryl (such as pyridinyl, pyrimidinyl, pyridazinyl, pyrazinyl), and is optionally substituted as described above. In certain preferred embodiments, R¹ is pyrazine, and is optionally substituted as described above. In certain preferred embodiments, R¹ is pyridin-2-yl substituted at the 4-position with R⁵. In certain preferred embodiments. R¹ is pyridin-2-yl substituted at the 4-position with R⁵, wherein R⁵ is not chloro. In certain preferred embodiments, R¹ is pyridin-2-yl substituted at the 4-position with R⁵, wherein R⁵ is trifluoromethyl, cyano, chloro, methoxy, amide, acetoxy, or hydroxyl. In certain preferred embodiments, R¹ is pyridin-2-yl substituted at the 4-position with R⁵, wherein R⁵ is trifluoromethyl, cyano, chloro, methoxy, amide, or hydroxyl. In certain preferred embodiments, R¹ is 4-fluoropyridin-2-yl, 4-chloropyridin-2-yl, 4-cyanopyridin-2-yl, 4-trifluoromethylpyridin-2-yl, 4-acetoxypyridin-2-yl, or 4-methoxypyridin-2-yl.

In certain preferred embodiments, R¹ is pyridin-4-yl substituted at the 2-position with R⁵. In certain preferred embodiments, R¹ is pyridine-4-yl substituted at the 2-position with R⁵, wherein R⁵ is chloro.

In certain embodiments, R¹ is a 9 membered heteroaryl. In certain preferred embodiments, R¹ is indolyl (such as indol-3-yl) or azaindolyl (such as 7-aza-indol-3-yl or 5-azaindol-6-yl), and is optionally substituted as described above. In certain preferred embodiments, R¹ is unsubstituted indol-3-yl. In certain preferred embodiments, R¹ is 6-fluoroindol-3-yl, 5-fluoro-7-azaindol-3-yl and 5-fluoroindol-3-yl.

In certain embodiments. R² is C₅₋₇ cycloalkyl. In certain embodiments, R² is C₅₋₆ cycloalkyl, such as cyclopentyl or cyclohexyl. In certain preferred embodiments, R² is cyclohexyl.

In certain embodiments, R² is 4-7 membered heterocyclyl, such as tetrahydropyranyl, piperidinyl, pyrrolidinyl, tetrahydrofuranyl, or azabicyclo[2.2.1]heptanyl. In certain preferred embodiments, R² is piperidinyl or tetrahydropyranyl.

In certain embodiments. R² is C₁₋₆ alkyl, such as tert-butyl, neopentyl, methyl, or ethyl, such as tert-butyl or neopentyl. In certain preferred embodiments, R² is tert-butyl.

In certain embodiments, R² is heteroaralkyl, preferably pyrrolylmethyl.

In certain embodiments, R² is carbocyclylalkyl, such as cyclobutylmethyl or cyclopropylmethyl, preferably cyclopropylmethyl.

In certain embodiments, R² is heterocyclylalkyl, such as 1,2-oxaborolanylmethyl or piperidinylmethyl, preferably piperidinylmethyl.

In certain embodiments. R² is C₁₋₆ alkenyl, such as butenyl.

In certain embodiments, R² is C₁₋₆ cycloalkenyl, such as cyclohexenyl.

In certain embodiments, R² is H.

In certain embodiments, R² is unsubstituted, such as unsubstituted cyclopentyl.

In certain embodiments, R² is substituted with one or more R⁷, and each R⁷ is independently selected from alkyl, such as haloalkyl, cycloalkyl, halo, hydroxyl, oxo, alkoxy, cycloalkyloxy, cyano, alkylthio, hydroxyalkyl, amino, ester, and carbamate. In certain embodiments, each R⁷ is independently selected from C₁₋₃ alkyl, C₁₋₃ haloalkyl, hydroxyl, or halo. In certain embodiments, each R⁷ is independently selected from C₁₋₃ alkyl, C₁₋₃ haloalkyl, or halo. In certain embodiments, each R⁷ is independently selected from hydroxymethyl, amino, dimethylamino, methoxycarbonyl, butyloxycarbonyl (such as tert-butyloxycarbonyl), and butyloxycarbonylamino (such as tert-butyloxycarbonylamino).

In certain embodiments, R² is substituted by one or more R⁷ selected from fluoro, hydroxyl, hydroxymethyl, butyloxycarbonylamine, butyloxycarbonyl, amino, trifluoromethyl, methoxycarbonyl, dimethylamine, methoxy, methyl, methylamino, boronic acid, ethoxycarbonyl, carboxy, or oxo. In certain preferred embodiments, each R⁷ is independently selected from methyl, trifluoromethyl, hydroxyl, chloro, or fluoro. In certain preferred embodiments, each R⁷ is independently selected from methyl, trifluoromethyl, chloro, or fluoro. In certain preferred embodiments, each R⁷ is fluoro.

In certain embodiments. R² is selected from dimethylcyclohexyl, cyclohexanoyl, aminocyclopentyl, methylcyclohexyl, dimethylaminocyclohexyl, methoxycyclohexyl, trifluoromethylcyclohexyl, methoxycarbonylcyclohexyl, hydroxycyclohexyl, hydroxymethylcyclohexyl, difluorocyclohexyl, fluorocyclohexyl, hydroxycyclopentyl, (butyloxycarbonyl)aminocyclopentyl, methylaminocyclohexyl, difluorohydroxycyclohexyl, oxocyclohexyl, and aminocyclohexyl.

In certain embodiments. R² is cyclopentyl or cyclohexyl, and is substituted by one or more R⁷ selected from haloalkyl, ester, and carbamate.

In certain embodiments, the R⁷ substituent has a cis configuration relative to the pyrazolo[3,4-d]pyrimidine core.

In certain embodiments, the R⁷ substituent has a trans configuration relative to the pyrazolo[3,4-d]pyrimidine core.

In certain embodiments, R⁶ is methylene.

In certain embodiments, the present disclosure provides compounds of formula (Ia):

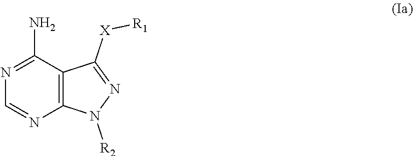

wherein X is R⁶ or O;
R¹ is phenyl or 6-membered heteroaryl optionally substituted with one or more R⁵ independently selected from C₁₋₃ alkyl, C₁₋₃ haloalkyl, cyano, acyloxy, hydroxyl, alkoxy, or halo;
R² is C₅₋₇ cycloalkyl, 4-7 membered heterocyclyl. C₁₋₆ alkyl, heteroaralkyl, carbocyclylalkyl, heterocyclylalkyl, C₂₋₆ alkenyl, C₄₋₆ cycloalkenyl, or H; and
R⁶ is C₁₋₃ alkylene;
   or a pharmaceutically acceptable salt thereof.

In some such embodiments. R¹ is phenyl optionally substituted with one or more R⁵ independently selected from C₁₋₃ alkyl, cyano, and halo. In other such embodiments, R¹ is pyridinyl optionally substituted with one or more R⁵.

In certain embodiments. R¹ is chlorophenyl, such as 3-chlorophenyl.

In certain preferred embodiments, R² is 4-7 membered heterocyclyl, such as piperidinyl, e.g., piperidin-3-yl or piperidin-4-yl.

In certain preferred embodiments, the present disclosure provides compounds of formula (Ib):

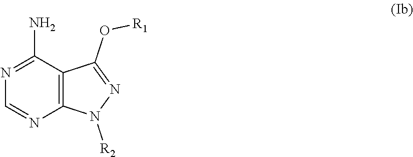

wherein
R¹ is pyridinyl substituted with one or more R⁵; and
R² is C₅₋₇ cycloalkyl, 4-7 membered heterocyclyl, C₁₋₆ alkyl, heteroaralkyl, carbocyclylalkyl, C₂₋₆ alkenyl, C₄₋₆ cycloalkenyl, or heterocyclylalkyl; or a pharmaceutically acceptable salt thereof.

In certain preferred embodiments, R¹ is pyridinyl substituted with one or more R⁵ independently selected from trifluoromethyl, cyano, chloro, hydroxyl, methoxy, acetoxy, and amide. In some such embodiments, R¹ is pyridinyl substituted with one or more R⁵ independently selected from trifluoromethyl, cyano, chloro, hydroxyl, methoxy, and amide.

In certain embodiments. R² is C₅₋₇ cycloalkyl. In certain embodiments, R² is C₅₋₆ cycloalkyl, such as cyclopentyl or cyclohexyl.

In certain embodiments, R² is carbocyclylalkyl, such as cyclopropylmethyl or cyclobutylmethyl, such as cyclopropylmethyl.

In certain embodiments, R² is heterocyclylalkyl, such as piperidinylmethyl or 1,2-oxaborolanylmethyl, such as piperidinylmethyl.

In certain embodiments. R² is C₁₋₆ alkyl, such as tert-butyl, neopentyl, methyl, or ethyl. In some such embodiments, R² is tert-butyl or neopentyl.

In certain embodiments, R² is C₁₋₆ alkenyl, such as butenyl.

In certain embodiments, R² is cycloalkenyl, such as cyclohexenyl.

In certain embodiments, R² is 4-7 membered heterocyclyl, such as azabicyclo[2.2.1]heptanyl, tetrahydropyranyl, tetrahydrofuranyl, piperidinyl, or pyrrolidinyl.

In certain embodiments. R² is cyclohexyl, cyclopentyl, piperidinylmethyl, pyrrolidinylmethyl, cyclopropylmethyl, tert-butyl, neopentyl, tetrahydropyranyl, pyrrolidinyl, piperidinyl, ethyl, methyl, cyclohexenyl, butenyl, 1,2-oxaborolanylmethyl, cyclobutylmethyl, or azabicyclo[2.2.1]heptanyl, substituted by one or more R⁷ selected from hydroxyl, fluoro, hydroxymethyl, butyloxycarbonylamino, amino, trifluoromethyl, methoxycarbonyl, dimethylamino, butyloxycarbonyl, methoxy, methyl, methylamino, boronic acid, and oxo. In some such embodiments, R² is cyclohexyl, cyclopentyl, piperidinylmethyl, pyrrolidinylmethyl, cyclopropylmethyl, tert-butyl, neopentyl, tetrahydropyranyl, pyrrolidinyl, piperidinyl, or azabicyclo[2.2.1]heptanyl, substituted by one or more R⁷ selected from hydroxyl, fluoro, hydroxymethyl, butyloxycarbonylamino, amino, trifluoromethyl, methoxycarbonyl, dimethylamino, butyloxycarbonyl, methoxy, methyl, and oxo.

In certain embodiments, the compound is:

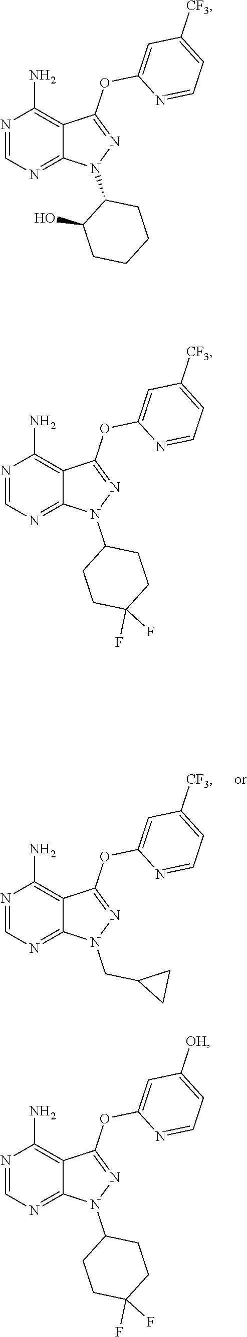 [SMILES: Nc1ncnc2c1c(Oc1cc(C(F)(F)F)ccn1)nn2[C@@H]1CCCC[C@H]1O ; Nc1ncnc2c1c(Oc1cc(C(F)(F)F)ccn1)nn2C1CCC(F)(F)CC1 ; Nc1ncnc2c1c(Oc1cc(C(F)(F)F)ccn1)nn2CC1CC1 ; Nc1ncnc2c1c(Oc1cc(O)ccn1)nn2C1CCC(F)(F)CC1]

or a pharmaceutically acceptable salt thereof.

In certain embodiments, the compound is:

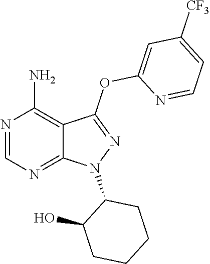 [SMILES: Nc1ncnc2c1c(Oc1cc(C(F)(F)F)ccn1)nn2[C@@H]1CCCC[C@H]1O]

or a pharmaceutically acceptable salt thereof.

In certain embodiments, the compound is:

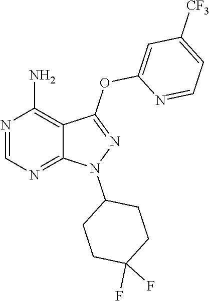 [SMILES: Nc1ncnc2c1c(Oc1cc(C(F)(F)F)ccn1)nn2C1CCC(F)(F)CC1]

or a pharmaceutically acceptable salt thereof.

In certain embodiments, the compound is:

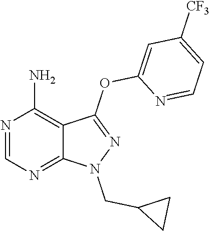 [SMILES: Nc1ncnc2c1c(Oc1cc(C(F)(F)F)ccn1)nn2CC1CC1]

or a pharmaceutically acceptable salt thereof.

In certain embodiments, the compound is:

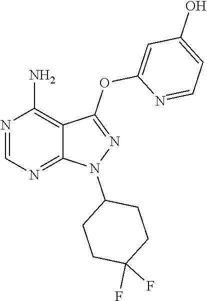 [SMILES: Nc1ncnc2c1c(Oc1cc(O)ccn1)nn2C1CCC(F)(F)CC1]

or a pharmaceutically acceptable salt thereof.

In some embodiments, the present invention relates to a compound selected from:

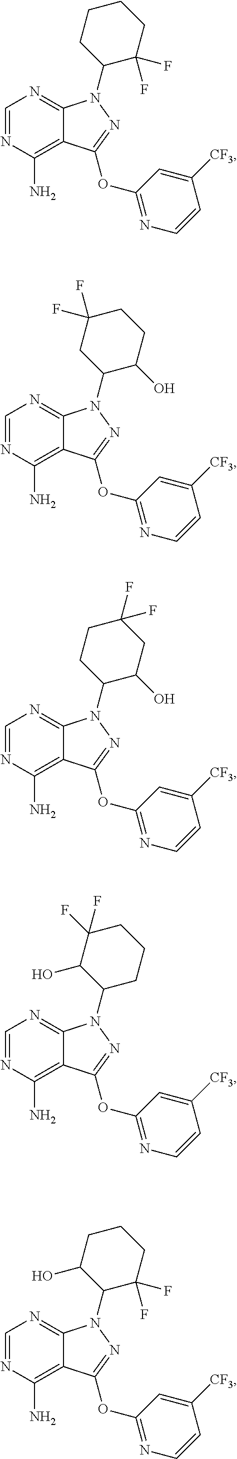 [SMILES: Nc1ncnc2c1c(Oc1cc(C(F)(F)F)ccn1)nn2C1CCCCC1(F)F ; Nc1ncnc2c1c(Oc1cc(C(F)(F)F)ccn1)nn2C1CC(F)(F)CCC1O ; Nc1ncnc2c1c(Oc1cc(C(F)(F)F)ccn1)nn2C1CCC(F)(F)CC1O ; Nc1ncnc2c1c(Oc1cc(C(F)(F)F)ccn1)nn2C1CCCC(F)(F)C1O ; Nc1ncnc2c1c(Oc1cc(C(F)(F)F)ccn1)nn2C1C(O)CCCC1(F)F]

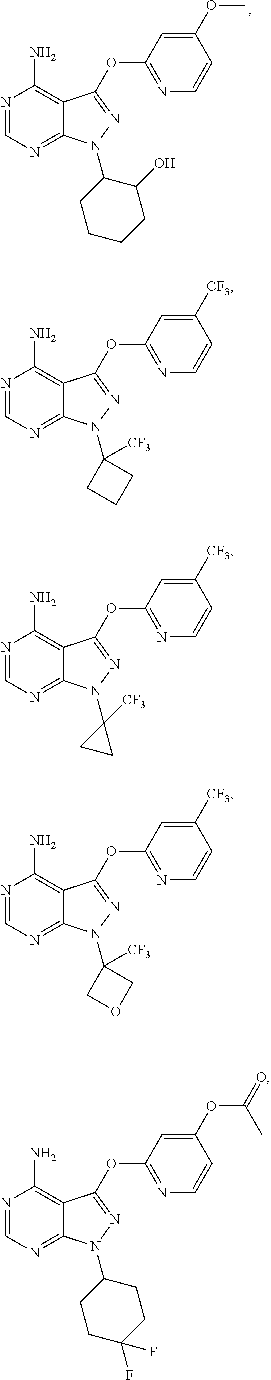 [SMILES: COc1ccnc(Oc2nn(C3CCCCC3O)c3ncnc(N)c23)c1 ; Nc1ncnc2c1c(Oc1cc(C(F)(F)F)ccn1)nn2C1(C(F)(F)F)CCC1 ; Nc1ncnc2c1c(Oc1cc(C(F)(F)F)ccn1)nn2C1(C(F)(F)F)CC1 ; Nc1ncnc2c1c(Oc1cc(C(F)(F)F)ccn1)nn2C1(C(F)(F)F)COC1 ; CC(=O)Oc1ccnc(Oc2nn(C3CCC(F)(F)CC3)c3ncnc(N)c23)c1]

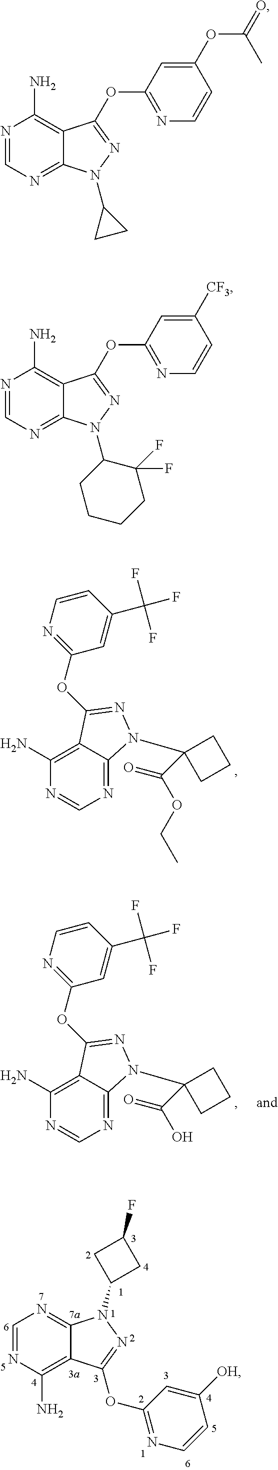 [SMILES: CC(=O)Oc1ccnc(Oc2nn(C3CC3)c3ncnc(N)c23)c1 ; Nc1ncnc2c1c(Oc1cc(C(F)(F)F)ccn1)nn2C1CCCCC1(F)F ; CCOC(=O)C1(n2nc(Oc3cc(C(F)(F)F)ccn3)c3c(N)ncnc32)CCC1 ; Nc1ncnc2c1c(Oc1cc(C(F)(F)F)ccn1)nn2C1(C(=O)O)CCC1 ; Nc1ncnc2c1c(Oc1cc(O)ccn1)nn2[C@H]1C[C@H](F)C1]

or a pharmaceutically acceptable salt thereof.

In some embodiments, the present invention relates to a compound selected from:

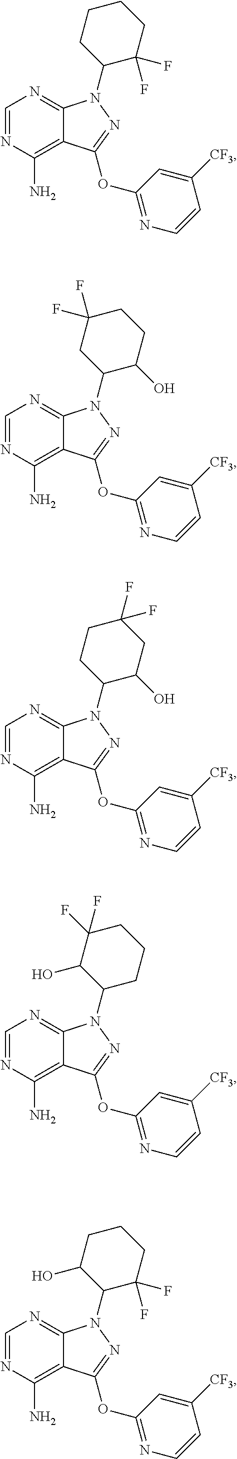 [SMILES: Nc1ncnc2c1c(Oc1cc(C(F)(F)F)ccn1)nn2C1CCCCC1(F)F ; Nc1ncnc2c1c(Oc1cc(C(F)(F)F)ccn1)nn2C1CC(F)(F)CCC1O ; Nc1ncnc2c1c(Oc1cc(C(F)(F)F)ccn1)nn2C1CCC(F)(F)CC1O ; Nc1ncnc2c1c(Oc1cc(C(F)(F)F)ccn1)nn2C1CCCC(F)(F)C1O ; Nc1ncnc2c1c(Oc1cc(C(F)(F)F)ccn1)nn2C1C(O)CCCC1(F)F]

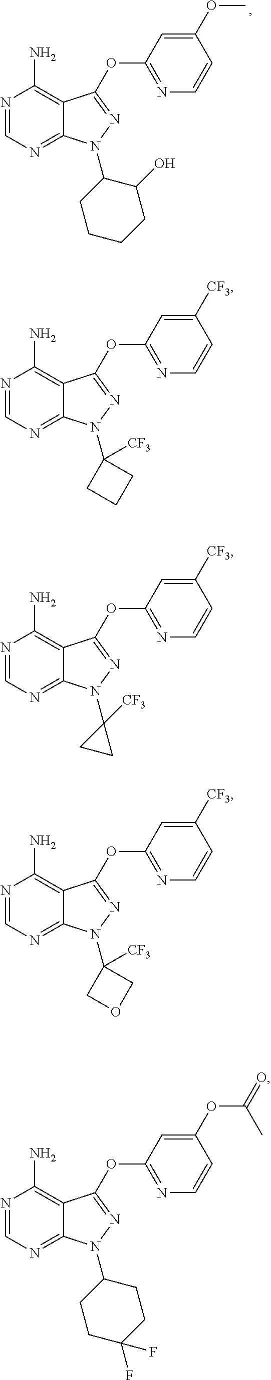 [SMILES: COc1ccnc(Oc2nn(C3CCCCC3O)c3ncnc(N)c23)c1 ; Nc1ncnc2c1c(Oc1cc(C(F)(F)F)ccn1)nn2C1(C(F)(F)F)CCC1 ; Nc1ncnc2c1c(Oc1cc(C(F)(F)F)ccn1)nn2C1(C(F)(F)F)CC1 ; Nc1ncnc2c1c(Oc1cc(C(F)(F)F)ccn1)nn2C1(C(F)(F)F)COC1 ; CC(=O)Oc1ccnc(Oc2nn(C3CCC(F)(F)CC3)c3ncnc(N)c23)c1]

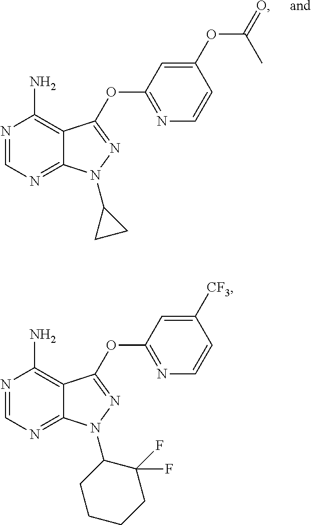 [SMILES: CC(=O)Oc1ccnc(Oc2nn(C3CC3)c3ncnc(N)c23)c1 ; "and" ; Nc1ncnc2c1c(Oc1cc(C(F)(F)F)ccn1)nn2C1CCCCC1(F)F]

or a pharmaceutically acceptable salt thereof.

In certain embodiments, the compound is selected from

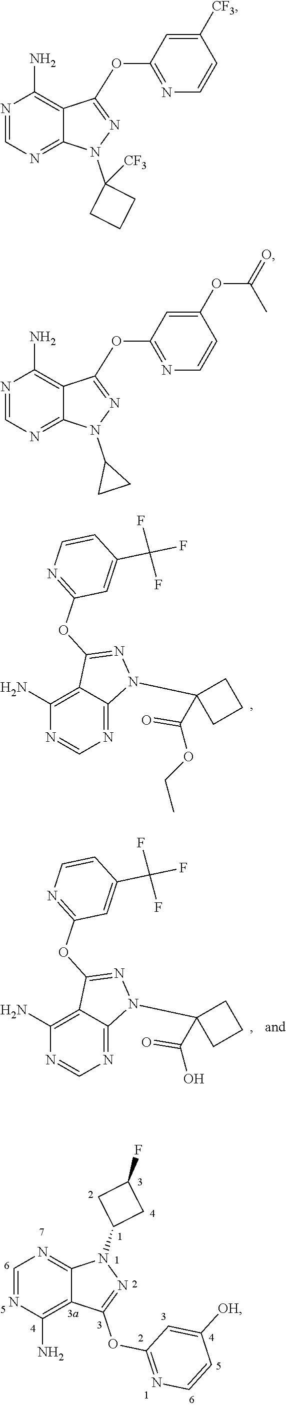

or a pharmaceutically acceptable salt thereof.

In certain embodiments, the compound is selected from

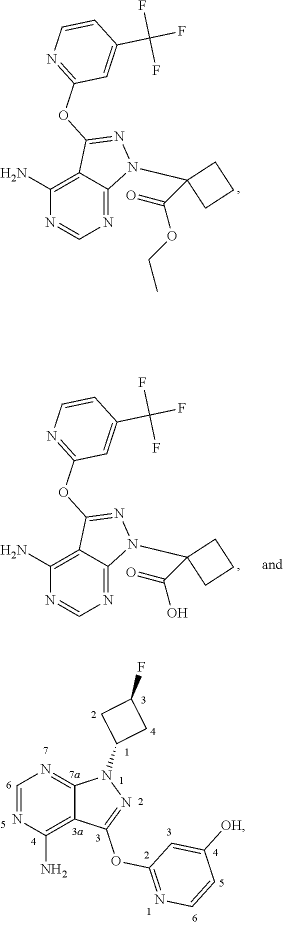 [SMILES: CCOC(=O)C1(n2nc(Oc3cc(C(F)(F)F)ccn3)c3c(N)ncnc32)CCC1 ; Nc1ncnc2c1c(Oc1cc(C(F)(F)F)ccn1)nn2C1(C(=O)O)CCC1 ; Nc1ncnc2c1c(Oc1cc(O)ccn1)nn2[C@H]1C[C@H](F)C1]

or a pharmaceutically acceptable salt thereof.

In certain embodiments, the compound is

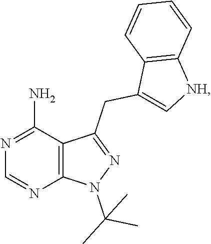 [SMILES: CC(C)(C)n1nc(Cc2c[nH]c3ccccc23)c2c(N)ncnc21]

or a pharmaceutically acceptable salt thereof.

In another aspect the present invention relates to a pharmaceutical composition comprising a compound as disclosed herein.

In yet another aspect, the present invention relates to a method of preventing or inhibiting the growth or proliferation of a microorganism using a compound of formula (I). In certain embodiments, the microorganism is a protozoan. In certain embodiments, the microorganism is a protozoan, in certain embodiments, the protozoan is an Apicomplexan, for instance of genera Toxoplasma, Cryptosporidium, or Plasmodium. In certain embodiments, the microorganism is T gondii, or is of genera Cryptosporidium or Plasmodium. In certain preferred embodiments, the microorganisms are T gondii, P. falciparum, C. hominis, or C. parvum.

In certain embodiments inhibiting the growth or proliferation of a microorganism comprises applying a compound having the structure of formula (I) to a location. The compound may be applied in the form of a spray (e.g., from a spray bottle) or by wiping (e.g., with a pre-soaked wipe, a mop, or a sponge). In certain embodiments, the location is one where the microorganism is known or suspected to be present. In certain embodiments, the location is one that is at risk for the presence of the microorganism. In certain embodiments, the compound of formula (I) is applied prophylactically. In certain embodiments, the compound of formula (I) is applied after suspected contamination by the protozoan. In certain embodiments, the location may be a surface, such as a cooking surface or a surface that has contact with material suspected of containing the microorganism, such as a surface that has had contact with raw meat or animal (such as cat) feces. In certain embodiments, the cooking surface is a cutting board, a counter, or a utensil, such as a knife or fork. In certain embodiments, the location may be the surface or interior of a food, such as a meat or a vegetable. In certain embodiments, the location may be a liquid, such as water, for instance drinking water. In certain embodiments, the location may be soil. In certain embodiments, the location may be a place where a cat has defecated or will defecate, or an area where cat feces or cat litter is likely to spread or to have been spread. In further embodiments, the location is a litterbox or the area around a litterbox. In certain embodiments, the location is a body surface, such as a hand.

In certain embodiments, the compound of formula (I) is used to prevent transmission of the microorganism between people and/or animals. In further embodiments, the transmission is congenital transmission. In further embodiments, the compound of formula (I) is administered to a mother, administered to an infant, applied to the skin of the mother, or applied to the skin of the infant. In certain embodiments, the compound of formula (I) is applied to blood, such as blood intended for transfusion. In certain embodiments, the compound of formula (I) is applied to an organ, such as an organ intended for transplant. In certain embodiments, the compound of formula (I) is administered to an organ donor prior to transplant. In certain embodiments, the compound of formula (I) is administered to an animal, such as a cat or a mouse.

In yet another aspect, the present invention relates to a method of treating an infection, comprising administering a compound having the structure of formula (I), a pharmaceutically acceptable salt or prodrug thereof, or a pharmaceutical composition comprising such a compound, salt, or prodrug. In certain embodiments, the infection is caused by a protozoan. In certain embodiments, the protozoan is an Apicomplexan, few instance of genera Toxoplasma, Cryptosporidium, or Plasmodium. In certain embodiments, the microorganism is T. gondii, or is of genera Cryptosporidium or Plasmodium. In certain preferred embodiments, the microorganisms are T. gondii, P. falciparum, C. hominis, or C. parvum.

In yet another aspect, the present invention relates to one of the compounds or compositions disclosed herein, a pharmaceutically acceptable salt or prodrug thereof, or a pharmaceutical composition comprising such a compound, salt, or prodrug, for use in the treatment of an infection. In certain embodiments, the infection is caused by a protozoan, such as an Apicomplexan protozoan. In certain embodiments, the protozoan is of genus Toxoplasma, Cryptosporidium, or Plasmodium. In certain embodiments, the protozoan is an Apicomplexan, for instance of genera Toxoplasma, Cryptosporidium, or Plasmodium.

In certain embodiments, the microorganism is T. gondii, or is of genera Cryptosporidium or Plasmodium. In certain preferred embodiments, the microorganisms are T. gondii, P. falciparum, C. hominis, or C. parvum.

In still another aspect, the present invention relates to a compound having the structure of formula (I), a pharmaceutically acceptable salt or prodrug thereof, or a pharmaceutical composition comprising such a compound, salt, or prodrug for use in the treatment of an infection.

### Discussion

Apicomplexans contain from 6-11 related calcium dependent protein kinases (CDPKs), depending on the species. In addition to TgCDPK1, which controls invasion and egress, TgCDPK2 and TgCPDK6 have also been shown to play essential roles in bradyzoite development and cell division, respectively. CDPKs differ substantially in their ATP binding pocket from human kinases. In particular. CDPK1 from Toxoplasma gondii, Neospora caninum, Sarcocystis neurona, and Cryptosporidium spp. contain a glycine gatekeeper, predicting that they will be sensitive to the compounds described herein. Other CDPKs contain different substitutions in their ATP binding pocket, and they may also be targeted by the inhibitors described herein. Plasmodium also contains a number of CDPKs that are important in infection of red blood cells, as well as development in the mosquito during transmission. Additional roles for CDPKs in related parasites may be defined by future studies and some of these enzymes may also be inhibited by the compounds described herein.

The compounds disclosed herein inhibit CDPK1, and can prevent or ameliorate infections, including toxoplasmosis. In certain embodiments, the compounds herein preferentially inhibit protozoan CDPK1 relative to other human kinases. In certain embodiments, the protozoan is an Apicomplexan, for instance of genera Toxoplasma, Cryptosporidium, or Plasmodium. In certain embodiments, the microorganism is T gondii, or is of genera Cryptosporidium or Plasmodium. In certain preferred embodiments, the microorganisms are T. gondii, P. falciparum, C. hominis, or C. parvum In certain such embodiments, the selectivity of the compounds herein for protozoan CDPK1 (such as T. gondii, P. falciparum, C. hominis, or C. parvum) versus human SRC kinase (as determined by the ratio of the compound's IC₅₀ against each enzyme) is greater than 3-fold, greater than 10-fold, greater than 30-fold, greater than 50-fold, greater than 75-fold, greater than 100-fold, or greater than 300-fold. In certain embodiments, the compounds herein have an IC₅₀ for protozoan CDPK1 (such as T. gondii, P. falciparum, C. hominis, or C. parvum) less than 3000, less than 1500, less than 1000 nM, or less than 300, preferably less than 100 nM or less than 30 nM. In certain embodiments, the selectivity of the compounds herein for T. gondii, P. falciparum, C. hominis, or C. parvum versus human SRC kinase (as determined by the ratio of the compound's IC₅₀ against each kinase) is greater than 3-fold, greater than 10-fold, greater than 30-fold, greater than 50-fold, greater than 75-fold, greater than 100-fold, or greater than 300-fold. In certain embodiments, the compounds herein have an IC₅₀ for T. gondii, P. falciparum, C. hominis, or C. parvum CDPK1 of less than nM or less than 100 nM, preferably less than 10 nM.

In certain embodiments, compounds of the invention may be prodrugs of the compounds disclosed herein, e.g., wherein a hydroxyl in the parent compound is presented as an ester or a carbonate, or a carboxylic acid present in the parent compound is presented as an ester. In certain such embodiments, the prodrug is metabolized to the active parent compound in vivo (e.g., the ester is hydrolyzed to the corresponding hydroxyl, or carboxylic acid).

In certain embodiments, compounds of the invention may be racemic. In certain embodiments, compounds of the invention may be enriched in one enantiomer. For example, a compound of the invention may have greater than 30% ee, 40% ee, 50% ee, 60% ee, 70% ee, 80% ee, 90% ee, or even 95% or greater ee. In certain embodiments, compounds of the invention may have more than one stereocenter. In certain such embodiments, compounds of the invention may be enriched in one or more diastereomers. For example, a compound of the invention may have greater than 30% de, 40% de, 50% de, 60% de, 70% de, 80% de, 90% de, or even 95% or greater de.

In certain embodiments, the present invention relates to methods of treatment with a compound disclosed herein, or a pharmaceutically acceptable salt thereof. In certain embodiments, the therapeutic preparation may be enriched to provide predominantly one enantiomer of a compound. An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, the therapeutic preparation may be enriched to provide predominantly one diastereomer of a compound. A diastereomerically enriched mixture may comprise, for example, at least 60 mol percent of one diastereomer, or more preferably at least 75, 90, 95, or even 99 mol percent.

In certain embodiments, the present invention provides a pharmaceutical preparation suitable for use in a human patient, comprising any of the compounds shown above (e.g., a compound of the invention), and one or more pharmaceutically acceptable excipients. In certain embodiments, the pharmaceutical preparations may be for use in treating or preventing a condition or disease as described herein. In certain embodiments, the pharmaceutical preparations have a low enough pyrogen activity to be suitable for use in a human patient.

Compounds of any of the above structures may be used in the manufacture of medicaments for the treatment of any diseases or conditions disclosed herein.

### Definitions

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, trifluoromethoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen (e.g., fluoro), a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain, including haloalkyl groups. Preferred haloalkyl groups include trifluoromethyl, difluoromethyl, 2,2,2-trifluoroethyl, and pentafluoroethyl. C₀ alkyl indicates a hydrogen where the group is in a terminal position, a bond if internal. The terms “C_{x-y} alkenyl” and “C_{x-y} alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

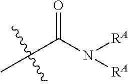

wherein each R^{A} independently represent a hydrogen or hydrocarbyl group, or two R^{A} are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

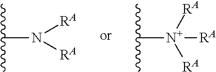

wherein each R^{A} independently represents a hydrogen or a hydrocarbyl group, or two R^{A} are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 6- or 10-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “boronic acid” as used herein is art-recognized and refers to a group

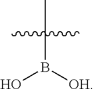 [SMILES: *B(O)O]

The term “carbamate” is art-recognized and refers to a group

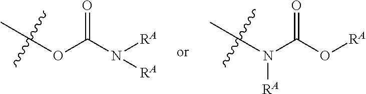

wherein each R^{A} independently represent hydrogen or a hydrocarbyl group, such as an alkyl group, or both R^{A} taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R^{A}, wherein R^{A} represents a hydrocarbyl group.

The term “carboxy”, as used herein, refers to a group represented by the formula —CO₂H.

The term “ester”, as used herein, refers to a group —C(O)OR^{A} wherein R^{A} represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are boron, nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, tetrahydropyran, tetrahydrofuran, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that docs not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The term “oxaborolanylmethyl” as used herein is art-recognized and refers to a 5-membered cyclopentylmethyl ring in which two of the cyclopentyl carbon atoms have been replaced with a boron atom and an oxygen atom. For example, a “1,2-oxaborolan-S-ylmethyl” group refers to a group

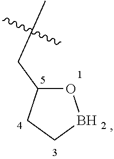

and a “2-hydroxy-1,2-oxaborolan-5-ylmethyl” group refers to a group

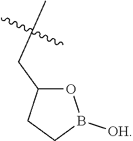 [SMILES: *CC1CCB(O)O1]

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

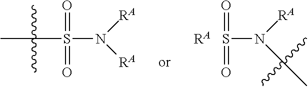

wherein each R^{A} independently represents hydrogen or hydrocarbyl, such as alkyl, or both R^{A} taken together with the intervening atom(s) complete a heterocycle having from 4 to 8 atoms in the ring structure.

The term “sulfoxide” is art-recognized and refers to the group —S(O)—R^{A}, wherein R^{A} represents a hydrocarbyl.

The term “sulfonate” is art-recognized and refers to the group SO₃H, or a pharmaceutically acceptable salt thereof.

The term “sulfone” is art-recognized and refers to the group —S(O)₂—R^{A}, wherein R^{A} represents a hydrocarbyl.

The term “thioalkyl”, as used herein, refers to an alkyl group substituted with a thiol group.

The term “thioester”, as used herein, refers to a group —C(O)SR^{A} or —SC(O)R^{A} wherein R^{A} represents a hydrocarbyl.

The term “thioether”, as used herein, is equivalent to an ether, wherein the oxygen is replaced with a sulfur.

The term “urea” is art-recognized and may be represented by the general formula

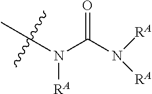

wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered cither in the same formulation or in a separate formulation, either concomitantly or sequentially. In certain embodiments, the different therapeutic compounds can be administered within one hour, 12 hours, 24 hours, 36 hours, hours, 72 hours, or a week of one another. Thus, an individual who receives such treatment can benefit from a combined effect of different therapeutic compounds.

The term “prodrug” is intended to encompass compounds which, under physiologic conditions, are converted into the therapeutically active agents of the present invention. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds of the invention in a formulation represented above can be replaced with the corresponding suitable prodrug, e.g., wherein a hydroxyl in the parent compound is presented as an ester or a carbonate or carboxylic acid present in the patent compound is presented as an ester.

### Use of CPPK1 Inhibitors

Another embodiment of the invention is the use of the compounds described herein for the treatment of infections (e.g., parasitic infections, such as toxoplasmosis). In certain embodiments, the compounds described herein may be used conjointly with other compounds useful for that purpose, such as sulfadiazine, sulfamethoxazole, clindamycin, spiramycin, atovaquone. DHFR inhibitors, or cytochrome BC₁ inhibitors.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (S) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by cither providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benethamine, benzathine, betaine, calcium hydroxide, choline, decanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, L-ascorbic acid, L-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, D-glucoheptonic acid, D-gluconic acid, D-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, L-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid. L-pyroglutamic acid, salicylic acid, sebacic acid, stearic acid, succinic acid, sulfuric acid, L-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylendiamine tetraacetic acid (EDTA), sorbitol, tartaric acid, phosphoric acid, and the like.

### EXAMPLES

### Example 1: General Methods

NMR spectra were recorded on a Varian 400 MHz for ¹H NMR. LCMS were taken on a quadrupole Mass Spectrometer on Shimadzu LCMS 2010 (Column: sepax ODS 50×2.0 mm, 5 um) or Agilent 1200 HPLC, 1956 MSD (Column: Shim-pack XR-ODS 30×3.0 mm, 2.2 um) operating in ES (+) ionization mode.

### Example 2: Synthetic Methods

### Synthesis Method A: the General Procedure of Method a is Represented by the Preparation of 3-(3-chlorobenzyl)-1-cyclopropyl-1H-pyrazolo[3,4-d]pyrimidin-4-amine (R₁ is cyclopropyl and R₂ is 3-chlorophenyl)

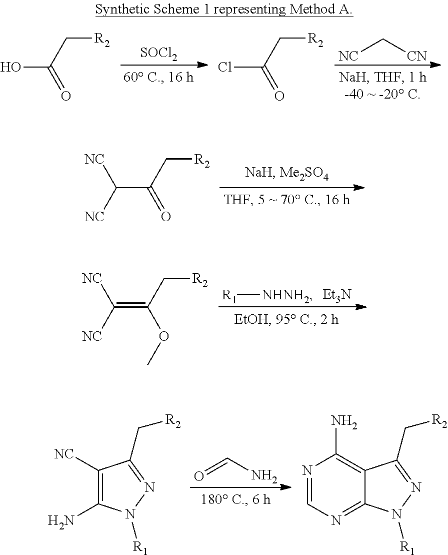

### Step 1

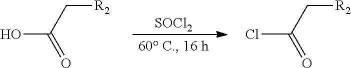

A solution of 2-(3-chlorophenyl)acetic acid (50.0 g, 293.1 mmol, 1.0 eq) in SOCl₂ (300.0 mL) was stirred at 60° C. for about 16 h. TLC (Petroleum ether/Ethyl acetate=3/1) showed the starting material was consumed completely (quenched by methanol). Then the mixture was concentrated by rotary evaporator to give 2-(3-chlorophenyl)acetyl chloride (55.4 g. crude) as light yellow liquid.

### Step 2

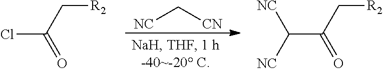

To a solution of propanedinitrile (19.4 g, 293.1 mmol, 1.0 eq) in THF (500.0 mL) was added NaH (14.1 g, 351.7 mmol, 60% purity, 1.2 eq) in portions at −40° C.˜−20° C., stirred for about 20 min and then a solution of 2-(3-chlorophenyl)acetyl chloride (55.4 g, crude, 1.0 eq) in THF (500.0 mL) was added while maintaining the temperature between −40° C. and −20° C. Stirring continued at this temperature for about 40 min. TLC (petroleum ether/ethyl acetate=2/1; product R_{f}=0.4) indicated the reaction was complete, and the reaction was quenched by addition of 1 L of water, extracted with 3×500 mL of ethyl acetate and the combined organic fractions were dried (sodium sulfate) and concentrated. Purification by column chromatography (SiO₂, Petroleum ether/Ethyl acetate=5/1 to 2/1) provided 2-(2-(3-chlorophenyl)actyl)malononitrile (21.0 g, 96.0 mmol, 32.8% yield) as red oil.

### Step 3

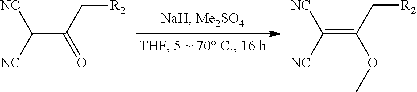

To a solution of 2-(2-(3-chlorophenyl)acetyl)malononitrile (6.7 g, 30.5 mmol, 1.0 eq) in THF (70.0 mL) was added NaH (1.8 g, 45.8 mmol, 60% purity, 1.5 eq) in portions at 5° C. After stirring at 5° C. for about 15 min, Me₂SO₄ (15.4 g, 122.0 mmol, 4.0 eq) was added dropwise and then the reaction mixture was heated to 70° C. for about 16 h. The reaction was quenched by addition of 300 mL of water, extracted with 3×200 mL of ethyl acetate and the combined organic fractions were dried (sodium sulfate) and concentrated. Purification by column chromatography (SiO₂, petroleum ether/ethyl acetate=10/1 to 3/1) provided 2-(2-(3-chlorophenyl)-1-methoxyethylidene)malononitrile (14.0 g, 60.2 mmol, 65.7% yield) as a yellow oil. ¹H NMR (400 MHz, CDCl₃) δ=7.35 (d, J=5.2 Hz, 2H), 7.25 (d, J=9.6 Hz, 1H), 7.16 (t, J=3.6 Hz, 1H), 4.09 (s, 3H), 3.98 (s, 2H).

### Step 4

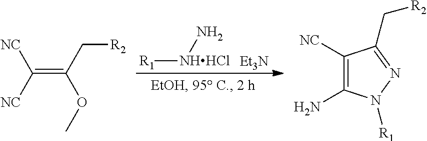

To a mixture of 2-(2-(3-chlorophenyl)-1-methoxyethylidene)malononitrile (4.0 g, 17.2 mmol, 1.0 eq) and cyclopropylhydrazine (3.73 g, 34.4 mmol, 2.0 eq, HCl) in ethanol (50.0 mL) was added triethylamine (6.9 g, 68.7 mmol, 4.0 eq). After stirring at 95° C. for 2 h under nitrogen atmosphere the reaction was deemed complete by TLC (Petroleum ether/Ethyl acetate=1/1; product R_{f} 0.4) and concentrated under reduced pressure. The residue was purified by column chromatography (SiO₂. Petroleum ether/Ethyl acetate=5/1 to 3/1) to give 5-amino-3-(3-chlorobenzyl)-1-cyclopropyl-1H-pyrazole-4-carbonitrile (4.0 g, 14.6 mmol, 85.3% yield) as a yellow solid. ¹H NMR: (400 MHz, CDCl₃) δ=7.27 (s, 1H), 7.24-7.17 (m, 3H), 4.63 (s, 2H), 3.86 (s, 2H), 3.10-3.05 (m, 1H), 1.14-1.08 (m, 4H).

### Step 5

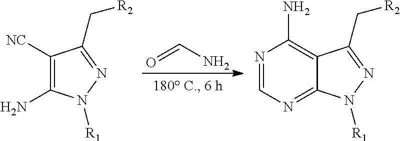

5-Amino-3-(3-chlorobenzyl)-1-cyclopropyl-1H-pyrazole-4-carbonitrile (400.0 mg, 1.5 mmol, 1.0 eq) and formamide (9.0 g, 200.7 mmol, 8.0 mL, 136.8 eq) were stirred at 180° C. for about 6 h. Reaction progress was monitored by TLC (Dichloromethane/Methanol=10/1, R_{f}=0.55) and upon completion, the mixture was poured into about 15 mL of water and extracted with 3×20 mL of ethyl acetate. The combined organic fractions were dried (Na2SO4), concentrated and the remaining residue purified by column chromatography (SiO₂, DCM/Methanol 30/1 to 20/1) to provide 420 mg of product as a yellow solid. Further purification of 70 mg crude product by HPLC (condition: neutral) gave 21.4 mg of 3-(3-chlorobenzyl)-1-cyclopropyl-1H-pyrazolo[3,4-d]pyrimidin-4-amine (Compound 1) as a white solid. ¹H NMR: (400 MHz, CDCl₃) δ=8.34 (s, 1H), 7.25 (d, J=1.2 Hz, 2H), 7.20 (s, 1H), 7.09 (d, J=5.6 Hz, 1H), 4.94 (s, 2H), 4.26 (s, 2H), 3.75-3.71 (m, 1H), 1.34-1.30 (m, 2H), 1.19-1.14 (m, 2H). LCMS: (M+H)⁺: 300.1, Rt: 2.254 min. LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS).

The following compounds were prepared in a similar manner as for method A using different starting materials.

TABLE 1
Compounds Prepared by Method A

| | | | | |
|---|---|---|---|---|
| | | | LC/MS | |
| Compound | | | Observed | |
| No. | IUPAC Name | MW | (M + H) | ¹H NMR (400 MHz) |

| | | | | |
|---|---|---|---|---|
| 7 | 3-(4-chlorobenzyl)- | 299.76 | 300.0 | (CHLOROFORM-d) δ = 8.33 (s, 1H), |
| | 1-cyclopropyl-1H- | | | 7.33-7.26 (m, 2H), 7.17-7.14 (m, 2H), |
| | pyrazolo[3,4-d] | | | 4.91 (s, 2H), 3.74-3.72 (m, 1H), |
| | pyrimidin-4-amine | | | 1.32-1.31 (m, 2H), 1.19-1.16 (m, 2H) |
| 3 | 3-(3-chlorobenzyl)- | 299.76 | 300.1 | (CHLOROFORM-d) δ = 8.34 (s, 1H), |
| | 1-cyclopropyl-1H- | | | 7.25 (d, J = 1.2 Hz, 2H), 7.20 (s, 1H), |
| | pyrazolo[3,4-d] | | | 7.09 (d, J = 5.6 Hz, 1H), 4.94 (s, 2H), |
| | pyrimidin-4-amine | | | 4.26 (s, 2H), 3.75-3.71 (m, 1H), |
| | | | | 1.34-1.30 (m, 2H), 1.19-1.14 (m, 2H) |
| 4 | 3-(3-chloro-5- | 317.75 | 318.1 | (CHLOROFORM-d) δ = 8.38 (s, 1H) |
| | fluorobenzyl)-1- | | | 7.03 (d, J = 6.8 Hz, 2H), |
| | cyclopropyl-1H- | | | 6.82 (d, J = 9.2 Hz, 1H), |
| | pyrazolo[3,4-d] | | | 4.96 (s, 2H), 4.27 (s, 2H), |
| | pyrimidin-4-amine | | | 3.76 (d, J = 3.6 Hz, 1H), |
| | | | | 1.34 (s, 2H), |
| | | | | 1.20 (t, J = 6.2 Hz, 2H) |
| 5 | 1-cyclopropyl-3-(3- | 283.30 | 284.2 | (DMSO-d6) δ = 8.14 (s, 1H), |
| | fluorobenzyl)-1H- | | | 7.35-7.28 (m, 1H), |
| | pyrazolo[3,4-d] | | | 7.09-7.02 (m, 2H), |
| | pyrimidin-4-amine | | | 7.02-6.97 (m, 1H), |
| | | | | 4.34 (s, 2H), 3.77-3.70 (m, 1H) |
| | | | | 3.31-3.28 (m, 2H), |
| | | | | 1.15-0.98 (m, 4H) |
| 52 | 3-((4-chloropyridin- | 300.1 | 301.1 | (ACETONITRILE-d3 + D20) |
| | 2-yl)methyl)-1- | | | δ = 8.45 (d, J = 5.6, 1H), |
| | cyclopropyl-1H- | | | 8.13 (s, 1H), 7.56 (d, J = 1.6 Hz, |
| | pyrazolo[3,4-d] | | | 1H), 7.37 (d, J = 5.2, 1H), |
| | pyrimidin-4-amine | | | 4.70 (s, 2H), 3.88-3.83 (m, 1H), |
| | | | | 1.29-1.20 (m, 4H) |

### Synthesis Method B: The General Procedure of Method B is Represented by the Preparation of 3-(3-chlorobenzyl)-1-cyclobutyl-1H-pyrazolo[3,4-d]pyrimidin-4-amine

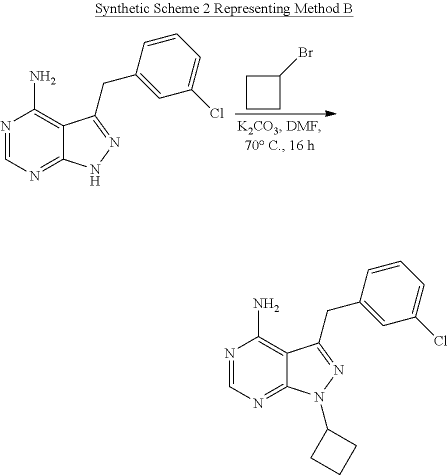

To a suspension of 3-(3-chlorobenzyl)-1H-pyrazolo[3,4-d]pyrimidin-4-amine (100.0 mg, 385.1 μmol, 1.0 eq) and K₂CO₃ (106.4 mg, 770.1 μmol, 2.0 eq; prepared as described in method A with R1=H) in anhydrous DMF (4.0 mL) under nitrogen was added bromocyclobutane (104.0 mg, 770.1 μmol, 2.0 eq), and the mixture was stirred at 70° C. for 16 h. The reaction mixture was filtered and the filtrate was purified by prep-HPLC (condition: neutral) to give 3-(3-chlorobenzyl)-1-cyclobutyl-1H-pyrazolo[3,4-d]pyrimidin-4-amine (Compound 6) (45.5 mg, 145.0 μmol, 37.7% yield) as an off-white solid. ¹H NMR: (400 MHz, DMSO-d₆) δ=8.13 (s, 1H), 7.36-7.19 (m, 5H), 5.27-5.19 (m, 1H), 4.40 (s, 2H), 2.69-2.62 (m, 2H), 2.36-2.34 (m, 2H), 1.87-1.81 (m, 2H). LCMS: (M+H)⁺: 314.3, Rt: 2.471 min. LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS).

The following compound was prepared in a similar manner as for method A using different starting materials.

| | | | LC/MS | |
|---|---|---|---|---|
| Compound | | | Observed | |
| No. | IUPAC Name | MW | (M + H) | 1H NMR (400 MHz) |
| 53 | 3-(3-chlorobenzyl)- | 349 | 350.1 | (METHANOL-d4) δ = 8.31 |
| | 1-(3,3- | | | (s, 1H), 7.31-7.26 (m, 3H), 7.22 |
| | difluorocyclobutyl)- | | | (d, J = 15.6 Hz, 1H), 5.38-5.29 |
| | 1H-pyrazolo[3,4-d] | | | (m, 1H), 4,45 (s, 2H), 3.48-3.32 |
| | pyrimidin-4-amine | | | (m, 2H), 3.18-3.15 (m, 2H) |

### Synthesis Method C: General Procedure Represented by the Preparation of 1-cyclopropyl-3-(3-(pyridin-2-yl)benzyl)-1H-pyrazolo[3,4-d]pyrimidin-4-amine

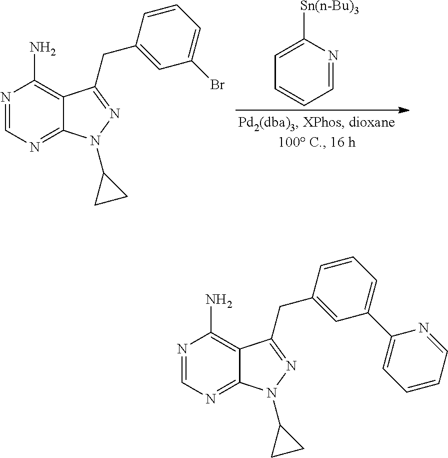

A mixture of 3-(3-bromobenzyl)-1-cyclopropyl-1H-pyrazolo[3,4-d]pyrimidin-4-amine (100 mg, 290.52 μmol, 1.00 eq), tributyl(2-pyridyl)stannane (106.95 mg, 290.52 μmol, 1.00 eq), Pd₂(dba)₃ (7.98 mg, 8.72 μmol, 0.03 eq), XPhos (23.54 mg, 49.39 μmol, 0.17 eq) in dioxane (2.00 mL) was stirred at 100° C. for about 16 h under nitrogen atmosphere. The reaction was monitored by LCMS and upon completion the reaction mixture was filtered and the filtrate purified by prep-HPLC (condition: TFA) to give 1-cyclopropyl-3-(3-(pyridin-2-yl)benzyl)-1H-pyrazolo[3,4-d]pyrimidin-4-amine (Compound 7) (16.52 mg, 36.19 μmol, 12.46% yield) as a white solid. ¹H NMR: (400 MHz, METHANOL-d₄) δ=8.73 (d, J=5.2 Hz, 1H), 8.38-8.31 (m, 2H), 8.13 (d, J=8.0 Hz, 1H), 7.87-7.80 (m, 2H), 7.78-7.73 (m, 1H), 7.60-7.54 (t, J=7.6 Hz, 1H), 7.51-7.46 (m, 1H), 4.54 (s, 2H), 3.92 (m, 1H), 1.32-1.22 (m, 2H), 1.20-1.10 (m, 2H). LCMS: Obtained M+H 343.1, expected M+H 343.2. LC/MS conditions (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS).

### Alternative Synthesis Method C: General Procedure Represented by the Preparation of 3-([1,1′-biphenyl]-3-ylmethyl)-1-cyclopropyl-1H-pyrazolo[3,4-d]pyrimidin-4-amine

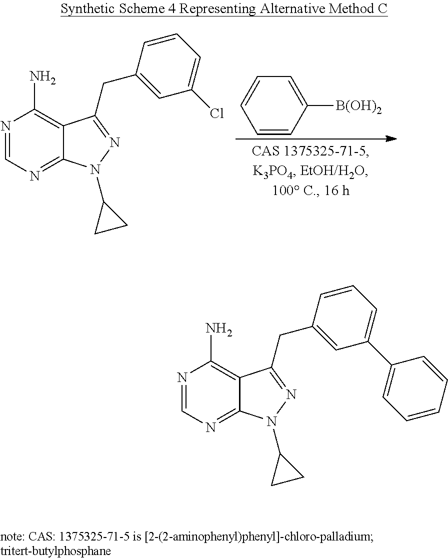

A mixture of 3-(3-chlorobenzyl)-1-cyclopropyl-1H-pyrazolo[3,4-d]pyrimidin-4-amine (50.0 mg, 166.8 μmol, 1.0 eg), phenylboronic acid (30.5 mg, 250.2 μmol, 1.5 eq), K₃PO₄ (70.8 mg, 333.6 μmol, 2.0 eq), chloro[(tri-tert-butylphosphine)-2-(2-aminobiphenyl)]palladium(II) (CAS:1375325-71-5) (8.5 mg, 16.7 μmol, 0.1 eq) in ethanol (4.0 mL) and H₂O (1.0 mL) was stirred at 100° C. for about 16 h under nitrogen atmosphere. The mixture was filtered and the filtrate was concentrated by rotary evaporator and the resulting residue was purified by prep-HPLC (condition: neutral) to afford 3-([1,1′-biphenyl]-3-ylmethyl)-1-cyclopropyl-1H-pyrazolo[3,4-d]pyrimidin-4-amine (Compound 8) (23.3 mg, 68.2 μmol, 40.9% yield) as a white solid. ¹H NMR: (400 MHz, CDCl₃) δ=8.34 (s, 1H), 7.54-7.46 (m, 3H), 7.44-7.41 (m, 4H), 7.37-7.36 (m, 1H), 7.34-7.20 (m, 1H), 4.90 (s, 2H), 4.37 (s, 2H), 3.74 (d, J=3.6 Hz, 1H), 1.35 (s, 2H), 1.20-1.16 (m, 2H). LCMS: (M+H)⁺: 242.2, Rt: 2.519 min. LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C₁₋₈ column (5 μm particles). Detection methods are diode array (DAD) and evaporative light scattering (ELSD) detection as well as positive electrospray ionization (MS).

The following compounds were prepared in a similar fashion as described above for method C using different starting materials.

TABLE 2
Compounds Prepared by Method C

| | | | LC/MS | |
|---|---|---|---|---|
| Compound | | | Observed | |
| No. | IUPAC Name | MW | (M +H) | 1H NMR (400 MHz) |
| 9 | 3-([1,1'-biphenyl]-3- | 341.41 | 342.2 | CHLOROFORM-d) δ = 8.34 |
| | ylmethyl)-1- | | | (s, 1H), 7.54-7.46 (m, 3H), |
| | cyclopropyl-1H- | | | 7.44-7.41 (m, 4H), 7.37-7.36 |
| | pyrazolo[3,4-d] | | | (m, 1H), 7.34-7.20 (m, 1H), |
| | pyrimidin-4-amine | | | 4.90 (s, 2H), 4.37 (s, 2H), 3.74 |
| | | | | (d, J = 3.6 Hz, 1H), 1.35 (s, 2H), |
| | | | | 1.20-1.16 (m, 2H) |
| 10 | 1-cyclopropyl-3-(3- | 343.39 | 344.1 | (DMSO-d6) δ = 9.18 (s, 1H), |
| | (pyrimidin-5-yl) | | | 9.08 (s, 2H), 8.15 (s, 1H), 7.80 |
| | benzyl)-1H- | | | (s, 1H), 7.64 (d, J = 7.6 Hz, 1H), |
| | pyrazolo[3,4-d] | | | 7.43 (t, J = 7.6 Hz, 1H), |
| | pyrimidin-4-amine | | | 7.27 (d, J = 7.2 Hz, 1H), 7.02 |
| | | | | (s, 1H), 4.41 (s, 2H), 3.76-3.70 |
| | | | | (m, 1H), 1.15-1.11 (m, 2H), |
| | | | | 1.04-0.99 (m, 2H) |
| 11 | 1-cyclopropyl-3-(3- | 342.40 | 343.2 | (DMSO-d6) δ = 8.63 (s, 2H), |
| | (pyridin-4- | | | 8.15 (s, 1H), 7.79 (s, 1H), 7.63 |
| | yl)benzyl)-1H- | | | (s, 3H), 7.41 (s, 1H), 7.28 (s, 1H), |
| | pyrazolo[3,4-d] | | | 4.41 (s, 2H), 3.74 (s, 1H), |
| | pyrimidin-4-amine | | | 1.12 (d, J = 40.4 Hz, 4H) |
| 12 | 1-cyclopropyl-3-(3- | 342.40 | 343.2 | (METHANOL-d4) δ = 9.00 (s, |
| | (pyridin-3- | | | 1H), 8.73 (d, J = 5.2 Hz, 1H), |
| | yl)benzyl)-1H- | | | 8.59 (d, J = 7.2 Hz, 1H), 8.32 |
| | pyrazolo[3,4-d] | | | (s, 1H), 7.97-7.91 (m, 1H), |
| | pyrimidin-4-amine | | | 7.71-7.63 (m, 2H), 7.52 (t, J = |
| | | | | 7.6 Hz, 1H), 7.39 (d, J = 7.6 Hz |
| | | | | 1H), 4.51 (s, 2H), 3.89 (m, 1H), |
| | | | | 1.31-1.24 (m, 2H), 1.18-1.11 |
| | | | | (m, 2H) |
| 13 | 1-cyclopropyl-3-(3- | 342.40 | 343.1 | (METHANOL-d4) δ = 8.73 (d, |
| | (pyridin-2- | | | J = 5.2 Hz, 1H), 8.38-8.31 (m, |
| | yl)benzyl)-1H- | | | 2H), 8.13 (d, J = 8.0 Hz, 1H), |
| | pyrazolo[3,4-d] | | | 7.87-7.80 (m, 2H), 7.78-7.73 |
| | pyrimidin-4-amine | | | (m, 1H), 7.60-7.54 (t, J = |
| | | | | 7.6 Hz, 1H), 7.51-7.46 (m, 1H), |
| | | | | 4.54 (s, 2H), 3.92 (m, 1H), |
| | | | | 1.32-1.22 (m, 2H), 1.20-1.10 |
| | | | | (m, 2H) |
| 14 | 1-cyclopropyl-3-(3- | 343.39 | 344.1 | (METHANOL-d4) δ = 8.82 (d, |
| | (pyrimidin-2- | | | J = 5.2 Hz, 2H), 8.34-8.25 (m, |
| | yl)benzyl)-1H- | | | 3H), 7.50-7.44 (m, 1H), 7.44- |
| | pyrazolo[3,4-d] | | | 7.38 (m, 1H), 7.36 (t, J = 4.8 Hz, |
| | pyrimidin-4-amine | | | 1H), 4.50 (s, 2H), 3.91 (m, 1H), |
| | | | | 1.35-1.27 (m, 2H), |
| | | | | 1.21-1.11 (m, 2H) |
| 54 | 1-cyclopropyl-3-((4- | 344.1 | 345.1 | (METHANOL-d4) δ = 8.32 (s, |
| | phenylpyridin-2- | | | 1H), 8.25 (d, J = 5.2 Hz, 1H), |
| | yl)oxy)-1H- | | | 7.80-7.78 (m, 2H), 7.69 |
| | pyrazolo[3,4-d] | | | (s, 1H), 7.57-7.48 (m, 4H), |
| | pyrimidin-4-amine | | | 3.83-3.78 (m, 1H), 1.27-1.21 |
| | | | | (m, 2H), 1.18-1.11 (m, 2H) |
| 55 | 3-([3,4'-bipyridin]- | 345.1 | 346.1 | (METHANOL-d4) δ = 9.05 (d, |
| | 2'-yloxy)-1- | | | J = 2.0 Hz, 1H), 8.75-8.73 (m, |
| | cyclopropyl-1H- | | | 1H), 8.43-8.40 (m, 1H), 8.35- |
| | pyrazolo[3,4-d] | | | 8.32 (m, 2H), 7.77-7.73 (m, 2H), |
| | pyrimidin-4-amine | | | 7.65-7.64 (m, 1H), 3.88-3.83 |
| | | | | (m, 1H), 1.26-1.22 (m, 2H), |
| | | | | 1.15-1.12 (m, 2H) |
| 56 | 1-cyclopropyl-3-((5- | 363.1 | 364.1 | (DMSO-d6) δ = 8.98 (s, 1H), |
| | fluoro-[3,4'- | | | 8.71 (d, J = 2.8 Hz, 1H), 8.29- |
| | bipyridin]-2'-yl)oxy)- | | | 8.26 (m, 1H), 8.23-8.21 (m, 1H), |
| | 1H-pyrazolo[3,4-d] | | | 8.19 (s, 1H), 7.69 (s, 1H), |
| | pyrimidin-4-amine | | | 7.66-7.64 (m, 1H), 3.72-3.67 |
| | | | | (m, 1H), 1.08-0.99 (m, 4H) |
| 57 | 1-cyclopropyl-3-((4- | 350.1 | 351.1 | (METHANOL-d4) δ = 8.33 |
| | (3,6-dihydro-2H- | | | (s, 1H), 8.13 (d, J = 5.6 Hz, 1H), |
| | pyran-4-yl)pyridin-2- | | | 7.43 (s, 1H), 7.37-7.36 (m, 1H), |
| | yl)oxy)-1H- | | | 6.61-6.60 (m, 1H), 4.35-4.33 |
| | pyrazolo[3,4-d] | | | (m, 2H), 3.94 (t, J = 5.6 |
| | pyrimidin-4-amine | | | Hz, 2H), 3.84-3.81 (m, 1H), |
| | | | | 2.58-2.52 (m, 2H), 1.25-1.09 |
| | | | | (m, 4H) |
| 58 | 1-cyclopropyl-3((2- | 345.1 | 345.1 | (METHANOL-d4) δ = 8.76 |
| | phenylpyridin-4- | | | (d, J = 6.4 Hz, 1H), 8.37 (s, 1H), |
| | yl)oxy)-1H- | | | 8.19 (d, J = 2.4 Hz, 1H), |
| | pyrazolo[3,4-d] | | | 7.97-7.93 (m, 2H), 7.84-7.82 |
| | pyrimidin-4-amine | | | (m, 1H), 7.64-7.61 (m, 3H), |
| | | | | 3.91-3.86 (m, 1H), 1.28-1.26 |
| | | | | (m, 2H), 1.17-1.15 (m, 2H) |
| 59 | 3-([2,3'-bipyridin]- | 345.1 | 346.1 | (METHANOL-d4) δ = 9.41 |
| | 4-yloxy)-1- | | | (s, 1H), 8.95-8.93 (m, 1H) |
| | cyclopropyl-1H- | | | 8.83 (d, J = 5.2 Hz, 1H), |
| | pyrazolo[3,4-d] | | | 8.77 (d, J = 6.0 Hz, 1H), |
| | pyrimidin-4-amine | | | 8.37 (s, 1H), |
| | | | | 8.15 (d, J = 2.0 Hz, 1H), |
| | | | | 8.00-7.97 (m, 1H), |
| | | | | 7.62-7.60 (m, 1H), |
| | | | | 3.89-3.83 (m, 1H), |
| | | | | 1.27-1.23 (m, 2H), |
| | | | | 1.15-1.13 (m, 2H) |
| 60 | 1-cyclopropyl-3- | 363.1 | 364.1 | (DMSO-d6) δ = 9.16 (s, 1H), |
| | ((5'-fluoro-[2,3′- | | | 8.70-8.65 (m, 2H), |
| | bipyridin]-4-yl)oxy)- | | | 8.35-8.31 (m, 1H), |
| | 1H-pyrazolo[3,4-d] | | | 8.23 (s, 1H), |
| | pyrimidin-4-amine | | | 8.13 (d, J = 2.0 Hz, 1H), |
| | | | | 7.43-7.41 (m, 1H), |
| | | | | 3.73-3.68 (m, 1H), |
| | | | | 1.15-1.02 (m, 4H) |
| 61 | 1-cyclopropyl-3-((2- | 350.1 | 351.1 | (METHANOL-d4) δ = 8.68 |
| | (3,6-dihydro-2H- | | | (d, J = 6.8 Hz, 1H), 8.38 (s, 1H), |
| | pyran-4-yl)pyridin- | | | 8.00 (d, J = 2.0 Hz, 1H), |
| | 4-yl)oxy)-1H- | | | 7.88-7.86 (m, 1H), |
| | pyrazolo[3,4-d] | | | 6.91-6.90 (m, 1H), |
| | pyrimidin-4-amine | | | 4.42-4.40 (m, 2H), |
| | | | | 3.97 (t, J = 5.2 Hz, 2H), |
| | | | | 3.92-3.89( m, 1H), |
| | | | | 2.66-2.61 (m, 2H), |
| | | | | 1.28-1.15 (m, 4H) |

### Synthesis Method D: General Procedure Represented by the Preparation of (4-amino-1-cyclopropyl-1H-pyrazolo[3,4-d]pyrimidin-3-yl)(phenyl)methanol and 3-benzyl-1-cyclopropyl-1H-pyrazolo[3,4-d]pyrimidin-4-amine

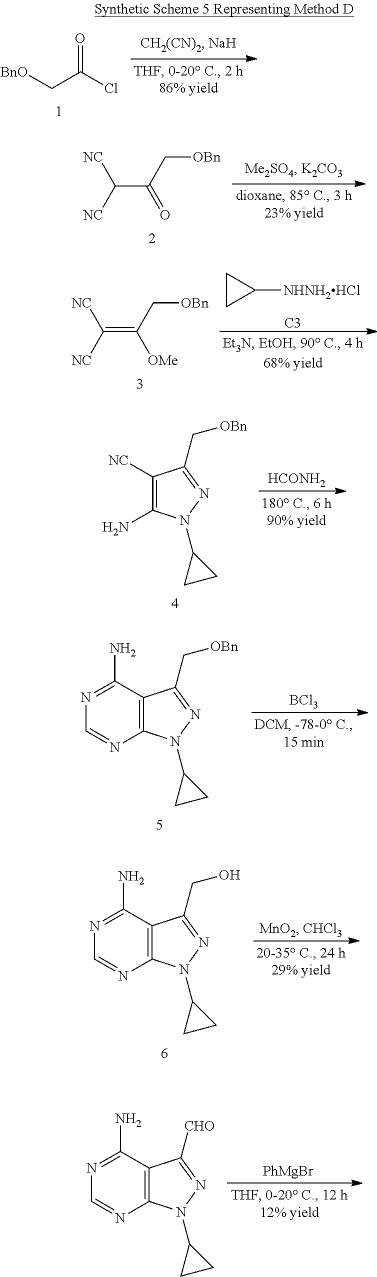

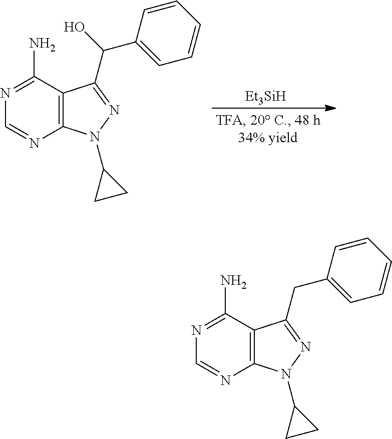 [SMILES: "Et3SiH" ; Nc1ncnc2c1c(C(O)c1ccccc1)nn2C1CC1 ; Nc1ncnc2c1c(Cc1ccccc1)nn2C1CC1]

### Step 1

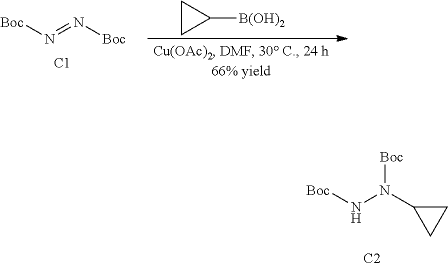

Di-tert-butyl (E)-diazene-1,2-dicarboxylate (200.0 g, 868.5 mmol, 1.0 eq), cyclopropylboronic acid (149.2 g, 1.7 mol, 2.0 eq) and Cu(OAc)₂ (15.7 g, 86.8 mmol, 0.1 eq) were combined in DMF (2.0 L), degassed and purged with N₂ three times, and then stirred at 30° C. for 24 h under N₂ atmosphere. The mixture was concentrated under reduced pressure and partitioned between EtOAc (2 L) and H₂O (2 L). The organic phase was separated, washed with brine (2 L), dried over Na₂SO₄, filtered and concentrated under reduced pressure to give a residue. The residue was taken up in 2 L of petroleum ether, stirred for 16 h and filtered to collect the solid to afford di-tert-butyl 1-cyclopropylhydrazine-1,2-dicarboxylate (470.0 g, 66.0% yield) as a white solid. ¹H NMR (400 MHz, CDCl₃) δ=3.12-3.04 (m, 1H), 1.63 (s, 1H), 0.87-0.80 (m, 4H)

### Step 2

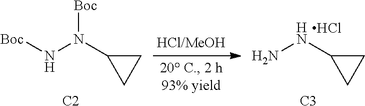

Di-tert-butyl 1-cyclopropylhydrazine-1,2-dicarboxylate (20.0 g, 73.4 mmol, 1.0 eq) was stirred in HCl/MeOH (200.0 mL) at 20° C. for 2 h. The mixture was concentrated under reduced pressure to give cyclopropylhydrazine (10.0 g, 68.9 mmol, 93.8% yield) without further purification.

### Step 3

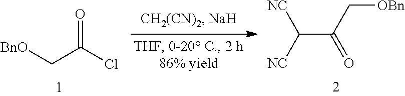 [SMILES: O=C(Cl)COCc1ccccc1 ; N#CC(C#N)C(=O)COCc1ccccc1]

Malononitrile (12.5 g, 189.5 mmol, 1.0 eq) was dissolved in THF (600.0 mL) and the solution stirred at 0-5° C. while NaH (15.1 g, 379.1 mmol, 60% purity, 2.0 eq) was added in portions followed by drop-wise addition of 2-(benzyloxy)acetyl chloride (35.0 g, 189.5 mmol, 29.4 mL, 1.0 eq) in THF (70.0 mL). The solution was stirred at 20° C. for 2 h. The reaction mixture was poured into 1 M HCl (0.5 L), and extracted with 3×100 mL of EtOAc. The combined organic fractions were washed with brine (250 mL), dried over Na₂SO₄, filtered and concentrated under reduced pressure. The remaining residue was triturated with petroleum ether (250 mL) to give 2-(2-(benzyloxy)acetyl)malononitrile (37.5 g, 165 mmol, 86.7% yield) as a yellow solid.

### Step 4

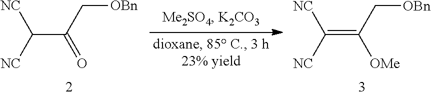 [SMILES: N#CC(C#N)C(=O)COCc1ccccc1 ; COC(COCc1ccccc1)=C(C#N)C#N]

A mixture of 2-(2-(benzyloxy)acetyl)malononitrile (35.0 g, 163.3 mmol, 1.0 eq), Me₂SO₄ (28.8 g, 228.7 mmol, 21.6 mL, 1.4 eq) and K₂CO₃ (38.3 g, 277.7 mmol, 1.7 eq) in dioxane (500.0 mL) was degassed and purged with N₂ three times and then stirred at 85° C. for 3 h under N₂ atmosphere. The mixture was concentrated under reduced pressure and the residue purified by column chromatography (SiO₂, petroleum ether/ethyl acetate=1/1) to afford 2-(2-(benzyloxy)-1-methoxyethylidene)malononitrile (17.0 g, 38.7 mmol, 23.6% yield) as a yellow oil. ¹H NMR: (400 MHz, CDCl₃) δ=7.41-7.35 (m, 5H), 4.63 (s, 2H), 4.45 (s, 2H), 4.20 (s, 3H).

### Step 5

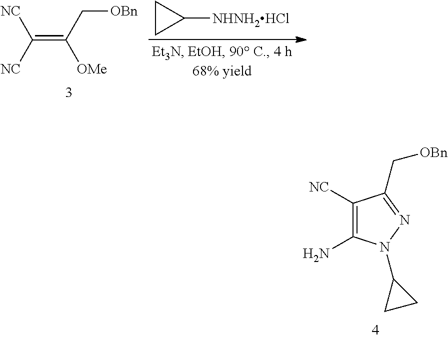

A mixture of 2-(2-(benzyloxy)-1-methoxyethylidene)malononitrile (20.0 g, 87.6 mmol, 1.0 eq), cyclopropylhydrazine (10.4 g, 96.3 mmol, 1.1 eq, HCl), Et₃N (11.5 g, 113.9 mmol, 15.7 mL, 1.3 eq) in EtOH (400.0 mL) was degassed and purged with N₂ three times and then stirred at 90° C. for 4 h under N₂ atmosphere. The mixture was concentrated under reduced pressure and the remaining residue was purified by column chromatography (SiO₂, petroleum ether/ethyl acetate=1/2) to afford 5-amino-3-((benzyloxy)methyl)-1-cyclopropyl-1H-pyrazole-4-carbonitrile (16.0 g, 59.6 mmol, 68.0% yield) as a yellow solid. ¹H NMR: (400 MHz, CDCl₃) δ=7.44-7.39 (m, 2H), 7.35 (t, 7=7.2 Hz, 2H), 7.32-7.27 (m, 1H), 4.67 (s, 2H), 4.61 (s, 2H), 4.47 (s, 2H), 3.12-3.04 (m, 1H), 1.16-1.05 (m, 4H).

### Step 6

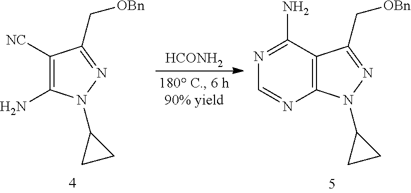 [SMILES: N#Cc1c(COCc2ccccc2)nn(C2CC2)c1N ; "HCONH2" ; Nc1ncnc2c1c(COCc1ccccc1)nn2C1CC1]

A mixture of 5-amino-3-((benzyloxy)methyl)-1-cyclopropyl-1H-pyrazole-4-carbonitrile (15.0 g, 55.9 mmol, 1.0 eq) and formamide (254.2 g, 5.6 mol, 225.0 mL, 100.9 eq) was degassed and purged with N₂ three times, and then stirred at 180° C. for 6 h under N₂ atmosphere. The solution stood for 12 h at 20° C. and the deposited crystalline material was separated by filtration and washed with formamide (30 mL), water (100 mL) and dried under reduced pressure to give 3-((benzyloxy)methyl)-1-cyclopropyl-1H-pyrazolo[3,4-d]pyrimidin-4-amine (15.0 g, 50.6 mmol, 90.5% yield) as a yellow solid. ¹H NMR: (400 MHz, CDCl₃) δ=8.33 (s, 1H), 8.22 (d, J=13.6 Hz, 1H), 7.39-7.28 (m, 5H), 4.86 (s, 2H), 4.59 (s, 2H), 3.72-3.66 (m, 1H), 1.30-1.23 (m, 2H), 1.18-1.09 (m, 2H).

### Step 7

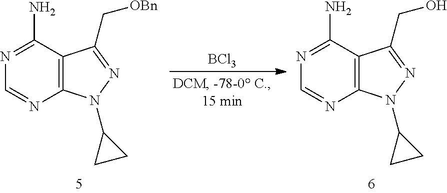 [SMILES: Nc1ncnc2c1c(COCc1ccccc1)nn2C1CC1 ; "BCl3" ; Nc1ncnc2c1c(CO)nn2C1CC1]

To a solution of 3-((benzyloxy)methyl)-1-cyclopropyl-1H-pyrazolo[3,4-d]pyrimidin-4-amine (13.0 g, 44.0 mmol, 1.0 eq) in DCM (390.0 mL) was added BCl₃ (1 M, 176.0 mL, 4.0 eq) dropwise at −78° C., then the reaction was warmed to 0° C., and stirred at 0° C. for 13 min. TLC (DCM/MeOH=10/1) indicated no starting material remained and one major new spot with larger polarity was detected. The reaction was quenched with MeOH (100 mL) at −78° C. and then the pH was adjusted to 7 by addition of NH₃.H₂O at 0° C. The mixture was filtered and the filtrate was concentrated under reduced pressure. The remaining residue was precipitated by addition of petroleum ether (100 mL), Altered and the filter cake was concentrated under reduced pressure to give (4-amino-1-cyclopropyl-1H-pyrazolo[3,4-d]pyrimidin-3-yl)methanol (15.0 g, crude) as a brown solid. ¹H NMR: (400 MHz, METHANOL-d₄) δ=8.18 (s, 1H), 8.05 (s, 1H), 4.82 (s, 2H), 3.70-3.59 (m, 1H), 1.19-1.07 (m, 4H).

### Step 8

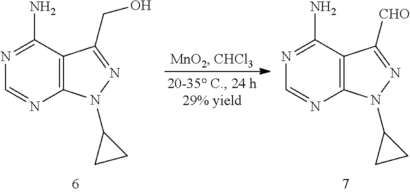

A mixture of (4-amino-1-cyclopropyl-1H-pyrazolo[3,4-d]pyrimidin-3-yl)methanol (5.0 g, 24.3 mmol, 1.0 eq) and MnO₂ (21.1 g, 243.6 mmol, 10.0 eq) in CHCl₃ (20.0 mL) was degassed and purged with N₂ three times, and then stirred at 20-35° C. for 24 h under N₂ atmosphere. The mixture was filtered and the filtrate was concentrated under reduced pressure to give 4-amino-1-cyclopropyl-1H-pyrazolo[3,4-d]pyrimidine-3-carbaldehyde (2.0 g, 7.0 mmol, 29.0% yield) as a yellow solid without further purification. ¹H NMR: (400 MHz, METHANOL-d₄) δ=9.91 (s, 1H), 8.29 (s, 1H), 4.04-3.98 (m, 1H), 1.36-1.34 (m, 2H), 1.21-1.19 (m, 2H).

### Step 9

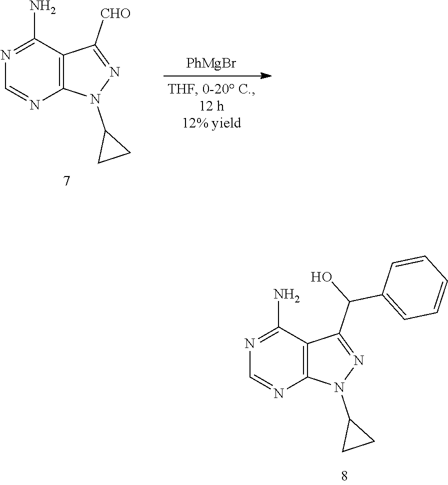 [SMILES: "PhMgBr" ; Nc1ncnc2c1c(C=O)nn2C1CC1 ; Nc1ncnc2c1c(C(O)c1ccccc1)nn2C1CC1]

To a solution of 4-amino-1-cyclopropyl-1H-pyrazolo[3,4-d]pyrimidine-3-carbaldehyde (200.0 mg, 984.2 μmol, 1.0 eq) in THF (10.0 mL) was added bromo(phenyl)magnesium (3 M, 656.1 μL, 2.0 eq) at 0° C. The mixture was warmed to 20° C. and stirred at 20° C. for 12 h, then quenched with saturated NH₄Cl aq. (10 mL) and extracted with DCM (2×5 mL). The combined organic layers were dried over Na₂SO₄, filtered and concentrated under reduced pressure. The remaining residue was purified by prep-HPLC (condition: neutral) to give (4-amino-1-cyclopropyl-1H-pyrazolo[3,4-d]pyrimidin-3-yl)(phenyl)methanol (39.0 mg, 125.1 μmol, 12.7% yield, 90.2% purity) as a white solid. ¹H NMR: (400 MHz, METHANOL-d₄) δ=8.18 (s, 1H), 7.41-7.36 (m, 2H), 7.31 (t, J=7.6 Hz, 2H), 7.26-7.20 (m, 1H), 6.02 (s, 1H), 3.71-3.65 (m, 1H), 1.20-1.18 (m, 2H), 1.13-1.10 (m, 2H). LCMS: (M+H)⁺: 282.1, Rt: 2.267 min. LC/MS (The gradient was 1-90% B in 3.4 min. 90-100% B in 0.45 min, 100-1% B in 0.01 min, and then held at 1% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS).

### Step 10

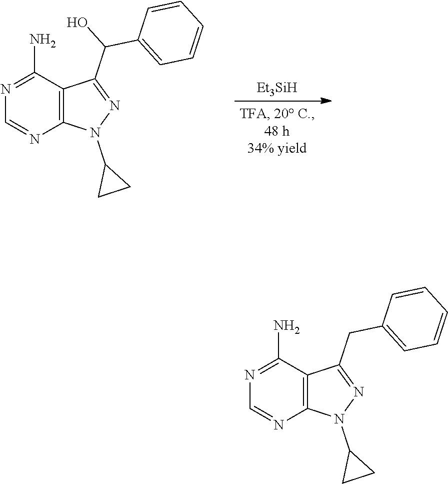 [SMILES: "Et3SiH" ; Nc1ncnc2c1c(C(O)c1ccccc1)nn2C1CC1 ; Nc1ncnc2c1c(Cc1ccccc1)nn2C1CC1]

To a solution of (4-amino-1-cyclopropyl-1H-pyrazolo[3,4-d]pyrimidin-3-yl)(phenyl)methanol (19.0 mg, 67.5 μmol, 1.0 eq) in TFA (500.0 μL) was added Et₃SiH (27.4 mg, 236.3 μmol, 37.6 μL, 3.5 eq). The mixture was stirred at 20° C. for 48 h, concentrated under reduced pressure and purified by prep-HPLC (condition: TFA) to give 3-benzyl-1-cyclopropyl-1H-pyrazolo[3,4-d]pyrimidin-4-amine (Compound 15) (6.2 mg, 23.0 μmol, 34.1% yield, 98.7% purity) as a white solid. ¹H NMR: (400 MHz, METHANOL-d₄) δ=8.29 (s, 1H), 7.34-7.27 (m, 2H), 7.27-7.16 (m, 3H), 4.38 (s, 2H), 3.91-3.85 (m, 1H), 1.32-1.26 (m, 2H), 1.19-1.13 (m, 2H). LCMS: (M+H)⁺: 266.1, Rt: 1.983 min. LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS).

### Alternative Step 9. represented by the preparation of (4-amino-1-cyclopropyl-1H-pyrazolo[3,4-d]pyrimidin-3-yl)(pyridin-3-yl)methanol

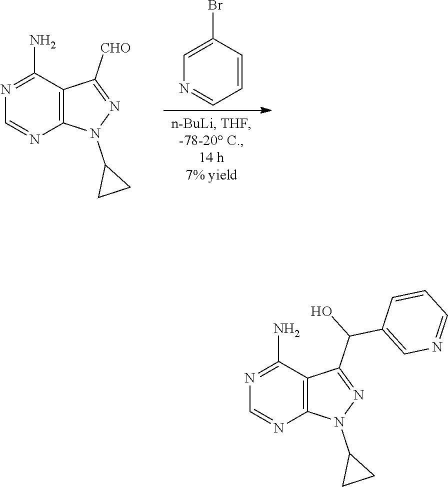 [SMILES: Nc1ncnc2c1c(C=O)nn2C1CC1 ; Brc1cccnc1 ; Nc1ncnc2c1c(C(O)c1cccnc1)nn2C1CC1]

To a solution of 3-bromopyridine (467.6 mg, 2.9 mmol, 285.1 μL, 2.0 eq) in THF (20.0 mL) was added drop-wise n-BuLi (2.5 M, 1.3 mL, 2.2 eq) at −78° C., followed by addition of 4-amino-1-cyclopropyl-1H-pyrazolo[3,4-d]pyrimidine-3-carbaldehyde (300.0 mg, 1.4 mmol, 1.0 eq). The mixture was stirred at −78° C. for 2 h and then warmed to 20° C. and stirred for 12 h. The reaction was quenched with aq. NH₄Cl (10 mL), extracted with DCM (2×5 mL) and the combined organic layers were dried over Na₂SO₄, filtered and concentrated under reduced pressure. The residue was purified by prep-HPLC (condition: TFA) to give (4-amino-1-cyclopropyl-1H-pyrazolo[3,4-d]pyrimidin-3-yl)(pyridin-3-yl)methanol (Compound 18) (30.0 mg, 105.4 μmol, 7.1% yield, 99.2% purity) as a yellow solid. ¹H NMR: (400 MHz, METHANOL-d₄) δ=8.87 (d, J=1.6 Hz, 1H), 8.70 (d, J=5.6 Hz, 1H), 8.39 (d, J=8.0 Hz, 1H), 8.36 (s, 1H), 7.86 (dd, J=5.6, 8.0 Hz, 1H), 6.34 (s, 1H), 3.95-3.89 (m, 1H), 1.28-1.22 (m, 2H), 1.18-1.11 (m, 2H). LCMS: (M+H)⁺: 283.1, Rt: 2.037 min. LC/MS (The gradient was 0-80% B in 3.4 min, 80-100% B in 0.45 min, 100-0% B in 0.01 min, and then held at 0% B for 0.65 min (0.6 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS).

The following compounds were prepared in a similar manner as for method D using different starting materials.

TABLE 3
Compounds Prepared by Method D

| | | | LC/MS | |
|---|---|---|---|---|
| Compound | | | Observed | |
| No. | IUPAC Name | MW | (M + H) | 1H NMR (400 MHz) |
| 16 | (4-amino-1- | 281.31 | 282.1 | (METHANOL-d4) δ = 8.18 |
| | cyclopropyl- | | | (s, 1H), 7.41-7.36 (m, 2H), |
| | 1H-pyrazolo[3,4-d] | | | 7.31 (t, J = 7.6 Hz, 2H), |
| | pyrimidin-3-yl) | | | 7.26-7.20 (m, 1H), 6.02 (s, 1H), |
| | (phenyl)methanol | | | 3.71-3.65 (m, 1H), |
| | | | | 1.20-1.18 (m, 2H), |
| | | | | 1.13-1.10 (m, 2H) |
| 17 | 3-benzyl-1-cyclopropyl- | 265.31 | 266.1 | (METHANOL-d4) δ = 8.29 |
| | 1H-pyrazolo[3,4-d] | | | (s, 1H), 7.34-7.27 (m, 2H), |
| | pyrimidin-4-amine | | | 7.27-7.16 (m, 3H), 4.38 (s, 2H), |
| | | | | 3.91-3.85 (m, 1H), 1.32-1.26 |
| | | | | (m, 2H), 1.19-1.13 (m, 2H) |
| 18 | (4-amino-1- | 282.30 | 283.1 | (METHANOL-d4) δ = 8.87 |
| | cyclopropyl-1H- | | | (d, J = 1.6 Hz, 1H), 8.70 |
| | pyrazolo[3,4-d] | | | (d, J = 5.6 Hz, , 1H), |
| | pyrimidin-3-yl) | | | 8.39 (d, J = 8.0 Hz, 1H), |
| | (pyridin-3-yl) | | | 8.36 (s, 1H), |
| | methanol | | | 7.86 (dd, J = 5.6, 8.0 Hz, 1H), |
| | | | | 6.34 (s, 1H), |
| | | | | 3.95-3.89 (m, 1H), |
| | | | | 1.28-1.22 (m, 2H), |
| | | | | 1.18-1.11 (m, 2H) |

### Synthesis Method E: General Procedure Represented by the Preparation of 3-(3-chlorophenethyl)-1-cyclopropyl-1H-pyrazolo[3,4-d]pyrimidin-4-amine and 1-cyclopropyl-3-(3-fluorophenethyl)-1H-pyrazolo[3,4-d]pyrimidin-4-amine

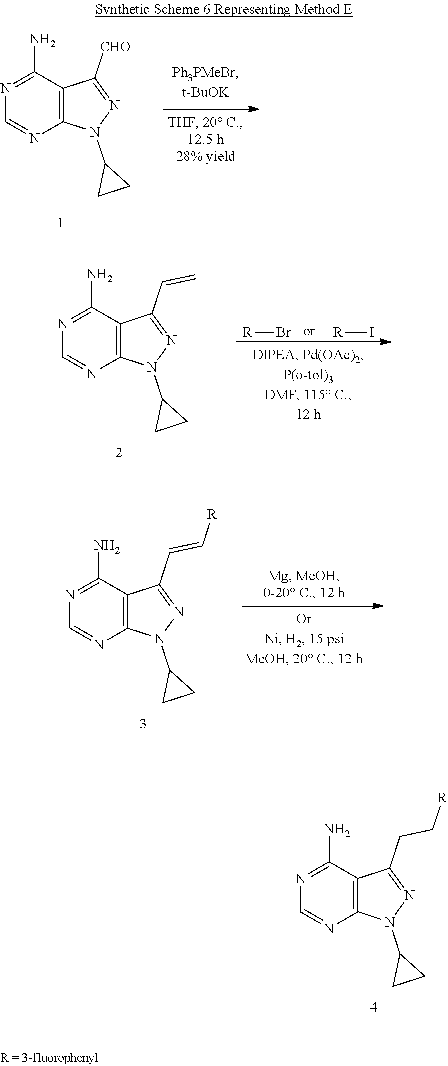

### Step 1

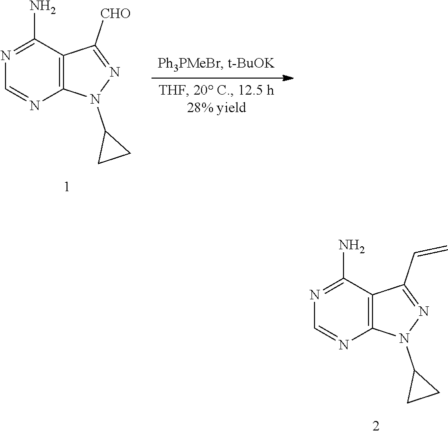

To a solution of bromo-methyl-triphenyl-phosphane (2.6 g, 7.3 mmol, 1.5 eq) in THF (40.0 mL) was added t-BuOK (1.3 g, 12.3 mmol, 2.5 eq) at 20° C. in one portion. After addition, the mixture was stirred at this temperature for 0.5 h, and then 4-amino-1-cyclopropyl-1H-pyrazolo[3,4-d]pyrimidine-3-carbaldehyde (1.0 g, 4.9 mmol, 1.0 eq) was added at 20° C. The resulting mixture was stirred at 20° C. for 12 h. The mixture was filtered and the filtrate was concentrated under reduced pressure to give a residue. The residue was purified by column chromatography (SiO₂, DCM/MeOH=20/1) to give 1-cyclopropyl-3-vinyl-1H-pyrazolo[3,4-d]pyrimidin-4-amine (450.0 mg, 1.3 mmol, 28.1% yield) as a white solid. ¹H NMR: (400 MHz, METHANOL-d₄) δ=8.19 (s, 1H), 7.07 (dd, J=11.2, 17.2 Hz, 1H), 6.05 (dd. J=1.6, 17.2 Hz, 1H), 5.55-5.49 (m, 1H), 3.76-3.70 (m, 1H), 1.28-1.21 (m, 2H), 1.16-1.10 (m, 2H).

### Step 2

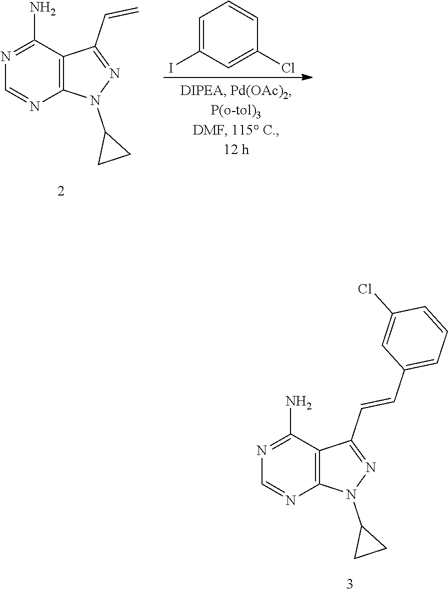 [SMILES: C=Cc1nn(C2CC2)c2ncnc(N)c12 ; Clc1cccc(I)c1 ; Nc1ncnc2c1c(/C=C/c1cccc(Cl)c1)nn2C1CC1]

1-Cyclopropyl-3-vinyl-1H-pyrazolo[3,4-d]pyrimidin-4-amine (125.0 mg, 621.1 μmol, 1.0 eq), 3-chloroiodobenzene (148.1 mg, 621.1 μmol, 76.7 μL, 1.0 eq), Pd(OAc)₂ (1.3 mg, 6.2 μmol, 0.01 eq), tri-ortho-tolylphosphine (56.7 mg, 186.3 μmol, 0.3 eq) and DIPEA (120.4 mg, 931.7 μmol, 162.7 μL, 1.5 eq) were combined in DMF (1.5 mL) and degassed and purged with N₂ three times, then stirred at 115° C. for 12 h under N₂ atmosphere. The mixture was Altered over celite and the filtrate was concentrated under reduced pressure. The residue was purified by prep-HPLC (condition: TFA) to give (E)-3-(3-chlorostyryl)-1-cyclopropyl-1H-pyrazolo[3,4-d]pyrimidin-4-amine (50.0 mg, 110.3 μmol, 17.7% yield, 94% purity, TFA) as a brown solid. ¹H NMR: (400 MHz, METHANOL-d₄) δ=8.31 (s, 1H), 7.81 (s, 1H), 7.60-7.56 (m, 3H), 7.36 (td, J=8.0, 16.0 Hz, 2H), 3.98 (m, 1H), 1.40-1.33 (m, 2H), 1.23-1.15 (m, 2H). LCMS: (M+H)⁺: 312.1, Rt: 2.445 min. LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C₁₋₈ column (5 μm particles). Detection methods are diode array (DAD) and evaporative light scattering (ELSD) detection as well as positive electrospray ionization (MS).

### Step 3

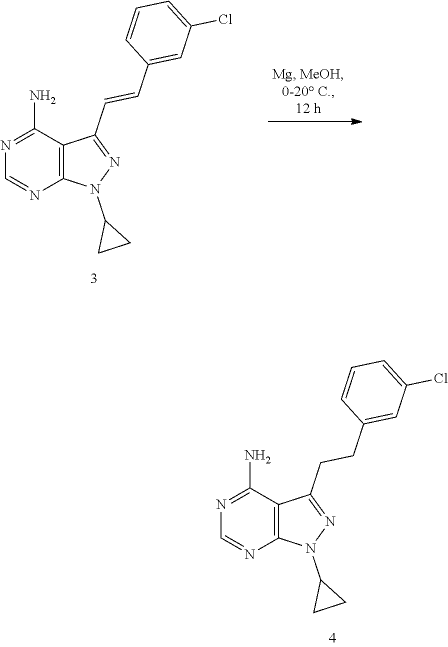 [SMILES: Nc1ncnc2c1c(/C=C/c1cccc(Cl)c1)nn2C1CC1 ; Nc1ncnc2c1c(CCc1cccc(Cl)c1)nn2C1CC1]

To a solution of (E)-3-(3-chlorostyryl)-1-cyclopropyl-1H-pyrazolo[3,4-d]pyrimidin-4-amine (15.0 mg, 53.9 μmol, 1.0 eq) in MeOH (10.0 mL) was added Mg (26.2 mg, 1.0 mmol, 20.0 eq) at 0° C. The mixture was warmed to 20° C. and stirred at 20° C. for 12 h. The mixture was quenched with sat. NH₄Cl aq. (10 mL), extracted with DCM (2×5 mL). The combined organic extracts were dried over Na₂SO₄, filtered and concentrated under reduced pressure. The residue was purified by prep-HPLC (condition: neutral) to give 3-(3-chlorophenethyl)-1-cyclopropyl-1H-pyrazolo[3,4-d]pyrimidin-4-amine (Compound 19) (3.1 mg, 10.8 μmol, 20.1% yield, 98% purity) as a white solid. ¹H NMR: (400 MHz, METHANOL-d₄) δ=8.27 (s, 1H), 7.19 (s, 3H), 7.11 (s, 1H), 3.84 (s, 1H), 3.39-3.35 (m, 2H), 3.09 (d, J=8.0 Hz, 2H), 1.20 (s, 2H), 1.13 (s, 2H). LCMS: (M+H)⁺: 314.0, RT: 2.382 min. LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS).

### Step 14

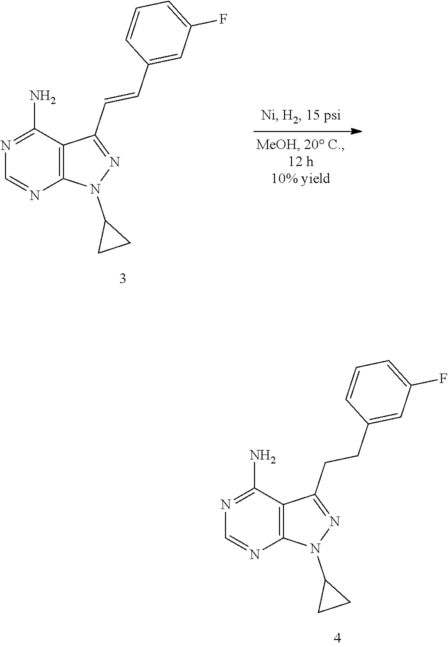 [SMILES: Nc1ncnc2c1c(/C=C/c1cccc(F)c1)nn2C1CC1 ; Nc1ncnc2c1c(CCc1cccc(F)c1)nn2C1CC1]

To a solution of (E)-1-cyclopropyl-3-(3-fluorostyryl)-1H-pyrazolo[3,4-d]pyrimidin-4-amine (30.0 mg, 101.5 μmol, 1.0 eq) in MeOH (5.0 mL) was added Raney-Ni (0.6 g). The suspension was degassed and purged with H₂ three times and then stirred under H₂ (15 Psi) at 20° C. for 12 h, filtered over celite and concentrated under reduced pressure. The residue was purified by prep-HPLC (condition: TFA) to give 1-cyclopropyl-3-(3-fluorophenethyl)-1H-pyrazolo[3,4-d]pyrimidin-4-amine (Compound 20) (3.1 mg, 10.3 μmol, 10.1% yield, 99.3% purity) as a white solid. ¹H NMR: (400 MHz, METHANOL-d₄) δ=8.27 (s, 1H), 7.24 (d, J=7.6 Hz, 1H), 7.00-6.86 (m, 3H), 3.83 (s, 1H), 3.15-3.06 (m, 2H), 1.19 (s, 2H), 1.12 (d, J=6.4 Hz, 2H). LCMS: (M+H)⁺: 298.1, RT: 2.203 min. LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS).

The following compounds were prepared in a similar manner as described in method E using different starting materials.

TABLE 4
Compounds Prepared by Method E

| | | | LC/MS | |
|---|---|---|---|---|
| Compound | | | Observed | |
| No. | IUPAC Name | MW | (M + H) | 1H NMR (400 MHz) |
| 21 | (E)-1-cyclopropyl-3-(2- | 278.31 | 279.1 | (METHANOL-d4) δ = 8.99 (s, |
| | (pyridin-3-yl)vinyl)-1H- | | | 1H), 8.69-8.56 (m, 2H), 8.34 (s, |
| | pyrazolo[3,4- | | | 1H), 7.87-7.77 (m, 2H), 7.75- |
| | dlpyrimidin-4-amine | | | 7.66 (m, 1H), 4.03 (m, 1H), 1.40- |
| | | | | 1.34 (m, 2H), 1.24-1.17 (m, |
| | | | | 2H) |
| 22 | 1-cyclopropyl-3-(3- | 297.33 | 298.1 | (METHANOL-d4) δ = 8.27 (s, |
| | fluorophenethyl)-1H- | | | 1H), 7.24 (d, J = 7.6 Hz, 1H), |
| | pyrazolo[3,4- | | | 7.00-6.86 (m, 3H), 3.83 (s, 1H), |
| | d]pyrimidin-4-amine | | | 3.15-3.06 (m, 2H), 1.19 (s, 2H), |
| | | | | 1.12 (d, J = 6.4 Hz, 2H) |
| 23 | (E)-3-(3-chlorostyryl)- | 311.77 | 312.1 | (METHANOL-d4) δ = 8.31 (s, |
| | 1-cyclopropyl-1H- | | | 1H), 7.81 (s, 1H), 7.60-7.56 (m, |
| | pyrazolo[3,4- | | | 3H), 7.36 (td, J = 8.0, 16.0 Hz, |
| | d]pyrimidin-4-amine | | | 2H), 3.98 (m, 1H), 1.40-1.33 |
| | | | | (m, 2H), 1.23-1.15 (m, 2H) |
| 24 | (E)-3-(4-chlorostyryl)- | 311.77 | 312.1 | (METHANOL-d4) δ = 8.31 (s, |
| | 1-cyclopropy1-114- | | | 1H), 7.68 (d, J = 8.4 Hz, 2H), |
| | pyrazolo+3,4- | | | 7.62-7.48 (m, 2H), 7.40 (d, J = |
| | d+pyrimidin-4-amine | | | 8.4 Hz, 2H), 4.00-3.91 (m, 1H), |
| | | | | 1.39-1.33 (m, 2H), 1.22-1.15 |
| | | | | (m, 2H) |
| 25 | (E)-1-cyclopropyl-3-(3- | 295.31 | 296.1 | (METHANOL-d4) δ = 8.32 (s, |
| | fluorostyryl)-1H- | | | 1H), 7.64-7.51 (m, 3H), 7.49- |
| | pyrazolo[3,4- | | | 7.36 (m, 2H), 7.11-7.03 (m, |
| | d]pyrimidin-4-amine | | | 1H), 3.99 (m, 1H), 1,41-1.33 |
| | | | | (m, 2H), 1.24-1.16 (m, 2H) |
| 26 | 3-(3-chlorophenethyl)- | 313.78 | 314.0 | (METHANOL-d4) δ = 8,27 (s, |
| | 1-cyclopropyl-1H- | | | 1H), 7.19 (s, 3H), 7.11 (s, 1H), |
| | pyrazolo[3,4- | | | 3.84 (s, 1H), 3.39-3.35 (m, 2H), |
| | d]pyrimidin-4-amine | | | 3.09 (d, J = 8.0 Hz, 2H), 1.20 (s, |
| | | | | 2H), 1.13 (s, 2H) |
| 27 | 3-(4-chlorophenethyl- | 313.78 | 314.1 | (METHANOL-d4) δ = 8.27 (s, |
| | 1-cyclopropyl-1H- | | | 1H), 7.25-7.21 (m, 2H), 7.17- |
| | pyrazolo[3,4- | | | 7.12 (m, 2H), 3.88-3.79 (m, |
| | d]pyrimidin-4-amine | | | 1H), 3.28 (s, 2H), 3.11-3.04 (m, |
| | | | | 2H), 1.18 (d, J = 2.4 Hz, 2H), |
| | | | | 1.15-1.09 (m, 2H) |
| 28 | 1-cyclopropyl-3-(2- | 280.33 | 281.1 | (METHANOL-d4) δ = 8.32 (d, J = |
| | (pyridin-3-yl)ethyl)-1H- | | | 13.6 Hz, 2H), 8.16 (s, 1H), 7.66 |
| | pyrazolo[3,4- | | | (d, J = 8.0 Hz, 1H), 7.32 (s, 1H), |
| | d]pyrimidin-4-amine | | | 3.63 (s, 1H), 3.29-3.27 (m, 2H), |
| | | | | 3.12 (d, J = 7.2 Hz, 2H), 1.09 (s, |
| | | | | 4H) |
| 62 | (E)-1-cyclopropyl-3-(2- | 292.1 | 293.1 | (DMSO-d6) δ = 9.01 (s, 1H), |
| | (6-methylpyridin-3- | | | 8.72 (d, J = 7.2 Hz, 1H), 8.37 (s, |
| | yl)vinyl)-1H- | | | 1H), 7.88-7.84 (m, 1H), 7.82- |
| | pyrazolo[3,4- | | | 7.79 (m, 1H), 7.61-7.56 (m, |
| | d]pyrimidin-4-amine | | | 1H), 3.96-3.90 (m, 1H), 2.67 (s, |
| | | | | 3H), 1.26-1.22 (m, 2H), 1.16- |
| | | | | 1.13(m, 2H) |
| 63 | 1-cyclopropyl-3-(2-(6- | 293.9 | 295.1 | (DMSO-d6) δ = 8.65 (s, 1H), |
| | methylpyridin-3- | | | 8.30-8.26 (m, 2H), 7.77 (d, J = |
| | yl)ethyl)-1H- | | | 8.4 Hz, 1H), 3.79-3.74 (m, 1H), |
| | pyrazolo[3,4- | | | 3.35-3.31 (m, 2H), 3.14-3.10 |
| | d]pyrimidin-4-amine | | | (m, 2H), 2.65 (s, 3H), 1.05-1.04 |
| | | | | (m, 4H) |

### Synthesis Method F: General Procedure Represented by the Preparation of 3-((6-chloropyridin-2-yl)oxy)-1-cyclopropyl-1H-pyrazolo[3,4-d]pyrimidin-4-amine

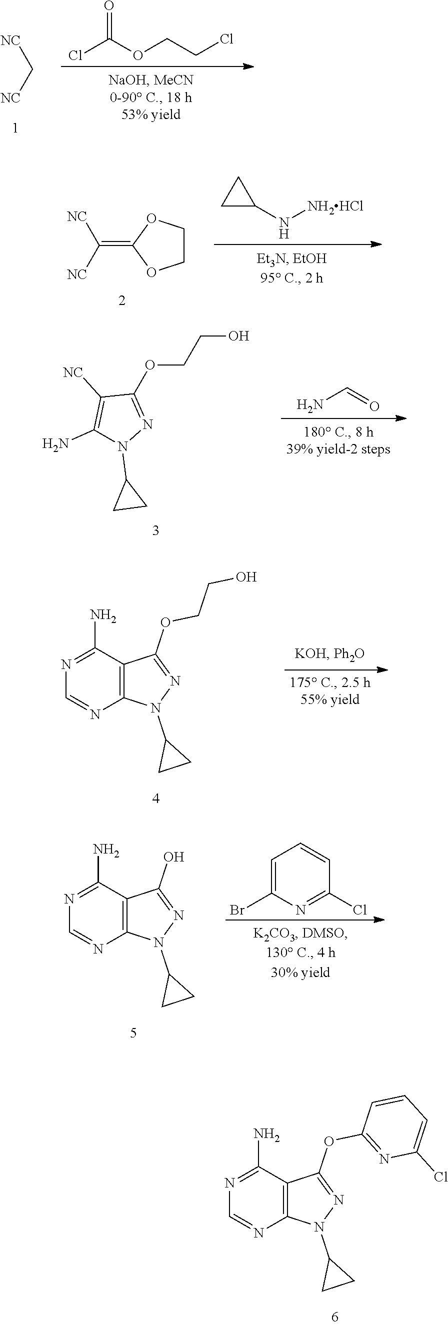

### Step 1

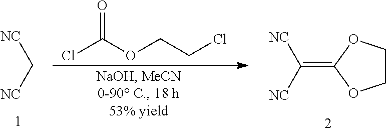

Malononitrile (20.0 g, 302.8 mmol, 1.0 eq) and NaOH (24.2 g, 605.5 mmol, 2.0 eq) were combined in MeCN (500.0 mL), degassed and purged with nitrogen three times, and stirred at 25° C. for about 2 h under nitrogen atmosphere. The reaction mixture was filtered and the solid collected, resuspended in MeCN (500.0 mL) and 2-chloroethyl carbonochloridate (43.3 g, 302.8 mmol, 1.0 eq), diluted in 100 mL MeCN, was added dropwise at 0° C. The reaction was stirred at 90° C. for about 16 h, concentrated under reduced pressure and purified by column chromatography (SiO₂, DCM/MeOH=10/1 to 4/1) to give 22.0 g (53.4% yield) of 2-(1,3-dioxolan-2-ylidene)malononitrile as a light yellow solid.

### Step 2

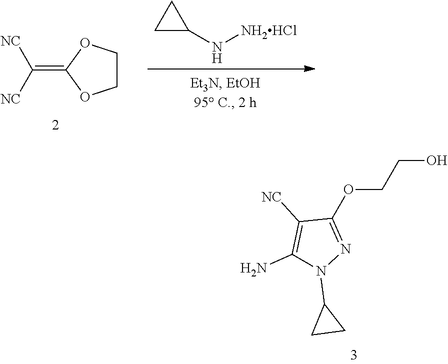

2-(1,3-Dioxolan-2-ylidene)malononitrile (16.0 g, 117.5 mmol, 1.0 eq), cyclopropylhydrazine (20.5 g, 141.1 mmol, 1.2 eq, HCl) and triethylamine (47.6 g, 470.2 mmol, 4.0 eq) were combined in ethanol (200.0 mL) and stirred at 95° C. for about 2 h under nitrogen atmosphere. The reaction mixture was concentrated by rotary evaporator to give 5-amino-1-cyclopropyl-3-(2-hydroxyethoxy)-1H-pyrazole-4-carbonitrile (40 g, crude) as a yellow solid which was used for next step directly.

### Step 3

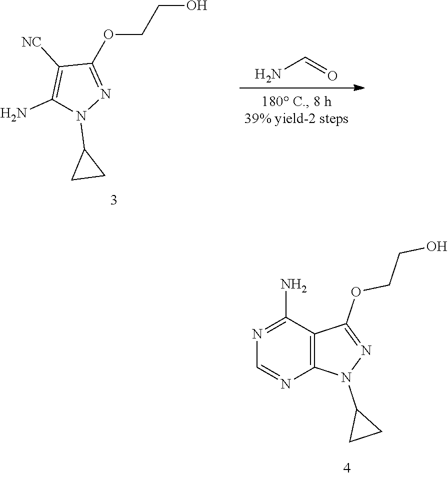

5-Amino-1-cyclopropyl-3-(2-hydroxyethoxy)-1H-pyrazole-4-carbonitrile (30.0 g, crude) was stirred in formamide (150.0 mL) at 180° C. for about 8 h. The reaction mixture was purified by prep-HPLC (condition: neutral) to give 2-((4-amino-1-cyclopropyl-1H-pyrazolo[3,4-d]pyrimidin-3-yl)oxy)ethan-1-ol (9 g, 38.3 mmol) as a yellow solid. ¹H NMR: (400 MHz, DMSO-d₆) δ=8.11 (s, 1H), 7.68 (s, 1H), 6.72 (s, 1H), 5.03 (t, J=6.4 Hz, 1H), 4.20 (t, J=4.4 Hz, 2H), 3.74-3.71 (m, 2H), 3.55-3.51 (m, 1H), 1.07-1.04 (m, 2H), 0.98-0.95 (m, 2H).

### Step 4

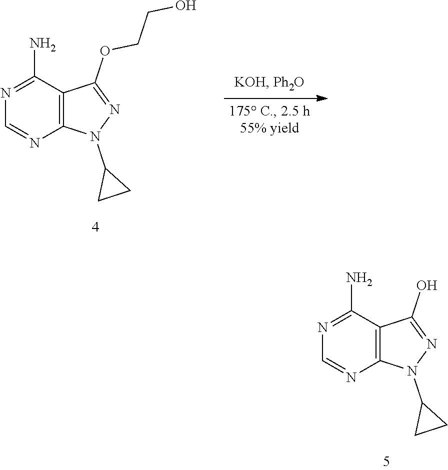 [SMILES: Nc1ncnc2c1c(OCCO)nn2C1CC1 ; Nc1ncnc2c1c(O)nn2C1CC1]

2-((4-Amino-1-cyclopropyl-1H-pyrazolo[3,4-d]pyrimidin-3-yl)oxy)ethan-1-ol (6.0 g, 25.5 mmol, 1.0 eq) and KOH (17.2 g, 306.1 mmol, 12.0 eq) were stirred in diphenyl ether (15.0 mL) at 175° C. for about 2.5 h. The reaction mixture was washed with 30 mL of petroleum ether, filtered and the solid was dissolved in about 15 mL of water. The pH was adjusted to between 6 and 7 with HCl and the newly formed precipitate was collected by filtration and dried under reduced pressure to give 4-amino-1-cyclopropyl-1H-pyrazolo[3,4-d]pyrimidin-3-ol (3.0 g, 55.4% yield, 90% purity) as a white solid used without further purification. ¹H NMR: (400 MHz, DMSO-d₆) δ=11.18 (s, 1H), 8.08 (s, 1H), 7.51-7.37 (m, 1H), 6.61 (s, 1H), 3.47-3.42 (m, 1H), 1.04-1.00 (m, 2H), 0.94-0.91 (m, 2H).

### Step 5

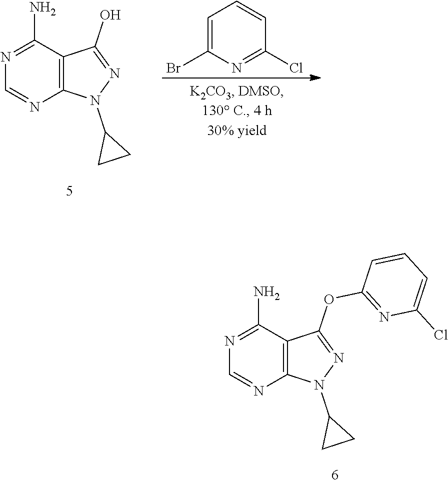 [SMILES: Nc1ncnc2c1c(O)nn2C1CC1 ; Clc1cccc(Br)n1 ; Nc1ncnc2c1c(Oc1cccc(Cl)n1)nn2C1CC1]

4-Amino-1-cyclopropyl-1H-pyrazolo[3,4-d]pyrimidin-3-ol (0.2 g, 1.1 mmol, 1 eq), 2-bromo-6-chloro-pyridine (402.6 mg, 2.1 mmol, 2 eq) and K₂CO₃ (173.5 mg, 1.3 mmol, 1.2 eq) were combined in DMSO (4 mL) was stirred at 130° C. for about 4 h. The mixture was filtered and the filtrate was purified by prep-HPLC (condition: TFA) to give 3-((6-chloropyridin-2-yl)oxy)-1-cyclopropyl-1H-pyrazolo[3,4-d]pyrimidin-4-amine (Compound 29) (97.7 mg, 30.1% yield, 97.5% purity) as a light yellow solid. ¹H NMR: (400 MHz, DMSO-d₆) δ=8.59-8.45 (m, 1H), 8.34 (s, 1H), 7.98 (t, J=8.0 Hz, 1H), 7.37 (d, J=7.6 Hz, 1H), 7.24 (d, J=8.0 Hz, 1H), 3.80-3.75 (m, 1H), 1.08-1.04 (m, 4H). LCMS: (M+H)⁺: 303.1, Rt: 2.356 min. LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS).

The following compounds were prepared in a similar manner as described in method F using different starting materials.

TABLE 5
Compounds Prepared by Method F

| | | | LC/MS | |
|---|---|---|---|---|
| Compound | | | Observed | |
| No. | IUPAC Name | MW | (M + H) | 1H NMR (400 MHz) |
| 30 | 3-((6-chloropyridin-2- | 302.71904 | 303.1 | (DMSO-d6) δ = 8.59- |
| | yl)oxy)-1-cyclopropyl-1H- | | | 8.45 (m, 1H), 8.34 (s, |
| | pyrazolo[3,4-d]pyrimidin- | | | 1H), 7.98 (t, J = 8.0 Hz, |
| | 4-amine | | | 1H), 7.37 (d, J = 7.6 Hz, |
| | | | | 1H), 7.24 (d, J = 8.0 Hz, |
| | | | | 1H), 3.80-3.75 (m, |
| | | | | 1H), 1.08-1.04 (m, 4H) |
| 31 | 3-((6-bromopyridin-2- | 347.17004 | 347.0/349.0 | (DMSO-d6) δ = 8.60 (s, |
| | yl)oxy)-1-cyclopropyl-1H- | | | 1H), 8.35 (s, 1H), 7.87 |
| | pyrazolo[3,4-d]pyrimidin- | | | (t, J = 7.8 Hz, 1H), 7.50 |
| | 4-amine | | | (d, J = 7.6 Hz, 1H), 7.27- |
| | | | | 7.22 (m, 1H), 6.33 (s, |
| | | | | 1H), 3.77 (d, J = 4.0 Hz, |
| | | | | 1H), 1.08-1.03 (m, 4H) |
| 37 | 3-((5-chloropyridin-3- | 302.72 | 303.1 | (DMSO-d6) δ = 8.70 (s, |
| | yl)oxy)-1-cyclopropyl-1H- | | | 1H), 8.48 (s, 1H), 8.20 |
| | pyrazolo[3,4-d]pyrimidin- | | | (m, 1H), 8.10 (s, 1H), |
| | 4-amine | | | 7.82 (s, 1H), 7.14 (s, |
| | | | | 1H), 3.62 (d, J = 4.0 Hz, |
| | | | | 1H), 1.06 (d, J = 2.8 Hz, |
| | | | | 2H), 1.00 (d, J = 5.6 Hz, |
| | | | | 2H) |
| 38 | 1-cyclopropyl-3-(pyridin- | 268.27 | 269.1 | (DMSO-d6) δ = 8.20 (s, |
| | 2-yloxy)-1H-pyrazolo[3,4- | | | 1H), 8.14 (d, J = 3.6 Hz, |
| | d]pyrimidin-4-amine | | | 1H), 7.90 (t, J = 6.8 Hz, |
| | | | | 1H), 7.20 (d, J = 8.0 Hz, |
| | | | | 2H), 3.72-3.66 (m, |
| | | | | 1H), 1.06 (d, J = 3.2 Hz, |
| | | | | 2H), 1.02-0.97 (m, 2H) |
| 39 | 1-cyclopropyl-3-(pyrazin- | 269.26 | 270.1 | (DMSO-d6) δ = 8.65 (s, |
| | 2-yloxy)-1H-pyrazolo[3,4- | | | 1H), 8.42 (d, J = 2.8 Hz, |
| | d]pyrimidin-4-amine | | | 1H), 8.20 (s, 2H), 7.59- |
| | | | | 7.12 (m, 2H), 3.74-3.68 |
| | | | | (m, 1H), 1.09-1.05 (m, |
| | | | | 2H), 1.03-0.98 (m, 2H) |
| 40 | 1-cyclopropyl-3-((6- | 282.30 | 283.1 | (DMSO-d6) δ = 8.19 (s, |
| | methylpyridin-2-yl)oxy)- | | | 1H), 7.77 (t, J = 7.6 Hz, |
| | 1H-pyrazolo[3,4- | | | 1H), 7.06 (d, J = 6.8 Hz, |
| | dlpyrimidin-4-amine | | | 1H), 6.97 (d, J = 8.4 Hz, |
| | | | | 1H), 3.68 (d, J = 4.0 Hz, |
| | | | | 1H), 2.31 (s, 3H), 1.05 |
| | | | | (s, 2H), 1.00 (d, J = 6.8 |
| | | | | Hz, 2H) |
| 41 | 1-cyclopropyl-3-((5- | 286.26 | 287.1 | (DMSO-d6) δ = 8.62 (s, |
| | fluoropyridin-3-yl)oxy)- | | | 1H), 8.46 (d, J = 2.8 Hz, |
| | 1H-pyrazolo[3,4- | | | 1H), 8.20 (s, 1H), 7.95- |
| | d]pyrimidin-4-amine | | | 7.92 (m, 1H), 7.91 (s, |
| | | | | 1H), 7.13 (s, 1H), 3.65- |
| | | | | 3.59 (m, 1H), 1.09-1.05 |
| | | | | (m, 2H), 1.02-0.98 (m, |
| | | | | 2H) |
| 42 | 3-((4-chloropyridin-2- | 302.72 | 303.1 | (DMSO-d6) δ = 8.20 (s, |
| | yl)oxy)-1-cyclopropyl-1H- | | | 1H), 8.12 (d, J = 5.6 Hz, |
| | pyrazolo[3,4-d]pyrimidin- | | | 1H), 7.38 (d, J = 1.6 Hz, |
| | 4-amine | | | 1H), 7.32 (t, J = 3.6 Hz, |
| | | | | 1H), 3.73-3.68 (m, |
| | | | | 1H), 1,09-1.05 (m, |
| | | | | 2H), 1.03-1.00 (m, 2H) |
| 43 | 3-((2-chloropyridin-4- | 302.72 | 303.1 | (DMSO-d6) δ = 8.38 (d, |
| | yl)oxy)-1-cyclopropyl-1H- | | | J = 6.0 Hz, 1H), 8.22 (s, |
| | pyrazolo[3,4-d]pyrimidin- | | | 1H), 7.86 (br s, 1H), |
| | 4-amine | | | 7.54 (d, J = 2.0 Hz, 1H), |
| | | | | 7.43-7.41 (m, 1H 7.14- |
| | | | | 7.02 (m, 1H), 3.72- |
| | | | | 3.67 (m, 1H), 1.13-1.10 |
| | | | | (m, 2H), 1.05-1.02 (m, |
| | | | | 2H) |
| 44 | 1-cyclopropyl-3-((2- | 282.30 | 283 | (DMSO-d6) δ = 8.38 (d, |
| | methylpyridin-4-yl)oxy)- | | | J = 6.0 Hz, 1H), 8.21 (s, |
| | 1H-pyrazolo[3,4- | | | 1H), 7.17 (s, 1H), 7.13 |
| | d]pyrimidin-4-amine | | | (d, J = 5.2 Hz, 1H), 3.70- |
| | | | | 3.67 (m, 1H), 2.45 (s, |
| | | | | 3H), 1.10 (d, J = 3.2 Hz, |
| | | | | 2H), 1.02 (d, J = 5.2 Hz, |
| | | | | 2H) |
| 64 | 3-((5-chloropyridin-3- | 302.07 | 303.1 | (DMSO-d6) δ = 8.70 (s, |
| | yl)oxy)-1-cyclopropyl-1H- | | | 1H), 8.48 (s, 1H), 8.20 |
| | pyrazolo[3,4-d]pyrimidin- | | | (s, 1H), 8.10 (s, 1H), |
| | 4-amine | | | 7.83 (s, 1H), 7.14 (s, |
| | | | | 1H), 3.62 (d, J = 4.0 Hz, |
| | | | | 1H), 1.07 (d, J = 2.4 Hz, |
| | | | | 2H), 1.00 (d, J = 5.6 Hz, |
| | | | | 2H) |
| 66 | 1-cyclopropyl-3-(pyrazin- | 269.1 | 270.1 | (DMSO-d6) δ = 8.65 (s, |
| | 2-yloxy)-1H-pyrazolo[3,4- | | | 1H), 8.43 (d, J = 2.4 Hz, |
| | d]pyrimidin-4-amine | | | 1H), 8.21 (s, 2H), 7.86- |
| | | | | 6.80 (m, 2H), 3.75-3.67 |
| | | | | (m, 1H), 1.10-1.04 (m, |
| | | | | 2H), 1.04-0.98 (m, 2H) |
| 67 | 1-cyclopropyl-3-((6- | 282.12 | 283.1 | (DMSO-d6) δ = 8.19 (s, |
| | methylpyridin-2-yl)oxy)- | | | 1H), 7.78 (t, J = 7.6 Hz, |
| | 1H-pyrazolo[3,4- | | | 1H), 7.07 (d, J = 7.2 Hz, |
| | d]pyrimidin-4-amine | | | 1H), 6.97 (d, J = 8.4 Hz, |
| | | | | 114), 3.68 (d, J = 4.0 Hz, |
| | | | | 1H), 2.32 (s, 3H), 1.05 |
| | | | | (s, 2H), 1.00 (d, J = 7.2 |
| | | | | Hz, 2H) |
| 68 | 1-cyclopropyl-3-((5- | 286.1 | 287.1 | (DMSO-d6) δ = 8.62 (s, |
| | fluoropyridin-3-yl)oxy)- | | | 1H), 8.46 (d, J = 2.4 Hz, |
| | 1H-pyrazolo[3,4- | | | 1H), 8.21 (s, 1H), 7.94 |
| | d]pyrimidin-4-amine | | | (td, J = 2.4, 10.4 Hz, |
| | | | | 1H), 7.88-7.64 (m, |
| | | | | 1H), 7.14 (s, 1H), 3.62 |
| | | | | (tt, J = 3.6, 7.2 Hz, 1H), |
| | | | | 1.11-1.05 (m, 2H), 1.03- |
| | | | | 0.96 (m, 2H) |
| 70 | 3-((2-chloropyridin-4- | 302.07 | 303.1 | (DMSO-d6) δ = 8.38 (d, |
| | yl)oxy)-1-cyclopropyl-1H- | | | J = 6.0 Hz, 1H), 8.22 (s, |
| | pyrazolo[3,4-d]pyrimidin- | | | 1H), 7.86 (s, 1H), 7.55 |
| | 4-amine | | | (d, J = 2.4 Hz, 1H), 7.43 |
| | | | | (dd, J = 2.4, 5.6 Hz, 1H), |
| | | | | 7.33-6.98 (m, 1H), 3.70 |
| | | | | (tt, J = 3.6, 7.2 Hz, 1H), |
| | | | | 1.16-1.09 (m, 2H), 1.07- |
| | | | | 0.99 (m, 2H) |
| 71 | 1-cyclopropyl-3-((2- | 282.12 | 283 | (DMSO-d6) δ = 8.38 (d, |
| | methylpyridin-4-yl)oxy)- | | | J = 6.0 Hz, 1H), 8.21 (s, |
| | 1H-pyrazolo[3,4- | | | 1H), 7.17 (s, 1H), 7.13 |
| | d]pyrimidin-4-amine | | | (d, J = 5.2 Hz, 1H), 3.72- |
| | | | | 3.62 (m, 1H), 2.45 (s, |
| | | | | 3H), 1,10 (d, J = 3.2 Hz, |
| | | | | 2H), 1.02 (d, J = 5.2 Hz, |
| | | | | 2H) |
| 72 | 1-cyclopropyl-3- | 269.1 | 270 | (DMSO-d6) δ = 9.45 (s, |
| | (pyrimidin-2-yloxy)-1H- | | | 1H), 9.16 (s, 1H), 8.67 |
| | pyrazolo[3,4-d]pyrimidin- | | | (d, J = 4.8 Hz, 2H), 8.49 |
| | 4-amine | | | (s, 1H), 7.35 (t, J = 4.8 |
| | | | | Hz, 1H), 7.39-7.32 (m, |
| | | | | 1H), 3.88 (tt, J = 4.0, 7.2 |
| | | | | Hz, 1H), 1.15-1.05 (m, |
| | | | | 4H) |
| 73 | 1-cyclopropyl-3-((2- | 298.12 | 299.1 | (METHANOL-d4) δ = |
| | methoxypyridin-4-yl)oxy)- | | | 8.35 (s, 1H), 8.16 (d, J = |
| | 1H-pyrazolo[3,4- | | | 6.0 Hz, 1H), 7.12-7.11 |
| | d]pyrimidin-4-amine | | | (m, 1H), 6.96 (d, J = 2.0 |
| | | | | Hz, 1H), 3.97 (s, 3H), |
| | | | | 3.89-3.85 (m, 1H), 1.26- |
| | | | | 1.22 (m, 2H), 1.15- |
| | | | | 1.12 (m, 2H) |
| 74 | 1-cyclopropyl-3-((6- | 298.1 | 299.1 | (DMSO-d6) δ = 8.48 (s, |
| | methoxypyridin-2-yl)oxy)- | | | 1H), 8.09 (d, J = 8.0 Hz, |
| | 1H-pyrazolo[3,4- | | | 1H), 7.77-7.73 (m, |
| | d]pyrimidin-4-amine | | | 1H), 6.54 (d, J = 8.0 Hz, |
| | | | | 1H), 3.83 (s, 3H), 3.53 |
| | | | | (s, 1H), 1.11-0.94 (m, |
| | | | | 4H) |
| 75 | 1-cyclopropyl-3-((5- | 336 | 337 | (METHANOL-d4) δ = |
| | (trifluoromethyl)pyridin-3- | | | 9.20 (s, 1H), 8.82 (s, |
| | yl)oxy)-1H-pyrazolo[3,4- | | | 1H), 8.62 (s, 1H), 8.45 |
| | d]pyrimidin-4-amine | | | (s, 1H), 3.43-3.37 (m, |
| | | | | 1H), 1.20-1.06 (m, 4H) |
| 76 | 2-((4-amino-1- | 312.1 | 313.1 | (METHANOL-d4) δ = |
| | cyclopropyl-1H- | | | 8.22-8.21 (m, 2H), 7.73 |
| | pyrazolo[3,4-d]pyrimidin- | | | (s, 1H), 7.66 (d, J = 5.2 |
| | 3-yl)oxy)isonicotinic acid | | | Hz, 1H), 3.65 (tt, J = |
| | | | | 3.6, 7.2 Hz, 1H), 1.22- |
| | | | | 1.17 (m, 2H), 1.09-1.07 |
| | | | | (m, 2H) |
| 77 | 1-cyclopropyl-3-((4- | 336.09 | 337 | (METHANOL-d4) δ = |
| | (trifluoromethyl)pyridin-2- | | | 8.41 (d, J = 5.2 Hz, 1H), |
| | yl)oxy)-1H-pyrazolo[3,4- | | | 8.34 (s, 1H), 7.68 (s, |
| | d]pyrimidin-4-amine | | | 1H), 7.54 (d, J = 5.2 Hz, |
| | | | | 1H), 3.88-3.83 (m, |
| | | | | 1H), 1.26-1.20 (m, |
| | | | | 2H), 1.14-1.13 (m, 2H) |
| 78 | 2-((4-amino-1- | 293.1 | 294.1 | (DMSO-d6) δ = 8.36 (d, |
| | cyclopropyl-1H- | | | J = 5.2 Hz, 1H), 8.30 (s, |
| | pyrazolo[3,4-d]pyrimidin- | | | 1H), 7.80 (s, 1H), 7.66- |
| | 3-yl)oxy)isonicotinonitrile | | | 7.64 (m, 1H), 3.78-3.74 |
| | | | | (m, 1H), 1.07-1.00 (m, |
| | | | | 4H) |
| 79 | 1-cyclopropyl-3-((3,5- | 336.02 | 337 | (METHANOL-d4) δ = |
| | dichloropyridin-2-yl)oxy)- | | | 8.32 (s, 1H), 8.17 (d, J = |
| | 1H-pyrazolo[3,4- | | | 2.8 Hz, 1H), 8.11 (d, J = |
| | d]pyrimidin-4-amine | | | 2.0 Hz, 1H), 3.82 (tt, J = |
| | | | | 3.6, 7.2 Hz, 1H), 1.22- |
| | | | | 1.18 (m, 2H), 1.12-1.08 |
| | | | | (m, 2H) |
| 80 | 1-cyclopropyl-3-((5- | 336 | 337 | (DMSO-d6) δ = 8.58 (s, |
| | (trifluoromethyl)pyridin-2- | | | 1H), 8.30-8.28 (m, |
| | yl)oxy)-1H-pyrazolo[3,4- | | | 1H), 8.22-8.19 (m, |
| | d]pyrimidin-4-amine | | | 1H), 7.39 (d, J = 8.8 Hz, |
| | | | | 1H), 3.75-3.70 (m, |
| | | | | 1H), 1.10-1.05 (m, |
| | | | | 2H), 1.04-1.00 (m, 2H) |
| 81 | 1-cyclopropyl-3-((6- | 337 | 338 | (DMSO-d6) δ = 9.34 (s, |
| | (trifluoromethyl)pyrimidin- | | | 1H), 8.41 (s, 1H), 8.39 |
| | 4-yl)oxy)-1H- | | | (s, 1H), 8.35 (s, 1H), |
| | pyrazolo[3,4-d]pyrimidin- | | | 7.42 (s, 11), 3.51-3.46 |
| | 4-amine | | | (m, 1H), 0.97-0.92 (m, |
| | | | | 2H), 0.87-0.84 (m, 2H) |
| 82 | 1-cyclopropyl-3-((4- | 286.1 | 287.1 | (DMSO-d6) δ = 9.78 (s, |
| | fluoropyridin-2-yl)oxy)- | | | 1H), 9.12 (s, 1H), 8.42 |
| | 1H-pyrazolo[3,4- | | | (s, 1H), 8.23 (d, J = 6.0 |
| | d]pyrimidin-4-amine | | | Hz, 1H), 7.38-7.37 (m, |
| | | | | 1H), 7.22-7.18 (m, |
| | | | | 1H), 3.81-3.75 (m, |
| | | | | 1H), 1.15-1.12 (m, |
| | | | | 2H), 1.09-1.06 (m, 2H) |
| 83 | 1-cyclopropyl-3-((4- | 282.1 | 283.1 | (DMSO-d6) δ = 8.38 (s, |
| | methylpyridin-2-yl)oxy)- | | | 1H), 8.01-8.00 (m, |
| | 1H-pyrazolo[3,4- | | | 1H), 7.11-7.06 (m, |
| | d]pyrimidin-4-amine | | | 2H), 3.80-3.71 (m, |
| | | | | 1H), 2.37 (s, 3H), 1.13- |
| | | | | 1.03 (m, 4H) |
| 84 | 1-cyclopropyl-3-((4- | 337 | 338 | (DMSO-d6) δ = 9.02 (d, |
| | (trifluoromethyl)pyrimidin- | | | J = 4.8, 1H), 8.34 (s, |
| | 2-yl)oxy)-1H- | | | 1H), 7.89 (d, J = 4.8 Hz, |
| | pyrazolo[3,4-d]pyrimidin- | | | 1H), 3.85-3.79 (m, |
| | 4-amine | | | 1H), 1.11-1.05 (m, 4H) |
| 85 | ethyl 2-((4-amino-1- | 340.1 | 341.1 | (DMSO-d6) δ = 8.34- |
| | cyclopropyl-1H- | | | 8.33 (m, 2H), 7.64-7.63 |
| | pyrazolo[3,4-d]pyrimidin- | | | (m, 2H), 4.40 (q, J = 7.2 |
| | 3-yl)oxy)isonicotinate | | | Hz, 2H), 3.81-3.77 (m, |
| | | | | 1H), 1.36 (t, J = 7.2 Hz, |
| | | | | 3H), 1.09-1.04 (m, 4H) |
| 86 | (2-((4-amino-1- | 298.1 | 299.1 | (METHANOL-d4) δ = |
| | cyclopropyl-1H- | | | 8.22 (s, 1H), 8.12 (d, J = |
| | pyrazole[3,4-d]pyrimidin- | | | 5.2 Hz, 1H), 7.37 (s, |
| | 3-yl)oxy)pyridin-4- | | | 1H), 7.21 (d, J = 5.2 Hz, |
| | yl)methanol | | | 1H), 4.71 (s, 2H), 3.67- |
| | | | | 3.62 (m, 1H) 1.21-1.16 |
| | | | | (m, 2H), 1,09-1.02 (m, |
| | | | | 2H) |
| 87 | 1-cyclopropyl-3-((5- | 282.1 | 283.1 | (METHANOL-d4) δ = |
| | methylpyridin-2-yl)oxy)- | | | 8.34 (s, 1H), 8.05 (s, |
| | 1H-pyrazole[3,4- | | | 1H), 7.81-7.79 (m, |
| | d]pyrimidin-4-amine | | | 1H), 7.34 (d, J = 8.4 Hz, |
| | | | | 1H), 3.85-3.79 (m, |
| | | | | 1H), 2.35 (s, 3H), 1.24- |
| | | | | 1.19 (m, 2H), 1.12-1.08 |
| | | | | (m, 2H) |
| 88 | 1-cyclopropyl-3-((4- | 298.1 | 299.1 | (METHANOL-d4) δ = |
| | methoxypyridin-2-yl)oxy)- | | | 8.34 (s, 1H), 8.02 (d, J = |
| | 1H-pyrazole[3,4- | | | 6.0 Hz, 1H), 7.00 (d, J = |
| | d]pyrimidin-4-amine | | | 2.0 Hz, 1H), 6.89-6.87 |
| | | | | (m, 1H), 3.95 (s, 3H), |
| | | | | 3.88-3.84 (m, 1H), 1.26- |
| | | | | 1.22 (m, 2H), 1.15- |
| | | | | 1.10 (m, 2H) |
| 89 | 3-((4-chloro-5- | 320 | 321 | (METHANOL-d4) δ = |
| | fluoropyridin-2-yl)oxy)-1- | | | 8.44 (d, J = 2.0 Hz, 1H), |
| | cyclopropyl-1H- | | | 8.34 (s, 1H), 7.64 (d, J = |
| | pyrazolo[3,4-d]pyrimidin- | | | 5.6 Hz, 1H), 3.81-3.75 |
| | 4-amine | | | (m, 1H), 1.18-1.16 (m, |
| | | | | 2H), 1.11-1.09 (m, 2H) |
| 90 | 3-((6-chloropyrimidin-4- | 303 | 304 | (METHANOL-d4) δ = |
| | yl)oxy)-1-cyclopropyl-1H- | | | 8.62 (s, 1H), 8.35 (s, |
| | pyrazolo[3,4-d]pyrimidin- | | | 1H), 7.52 (s, 1H), 3.91- |
| | 4-amine | | | 3.86 (m, 1H), 1.26-1.23 |
| | | | | (m, 2H), 1.17-1.14 (m, |
| | | | | 2H) |
| 91 | 3-((2-chloropyrimidin-4- | 303 | 304 | (DMSO-d6) δ = 8.72 (d, |
| | yl)oxy)-1-cyclopropyl-1H- | | | J = 6.4 Hz, 1H), 8.37 (s, |
| | pyrazolo[3,4-d]pyrimidin- | | | 1H), 7.38 (d, J = 5.6 Hz, |
| | 4-amine | | | 1H), 3.86-3.80 (m, |
| | | | | 1H), 1.11-1.09 (m, |
| | | | | 2H), 1.08-1.06 (m, 2H) |
| 92 | 1-cyclopropyl-3-((5- | 337 | 338 | (METHANOL-d4) δ = |
| | (trifluoromethyl)pyridazin- | | | 9.42 (s, 1H), 8.38 (s, |
| | 3-yl)oxy)-1H- | | | 1H), 8.12 (s, 1H), 3.92- |
| | pyrazolo[3,4-d]pyrimidin- | | | 3.86 (m, 1H), 1.28-1.22 |
| | 4-amine | | | (m, 2H), 1.18-1.13 (m, |
| | | | | 2H) |
| 93 | 3-((6-chloropyridazin-4- | 303 | 304 | (DMSO-d6) δ = 9.42 (d, |
| | yl)oxy)-1-cyclopropyl-1H- | | | J = 2.8 Hz, 1H), 8.36 (s, |
| | pyrazolo[3,4-d]pyrimidin- | | | 1H), 7.93 (d, J = 2.4 Hz, |
| | 4-amine | | | 1H), 3.79-3.74 (m, |
| | | | | 1H), 1.16-1.14 (m, |
| | | | | 2H), 1.09-1.06 (m, 2H) |
| 94 | 1-cyclopropyl-3-((5- | 286 | 287.1 | (DMSO-d6) δ = 8.32 (s, |
| | fluoropyridin-2-yl)oxy)- | | | 1H), 8.17 (d, J = 2.8 Hz, |
| | 1H-pyrazolo[3,4- | | | 1H), 7.92-7.88 (m, |
| | d]pyrimidin-4-amine | | | 1H), 7.32-7.29 (m, |
| | | | | 1H), 3.77-3.71 (m, |
| | | | | 1H), 1.08-1.02 (m, 4H) |
| 95 | 1-cyclopropyl-3-((3- | 286 | 287 | (DMSO-d6) δ = 8.36 (s, |
| | fluoropyridin-2-yl)oxy)- | | | 1H), 7.96-7.89 (m, |
| | 1H-pyrazolo[3,4- | | | 2H), 7.31-7.29 (m, |
| | d]pyrimidin-4-amine | | | 1H), 3.79-3.74 (m, |
| | | | | 1H), 1.10-1.03 (m, 4H) |
| 96 | 2-((4-amino-1- | 311 | 312 | (METHANOL-d4) δ = |
| | cyclopropyl-1H- | | | 8.77 (d, J = 2.4 Hz, 1H), |
| | pyrazolo[3,4-d]pyrimidin- | | | 8.34 (s, 1H), 8.15 (d, J = |
| | 3-yl)oxy)-5- | | | 6.4 Hz, 1H), 3.81-3.75 |
| | fluoroisonicotinonitrile | | | (m, 1H), 1.18-1.17 (m, |
| | | | | 2H), 1.11-1.10 (m, 2H) |
| 97 | 1-cyclopropyl-3-((6 | 336.09 | 337.1 | 1H NMR (400 MHz, |
| | (trifluoromethyl)pyridin-2- | | | DMSO-d6) δ = 8.33 (s, |
| | yl)oxy)-1H-pyrazolo[3,4- | | | 1H), 8.20 (m, J = 9.2 Hz, |
| | d]pyrimidin-4-amine | | | 1H), 7.75 (d, J = 7.6 Hz, |
| | | | | 1H), 7.54 (d, J = 8.4 Hz, |
| | | | | 1H), 3.81-3.75 (m, |
| | | | | 1H), 1.10-1.01 (m, 4H) |
| 98 | 1-cyclopropyl-3-((2- | 336.09 | 337.1 | 1H NMR (400 MHz, |
| | (trifluoromethyl)pyridin-4- | | | DMSO-d6) δ = 8.76 (d, |
| | yl)oxy)-1H-pyrazolo[3,4- | | | J = 5.6 Hz, 1H), 8.34 (s, |
| | d]pyrimidin-4-amine | | | 1H), 7.98 (d, J = 2.4 Hz, |
| | | | | 1H), 7.77-7.75 (m, |
| | | | | 1H), 3.80-3.75 (m, |
| | | | | 1H), 1.18-1.04 (m, 4H) |

### Synthesis Method G: General Procedure Represented by the Preparation of 3-((1H-indol-3-yl)methyl)-1-cyclopropyl-1H-pyrazolo[3,4-d]pyrimidin-4-amine

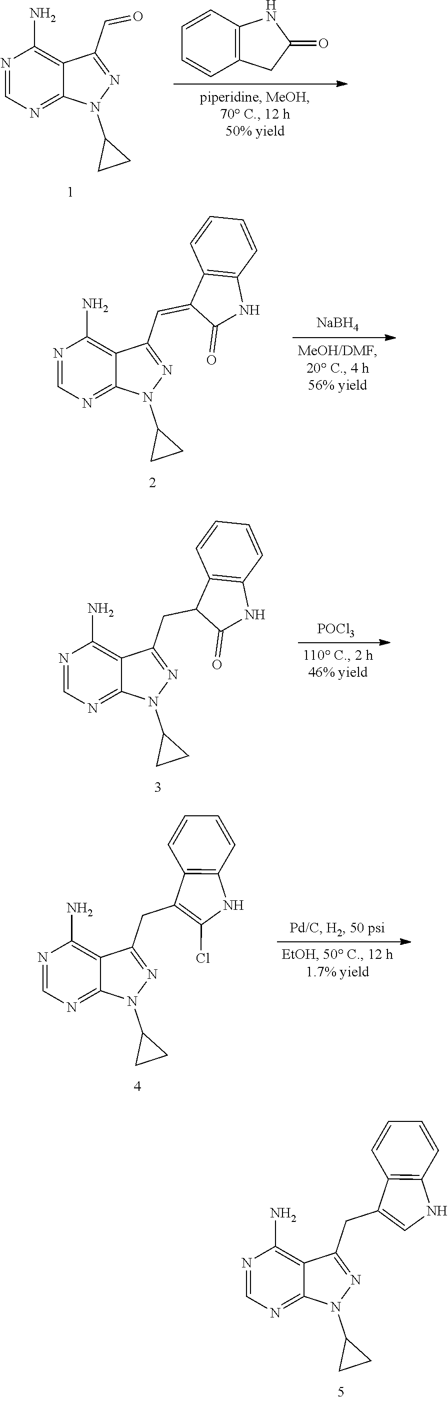

### Step 1

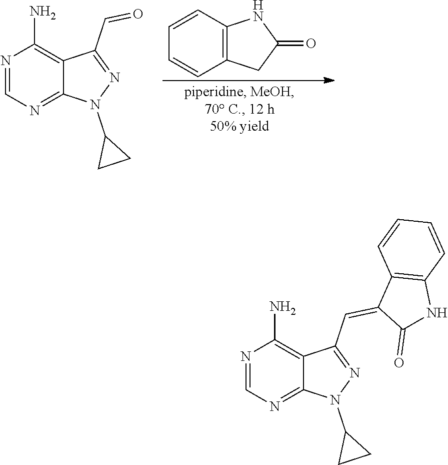

4-Amino-1-cyclopropyl-1H-pyrazolo[3,4-d]pyrimidine-3-carbaldehyde (100.0 mg, 492.1 μmol, 1.0 eq), indolin-2-one (65.5 mg, 492.1 μmol, 1.0 eq), piperidine (431.1 mg, 5.0 mmol, 0.5 mL, 10.2 eq) and MeOH (20.0 mL) were combined and degassed and purged with N₂ three times, and stirred at 70° C. for 12 h under N₂ atmosphere. The mixture was filtered and the filter cake was dried under reduced pressure to give 3-((4-amino-1-cyclopropyl-1H-pyrazolo[3,4-d]pyrimidin-3-yl)methylene)indolin-2-one (80.0 mg, 246.2 μmol, 50.0% yield, 98.7% purity) as a yellow solid without further purification. ¹H NMR: (400 MHz, DMSO-d₆) δ=10.60 (s, 1H), 8.68 (d, J=7.6 Hz, 1H), 8.29 (s, 1H), 7.74 (s, 1H), 7.57 (s, 2H), 7.29 (t, J=7.6 Hz, 1H), 7.00 (t, J=7.6 Hz, 1H), 6.88 (d, J=7.6 Hz, 1H), 4.08 (tt, J=3.6, 7.6 Hz, 1H), 3.37 (s, 1H), 1.33-1.28 (m, 2H), 1.23-1.17 (m, 2H). LCMS: (M+H)⁺: 319.1, Rt: 2.232 min. LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS).)

### Step 2

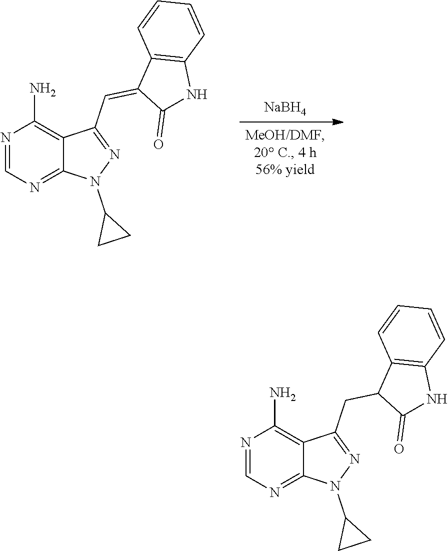 [SMILES: Nc1ncnc2c1c(/C=C1\C(=O)Nc3ccccc31)nn2C1CC1 ; "NaBH4" ; Nc1ncnc2c1c(CC1C(=O)Nc3ccccc31)nn2C1CC1]

To a solution of 3-((4-amino-1-cyclopropyl-1H-pyrazolo[3,4-d]pyrimidin-3-yl)methylene)indolin-2-one (0.5 g, 1.5 mmol, 1.0 eq) in MeOH (10 mL) and DMF (5 mL) was added NaBH₄ (594.2 mg, 15.7 mmol, 10.0 eq). The mixture was stirred at 20° C. for 4 h, quenched with water (10 mL), filtered and the filter cake was dried under reduced pressure to give a residue, which was purified by prep-HPLC (condition: TFA) to give 3-((4-amino-1-cyclopropyl-1H-pyrazolo[3,4-d]pyrimidin-3-yl)methyl)indolin-2-one (0.3 g, 889.6 μmol, 56.6% yield, 95% purity) as a white solid. ¹H NMR: (400 MHz, METHANOL-da) δ=8.27 (s, 1H), 7.22-7.16 (m, 1H), 7.10 (d, J=7.6 Hz, 1H), 6.97-6.91 (m, 1H), 6.89 (d, J=7.6 Hz, 1H), 3.97 (t, J=5.6 Hz, 1H), 3.91-3.84 (m, 1H), 3.76-3.68 (m, 1H), 3.56-3.47 (m, 1H), 1.16-1.08 (m, 1H), 1.07-0.99 (m, 3H). LCMS: (M+H)⁺: 321.1, Rt: 2.205 min. LC/MS (The gradient was 1-90% B in 3.4 min, 90-100% B in 0.45 min, 100-1% B in 0.01 min, and then held at 1% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C₁₋₈ column (5 μm particles). Detection methods are diode array (DAD) and evaporative light scattering (ELSD) detection as well as positive electrospray ionization (MS).

### Step 3

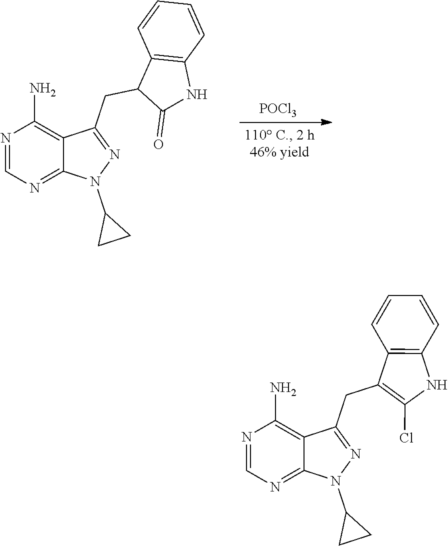

A mixture of 3-((4-amino-1-cyclopropyl-1H-pyrazolo[3,4-d]pyrimidin-3-yl)methyl)indolin-2-one (0.2 g, 624.3 μmol, 1.0 eq) in POCl₃ (8.2 g, 53.8 mmol, 5.0 mL, 86.1 eq) was degassed and purged with N₂ three times, stirred at 110° C. for 2 h under N₂ atmosphere and concentrated under reduced pressure. The residue was quenched with MeOH (2 mL) and purified by prep-HPLC (condition: TFA) to give 3-((2-chloro-1H-indol-3-yl)methyl)-1-cyclopropyl-1H-pyrazolo[3,4-d]pyrimidin-4-amine (0.1 g, 291.3 μmol, 46.6% yield, 98.7% purity) as a yellow solid. ¹H NMR: (400 MHz, CDCl₃) δ=7.44-7.39 (m, 2H), 7.35 (t, J=7.2 Hz, 2H), 7.32-7.27 (m, 1H), 4.67 (s, 2H), 4.61 (s, 2H), 4.47 (s, 2H), 3.12-3.04 (m, 1H), 1.16-1.05 (m, 4H). LCMS: (M+H)⁺: 339.1, Rt: 2.206 min. LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-column (5 μm particles). Detection methods are diode array (DAD) and evaporative light scattering (ELSD) detection as well as positive electrospray ionization (MS).

### Step 4

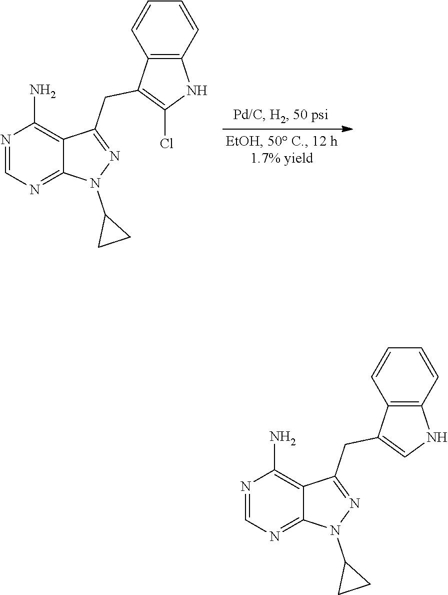 [SMILES: Nc1ncnc2c1c(Cc1c(Cl)[nH]c3ccccc13)nn2C1CC1 ; Nc1ncnc2c1c(Cc1c[nH]c3ccccc13)nn2C1CC1]

To a solution of 3-((2-chloro-1H-indol-3-yl)methyl)-1-cyclopropyl-1H-pyrazolo[3,4-d]pyrimidin-4-amine (0.1 g, 295.1 μmol, 1.0 eq) in EtOH (10 mL) was added Pd/C (10%, 0.1 g). The suspension was degassed and purged with H₂ three times and stirred under H₂ (50 Psi) at 20° C. for 12 h. The mixture was filtered over celite and the filtrate concentrated under reduced pressure to give a residue, which was purified by prep-HPLC (neutral condition) to afford 3-((1H-indol-3-yl)methyl)-1-cyclopropyl-1H-pyrazolo[3,4-d]pyrimidin-4-amine (Compound 32) (1.7 mg, 5.0 μmol, 1.7% yield, 91% purity) as a white solid. ¹H NMR: (400 MHz, METHANOL-d₄) δ=8.13 (s, 1H), 7.37 (dd, J=8.0, 12.0 Hz, 2H), 7.13-7.06 (m, 2H), 6.98-6.92 (m, 1H), 4.39 (s, 2H), 3.70 (tt, J=3.6, 7.2 Hz, 1H), 1.31-1.22 (m, 2H), 1.19-1.10 (m, 2H). LCMS: (M+H)⁺: 305.1, Rt: 2.063 min. LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS).

The following compounds were prepared in a similar manner as described in method G using different starting materials.

TABLE 6
Compounds Prepared by Method G

| | | | LC/MS | |
|---|---|---|---|---|
| Compound | | | Observed | |
| No. | IUPAC Name | MW | (M + H) | 1H NMR (400 MHz) |
| 33 | (Z)-3-((4-amino-1- | 318.33 | 319.1 | (DMSO-d6) δ = 10.60 (s, 1H), |
| | cyclopropyl-1H- | | | 8.68 (d, J = 7.6 Hz, 1H), 8.29 |
| | pyrazolo[3,4-d] | | | (s, 1H), 7.74 (s, 1H), 7.57 |
| | pyrimidin-3-yl) | | | (s, 2H), 7.29 (t, J = 7.6 Hz, 1H), |
| | methylene)indolin- | | | 7.00 (t, J = 7.6 Hz, 1H), |
| | 2-one | | | 6.88 (d, J = 7.6 Hz, 1H), |
| | | | | 4.08 (tt, J = 3.6, 7.6 Hz, 1H), |
| | | | | 3.37 (s, 1H), 1.33-1.28 (m, 2H), |
| | | | | 1.23-1.17 (m, 2H) |
| 34 | 3-((4-amino-1- | 320.35 | 321.1 | (METHANOL-d4) δ = 8.27 |
| | cyclopropyl-1H- | | | (s, 1H), 7.22-7.16 (m, 1H), |
| | pyrazolo[3,4-d] | | | 7.10 (d, J = 7.6 Hz, 1H), |
| | pyrimidin-3-yl) | | | 6.97-6.91 (m, 1H), |
| | methyl)indolin- | | | 6.89 (d, J = 7.6 Hz, 1H), |
| | 2-one | | | 3.97 (t, J = 5.6 Hz, 1H), |
| | | | | 3.91-3.84 (m, 1H), |
| | | | | 3.76-3.68 (m, 1H), |
| | | | | 3.56-3.47 (m, 1H), |
| | | | | 1.16-1.08 (m, 1H), |
| | | | | 1.07-0.99 (m, 3H) |
| 35 | 3-((2-chloro-1H-indol- | 337.79 | 339.1 | (CHLOROFORM-d) δ = 7.44- |
| | 3-yl)methyl)- | | | 7.39 (m, 2H), 7.35 (t, J = 7.2 Hz, |
| | 1-cyclopropyl- | | | 2H), 7.32-7.27 (m, 1H), |
| | 1H-pyrazolo[3,4-d] | | | 4.67 (s, 2H), 4.61 (s, 2H), |
| | pyrimidin-4-amine | | | 4.47 (s, 2H), 3.12-3.04 (m, 1H), |
| | | | | 1.16-1.05 (m, 4H) |
| 36 | 3-((1H-indol-3-yl) | 304.35 | 305.1 | (METHANOL-d4) δ = 8 (s, 1H), |
| | methyl)-1- | | | 7.37 (dd, J = 8.0, 12.0 Hz, 2H), |
| | cyclopropyl-1H- | | | 7.13-7.06 (m, 2H), |
| | pyrazolo[3,4-d] | | | 6.98-6.92 (m, 1H), |
| | pyrimidin-4-amine | | | 4.39 (s, 2H), |
| | | | | 3.70 (tt, J = 3.6, 7.2 Hz, 1H), |
| | | | | 1.31-1.22 (m, 2H), |
| | | | | 1.19-1.10 (m, 2H) |
| 45 | 1-cyclopropyl-3-((5- | 321.33 | 323.1 | (DMSO-d6) δ = 10.98 (s, 1H), |
| | fluoro-1H-indol-3- | | | 8.29 (s, 1H), 7.34-7.30 (m, 2H), |
| | yl)methyl)-1H- | | | 7.24 (dd, J = 2.4, 10.4 Hz, 1H), |
| | pyrazolo[3,4-d] | | | 6.88 (dt, J = 2.4, 9.2 Hz, 1H), |
| | pyrimidin- | | | 4.37 (s, 2H), 3.84 (tt, J = 3.6, |
| | 4-amine | | | 7.2 Hz, 1H), 1.16-1.17 (m, 2H), |
| | | | | 1.08 (dd, J = 4.8, 6.8 Hz, 2H) |
| 46 | (Z)-3-((4-amino-1- | 336.32 | 337.1 | (DMSO-d6) δ = 8.64 |
| | cyclopropyl-1H- | | | (d, J = 10.0 Hz, 1H), |
| | pyrazolo[3,4-d] | | | 8.31 (s, 1H), |
| | pyrimidin-3-yl) | | | 7.82-7.80 (m, 3H), |
| | methylene)-5- | | | 7.14 (t, J = 8.8 Hz, 1H), |
| | fluoroindolin-2-one | | | 6.84 (dd, J = 4.8, 8.0 Hz, 1H), |
| | | | | 4.08 (s, 1H), 1.28-1.19 |
| | | | | (m, 4H) |
| 47 | 3-((4-amino-1- | 338.34 | 339.1 | (DMSO-d6) δ = 10.48 (s, 1H), |
| | cyclopropyl-1H- | | | 8.29 (s, 1H), 6.95 (s, 2H), |
| | pyrazolo[3,4-d] | | | 6.79 (s, 1H), 3.93 (s, 1H), |
| | pyrimidin-3-yl) | | | 3.82 (s, 1H), 3.63-3.60 (m, 1H), |
| | methyl)-5- | | | 3.49-3.43 (m, 1H), |
| | fluoroindolin-2-one | | | 0.97 (d, J = 7.6 Hz, 3H), |
| | | | | 0.86 (s, 1H) |
| 48 | 3-((2-chloro-5-fluoro- | 355.78 | 357 | (DMSO-d6) δ = 8.32-8.29 (m, 1H), |
| | 1H-indol-3-yl) | | | 7.30-7.26 (m, 1H), |
| | methyl)-1- | | | 7.11 (d, J = 8.0 Hz, 1H), |
| | cyclopropyl-1H- | | | 6.97-6.93 (m, 1H), |
| | pyrazolo[3,4-d] | | | 4.37 (d, J = 4.0 Hz, 2H), |
| | pyrimidin-4-amine | | | 3.72-3.70 (m, 1H), |
| | | | | 1.01-1.00 (m, 4H) |
| 49 | (Z)-3-((4-amino-1- | 319.32 | 320.1 | (DMSO-d6) δ = 11.22 (s, 1H), |
| | cyclopropyl-1H- | | | 8.93 (d, J = 7.2 Hz, 1H), |
| | pyrazolo[3,4-d] | | | 8.28 (s, 1H), 813-8.12 (m, 1H), |
| | pyrimidin-3-yl) | | | 7.89 (s, 1H), 7.64 (s, 2H), |
| | methylene)-1,3- | | | 7.05 (dd, J = 5.2, 7.6 Hz, 1H), |
| | dihydro-2H- | | | 4.06 (dt, J = 3.2, 7.2 Hz, 1H), |
| | pyrrolo[2,3-b] | | | 1.29-1.27 (m, 2H), |
| | pyridin-2-one | | | 1.21-1.18 (m, 2H) |
| 50 | 3-((4-amino-1- | 321.34 | 322.1 | (METHANOL-d4) δ = 8.30 |
| | cyclopropyl-1H- | | | (s, 1H), 8.06 (dd, J = 1.6, 5.6 Hz, |
| | pyrazolo[3,4-d] | | | 1H), 7.54 (dd, J = 1.2, 7.6 Hz, |
| | pyrimidin-3-yl) | | | 1H), 6.97 (dd, J = 5.6, 7.2 Hz, |
| | methyl)-1,3-dihydro- | | | 1H), 3.91 (tt, J = 3.6, 7.2 Hz, |
| | 2H-pyrrolo[2,3-b] | | | 1H), 3.78-3.74 (m, 1H), |
| | pyridin-2-one | | | 3.66-3.62 (m, 1H), |
| | | | | 1.06-1.00 (m, 3H), |
| | | | | 1.00-0.87 (m, 1H) |
| 51 | 3-((2-chloro-1H- | 338.77 | 340.1 | (DMSO-d6) δ = 12.37 (s, 1H), |
| | pyrrolo[2,3-b]pyridin- | | | 8.17 (dd, J = 1.2, 4.8 Hz, 1H), |
| | 3-yl)methyl)-1- | | | 8.14 (s, 1H), 7.65 (dd, J = 1.6, |
| | cyclopropyl- | | | 8.0 Hz, 1H), 7.01 (dd, J = 4.8, |
| | 1H-pyrazolo[3,4-d] | | | 8.0 Hz, 1H), 4.37 (s, 2H), 3.65 |
| | pyrimidin-4-amine | | | (tt, J = 3.6, 7.2 Hz, 1H), |
| | | | | 1.04-0.95 (m, 4H) |
| 99 | 1-cyclopropyl-3-((5- | 322.1 | 323.1 | (DMSO-d6) δ = 10.96 (s, 1H), |
| | fluoro-1H-indol-3- | | | 8.27 (s, 1H), 7.34-7.28 (m, 2H), |
| | yl)methyl)-1H- | | | 7.22 (dd, J = 2.4, 10.4 Hz, 1H), |
| | pyrazolo[3,4-d] | | | 6.87 (dt, J = 2.4, 9.2 Hz, 1H), |
| | pyrimidin-4-amine | | | 4.35 (s, 2H), 3.82 (tt, J = 3.6, |
| | | | | 7.2 Hz, 1H), 1.17-1.11 (m, 2H), |
| | | | | 1.08 (dd, J = 4.8, 6.8 Hz, 2H) |
| 100 | (Z)-3-((4-amino-1- | 336.1 | 337.1 | (DMSO-d6 ) δ = 8.64 |
| | cyclopropyl-1H- | | | (d, J = 10.0 Hz, 1H), 8.31 (s, 1H), |
| | pyrazolo[3,4-d] | | | 7.82-7.80 (m, 3H), |
| | primidin-3-yl) | | | 7.14 (t, J = 8.8 Hz, 1H), |
| | methylene)-5- | | | 6.84 (dd, J = 4.4, 8.0 Hz, 1H), |
| | fluoroindolin-2-one | | | 4.08 (s, 1H), |
| | | | | 1.29-1.19 (m, 4H) |
| 101 | 3-((4-amino-1- | 338.1 | 339.1 | (DMSO-d6) δ =10.48 (s, 1H), |
| | cyclopropyl-1H- | | | 8.29 (s, 1H), 6.95 (s, 2H), 6.79 |
| | pyrazolo[3,4-d] | | | (s, 1H), 3.93 (s, 1H), 3.82 (s, 1H), |
| | primidin-3-yl) | | | 3.64-3.60 (m, 1H), 3.49-3.43 (m, 1H), |
| | methyl)-5 | | | 0.98 (d, J = 7.6 Hz, 3H), 0.86 (s, 1H) |
| | fluoroindolin-2-one | | | |
| 102 | 3-((2 chloro-5- | 356.1 | 357 | (DMSO-d6) δ = 8.32-8.30 |
| | fluoro-1H-indol- | | | (m, 1H), 7.30-7.26 (m, 1H), |
| | 3-yl)methyl)-1- | | | 7.11 (d, J = 10.0 Hz, 1H), |
| | cyclopropyl-1H- | | | 6.97-6.94 (m, 1H), |
| | pyrazolo[3,4-d] | | | 4.37 (d, J = 2.0 Hz, 2H), |
| | pyrimidin-4-amine | | | 3.72-3.71 (m, 1H), |
| | | | | 1.01-1.00 (m, 4H) |
| 103 | (Z)-3-((4-amino-1- | 319.1 | 320.1 | (DMSO-66) δ = 11.22 (s, 1H), |
| | cyclopropyl-1H- | | | 8.93 (d, J= 7.2 Hz, 1H), |
| | pyrazolo[3,4-d] | | | 8.28 (s, 1H), 8.15-8.10 (m, 1H), |
| | pyrimidin-3-yl) | | | 7.89 (s, 1H), 7.64 (s, 2H), |
| | methylene)-1,3- | | | 7.05 (dd, J = 5.2, 7.6 Hz, 1H), |
| | dihydro-2H-pyrrolo | | | 4.06 (dd, J = 3.2, 7.2 Hz, 1H), |
| | [2,3-b]pyridin-2-one | | | 1.33-1.25 (m, 2H), |
| | | | | 1.23-1.16 (m, 2H) |
| 104 | 3-((4-amino-1- | 321.1 | 322.1 | (METHANOL-d4) δ = 8.30 (s, 1H), |
| | cyclopropyl-1H- | | | 8.06 (dd, J = 1.6, 5.6 Hz, 1H), |
| | pyrazolo[3,4-d] | | | 7.54 (dd, J = 1.6, 7.2 Hz, 1H), |
| | pyrimidin-3-yl) | | | 6.97 (dd, J = 5.2, 7.2 Hz, 1H), |
| | methyl)-1,3-dihydro- | | | 3.91 (tt, J = 3.6, 7.2 Hz, 1H), |
| | 2H-pyrolo[2,3-b] | | | 3.78-3.74 (m, 1H), |
| | pyridin-2-one | | | 3.66-3.62 (m, 1H), |
| | | | | 1.06-101 (m, 3H), |
| | | | | 1.00-0.92 (m, 1H) |
| 105 | 3-((2-chloro-1H | 339.1 | 340.1 | (DMSO-66) δ = 12.37 (s, 1H), |
| | pyrrolo[2,3-b] | | | 8.17 (dd, J = 1.6, 4.8 Hz, 1H), |
| | pyridin-3-yl) | | | 8.14 (s, 1H), |
| | methyl)-1- | | | 7.65 (dd, J = 1.6, 8.0 Hz, 1H), |
| | cyclopropyl- | | | 7.01 (dd, 1 = 4.4, 8.0 Hz, 1H), |
| | 1H-pyrazolo[3,4-d] | | | 4.37 (s, 2H), |
| | pyrimidin-4-amine | | | 3.65 (tt, J = 3.6, 7.2 Hz, 1H), |
| | | | | 1.04-0.95 (m, 4H) |
| 106 | 3-((1H-pyrrolo[2,3-b] | 305.1 | 306.1 | (DMSO-d6) δ = 12.08 (s, 1H), |
| | pyridin-3-yl)methyl)- | | | 8.47 (s, 1H), 8.31 (d, J = 4.8 Hz, 1H), |
| | 1-cyclopropyl-1H- | | | 8.21 (d, J = 7.6 Hz, 1H), |
| | pyrazolo[3,4-d] | | | 7.53 (s, 1H), 7.27-7.24 (m, 1H), |
| | pyrimidin- | | | 4.53 (s, 2H), 3.91-3.85 (m, 1H), |
| | 4-amine | | | 1.14-1.09 (m, 4H) |

### Synthesis Method H: General Procedure Represented by the Preparation of 3-((4-chloropyridin-2-yl)oxy)-1-cyclobutyl-1H-pyrazolo[3,4-d]pyrimidin-4-amine

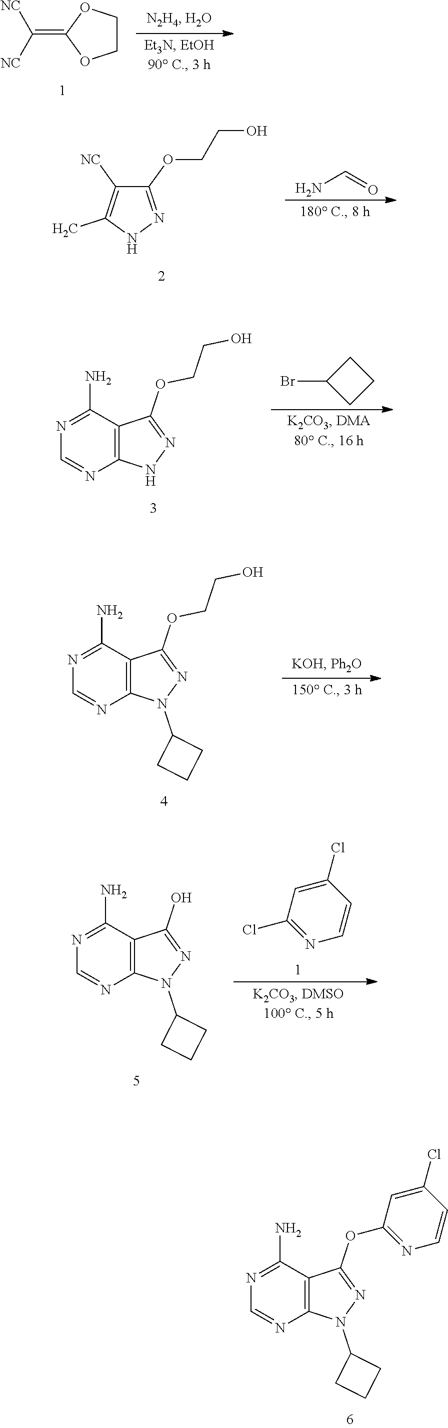

### Step 1. Procedure for preparation of 5-amino-3-(2-hydroxyethoxy)-1H-pyrazole-4-carbonitrile (2)

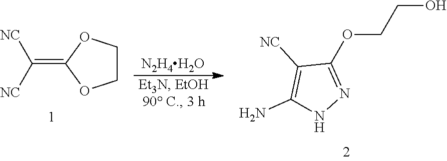

A mixture of 2-(1,3-dioxolan-2-ylidyne)malononitrile (1) (10 g, 73.5 mmol, 1.00 eq) and N₂H₄.H₂O (7.51 g, 147 mmol, 7.29 mL, 2 eq) in EtOH (80 mL) was stirred at 90° C. for 3 h. The mixture was concentrated under reduced pressure to afford 5-amino-3-(2-hydroxyethoxy)-1H-pyrazole-4-carbonitrile (2) (10 g, crude) as a yellow solid.

### Step 2. Procedure for preparation of 2-((4-amino-1H-pyrazolo[3,4-d]pyrimidin-3-yl)oxy)ethan-1-ol (3)

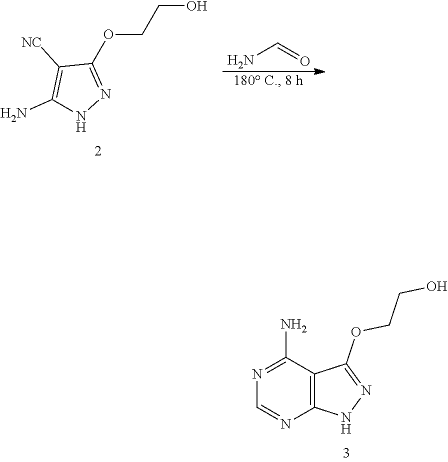

A mixture of 5-amino-3-(2-hydroxyethoxy)-1H-pyrazole-4-carbonitrile (2) (8 g, 47.58 mmol, 1 eq) in formamide (42.9 g, 952 mmol, 37.9 mL, 20 eq) was stirred at 180° C. for 8 h. The mixture was purified by reversed-phase column chromatography (neutral condition) to afford 2-((4-amino-1H-pyrazolo[3,4-d]pyrimidin-3-yl)oxy)ethan-1-ol (3) (6 g, 30.74 mmol, 64.6% yield) as a white solid.

### Step 3. Procedure for preparation of 2-((4-amino-1-cyclobutyl-1H-pyrazolo[3,4-d]pyrimidin-3-yl)oxy)ethan-1-ol (4)

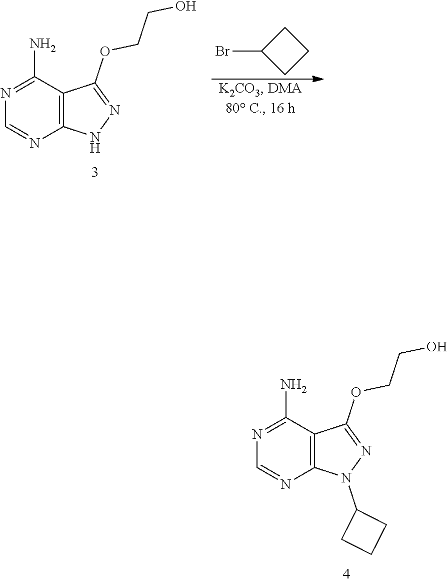

To a solution of 2-((4-amino-1H-pyrazolo[3,4-d]pyrimidin-3-yl)oxy)ethan-1-ol (3) (5 g, 25.6 mmol, 1 eq) in DMA (1 mL) was added K₂CO₃ (8.85 g, 64.0 mmol, 2.5 eq) and bromocyclobutane (6.92 g, 51.2 mmol, 4.84 mL, 2 eq). The mixture was stirred at 80° C. for 16 h, filtered and the filtrate was concentrated under reduced pressure. The residue was purified by silica gel chromatography (Petroleum ether/(Ethyl acetate/EtOH=3/1)=1/0˜1/1) to afford 2-((4-amino-1-cyclobutyl-1H-pyrazolo[3,4-d]pyrimidin-3-yl)oxy)ethan-1-ol (4) (3 g, 12.0 mmol, 47% yield) as a yellow solid.

### Step 4. Procedure for preparation of 4-amino-1-cyclobutyl-1H-pyrazolo[3,4-d]pyrimidin-3-0l (5)

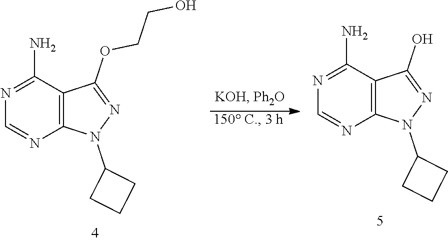 [SMILES: Nc1ncnc2c1c(OCCO)nn2C1CCC1 ; "150° C., 3 h" ; Nc1ncnc2c1c(O)nn2C1CCC1]

Two reactions were carried out in parallel. The mixture of 2-((4-amino-1-cyclobutyl-1H-pyrazolo[3,4-d]pyrimidin-3-yl)oxy)ethan-1-ol (4) (1 g, 4.01 mmol, 1 eq) and KOH (2.93 g, 52.2 mmol, 13 eq) in Ph₂O (10 mL) was stirred at 150° C. for 3 h. The reactions were combined for work up. Petroleum ether (15 mL) was added to the mixture, and the precipitate collected by filtration. The filter cake was collected, diluted with water and the pH was adjusted to between 5 and 6 using IN HCl. The mixture was filtered and the filter cake was dried under reduced pressure to afford 4-amino-1-cyclobutyl-1H-pyrazolo[3,4-d]pyrimidin-3-ol (5) (1.2 g, 5.85 mmol, 72.9% yield) as a light yellow solid. ¹H NMR: (400 MHz, DMSO-d₆) δ=8.02 (s, 1H), 5.11-5.02 (m, 1H), 2.58-2.53 (m, 2H), 2.26-2.24 (m, 2H), 1.78-1.71 (m, 2H).

### Step 5. Procedure for preparation of 3-((4-chloropyridin-2-yl)oxy)-1-cyclobutyl-1H-pyrazolo[3,4-d]pyrimidin-4-amine (6)

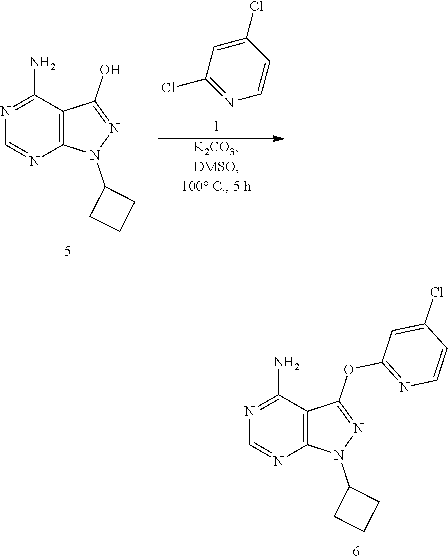 [SMILES: Nc1ncnc2c1c(O)nn2C1CCC1 ; Clc1ccnc(Cl)c1 ; "K2CO3,DMSO,100° C., 5 h" ; Nc1ncnc2c1c(Oc1cc(Cl)ccn1)nn2C1CCC1]

To a solution of 4-amino-1-cyclobutyl-1H-pyrazolo[3,4-d]pyrimidin-3-ol (5) (200 mg, 975 μmol, 1 eq) and K₂CO₃ (269 mg, 1.95 mmol, 2 eq) in DMSO (4 mL) was added 2,4-dichloropyridine (1) (288 mg, 1.95 mmol, 211 μL, 2 eq). The reaction mixture was stirred at 100° C. for about 5 h, filtered, and the filtrate was collected, concentrated and purified by prep-HPLC (TFA condition) to afford 3-((4-chloropyridin-2-yl)oxy)-1-cyclobutyl-1H-pyrazolo[3,4-d]pyrimidin-4-amine (6; Compound 107) (51.7 mg, 120 μmol, 12.3% yield, 100% purity, TFA) as a white solid. LCMS: (M+H)⁺: 317.0, Rt: 2.62 min. LC/MS (The gradient was 1-90% B in 3.4 min, 90-100% B in 0.45 min, 100-1% B in 0.01 min, and then held at 1% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS). NMR: (400 MHz, MeOD-da) δ=8.30 (s, 1H), 8.16 (d, J=5.2 Hz, 1H), 7.52 (s, 1H), 7.34 (d, J=5.6 Hz, 1H), 5.42-5.34 (m, 1H), 2.72-2.66 (m, 2H), 2.47-2.44 (m, 2H), 1.94-1.89 (m, 2H).

TABLE 7
Compounds Prepared by Method H

| | | Expected | LC/MS | |
|---|---|---|---|---|
| Compound | | MW | Observed | |
| No. | IUPAC Name | (M) | (M + H) | 1H NMR (400 MHz) |
| 108 | 3-((4-chloro-5- | 334 | 335 | (DMSO-d6) δ = 8.75 (s, 1H), |
| | fluoropyridin-2-yl) | | | 8.60 (d, J = 2.4 Hz, 1H), |
| | oxy)-1-cyclobutyl- | | | 8.36 (s, 1H), 8.21 (s, 1H), |
| | 1H-pyrazolo[3,4-d] | | | 7.58 (d, J = 6.0 Hz, 1H), |
| | pyrimidin-4-amine | | | 5.29-5.21 (m, 1H), |
| | | | | 2.54-2.53 (m, 2H), |
| | | | | 2.36-2.34 (m, 2H), |
| | | | | 1.84-1.76 (m, 2H) |
| 109 | 1-cyclobutyl-3-((4- | 350.1 | 351.1 | (METHANOL-d4) δ = |
| | (trifluoromethyl) | | | 8.42 (d, J = 5.2 Hz, 1H), |
| | pyridin-2-yl)oxy)-1H- | | | 8.34 (s, 1H), 7.71 (s, 1H), |
| | pyrazolo[3,4-d] | | | 7.54 (d, J = 4.8 Hz, 1H), |
| | pyrimidin-4-amine | | | 5.44-5.35 (m, 1H), |
| | | | | 2.70-2.65 (m, 2H), |
| | | | | 2.47-2.44 (m, 2H), |
| | | | | 1.93-1.88 (m, 2H) |
| 107 | 3-((4-chloropyridin- | 316 | 317 | (METHANOL-d4) δ = |
| | 2-yl)oxy)-1- | | | 8.30 (s, 1H), 8.16 (d, J = |
| | cyclobutyl-1H- | | | 5.2 Hz, 1H), 7.52 (s, 1H), |
| | pyrazolo[3,4-d] | | | 7.34 (d, J = 5.6 Hz, 1H), |
| | pyrimidin- | | | 5.42-5.34 (m, 1H), |
| | 4-amine | | | 2.72-2.66 (m, 2H), |
| | | | | 2.47-2.44 (m, 2H), |
| | | | | 1.94-1.89 (m, 2H) |
| 111 | 2-((4-amino-1- | 307.1 | 308.1 | (METHANOL-d4) δ = |
| | cyclobutyl- | | | 8.38 (d, J = 4.8 Hz, 1H), |
| | 1H-pyrazolo[3,4-d] | | | 8.32 (s, 1H), 7.75 (s, 1H), |
| | pyrimidin-3-yl) | | | 7.57 (d, J = 4.0 Hz, 1H), |
| | oxy)isonicotinonitrile | | | 5.43-5.35 (m, 1H), |
| | | | | 2.70-2.65 (m, 2H), |
| | | | | 2.46-1.42 (m, 2H), |
| | | | | 1.94-1.89 (m, 2H) |
| 112 | 1-cyclobuty1-3-((4- | 312.1 | 313.1 | (METHANOL-d4) δ = |
| | methoxypyridin-2- | | | 8.31 (s, 1H), |
| | yl)oxy)- | | | 8.04 (d, J = 6.0 Hz, 1H), |
| | 1H-pyrazolo[3,4-d] | | | 7.06 (d, J = 2.4 Hz, 1H), |
| | pyrimidin-4-amine | | | 6.90 (dd, J = 2.4, 6.0 Hz, |
| | | | | 1H), 5.43-5.34 (m, 1H), |
| | | | | 3.96 (s, 3H), |
| | | | | 2.72-2.67 (m, 2H), |
| | | | | 2.47-2.44 (m, 2H), |
| | | | | 1.95-1.90 (m, 2H) |

### Alternative synthesis Method H: General Procedure Represented by the Preparation of (trans)-3-(4-amino-3-((4-(trifluoromethyl)pyridin-2-yl)oxy)-1H-pyrazolo[3,4-d]pyrimidin-1-yl)cyclobutan-1-ol

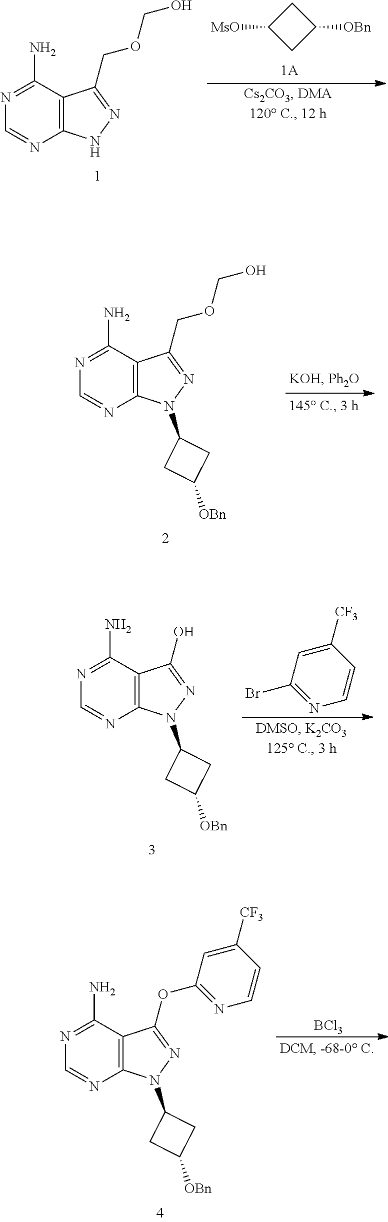

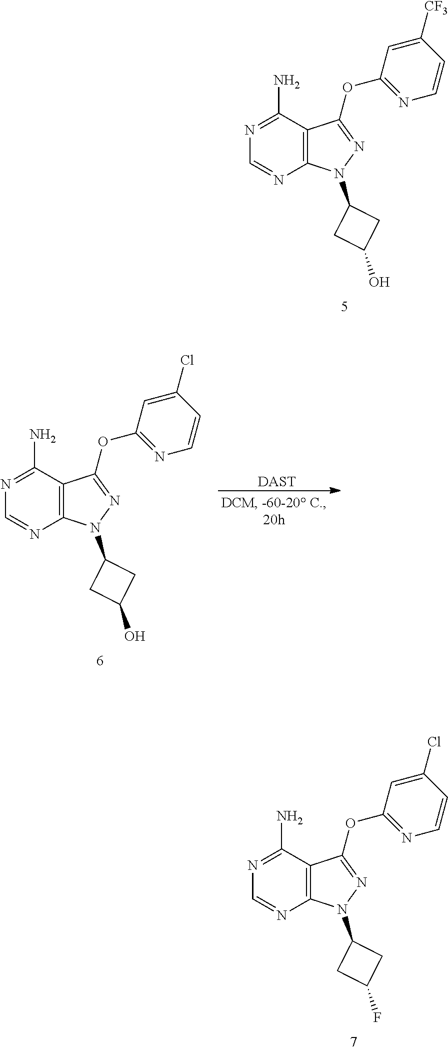

### Step 1. Procedure for preparation of 2-((4-amino-1-((trans)-3-(benzyloxy)cyclobutyl)-1H-pyrazolo[3,4-d]pyrimidin-3-yl)oxy)ethan-1-ol (2)

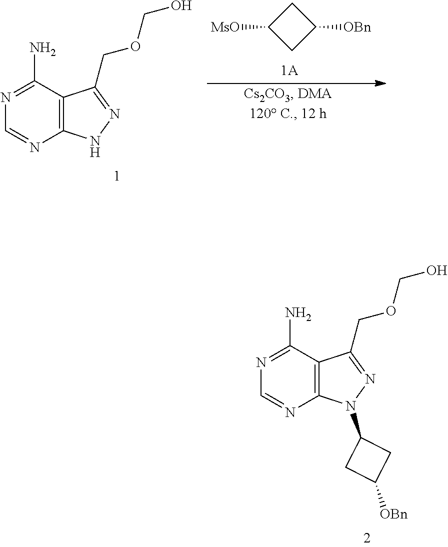 [SMILES: Nc1ncnc2[nH]nc(COCO)c12 ; CS(=O)(=O)O[C@H]1C[C@@H](OCc2ccccc2)C1 ; Nc1ncnc2c1c(COCO)nn2[C@H]1C[C@H](OCc2ccccc2)C1]

To a solution of 2-((4-amino-1H-pyrazolo[3,4-d]pyrimidin-3-yl)oxy)ethan-1-ol (1) (5 g, 25.6 mmol, 1 eq) in DMA (250 mL) was added (cis)-3-(benzyloxy)cyclobutyl methanesulfonate (1A) (8.55 g, 33.3 mmol, 1.3 eq) and Cs₂CO₃ (25.1 g, 76.9 mmol, 3 eq) and then the mixture was stirred at 120° C. for about 12 h. The reaction mixture was filtered and the filtrate concentrated to provide crude product, which was purified by silica gel chromatography (SiO₂, Petroleum ether/Ethyl acetate=20/1 to 0/1) to give 2-((4-amino-1-((trans)-3-(benzyloxy)cyclobutyl)-1H-pyrazolo[3,4-d]pyrimidin-3-yl)oxy)ethan-1-ol (2) (3.2 g, 9.00 mmol, 35.1% yield) as a white solid.

### Step 2. Procedure for preparation of 4-amino-1-((trans)-3-(benzyloxy)cyclobutyl)-1H-pyrazolo[3,4-d]pyrimidin-3-ol (3)

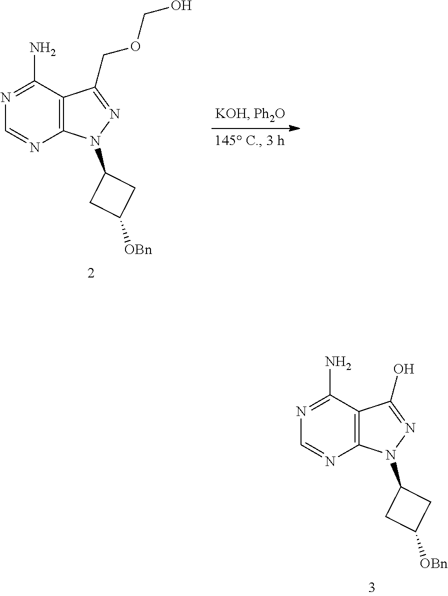 [SMILES: Nc1ncnc2c1c(COCO)nn2[C@H]1C[C@H](OCc2ccccc2)C1 ; Nc1ncnc2c1c(O)nn2[C@H]1C[C@H](OCc2ccccc2)C1]

A mixture of 2-((4-amino-1-((trans)-3-(benzyloxy)cyclobutyl)-1H-pyrazolo[3,4-d]pyrimidin-3-yl)oxy)ethan-1-ol (2) (3.2 g, 9.00 mmol, 1 eq) and KOH (6.06 g, 108.06 mmol, 12 eq) in diphenyl ether (20 mL) was stirred at 145° C. for about 3 h. The reaction mixture was washed with petroleum ether (30 mL) and then the mixture was filtered. The filter cake was collected, dissolved in about 15 mL of water and the pH was adjusted to between 6 and 7 with hydrogen chloride. The precipitate was collected by filtration and the filter cake was dried under vacuum to give 4-amino-1-((trans)-3-(benzyloxy)cyclobutyl)-1H-pyrazolo[3,4-d]pyrimidin-3-ol (3) (2 g, 5.20 mmol, 57.8% yield) as a white solid. ¹H NMR (400 MHz, METHANOL-d4) δ=8.10-8.03 (m, 1H), 7.40-7.24 (m, 5H), 4.53-4.39 (m, 3H), 3.77-3.62 (m, 1H), 2.75 (td, 7=6.4, 13.2 Hz, 2H), 2.58-2.49 (m, 2H).

### Step 3. Procedure for preparation of 1-((trans)-3-(benzyloxy)cyclobutyl)-3-((4-(trifluoromethyl)pyridin-2-yl)oxy)-1H-pyrazolo[3,4-d]pyrimidin-4-amine (4)

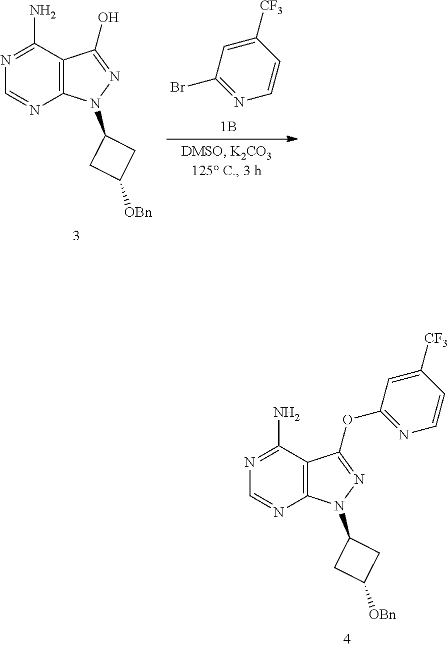 [SMILES: Nc1ncnc2c1c(O)nn2[C@H]1C[C@H](OCc2ccccc2)C1 ; FC(F)(F)c1ccnc(Br)c1 ; Nc1ncnc2c1c(Oc1cc(C(F)(F)F)ccn1)nn2[C@H]1C[C@H](OCc2ccccc2)C1]

To a solution of 4-amino-1-((trans)-3-(benzyloxy)cyclobutyl)-1H-pyrazolo[3,4-d]pyrimidin-3-ol (3) (200 mg, 642 μmol, 1 eq) in DMSO (10 mL) was added 2-bromo-4-(trifluoromethyl)pyridine (IB) (233 mg, 1.28 mmol, 2 eq) and K₂CO₃ (178 mg, 1.28 mmol, eq) and the mixture was stirred at 125° C. for about 3 h. The mixture was filtered and the filtrate was purified by prep-HPLC (TFA condition) to give 1-((trans)-3-(benzyloxy)cyclobutyl)-3-((4-(trifluoromethyl)pyridin-2-yl)oxy)-1H-pyrazolo[3,4-d]pyrimidin-4-amine (4) (150 mg, 263 μmol, 40.9% yield) as a white solid.

### Step 4. Procedure for preparation of (trans)-3-(4-amino-3-((4-(trifluoromethyl)pyridin-2-yl)oxy)-1H-pyrazolo[3,4-d]pyrimidin-1-yl)cyclobutan-1-ol (5)

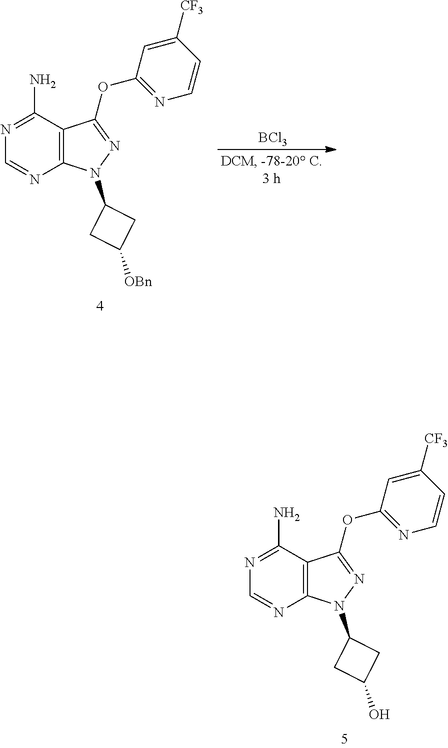 [SMILES: Nc1ncnc2c1c(Oc1cc(C(F)(F)F)ccn1)nn2[C@H]1C[C@H](OCc2ccccc2)C1 ; "BCl3" ; Nc1ncnc2c1c(Oc1cc(C(F)(F)F)ccn1)nn2[C@H]1C[C@H](O)C1]

To a mixture of 1-((trans)-3-(benzyloxy)cyclobutyl)-3-((4-(trifluoromethyl)pyridin-2-yl)oxy)-1H-pyrazolo[3,4-d]pyrimidin-4-amine (4) (50 mg, 110 μmol, 1 eq) in DCM (2 mL) at −78° C. was added BCl₃ (1 M, 876 μL, 8 eq) dropwise. Once addition was complete the mixture was stirred at 20° C. for about 3 h. The reaction was quenched by adding methanol (10 mL) at −60° C. and then the pH was adjusted to 7 using NH₃.H₂O at 0° C. The mixture was filtered and the filtrate was concentrated under reduced pressure to give a residue, which was purified by prep-HPLC (TFA condition) to give (trans)-3-(4-amino-3-((4-(trifluoromethyl)pyridin-2-yl)oxy)-1H-pyrazolo[3,4-d]pyrimidin-1-yl)cyclobutan-1-ol (5; Compound 113) (30 mg, 55% yield, 96.6% purity, TFA) as a white solid. LCMS: (M+H)⁺: 367.0, Rt: 2.344 min. ¹H NMR: (400 MHz, METHANOL-d₄) δ=8.42 (d, J=5.2 Hz, 1H), 8.30 (s, 1H), 7.73 (s, 1H), 7.55 (d, J=5.2 Hz, 1H), 5.58-5.50 (m, 1H), 4.63-4.56 (m, 1H), 2.85-2.77 (m, 2H), 2.54-2.50 (m, 2H).

### Step 5. (Optional) Procedure for preparation of (trans)-3-[(4-chloro-2-pyridyl)oxy]-1-(3-fluorocyclobutyl)pyrazolo[3,4-d]pyrimidin-4-amine (7)

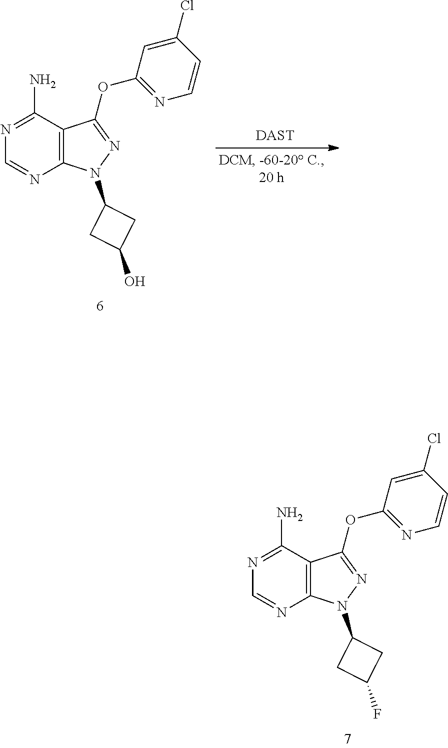

To convert the hydroxycycloalkyl to the fluoroalkyl, DAST may be used as follows. To the mixture of 3-[4-amino-3-[(4-chloro-2-pyridyl)oxy]pyrazolo[3,4-d]pyrimidin-1-yl]cyclobutanol (6) (150 mg, 335.75 umol, 1 eq, TFA) in DCM (5 mL) was added DAST (270.59 mg, 1.68 mmol, 221.80 uL, 5 eq) at −60° C. under nitrogen atmosphere. Then the mixture warmed to 20° C. slowly and stirred for 20 hrs. LCMS showed the reaction was completed. The mixture was poured into 7 mL of water, extracted with dichloromethane (3×10 mL). The combined organic layer was dried over anhydrous sodium sulfate and then concentrated to give the residue. The residue was purified by prep-HPLC (neutral condition) to afford 3-[(4-chloro-2-pyridyl)oxy]-1-(3-fluorocyclobutyl)pyrazolo[3,4-d]pyrimidin-4-amine (7) (60.6 mg, 177.42 umol, 52.84% yield, 98% purity) was obtained as a white solid. LCMS: (M+H)⁺: 335.0, Rt: 2.582 min.

LC/MS (The gradient was 1-90% B in 3.4 min, 90-100% B in 0.45 min, 100-1% B in 0.01 min. and then held at 1% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C₁₋₈ column (5 μm particles). Detection methods are diode array (DAD) and evaporative light scattering (ELSD) detection as well as positive electrospray ionization (MS)). ¹H NMR: (400 MHz, DMSO-d6) δ=8.19 (s, 1H), 8.12 (d, J=5.6 Hz, 1H), 7.41 (d, J=1.6 Hz, 1H), 7.34 (dd, J=1.6, 5.6 Hz, 1H), 5.50-5.45 (m, 1.5H), 5.35-5.31 (m, 0.5H), 2.82-2.67 (m, 4H)

TABLE 8a
Compounds Prepared by Alternative Method H

| | | Expected | LC/MS | |
|---|---|---|---|---|
| Compound | | MW | Observed | |
| No. | IUPAC Name | (M) | (M + H) | 1H NMR (400 MHz) |
| 113 | (trans)-3-(4-amino-3-((4- | 366.1 | 367 | (METHANOL-d4) δ = |
| | (trifluoromethyl)pyridin-2- | | | 8.42 (d, J = 5.2 Hz, 1H), |
| | yl)oxy)-1H-pyrazolo[3,4-d] | | | 8.30 (s, 1H), 7.73 (s, 1H), |
| | pyrimidin-1- | | | 7.55 (d, J = 5.2 Hz, 1H), |
| | yl)cyclobutan-1-ol | | | 5.58-5.50 (m, 1H), |
| | | | | 4.63-4.56 (m, 1H), |
| | | | | 2.85-2.77 (m, 2H), |
| | | | | 2.51 (ddd, J = 4.0, |
| | | | | 8.8, 13.6 Hz, 2H) |
| 115 | 2-((4-amino-1-((trans)-3- | 323.2 | 324.2 | (METHANOL-d4) δ = |
| | hydroxycyclobutyl)-1H- | | | 8.38 (dd, J = 0.8, 5.2 Hz, |
| | pyrazolo[3,4-d] | | | 1H), 8.30 (s, 1H), 7.77-7.74 |
| | pyrimidin-3-yl)oxy) | | | (m, 1H), 7.57 (dd, J = 1.2, |
| | isonicotinonitrile | | | 5.2 Hz, 1H), 5.59-5.52 |
| | | | | (m, 1H), 4.63-4.54 |
| | | | | (m, 1H), 2.87-2.76 (m, 2H), |
| | | | | 2.51 (ddd, J = 4.4, 8.8, |
| | | | | 13.2 Hz, 2H) |
| 116 | (trans)-3-(4-amino-3- | 332 | 333 | (METHANOL-d4) δ = |
| | ((4-chloropyridin- | | | 8.29 (s, 1H), 8.16 (d, J = |
| | 2-yl)oxy)-1H- | | | 5.6 Hz, 1H), 7.53 (d, J = |
| | pyrazolo[3,4-d] | | | 1.6 Hz, 1H), 7.34 (dd, J = |
| | pyrimidin-1-yl) | | | 1.6, 5.6 Hz, 1H), 5.57- |
| | cyclobutan-1-ol | | | 5.50 (m, 1H), 4.64-4.57 |
| | | | | (m, 1H), 2.83-2.78 (m, |
| | | | | 2H), 2.55-2.50 (m, 2H) |
| 117 | (trans)-3-(4-amino-3-((2- | 332 | 333 | (METHANOL-d4) δ = |
| | chloropyridin-4-yl)oxy)- | | | 8.40 (d, J = 5.6 Hz, 1H), |
| | 1H-pyrazolo[3,4-d] | | | 8.30 (s, 1H), 7.64 (d, J = |
| | pyrimidin-1-yl) | | | 2.0 Hz, 1H), 7.52 (dd, J = |
| | cyclobutan-1-ol | | | 2.4, 5.6 Hz, 1H), 5.53 (tt, |
| | | | | J = 5.6, 8.4 Hz, 1H), |
| | | | | 4.66-4.60 (m, 1H), |
| | | | | 2.84-2.77 (m, 2H), |
| | | | | 2.53 (ddd, J = 4.4, 8.8, |
| | | | | 13.6 Hz, 2H) |
| 118 | (trans)-3-(4-amino-3-((4- | 328.1 | 329.1 | (METHANOL-d4) δ = |
| | methoxypyridin-2-yl)oxy)- | | | 8.29 (s, 1H), 8.04 (d, J = |
| | 1H-pyrazolo[3,4-d] | | | 6.0 Hz, 1H), 7.07 (d, J = |
| | pyrimidin-1-yl) | | | 2.4 Hz, 1H), 6.89 (dd, J = |
| | cyclobutan-l-ol | | | 2.4, 5.6 Hz, 1H), 5.56-5.48 |
| | | | | (m, 1H), 4.65-4.59 (m, 1H), |
| | | | | 3.96 (s, 3H), 2.86-2.79 |
| | | | | (m, 2H), 2.55-2.50 |
| | | | | (m, 2H) |

TABLE 8b
Compounds Prepared by Alternative Method H, using (trans)-3-(benzyloxy) cyclobutyl
methanesulfonate as the step 1 starting material to generate the (cis)-3-cyclobutyl alcohols

| | | Expected | LC/MS | |
|---|---|---|---|---|
| Compound | | MW | Observed | |
| No. | IUPAC Name | (M) | (M + H) | 1H NMR (400 MHz) |
| 119 | 2-((4-amino-1-(cis)-3- | 323.1 | 324.1 | (METHANOL-d4) δ = |
| | hydroxycyclobutyl)-1H- | | | 8.40-8.38 (m, 1H), 8.31 |
| | pyrazolo[3,4-d]pyrimidin- | | | (s, 1H), 7.80 (s, 1H), 7.60 |
| | 3-yl)oxy)isonicotinonitrite | | | (dd, J = 1.2, 5.2 Hz, 1H), |
| | | | | 4.98-4.88 (m, 1H), |
| | | | | 4.18-4.11 (m, 1H), |
| | | | | 2.83-2.79 (m, 2H), |
| | | | | 2.62-2.55 (m, 2H) |
| 120 | 2-((4-amino-1-((cis)-3- | 341.1 | 342.1 | (METHANOL-d4) δ = |
| | hydroxycyclobutyl)-1H- | | | 8.34 (d, J = 5.2 Hz, 1H), |
| | pyrazolo[3,4-d] | | | 8.30 (s, 1H), 7.80 (s, 1H), |
| | pyrimidin-3-yl) | | | 7.66 (d, J = 4.8 Hz, 1H), |
| | oxy)isonicotinamide | | | 4.97-4.95 (m, 1H), 4.14 |
| | | | | (q, J = 7.2 Hz, 1H) |
| | | | | 2.83-2.79 (m, 2H), |
| | | | | 2.62-2.55 (m, 2H) |

### Synthesis Method I: General Procedure Represented by the Preparation of 3-(3-chlorophenoxy)-1-cyclobutyl-1H-pyrazolo[3,4-d]pyrimidin-4-amine

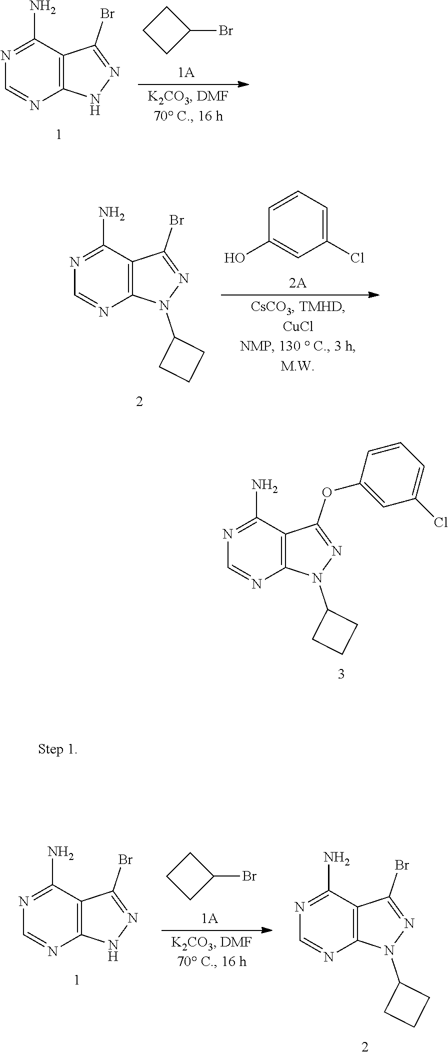

A mixture of 3-bromo-1H-pyrazolo[3,4-d]pyrimidin-4-amine (1) (4 g, 18.7 mmol, 1 eq), bromocyclobutane (1A) (5.05 g, 37.4 mmol, 3.53 mL, 2 eq) and K₂CO₃ (5.17 g, 37.4 mmol, 2 eq) in DMF (10 mL) was stirred at 70° C. for 16 h. The mixture was added into 100 mL H₂O, filtered and the collected solid was dried under reduced pressure to afford 3-bromo-1-cyclobutyl-1H-pyrazolo[3,4-d]pyrimidin-4-amine (2) (3.6 g, 13.4 mmol, 72% yield) as a yellow solid. ¹H NMR: (400 MHz, DMSO-d₆) δ=8.15 (s, 1H), 7.38 (s, 2H), 5.19 (s, 1H), 2.46 (s, 2H), 2.31 (s, 2H), 1.78 (s, 2H).

### Step 2

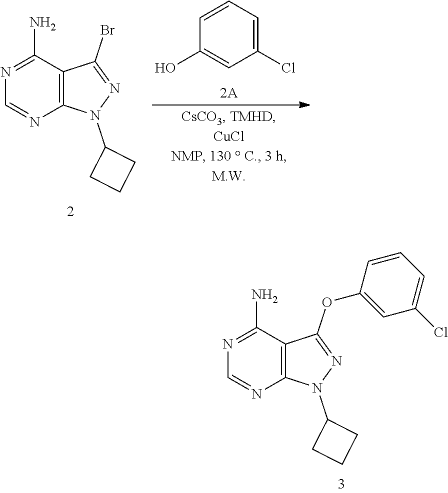 [SMILES: Nc1ncnc2c1c(Br)nn2C1CCC1 ; Oc1cccc(Cl)c1 ; Nc1ncnc2c1c(Oc1cccc(Cl)c1)nn2C1CCC1]

A mixture of 3-bromo-1-cyclobutyl-1H-pyrazolo[3,4-d]pyrimidin-4-amine (2) (100 mg, 373 μmol, 1 eq), 3-chlorophenol (2A) (95.9 mg, 746 μmol, 78.6 μL, 2 eq), Cs₂CO₃ (243 mg, 746 μmol, 2 eq). TMHD (155 mg, 843 μmol, 174 μL, 2.26 eq) and CuCl (38.8 mg, 392 μmol, 9.36 μL, 1-05 eq) in NMP (2 mL) was added to a 5 mL Biotage microwave vial with a Teflon coated stirring bar under nitrogen atmosphere. The vial was scaled and heated at 130° C. for 3 h in a microwave reactor. The mixture was filtered and the filtrate was concentrated under reduced pressure. The resulting residue was purified by prep-HPLC (TFA condition) to afford 3-(3-chlorophenoxy)-1-cyclobutyl-1H-pyrazolo[3,4-d]pyrimidin-4-amine (3; Compound 121) (14.6 mg, 34.0 μmol, 9.1% yield) as a white solid. LCMS: (M+H)⁺: 316.0, Rt: 2.586 min. LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF3CO2H in water, mobile phase B was 0.018% CF3CO2H in CH3CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS). ¹H NMR: (400 MHz, MeOD-d₄) δ=8.28 (s, 1H), 7.52 (s, 1H), 7.47-7.40 (m, 2H), 7.29 (d, J=8.0 Hz, 1H), 5.36-5.27 (m, 1H), 2.67-2.62 (m, 2H), 2.41 (s, 2H), 1.93-1.86 (m, 2H).

TABLE 9
Compounds Prepared by Method I

| | | Expected | LC/MS | |
|---|---|---|---|---|
| Compound | | MW | Observed | |
| No. | IUPAC Name | (M) | (M + H) | 1H NMR (400 MHz) |
| 121 | 3-(3-chlorophenoxy)-1- | 315.09 | 316 | (METHANOL-d4) δ = 8.28 |
| | cyclobutyl-1H-pyrazolo[3,4- | | | (s, 1H), 7.52 (s, 1H), 7.47- |
| | d]pyrimidin-4-amine | | | 7.40 (m, 2H), 7.29 (d, J = |
| | | | | 8.0 Hz, 1H), 5.36-5.27 (m, |
| | | | | 1H), 2.67-2.62 (m, 2H), |
| | | | | 2.41 (s, 2H), 1.93-1.86 (m, |
| | | | | 2H) |
| 123 | 1-cyclobutyl-3-((5- | 312.13 | 313.1 | (METHANOL-d4) δ = 8.35 |
| | methoxypyridin-3-yl)oxy)- | | | (s, 1H), 8.21 (s, 1H), 8.16 |
| | 1H-pyrazolo[3,4- | | | (s, 1H), 7.63 (t, J = 2.0 Hz, |
| | d]pyrimidin-4-amine | | | 1H), 5.27 (q, J = 8.4 Hz, |
| | | | | 1H), 3.92 (s, 3H), 2.70- |
| | | | | 2.57 (m, 2H), 2.46-2.35 |
| | | | | (m, 2H), 1.92-1.82 (m, |
| | | | | 2H) |

### Synthesis Method J: General Procedure Represented by the Preparation of (trans)-3-(4-Amino-3-(3-chlorobenzyl)-1H-pyrazolo[3,4-d]pyrimidin-1-yl)cyclobutan-1-ol

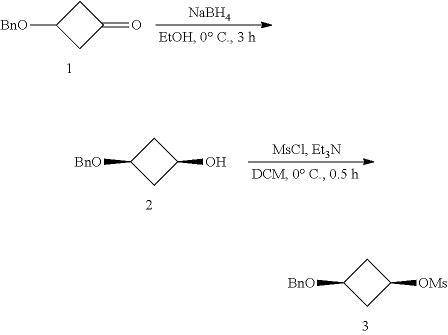

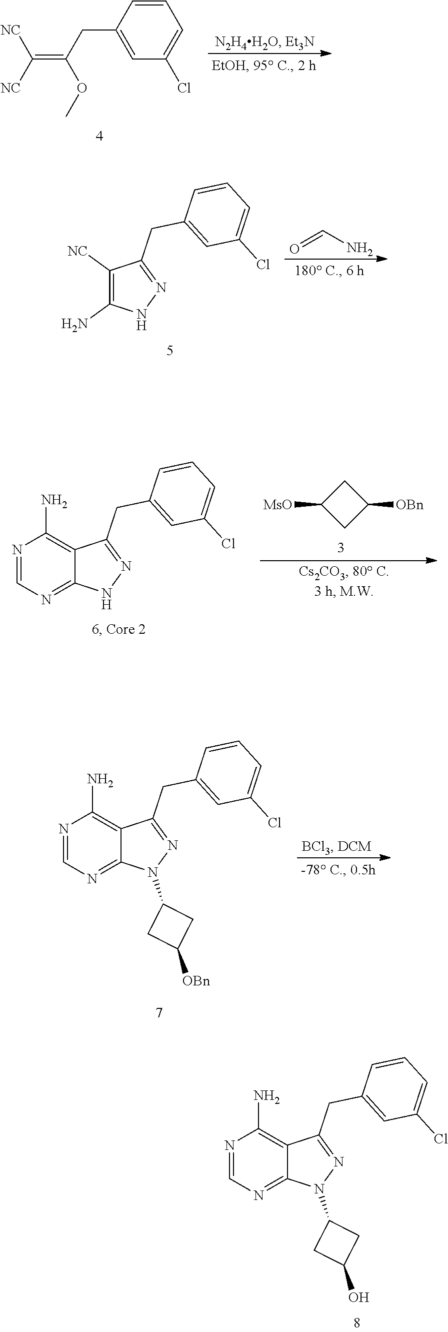

### Step 1

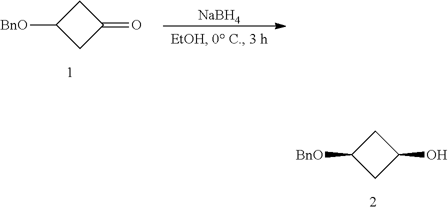 [SMILES: O=C1CC(OCc2ccccc2)C1 ; "EtOH, 0° C., 3 h" ; "NaBH4" ; O[C@H]1C[C@@H](OCc2ccccc2)C1]

To a solution of 3-(benzyloxy)cyclobutan-1-one (1) (2.5 g, 14.19 mmol, 1 eq) in EtOH (20 mL) was added NaBH₄ (536.75 mg, 14.19 mmol, 1 eq) at 0° C. and the reaction was stirred at 0° C. for 3 h. TLC (petroleum ether/ethyl acetate=2/1) showed the reaction was completed. The reaction was quenched by the addition of water (50 mL). The resulting mixture was extracted with Ethyl acetate (50 mL×3). The organic phase was dried over Na₂SO₄, filtered and concentrated under reduced pressure to give (cis)-3-(benzyloxy)cyclobutan-1-ol (2) (2.5 g, crude) as yellow oil.

### Step 2

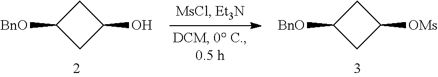 [SMILES: O[C@H]1C[C@@H](OCc2ccccc2)C1 ; CS(=O)(=O)O[C@H]1C[C@@H](OCc2ccccc2)C1]

To a solution of (m)-3-(benzyloxy)cyclobutan-1-ol (2) (400 mg, 2.24 mmol, 1 eq) and Et₃N (656 mg, 6.48 mmol, 902 μL, 1.5 eq) in DCM (7 mL) was added MsCl (742 mg, 6.48 mmol, 502 μL, 1.5 eq) at 0° C. under N₂ atmosphere. The mixture was stirred at 0° C. for 30 min and TLC (Petroleum ether/Ethyl acetate=2/1) showed the reaction was complete. The reaction was quenched by the addition of water (5 mL) and extracted with DCM (3×5 mL). The organic phase was dried over Na₂SO₄, filtered and concentrated under reduced pressure to give (cis)-3-(benzyloxy)cyclobutyl methanesulfonate (3) (550 mg, 2.15 mmol, 95.6% yield) as a yellow oil.

### Step 3

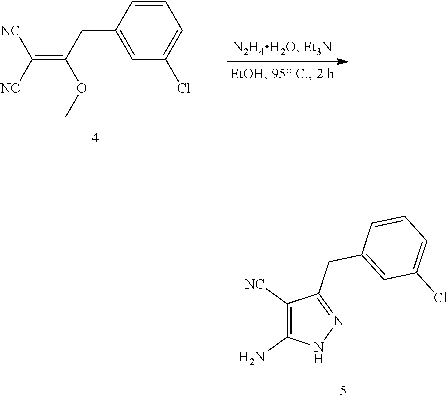 [SMILES: COC(Cc1cccc(Cl)c1)=C(C#N)C#N ; N#Cc1c(Cc2cccc(Cl)c2)n[nH]c1N]

A mixture of 2-(2-(3-chlorophenyl)-1-methoxyethylidene)malononitrile (4) (7.53 g, 32.4 mmol, 1.00 eq), Et₃N (13.1 g, 130 mmol, 18.03 mL, 4.00 eq) and N₂H₄.H₂O (1.78 g, 35.6 mmol, 1.73 mL, 1.1 eq) in EtOH (70 mL) was stirred at 95° C. for 2 h. The mixture was concentrated under reduced pressure to give 5-amino-3-(3-chlorobenzyl)-1H-pyrazole-4-carbonitrile (5) (8 g, crude) as a brown solid which was used to the next step without further purification.

### Step 4

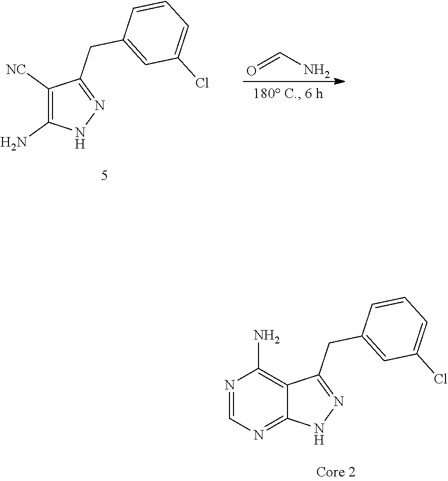

The mixture of 5-amino-3-(3-chlorobenzyl)-1H-pyrazole-4-carbonitrile (5) (8 g, 35.9 mmol, 1.00 eq) and formamide (31.6 g, 702 mmol, 28.0 mL, 19.6 eq) was stirred at 180° C. for 6 h. The mixture was added into water (50 mL) and the precipitate was collected by filtration. The filter cake was washed with water (50 mL) and dried under high vacuum to afford 3-(3-chlorobenzyl)-1H-pyrazolo[3,4-d]pyrimidin-4-amine (6, Core 2) (5 g, 19.3 mmol, 53.6% yield) as a brown solid.

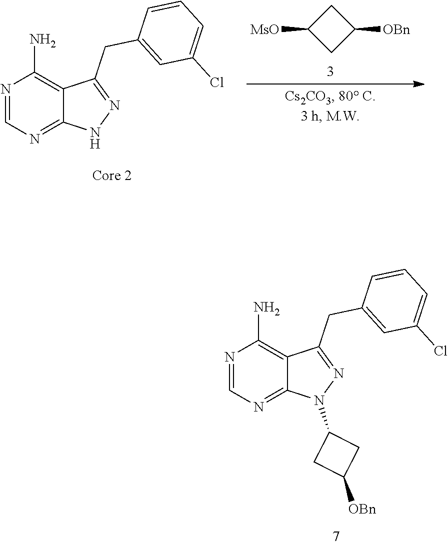 [SMILES: Nc1ncnc2[nH]nc(Cc3cccc(Cl)c3)c12 ; CS(=O)(=O)O[C@H]1C[C@@H](OCc2ccccc2)C1 ; Nc1ncnc2c1c(Cc1cccc(Cl)c1)nn2[C@H]1C[C@H](OCc2ccccc2)C1]

The mixture of 3-(3-chlorobenzyl)-1H-pyrazolo[3,4-d]pyrimidin-4-amine (7, Core_2) (500 mg, 1.93 mmol, 1 eq), (m)-3-(benzyloxy)cyclobutyl methanesulfonate (3) (543 mg, 2.12 mmol, 1.1 eq) and Cs₂CO₃ (1.25 g, 3.85 mmol, 2 eq) in DMF (2 mL) was stirred at 80° C. for 3 h in a microwave reactor. The mixture was filtered and the filtrate was concentrated under reduced pressure. The resulting residue was purified by prep-HPLC (neutral condition) to give 1-((trans)-3-(benzyloxy)cyclobutyl)-3-(3-chlorobenzyl)-1H-pyrazolo[3,4-d]pyrimidin-4-amine (7) (200 mg, 428.67 μmol, 22.26% yield, 90% purity) as a white solid.

### Step 6

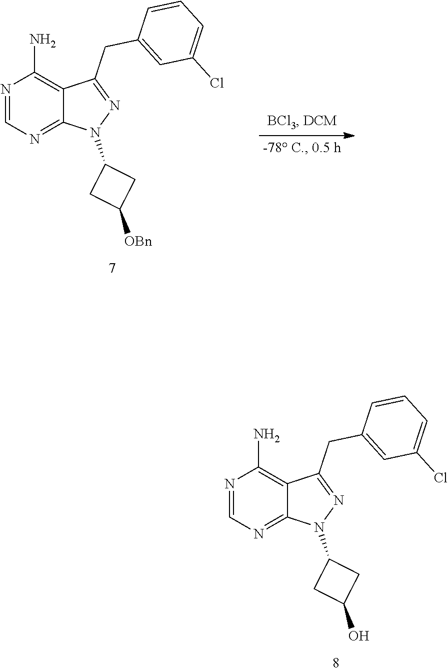 [SMILES: Nc1ncnc2c1c(Cc1cccc(Cl)c1)nn2[C@H]1C[C@H](OCc2ccccc2)C1 ; Nc1ncnc2c1c(Cc1cccc(Cl)c1)nn2[C@H]1C[C@H](O)C1]

To a solution of 1-((trans)-3-(benzyloxy)cyclobutyl)-3-(3-chlorobenzyl)-1H-pyrazolo[3,4-d]pyrimidin-4-amine (7) (100 mg, 238 μmol, 1 eq) in DCM (8 mL) was added trichloroborane (1 M, 1.43 mL, 6 eq) drop-wise at −78° C. under N₂ atmosphere. The mixture was stirred at −78° C. for 30 min and quenched with MeOH (4 mL) at −78° C. The pH was adjusted to 7 by addition of NH₃.H₂O at 0° C. The mixture was filtered and the filtrate concentrated under reduced pressure to give a residue. The residue was purified by prep-HPLC (neutral condition) to give (trans)-3-(4-amino-3-(3-chlorobenzyl)-1H-pyrazolo[3,4-d]pyrimidin-1-yl)cyclobutan-1-ol (8; Compound 124) 20 mg, 60.2 μmol, 25.3% yield, 99.2% purity) as a white solid. LC/MS (M+H)⁺: 330.0, Rt: 2.330 min. LC/MS (The gradient was 1-90% B in 3.4 min, 90-100% B in 0.45 min, 100-1% B in 0.01 min, and then held at 1% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS). ¹H NMR: (400 MHz, METHANOL-d₄) δ=8.13 (s, 1H), 7.29-7.23 (m, 3H), 7.17-7.15 (m, 1H), 5.49-5.41 (m, 1H), 4.71-4.65 (m, 1H), 4.38 (s, 2H), 2.90-2.84 (m, 2H), 2.54-2.48 (m, 2H).

TABLE 10
Compounds Prepared by Method J

| | | Expected | LC/MS | |
|---|---|---|---|---|
| Compound | | MW | Observed | |
| No. | IUPAC Name | (M) | (M + H) | 1H NMR (400 MHz) |
| 124 | (trans)-3-(4-amino-3-(3- | 329.1 | 330 | (METHANOL-d4) δ = |
| | chlorobenzyl)-1H- | | | 8.13 (s, 1H), 7.29-7.23 |
| | pyrazolo[3,4-d]pyrimidin- | | | (m, 3H), 7.17-7.15 (m, |
| | 1-yl)cyclobutan-l-ol | | | 1H), 5.49-5.41 (m, |
| | | | | 1H), 4.71-4.65 (m, |
| | | | | 1H), 4.38 (s, 2H), 2.90- |
| | | | | 2.84 (m, 2H), 2.54- |
| | | | | 2.48 (m, 2H) |
| 125 | (trans)-3-(4-amino-3-(3- | 363.1 | 364.1 | (METHANOL-d4) δ = |
| | (trifluoromethyl)benzyl)- | | | 8.27 (s, 1H), 7.65 (s, |
| | 1H-pyrazolo[3,4- | | | 1H), 7.56-7.48 (m, |
| | d]pyrimidin-1- | | | 3H), 5.56-5.49 (m, |
| | yl)cyclobutan-1-ol | | | 1H), 4.69-4.63 (m, |
| | | | | 1H), 4.54 (s, 2H), 2.85- |
| | | | | 2.81 (m, 2H), 2.55- |
| | | | | 2.51 (m, 2H) |
| 126 | 3-((4-amino-1-((trans)-3- | 320.1 | 321.1 | (METHANOL-d4) δ = |
| | hydroxycyclobutyl)-1H- | | | 8.14 (s, 1H), 7.65 (s, |
| | pyrazolo[3,4-d]pyrimidin- | | | 1H), 7.59 (d, J = 7.6 Hz, |
| | 3-yl)methyl)benzonitrile | | | 1H), 7.57-7.52 (m, |
| | | | | 1H), 7.51-7.45 (m, |
| | | | | 1H, 5.52-5.40 (m, |
| | | | | 1H), 4.72-4.63 (m, |
| | | | | 1H), 4,46 (s, 2H), 2.92- |
| | | | | 2.81 (m, 2H), 2.54- |
| | | | | 2.48 (m, 2H) |
| 127 | (trans)-3-(4-amino-3-(3- | 373.0/375.0 | 374.0/376.0 | (METHANOL-d4) δ = |
| | bromobenzyl)-1H- | | | 8.29 (s, 1H), 7.48 (s, |
| | pyrazolo[3,4-d]pyrimidin- | | | 1H), 7.43-7.37 (m, |
| | 1-yl)cyclobutan-1-ol | | | 1H), 7.24 (d, J = 4.8 Hz, |
| | | | | 2H), 5.55 (tt, J = 5.6, 8.4 |
| | | | | Hz, 1H), 4.71-4.66 (m, |
| | | | | 1H), 4.45 (s, 2H), 2.88- |
| | | | | 2.82 (m, 2H), 2.57- |
| | | | | 2.50 (m, 2H) |
| 128 | (trans)-3-(4-amino-3-(3- | 313.13 | 314.2 | (CDCl3) δ =8.14 (s, |
| | fluorobenzyl)-1H- | | | 1H), 7.43-7.37 (m, |
| | pyrazolo[3,4-d]pyrimidin- | | | 1H), 7.09-7.04 (m, |
| | 1-yl)cyclobutan-1-ol | | | 2H), 6.88 (d, J = 9.2 Hz, |
| | | | | 1H), 5.66-5.59 (m, |
| | | | | 1H), 4.92-4.86 (m, |
| | | | | 1H), 4.36 (s, 2H), 3.04- |
| | | | | 2.97 (m, 2H), 2.67- |
| | | | | 2.62 (m, 2H) |
| 129 | 3-((4-amino-1-((trans)-3- | 338.1 | 339.1 | (METHANOL-d4) δ = |
| | hydroxycyclobutyl)-1H- | | | 8.27 (s, 1H), 7.76-7.73 |
| | pyrazolo[3,4-d]pyrimidin- | | | (m, 2H), 7.47-7.40 (m, |
| | 3-yl)methyl)benzamide | | | 2H), 5.60-5.50 (m, |
| | | | | 1H), 4.72-4.66 (m, |
| | | | | 1H), 4.52 (s, 2H), 2.89- |
| | | | | 2.86 (m, 2H), 2.58- |
| | | | | 2.50 (m, 2H) |

### Synthesis Method K: General Procedure Represented by the Preparation of (cis)-3-(4-amino-3-(3-chlorobenzyl)-1H-pyrazolo[3,4-d]pyrimidin-1-yl)cyclobutan-1-ol

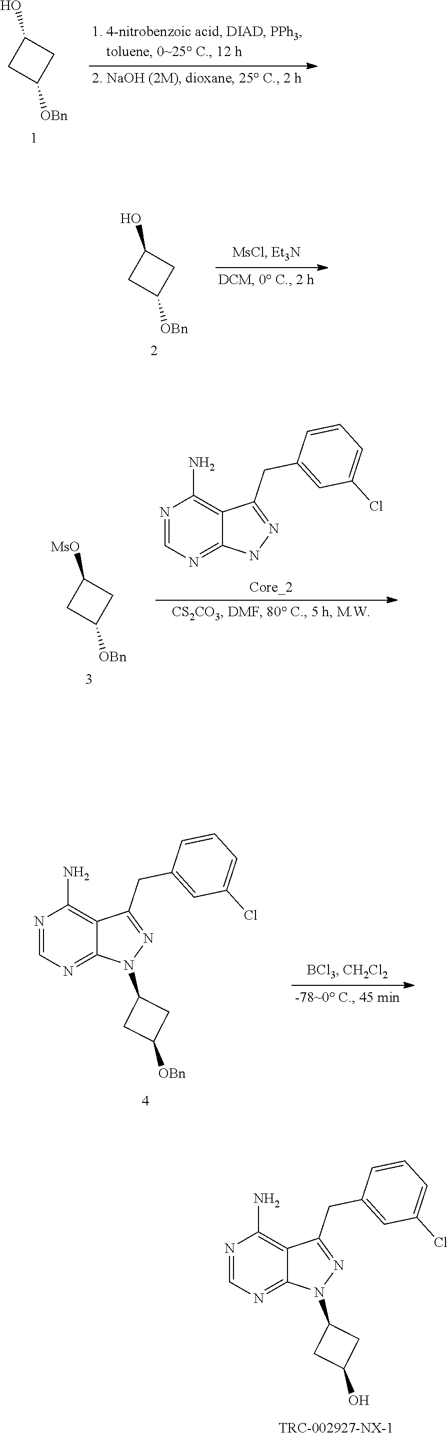 [SMILES: O[C@H]1C[C@@H](OCc2ccccc2)C1 ; O[C@H]1C[C@H](OCc2ccccc2)C1 ; CS(=O)(=O)O[C@H]1C[C@H](OCc2ccccc2)C1 ; Nc1ncnc2[nH]nc(Cc3cccc(Cl)c3)c12 ; Nc1ncnc2c1c(Cc1cccc(Cl)c1)nn2[C@H]1C[C@@H](OCc2ccccc2)C1 ; Nc1ncnc2c1c(Cc1cccc(Cl)c1)nn2[C@H]1C[C@@H](O)C1]

### Step 1

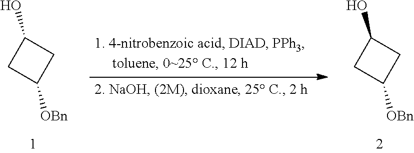 [SMILES: O[C@H]1C[C@@H](OCc2ccccc2)C1 ; O[C@H]1C[C@H](OCc2ccccc2)C1]

To a solution of (cu)-3-(benzyloxy)cyclobutan-1-ol (1) (1 g, 5.61 mmol, 1 eq), 4-nitrobenzoic acid (938 mg, 5.61 mmol, 1 eq) and PPh₃ (1.47 g, 5.61 mmol, 1 eq) in toluene (20 mL) at 0° C. was added DIAD (1.13 g, 5.61 mmol, 1.09 mL, 1 eq). The reaction was then stirred at 25° C. few 12 h, quenched with H₂O (50 mL), extracted with ethyl acetate (2×50 mL), dried (Na₂SO₄), filtered and concentrated under reduced pressure. The residue was purified by column chromatography (petroleum ether/ethyl acetate=5/1) to give the intermediate (1.7 g). The intermediate was dissolved in dioxane (8 mL) and NaOH (2 M, 2.81 mL, 1 eq), stirred at 25° C. for 2 h, quenched with H₂O (5 mL) and extracted with ethyl acetate (2×5 mL). The combined organic layers were dried (Na₂SO₄), filtered and concentrated under reduced pressure. The residue was purified by column chromatography (SiO₂, petroleum ether/ethyl acetate=3/l) to give (trans)-3-(benzyloxy)cyclobutan-1-ol (2) (0.6 g, 3.37 mmol, 60% yield) as colorless oil. ¹H NMR: (400 MHz, CDCl₃) 57.38-7.27 (m, 5H), 4.56 (tt, J=4.4, 6.8 Hz, 1H), 4.42 (s, 2H), 4.29 (tt, J=4.4, 6.8 Hz, 1H), 2.42-2.33 (m, 2H), 2.24-2.15 (m, 2H).

### Step 2

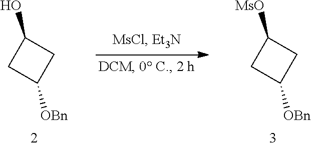 [SMILES: O[C@H]1C[C@H](OCc2ccccc2)C1 ; CS(=O)(=O)O[C@H]1C[C@H](OCc2ccccc2)C1]

To a solution of (trans)-3-(benzyloxy)cyclobutan-1-ol (2) (1.0 g, 5.61 mmol, 1 eq) and Et₃N (1.14 g, 11.2 mmol, 1.56 mL, 2 eq) in dichloromethane (10 mL) was added MsCl (964 mg, 8.42 mmol, 651 μL, 1.5 eq) at 0° C. under nitrogen atmosphere. Then the mixture was stirred at 0° C. for about 2 h. The reaction mixture was poured into H₂O (10 mL) slowly, and then extracted with dichloromethane (3×10 mL). The combined organic layers were dried over Na₂SO₄, filtered and concentrated under reduced pressure to give (trans)-3-(benzyloxy)cyclobutyl methanesulfonate (3) (1.21 g, crude) as a yellow oil which was used to the next step directly.

### Step 3

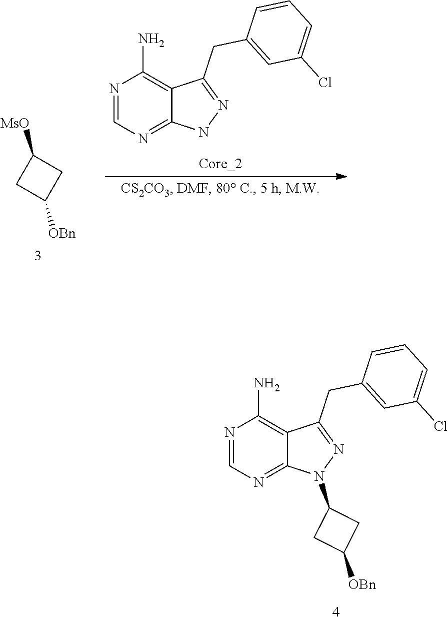 [SMILES: CS(=O)(=O)O[C@H]1C[C@H](OCc2ccccc2)C1 ; Nc1ncnc2[nH]nc(Cc3cccc(Cl)c3)c12 ; Nc1ncnc2c1c(Cc1cccc(Cl)c1)nn2[C@H]1C[C@@H](OCc2ccccc2)C1]

A mixture of (trans)-3-(benzyloxy)cyclobutyl methanesulfonate (3) (1.21 g, crude), 3-(3-chlorobenzyl)-1H-pyrazolo-[3,4-d]pyrimidin-4-amine (1 g, 3.85 mmol, 1 eq), Cs₂CO₃ (2.51 g, 7.70 mmol, 2 eq) and N,N-dimethylformamide (10 mL) was heated at 80° C. for 3 h under microwave irradiation. The mixture was filtered and the filtrate concentrated under reduced pressure. The resulting residue was purified by prep-HPLC (neutral condition) to give 1-((cis)-3-(benzyloxy)cyclobutyl)-3-(3-chlorobenzyl)-1H-pyrazolo-[3,4-d]-pyrimidin-4-amine (4) (1.5 g, 3.22 mmol, 42% yield, 90% purity) as a yellow solid. ¹H NMR: (400 MHz, CDCl₃) δ=8.27 (s, 1H), 7.41-7.36 (m, 7H), 7.30-7.27 (m, 1H), 7.22-7.12 (m, 1H), 5.07 (s, 2H), 5.00-4.92 (m, 1H), 4.54 (s, 2H), 4.32 (s, 2H), 4.06-3.98 (m, 1H), 2.88-2.84 (m, 4H).

### Step 4

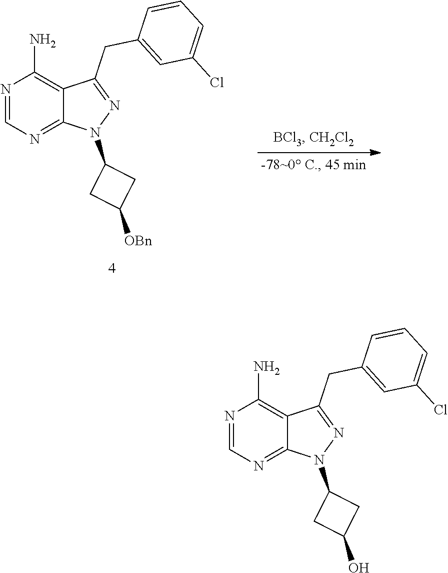 [SMILES: Nc1ncnc2c1c(Cc1cccc(Cl)c1)nn2[C@H]1C[C@@H](OCc2ccccc2)C1 ; Nc1ncnc2c1c(Cc1cccc(Cl)c1)nn2[C@H]1C[C@@H](O)C1]

To a solution of 1-((cis)-3-(benzyloxy)cyclobutyl)-3-(3-chlorobenzyl)-1H-pyrazolo-[3,4-d]-pyrimidin-4-amine (4) (35 mg, 83 μmol, 1 eq) in dichloromethane (5 mL) was added BCl₃ (1 M, 834 μL, 10 eq) at −78° C. under nitrogen atmosphere. The mixture was warmed to 0° C. for 45 min and quenched with MeOH (5 mL) at −78° C., warmed to 0° C. and the pH adjusted to 7 using 33% NH₃.H₂O. The mixture was filtered and the filtrate was concentrated under reduced pressure to give a residue that was purified by prep-HPLC (neutral condition) to give (cis)-3-(4-amino-3-(3-chlorobenzyl)-1H-pyrazolo[3,4-d]pyrimidin-1-yl)cyclobutan-1-ol (Compound 130) (3.7 mg, 11.1 μmol, 6.67% yield, 99% purity) as a white solid. LCMS: (M+H)⁺: 330.1, Rt: 2.253 min. LC/MS (The gradient was 1-90% B in 3.4 min, 90-100% B in 0.45 min, 100-1% B in 0.01 min, and then held at 1% B for 0.65 min (0.8 mL/min flow rate). Mobile phase A was 0.0375% CF3CO2H in water, mobile phase B was 0.018% CF3CO2H in CH3CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS). ¹H NMR: (400 MHz, MeOD-d₄) δ 8.15 (s, 1H), 7.31-7.25 (m, 2H), 7.25-7.20 (m, 1H), 7.17 (d, J=7.6 Hz, 1H), 4.85-4.79 (m, 1H), 4.39 (s, 2H), 4.23-4.12 (m, 1H), 2.85-2.82 (m, 2H), 2.70-2.67 (m, 2H).

TABLE 11
Compounds Prepared by Method K

| | | Expected | LC/MS | |
|---|---|---|---|---|
| Compound | | MW | Observed | |
| No. | IUPAC Name | (M) | (M + H) | 1H NMR (400 MHz) |
| 130 | (cis)-3-(4-amino-3-(3- | 329.1 | 330.1 | (METHANOL-d4) δ = 8.15 |
| | chlorobenzyl)-1H- | | | (s, 1H), 7.31-7.25 (m, 2H), |
| | pyrazolo[3,4-d] | | | 7.25-7.20 (m, 1H), 7.17 |
| | pyrimidin-1-yl) | | | (d, J = 7.6 Hz, 1H), |
| | cyclobutan-1-ol | | | 4.85-4.79 (m, 1H), |
| | | | | 4.39 (s, 2H), 4.23-4.12 |
| | | | | (m, 1H), 2.85-2.82 (m, 2H), |
| | | | | 2.70-2.67 (m, 2H) |
| 131 | (cis)-3-(4-amino-3-(3- | 373/375 | 374/376 | (METHANOL-d4) δ = 8.28 |
| | bromobenzyl)-1H- | | | (s, 1H), 7.46 (s, 1H), 7.43- |
| | pyrazolo[3,4-d] | | | 7.37 (m, 1H), 7.23 (d, J = |
| | pyrimidin-1-yl) | | | 4.8 Hz, 2H), 5.02-4.92 |
| | cyclobutan-1-ol | | | (m, 1H), 4.44 (s, 2H), 4.24- |
| | | | | 4.12 (m, 1H), 2.89-2.79 |
| | | | | (m, 2H), 2.74-2.62 (m, 2H) |
| 132 | (cis)-3-4-amino-3-(3- | 133.1 | 314.1 | (METHANOL-d4) δ = 8.28 |
| | fluorobenzyl)-1H- | | | (s, 1H), 7.35-7.30 (m, 1H), |
| | pyrazolo[3,4-d] | | | 7.08 (d, J = 7.2 Hz, 1H), |
| | pyrimidin-1-yl) | | | 7.02-6.98 (m, 2H), |
| | cyclobutan-1-ol | | | 5.00-4.94 (m, 1H), |
| | | | | 4.46 (s, 2H), |
| | | | | 4.22-4.15 (m, 1H), |
| | | | | 2.86-2.83 (m, 2H), |
| | | | | 2.69-2.66 (m, 2H) |
| 133 | (cis)-3-(4-amino-3- | 364.1 | 364.2 | (METHANOL-d4) δ = 8.18 |
| | (3-(trifluoromethyl) | | | (s, 1H), 7.61 (s, 1H), |
| | benzyl)-1H- | | | 7.55-7.47 (m, 3H), |
| | pyrazolo[3,4-d] | | | 4.92-4.89 (m, 1H), |
| | pyrimidin-1-yl) | | | 4.50 (s, 2H), |
| | cyclobutan-1-o1 | | | 4.22-4.13 (m, 1H), |
| | | | | 2.88-2.81 (m, 2H), |
| | | | | 2.68-2.65 (m, 2H) |
| 134 | 3-((4-amino-1-((cis)-3- | 320.1 | 321.1 | (ACETONITRILE-d3) δ = |
| | hydroxycyclobutyl)- | | | 8.18 (s, 1H), 7.61 (s, 2H) |
| | 1H-pyrazolo[3,4-d] | | | 7.54-7.52 (m, 1H), |
| | pyrimidin-3-yl) | | | 7.49-7.45 (m, 1H), |
| | methyl)benzonitrite | | | 5.72 (s, 2H), |
| | | | | 4.90-4.82 (m, 1H), |
| | | | | 4.38 (s, 2H), |
| | | | | 4.17-4.10 (m, 1H), |
| | | | | 3.47 (d, J = 6.4 Hz, 1H), |
| | | | | 2.82-2.77 (m, 2H), |
| | | | | 2.56 (q, J = 8.8 Hz, 2H) |

### Synthesis Method L: General Procedure Represented by the Preparation of 3-(3-chlorobenzyl)-1-((m)-3-fluorocyclobutyl)-1H-pyrazolo[3,4-d]pyrimidin-4-amine

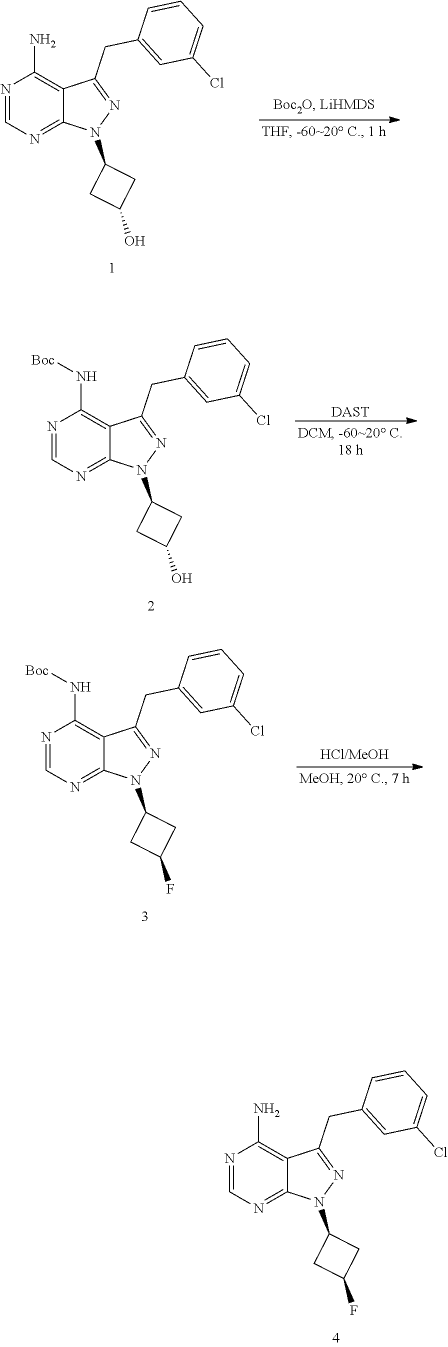

### Step 1

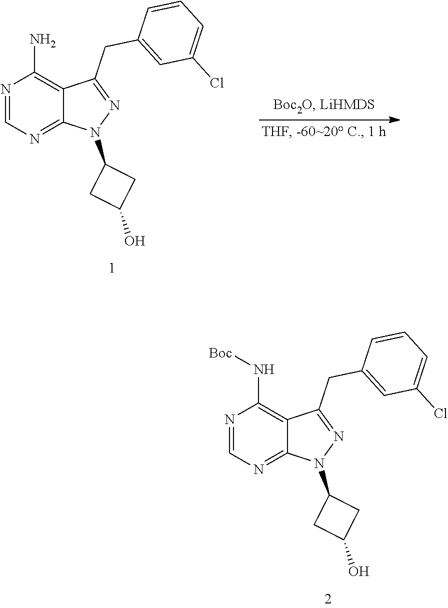 [SMILES: Nc1ncnc2c1c(Cc1cccc(Cl)c1)nn2[C@H]1C[C@H](O)C1 ; CC(C)(C)OC(=O)Nc1ncnc2c1c(Cc1cccc(Cl)c1)nn2[C@H]1C[C@H](O)C1]

To a solution of (trans)-3-(4-amino-3-(3-chlorobenzyl)-1H-pyrazolo[3,4-d]pyrimidin-1-yl)cyclobutan-1-ol (1) (200 mg, 606 μmol, 1 eq) in tetrahydrofuran (5 mL) was added LiHMDS (1 M, 1.33 mL, 2.2 eq) dropwise at −60° C. under nitrogen atmosphere. The resulting mixture was stirred at −60° C. for 30 min, then Boc₂O (172 mg, 788 μmol, 181 μL, 1.3 eq) was added and the reaction mixture was warmed slowly to 20° C. and stirred for 1h and then quenched by pouring into H₂O (10 mL). The mixture was extracted with ethyl acetate (3×10 mL) and the combined organic phase was dried (Na₂SO₄), concentrated under reduced pressure and purified by prep-TLC (petroleum ether/ethyl acetate=1/1) to give tert-butyl (3-(3-chlorobenzyl)-1-((trans)-3-hydroxycyclobutyl)-1H-pyrazolo[3,4-d]pyrimidin-4-yl)carbamate (2) (0.08 g, 149 μmol, 24.6% yield, 80% purity) as a white solid. ¹H NMR: (400 MHz, DMSO-d₆) δ=9.91 (s, 1H), 8.61 (s, 1H), 7.29-7.22 (m, 3H), 7.05-7.01 (m, 1H), 5.51-5.45 (m, 1H), 5.27-5.24 (m, 1H), 4.56-4.52 (m, 1H), 4.41 (s, 2H), 2.79-2.74 (m, 2H), 2.46-2.40 (m, 2H), 1.46 (s, 9H).

### Step 2

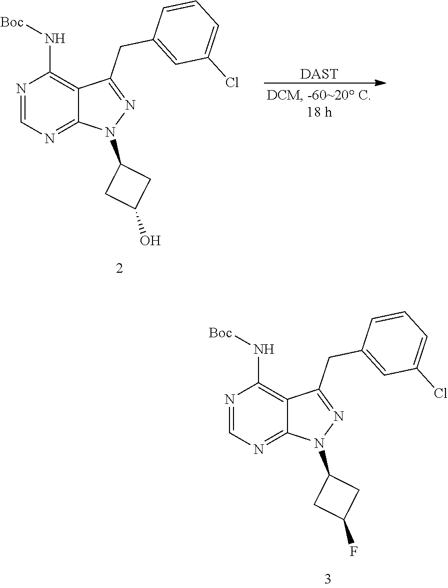

To a solution of tert-butyl (3-(3-chlorobenzyl)-1-((trans)-3-hydroxycyclobutyl)-1H-pyrazolo-[3,4-d]pyrimidin-4-yl)carbamate (2) (50 mg, 116 μmol, 1 eq) in DCM (3 mL) was added DAST (93.7 mg, 582 μmol, 76.8 μL, 5 eq) dropwise at −60° C. under nitrogen atmosphere. The mixture was slowly warmed to 20° C. and stirred for about 18 h. The reaction mixture was poured into 5 mL of water, extracted with DCM (3×5 mL), dried (sodium sulfate) and concentrated under reduced pressure to give tert-butyl (3-(3-chlorobenzyl)-1-((cis)-3-fluorocyclobutyl)-1H-pyrazolo-[3,4-d]pyrimidin-4-yl)carbamate (3) (50 mg, crude) as a black red solid which was used for next step directly.

### Step 3

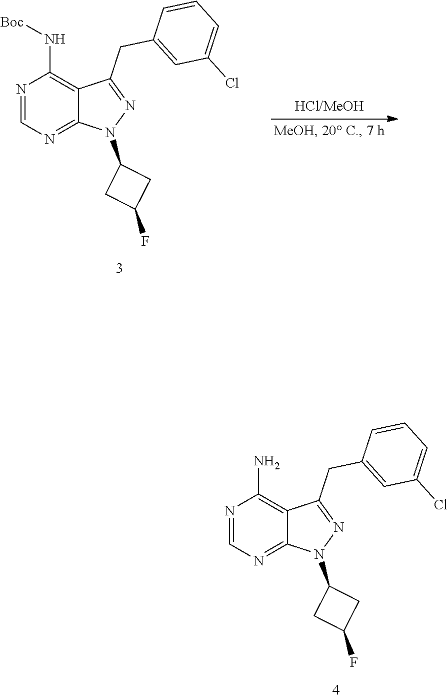 [SMILES: CC(C)(C)OC(=O)Nc1ncnc2c1c(Cc1cccc(Cl)c1)nn2[C@H]1C[C@@H](F)C1 ; Nc1ncnc2c1c(Cc1cccc(Cl)c1)nn2[C@H]1C[C@@H](F)C1]

To a solution of tert-butyl (3-(3-chlorobenzyl)-1-((cis)-3-fluorocyclobutyl)-1H-pyrazolo-[3,4-d]pyrimidin-4-yl)carbamate (3) (50 mg, crude) in methanol (3 mL) was added HCl/MeOH (4 M, 5 mL) at 20° C. The mixture was stirred at 20° C. for about 7 h, concentrated under reduced pressure and the remaining residue dissolved in 3 mL of DMSO and purified by prep-HPLC (basic condition) to give 3-(3-chlorobenzyl)-1-((cis)-3-fluorocyclobutyl)-1H-pyrazolo[3,4-d]pyrimidin-4-amine (4; Compound 135) (4.8 mg, 14.2 μmol, 12.2% yield, 97.8% purity) as a gray solid. LCMS: (M+H)⁺: 332.0, Rt: 2.318 min. LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS). ¹H NMR: (400 MHz, CD3CN) δ=8.21 (s, 1H), 7.35-7.28 (m, 3H), 7.21 (d, J=6.8 Hz, 1H), 5.78 (s, 2H), 5.13-4.89 (m, 2H), 4.36 (s, 2H), 3.02-2.88 (m, 4H).

TABLE 12
Compounds Prepared by Method L

| | | | LC/MS | |
|---|---|---|---|---|
| Compound | | | Observed | |
| No. | IUPAC Name | MW | (M + H) | 1H NMR (400 MHz) |
| 135 | 3-(3-chlorobenzyl)-1- | 331.1 | 332 | (ACETONITRILE-d3) δ = |
| | ((cis)-3- | | | 8.21 (s, 1H), 7.35-7.28 (m, |
| | fluorocyclobutyl)-1H- | | | 3H), 7.21 (d, J = 6.8 Hz, 1H), |
| | pyrazolo[3,4- | | | 5.78 (s, 2H), 5.13-5.09 (m, |
| | d]pyrimidin-4-amine | | | 0.5H) 4.99-4.89 (m, 1.5H), |
| | | | | 4.36 (s, 2H), 3.02-2.88 (m, |
| | | | | 4H) |
| 136 | 3-(3-chlorobenzyl)-1- | 331.1 | 332 | (ACETONITRILE-d3) δ = |
| | ((trans)-3- | | | 8.18 (s, 1H), 7.32-7.25 (m, |
| | fluorocyclobutyl)-1H- | | | 3H), 7.18 (d, J = 7.2 Hz, 1H), |
| | pyrazolo[3,4- | | | 5.68 (s, 2H), 5.56-5.51 (m, |
| | d]pyrimidin-4-amine | | | 1.5H), 5,50-5.42 (m, 0.5H) |
| | | | | 4.32 (s, 2H), 2.95-2.89 (m, |
| | | | | 2H), 2.84-2.76 (m, 2H) |
| 137 | 1-((Trans)-3- | 368.1 | 369 | (ACETONITRILE-d3) δ = |
| | fluorocyclobutyl)-3-((4- | | | 8.36 (d, J = 5.2 Hz, 1H), 8.23 |
| | (trifluoromethyl)pyridin- | | | (s, 1H), 7.56 (s, 1H), 7.46 (d, J = |
| | 2-yl)oxy)-1H- | | | 5.2 Hz, 1H), 6.01 (s, 2H), |
| | pyrazolo[3,4- | | | 5.56-5.51 (m, 1H), 5.47- |
| | d]pyrimidin-4-amine | | | 5.45 (m, 0.5H), 5.33-5.31 (m, |
| | | | | 0.5H), 2.88-2.75 (m, 4H) |
| 138 | 2-((4-amino-1-((trans)-3- | 325.1 | 326 | (METHANOL-d4) δ = 8.38 |
| | fluorocyclobutyl)-1H- | | | (d, J = 4.4 Hz, 1H), 8.30 (s, |
| | pyrazolo[3,4- | | | 1H), 7.74 (s, 1H), 7.56 (dd, J = |
| | d]pyrimidin-3- | | | 1.2, 5.2 Hz, 1H), 5.64-5.60 |
| | yl)oxy)isonicotinonitrile | | | (m, 1H), 5.44-5.43 (m, |
| | | | | 0.5H), 5.30-5.27 (m, 0.5H), |
| | | | | 2.95-2.81 (m, 4H) |
| 139 | 1-((trans)-3- | 365.3 | 366.1 | (ACETONITRILE-d3) δ = |
| | fluorocyclobutyl)-3-(3- | | | 8.21 (s, 1H), 7.64 (s, 1H), 7.59- |
| | (trifluoromethyl)benzyl)- | | | 7.58 (m, 1H), 7.52 (d, J = 4.4 |
| | 1H-pyrazolo[3,4- | | | Hz, 2H), 5.78 (s, 2H), 5.59- |
| | d]pyrimidin-4-amine | | | 5.52 (m, 1H), 5.47-5.39 (m, |
| | | | | 1H), 4.43 (s, 2H), 2.98-2.81 |
| | | | | (m, 4H) |

### Synthesis Method M: General Procedure Represented by the Preparation of 3-(4-amino-3-(3-chlorobenzyl)-1H-pyrazolo[3,4-d]pyrimidin-1-yl)cyclobutan-1-one

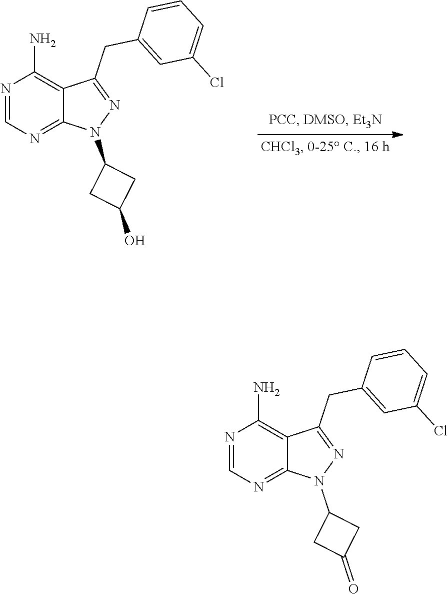 [SMILES: Nc1ncnc2c1c(Cc1cccc(Cl)c1)nn2[C@H]1C[C@@H](O)C1 ; Nc1ncnc2c1c(Cc1cccc(Cl)c1)nn2C1CC(=O)C1]

(Cis)-3-(4-amino-3-(3-chlorobenzyl)-1H-pyrazolo[3,4-d]pyrimidin-1-yl)-cyclobutan-1-ol (74.3 mg, 225 μmol, 1 eq, TFA), DMSO (194 mg, 2.48 mmol, 194 μL, 11 eq), Et₃N (137 mg, 1.35 mmol, 188 μL, 6 eq) and CHCl₃ (10 mL) were combined followed by the addition of PCC (194 mg, 901 μmol, 4 eq) at 0° C. The mixture was stirred at 25° C. for 16 h. filtered and the filtrate concentrated under reduced pressure to give a residue which was purified by prep-HPLC (neutral condition) to give 3-(4-amino-3-(3-chlorobenzyl)-1H-pyrazolo[3,4-d]pyrimidin-1-yl)cyclobutan-1-one (Compound 140) (11.6 mg, 34.6 μmol, 15.4% yield, 97.8% purity) as a white solid. LCMS: (M+H)⁺: 328.1, RT: 2.151 min LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C¹⁸ column (5 μm particles). Detection methods are diode array (DAD) and evaporative light scattering (ELSD) detection as well as positive electrospray ionization (MS). ¹H NMR: (400 MHz, DMSO-d₆) δ=8.15 (s, 1H), 7.35 (s, 1H), 7.30-7.22 (m, 2H), 7.16 (d, J=6.8 Hz, 1H), 5.50 (t, 7=6.4 Hz, 1H), 4.38 (s, 2H), 3.61 (d, 7=6.8 Hz, 4H).

TABLE 13
Compounds Prepared by Method M

| | | | LC/MS | |
|---|---|---|---|---|
| Compound | | | Observed | |
| No. | IUPAC Name | MW | (M + H) | 1H NMR (400 MHz) |
| 140 | 3-(4-amino-3-(3- | 327 | 328.1 | (DMSO-d6) δ = 8.15 (s, 1H), |
| | chlorobenzyl)-1H- | | | 7.35 (s, 1H), 7.30-7.22 (m, |
| | pyrazolo[3,4-d]pyrimidin- | | | 2H), 7.16 (d, J = 6.8 Hz, 1H), |
| | 1-yl)cyclobutan-1-one | | | 5.50 (t, J = 6.4 Hz, 1H), 4.38 |
| | | | | (s, 2H), 3.61 (d, J = 6.8 Hz, |
| | | | | 4H) |
| 141 | 3-(4-amino-3-((4- | 364.1 | 365.1 | (ACETONITRILE-d3) δ = |
| | (trifluoromethyl)pyridin- | | | 8.40 (d, J = 5.2 Hz, 1H), 8.30 |
| | 2-yl)oxy)-1H- | | | (s, 1H), 7.61 (s, 1H), 7.52 (d, |
| | pyrazolo[3,4-d]pyrimidin- | | | J = 5.2 Hz, 1H), 5.61 (q, J = |
| | 1-yl)cyclobutan-1-one | | | 7.2 Hz, 1H), 3.62 (d, J = 7.2 |
| | | | | Hz, 4H) |
| 142 | 2-((4-amino-1-(3- | 321.1 | 322.1 | (ACETONITRILE-d3) δ = |
| | oxocyclobutyl)-1H- | | | 10.61 (s, 1H), 8.37 (d, J = 5.2 |
| | pyrazolo[3,4-d]pyrimidin- | | | Hz, 1H), 8.30 (s, 1H), 7.66 (s, |
| | 3-yl)oxy)isonicotinonitrile | | | 1H), 7.54 (d, J = 5.2 Hz, 1H), |
| | | | | 7.15 (s, 1H), 5.61 (q, J = 7.2 |
| | | | | Hz, 1H), 3.62 (d, J = 6.8 Hz, |
| | | | | 4H) |
| 143 | 3-(4-amino-3-((4- | 330 | 331 | (ACETONITRILE-d3) δ = |
| | chloropyridin-2-yl)oxy)- | | | 8.28 (s, 1H), 8.12 (d, J = 5.6 |
| | 1H-pyrazolo[3,4- | | | Hz, 1H), 7.38 (d, J = 1.6 Hz, |
| | d]pyrimidin-1- | | | 1H), 7.29 (dd, J = 1.6, 5.6 Hz, |
| | yl)cyclobutan-1-one | | | 1H), 5.63-5.55 (m, 1H), 3.62- |
| | | | | 3.60 (m, 4H) |
| 144 | 3-(4-amino-3-((2- | 330 | 331 | (ACETONITRILE-d3) δ = |
| | chloropyridin-4-yl)oxy)- | | | 8.39 (d, J = 5.6 Hz, 1H), 8.29 |
| | 1H-pyrazolo[3,4- | | | (s, 1H), 7.48 (d, J = 1.6 Hz, |
| | d]pyrimidin-1- | | | 1H), 7.43 (dd, J = 2.4, 6.0 Hz, |
| | yl]cyclobutan-1-one | | | 1H), 5.62-5.55 (m, 1H), 3.66- |
| | | | | 3.61 (m, 4H) |

### Synthesis Method N General Procedure Represented by the Preparation of 1-cyclopropyl-3-((4-(tetrahydro-2H-pyran-4-yl)pyridin-2-yl)oxy)-1H-pyrazolo[3,4-d]pyrimidin-4-amine

### Synthetic Scheme 15 Representing Method N

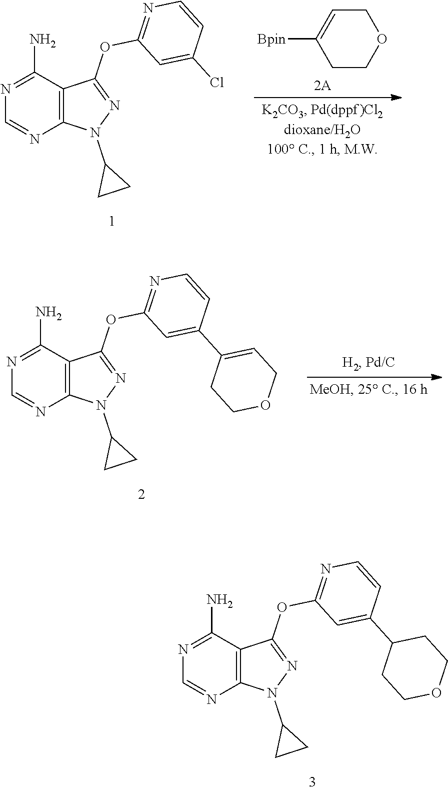

### Step 1

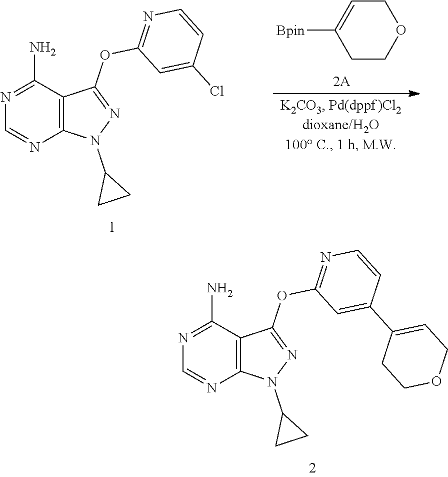

To a solution of 3-((4-chloropyridin-2-yl)oxy)-1-cyclopropyl-1H-pyrazolo[3,4-d]pyrimidin-4-amine (1) (400 mg, 1.32 mmol, 1 eq) in H₂O (2.5 mL) and dioxane (10 mL) was added the boronate ester of dihydropyran (2A) (333 mg, 1.58 mmol, 1.2 eq). K₂CO₃ (365 mg, 2.64 mmol, 2 eq) and Pd(dppf)C₁₋₂ (48.3 mg, 66.0 μmol, 0.05 eq) and the mixture was stirred at 80° C. for 3 h under microwave. The reaction was quenched by the addition of water (15 mL) and extracted with ethyl acetate (3×15 mL). The organic phase was dried over Na₂SO₄, Altered and concentrated under reduced pressure to give a red solid which was purified by prep-HPLC (TFA condition) to give 1-cyclopropyl-3-((4-(3,6-dihydro-2H-pyran-4-yl)pyridin-2-yl)oxy)-1H-pyrazolo[3,4-d]pyrimidin-4-amine (2) (150 mg, 427 μmol, 32.36% yield, 99.78% purity) as a white solid LCMS: (M+H)⁺: 351.1, Rt: 2.326 min. LC/MS (The gradient was 1-90% B in 3.4 min, 90-100% B in 0.45 min, 100-1% B in 0.01 min, and then held at 1% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS). ¹H NMR: (400 MHz, METHANOL-d₄) δ=8.33 (s, 1H), 8.13 (d, J=5.6 Hz, 1H), 7.43 (s, 1H), 7.37-7.36 (m, 1H), 6.61-6.60 (m, 1H), 4.35-4.33 (m, 2H), 3.94 (t, J=5.6 Hz, 2H), 3.84-3.81 (m, 1H), 2.58-2.52 (m, 2H), 1.25-1.09 (m, 4H).

### Step 2

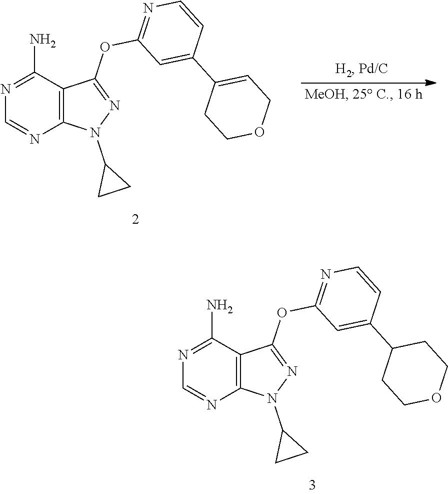 [SMILES: Nc1ncnc2c1c(Oc1cc(C3=CCOCC3)ccn1)nn2C1CC1 ; Nc1ncnc2c1c(Oc1cc(C3CCOCC3)ccn1)nn2C1CC1]

To a solution of 1-cyclopropyl-3-((4-(3,6-dihydro-2H-pyran-4-yl)pyridin-2-yl)oxy)-1H-pyrazolo[3,4-d]pyrimidin-4-amine (2) (100 mg, 215 μmol, 1 eq, TFA) in MeOH (20 mL) was added Pd/C (20 mg, 10% purity). The suspension was degassed under vacuum and purged with H₂ several times. The mixture was stirred under H₂ (15 psi) at 25° C. for 16 h, filtered and the filtrate concentrated in vacuum. The residue was purified by prep-HPLC (neutral condition) to give 1-cyclopropyl-3-((4-(tetrahydro-2H-pyran-4-yl)pyridin-2-yl)oxy)-1H-pyrazolo[3,4-d]pyrimidin-4-amine (3; Compound 145) (38.4 mg, 107 μmol, 49.6% yield, 98% purity) as a white solid. LCMS: (M+H)⁺: 353.1, Rt: 2.308 min. LC/MS (The gradient was 1-90% B in 3.4 min, 90-100% B in 0.45 min, 100-1% B in 0.01 min, and then held at 1% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS). ¹H NMR: (400 MHz, DMSO-d₆) δ=8.19 (s, 1H), 8.04 (d, J=5.2 Hz, 1H), 7.12 (d, J=5.2 Hz, 1H), 7.09 (s, 1H), 3.98-3.94 (m, 2H), 3.73-3.65 (m, 1H), 3.47-3.42 (m, 2H), 2.89-2.85 (m, 1H), 1.77-1.67 (m, 4H), 1.06-0.99 (m, 4H).

TABLE 14
Compounds Prepared by Method N

| | | | LC/MS | |
|---|---|---|---|---|
| Compound | | | Observed | |
| No. | IUPAC Name | MW | (M + H) | 1H NMR (400 MHz) |
| 146 | 1-cyclopropyl-3-((2- | 352.1 | 353.1 | (DMSO-d6) δ = 8.45 |
| | (tetrahydro-2H-pyran-4- | | | (d, J = 6.0 Hz, 1H), |
| | yl)pyridin-4-yl)oxy)-1H- | | | 8.21 (s, 1H), |
| | pyrazolo[3,4-d]pyrimidin- | | | 7.25 (d, J = 2.4 Hz, 1H), |
| | 4-amine | | | 7.20-7.18 (m, 1H), |
| | | | | 3.95-3.93 (m, 2H), |
| | | | | 3.71-3.69 (m, 1H), |
| | | | | 3.46-3.40 (m, 2H), |
| | | | | 2.94-2.91 (m, 1H), |
| | | | | 1.77-1.72 (m, 4H), |
| | | | | 1.12-1.01 (m, 4H) |

### Synthesis Method O General Procedure Represented by the Preparation of 7-cyclobutyl-5-(pyridin-2-ylmethyl)-7H-pyrrolo[2,3-d]pyrimidin-4-amine

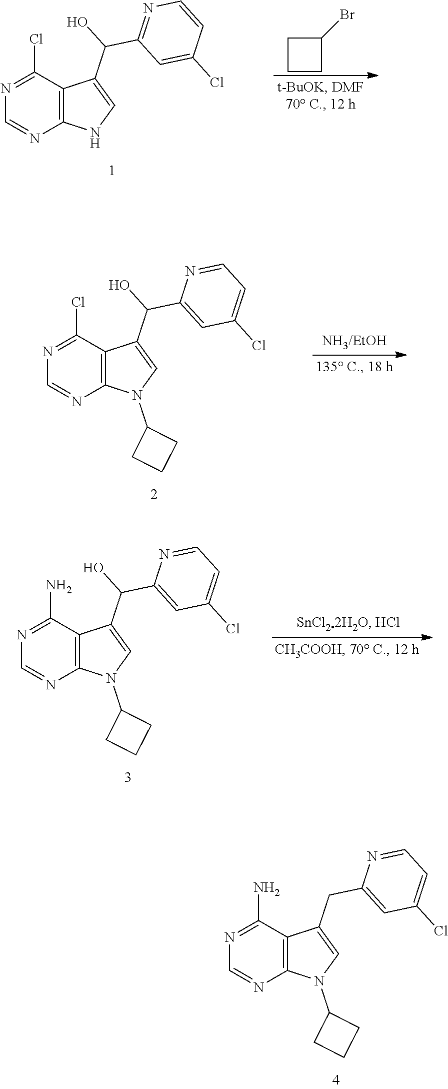 [SMILES: OC(c1cc(Cl)ccn1)c1c[nH]c2ncnc(Cl)c12 ; BrC1CCC1 ; OC(c1cc(Cl)ccn1)c1cn(C2CCC2)c2ncnc(Cl)c12 ; Nc1ncnc2c1c(C(O)c1cc(Cl)ccn1)cn2C1CCC1 ; Nc1ncnc2c1c(Cc1cc(Cl)ccn1)cn2C1CCC1]

### Step 1. Procedure for preparation of (4-chloro-7-cyclobutyl-7H-pyrrolo[2,3-d]pyrimidin-5-yl)(4-chloropyridin-2-yl)methanol (2)

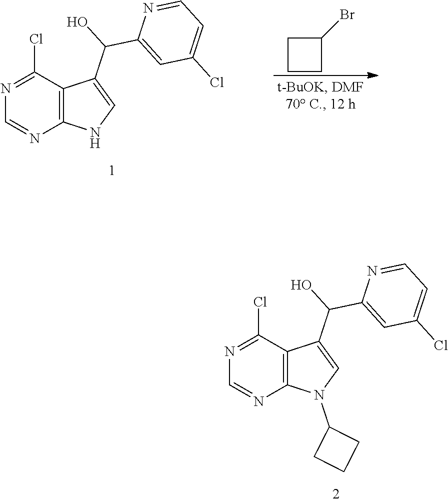 [SMILES: OC(c1cc(Cl)ccn1)c1c[nH]c2ncnc(Cl)c12 ; BrC1CCC1 ; OC(c1cc(Cl)ccn1)c1cn(C2CCC2)c2ncnc(Cl)c12]

A mixture of (4-chloro-7H-pyrrolo[2,3-d]pyrimidin-5-yl)(4-chloropyridin-2-yl)methanol (1) (1.0 g, 3.39 mmol, 1 eq), bromocyclobutane (915 mg, 6.78 mmol, 2 eq), t-BuOK (760 mg, 6.78 mmol, 2 eq) and DMF (5 mL) was stirred at 70° C. for 12 h. The reaction mixture was partitioned between water (200 mL) and EtOAc (100 mL) and the organic phase was separated. The aqueous phase was extracted with EtOAc (3×60 mL) and the combined organic phases were washed with brine, dried over Na₂SO₄ (2 g) and concentrated under reduced pressure. The product was purified by column chromatography (SiO₂, Petroleum ether/Ethyl acetate=50/1 to 1/1) to give (4-chloro-7-cyclobutyl-7H-pyrrolo[2,3-d]pyrimidin-5-yl)(4-chloropyridin-2-yl)methanol (2) (800 mg, 33.8% yield) as a yellow solid

### Step 2. Procedure for preparation of (4-amino-7-cyclobutyl-7H-pyrrolo[2,3-d]pyrimidin-5-yl)(4-chloropyridin-2-yl)methanol (3)

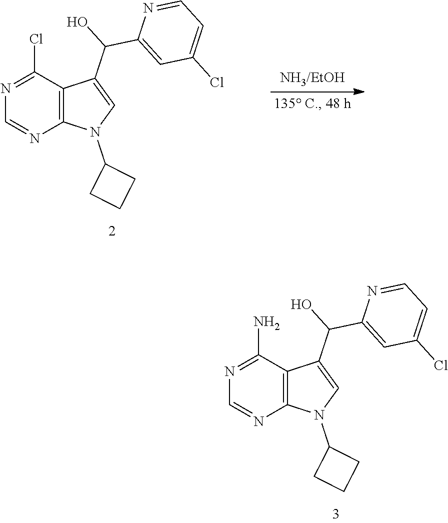 [SMILES: OC(c1cc(Cl)ccn1)c1cn(C2CCC2)c2ncnc(Cl)c12 ; Nc1ncnc2c1c(C(O)c1cc(Cl)ccn1)cn2C1CCC1]

A mixture of (4-chloro-7-cyclobutyl-7H-pyrrolo[2,3-d]pyrimidin-5-yl)(4-chloropyridin-2-yl)methanol (2) (300 mg, 859.07 μmol, 1 eq) and NH₃/EtOH (2 M, 5 mL) was stirred at 135° C. for 48 h in sealed tube. LCMS showed the reaction was completed. The reaction was concentrated to afford the crude product. The crude product was purified by prep-HPLC (neutral condition) to give (4-amino-7-cyclobutyl-7H-pyrrolo[2,3-d]pyrimidin-5-yl)(4-chloropyridin-2-yl)methanol (3) (36.5 mg, 12.9% yield) as a light yellow solid. LCMS: (M+H)⁺: 330.0, Rt: 2.493 min. ¹H NMR: (400 MHz, METHANOL-di) δ=8.43 (d, J=5.2 Hz, 1H), 8.05 (s, 1H), 7.68 (d, J=2.0 Hz, 1H), 7.36 (dd, J=2.0, 5.6 Hz, 1H), 7.26 (s, 1H), 6.05 (s, 1H), 5.12 (q, J=8.8 Hz, 1H), 2.50-2.43 (m, 4H), 1.94-1.86 (m, 2H).

### Step 3. Procedure for preparation of S-((4-chloropyridin-2-yl)methyl)-7-cyclobutyl-7H-pyrrolo[2,3-d]pyrimidin-4-amine (4)

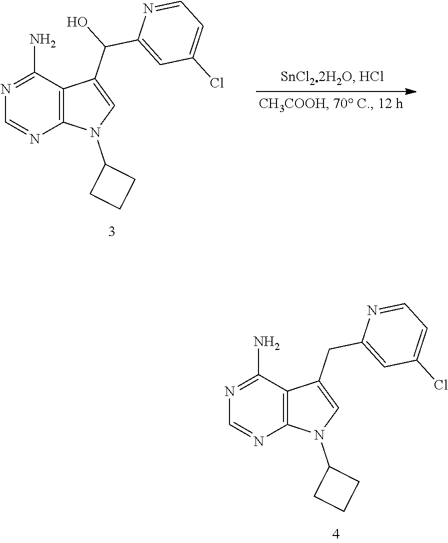 [SMILES: Nc1ncnc2c1c(C(O)c1cc(Cl)ccn1)cn2C1CCC1 ; Nc1ncnc2c1c(Cc1cc(Cl)ccn1)cn2C1CCC1]

To a solution of (4-amino-7-cyclobutyl-7H-pyrrolo[2,3-d]pyrimidin-5-yl)(4-chloropyridin-2-yl)methanol (3) (10 mg, 30.3 μmol, 1 eq) in CH₃COOH (2 mL) was added SnCl₂.2H₂O (54.7 mg, 243 μmol, 8 eq) and HCl (598 μg, 6.06 μmol, 0.59 μL, 37% purity, 0.2 eq). The mixture was stirred at 70° C. for 12 h and concentrated to afford the crude product, which was purified by prep-HPLC (HCl condition) to give 5-((4-chloropyridin-2-yl)methyl)-7-cyclobutyl-7H-pyrrolo[2,3-d]pyrimidin-4-amine (4; Compound 147) (2.8 mg, 4.20% yield) as a light yellow solid. LCMS: (M+H)⁺: 314.1, Rt: 2.777 min. NMR: (400 MHz, MeOD-d₄) δ=8.73 (d, J=6.0 Hz, 1H), 8.29 (s, 1H), 7.92 (d, J=6.0 Hz, 1H), 7.89 (s, 1H), 7.68 (s, 1H), 5.29 (q, J=8.8 Hz, 1H), 4.70 (s, 2H), 2.64-2.48 (m, 4H), 1.99-1.90 (m, 2H).

### Alternative Method O: General Procedure Represented by the Preparation of 7-cyclobutyl-5-(pyridin-2-ylmethyl)-7H-pyrrolo[2,3-d]pyrimidin-4-amine

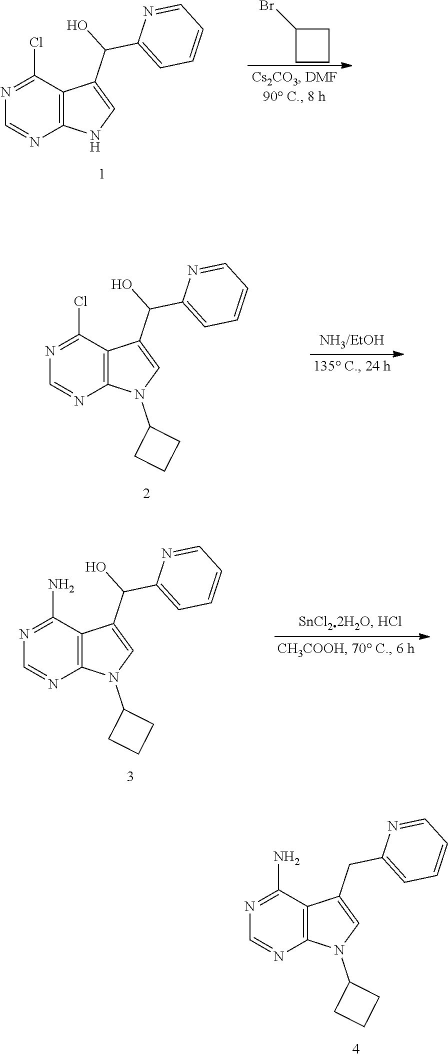 [SMILES: OC(c1ccccn1)c1c[nH]c2ncnc(Cl)c12 ; BrC1CCC1 ; OC(c1ccccn1)c1cn(C2CCC2)c2ncnc(Cl)c12 ; Nc1ncnc2c1c(C(O)c1ccccn1)cn2C1CCC1 ; Nc1ncnc2c1c(Cc1ccccn1)cn2C1CCC1]

### Step 1. Procedure for preparation of (4-chloro-7-cyclobutyl-7H-pyrrolo[2,3-d]pyrimidin-5-yl)(pyridin-2-yl)methanol (2)

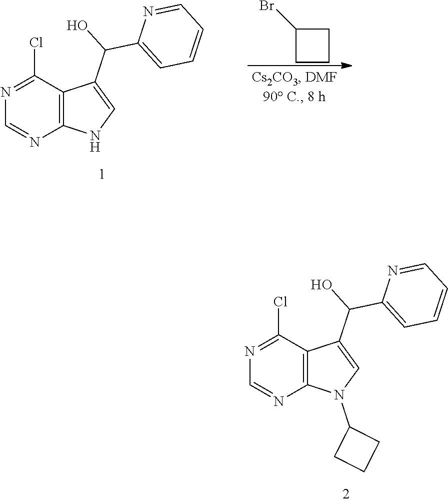 [SMILES: OC(c1ccccn1)c1c[nH]c2ncnc(Cl)c12 ; BrC1CCC1 ; OC(c1ccccn1)c1cn(C2CCC2)c2ncnc(Cl)c12]

A mixture of (4-chloro-7H-pyrrolo[2,3-d]pyrimidin-5-yl)(pyridin-2-yl)methanol (1) (1 g, 3.84 mmol, 1 eq), bromocyclobutane (1.04 g, 7.67 mmol, 724 μL, 2 eq), Cs₂CO₃ (2.50 g, 7.67 mmol, 2 eq) and DMF (10 mL) was stirred at 90° C. for 8 h. The reaction mixture was partitioned between water (200 mL) and Ethyl acetate (100 mL). The organic phase was separated and the aqueous phase was washed with 3×60 mL of EtOAc. The combined organic phases were washed with brine, dried over Na₂SO₄ (2 g), and concentrated under reduced pressure. The residue was purified by column chromatography (SiO₂, Petroleum ether/Ethyl acetate=50/1 to 1/1) to give (4-chloro-7-cyclobutyl-7H-pyrrolo[2,3-d]pyrimidin-5-yl)(pyridin-2-yl)methanol (2) (500 mg, 20.7% yield) as a yellow solid.

### Step 2. Procedure for preparation of (4-amino-7-cyclobutyl-7H-pyrrolo[2,3-d]pyrimidin-5-yl)(pyridin-2-yl)methanol (3)

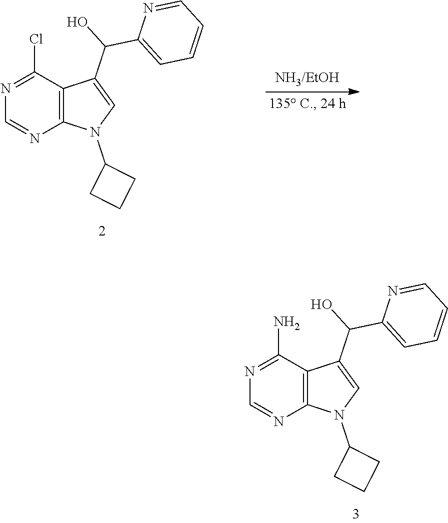 [SMILES: OC(c1ccccn1)c1cn(C2CCC2)c2ncnc(Cl)c12 ; Nc1ncnc2c1c(C(O)c1ccccn1)cn2C1CCC1]

A mixture of (4-chloro-7-cyclobutyl-7H-pyrrolo[2,3-d]pyrimidin-5-yl)(pyridin-2-yl)methanol (2) (250 mg, 794 μmol, 1 eq) and NH₃/EtOH (2 M, 3 mL) was stirred at 135° C. for 24 h in a sealed tube. The reaction mixture was concentrated and the crude product purified by prep-HPLC (neutral condition) to give (4-amino-7-cyclobutyl-7H-pyrrolo[2,3-d]pyrimidin-5-yl)(pyridin-2-yl)methanol (3) (23 mg, 9.51% yield, 97% purity) as a light yellow solid. LCMS: (M+H)⁺: 296.1, Rt: 2.227 min. ¹H NMR: (400 MHz, METHANOL-d₄) δ=8.50 (d, J=4.8 Hz, 1H), 8.04 (s, 1H), 7.87-7.81 (m, 1H), 7.63 (d, J=8.0 Hz, 1H), 7.31 (dd, J=4.8, 6.4 Hz, 1H), 7.18 (s, 1H), 6.05 (s, 1H), 5.12 (q, J=8.8 Hz, 1H), 2.49-2.42 (m, 4H), 1.93-1.84 (m, 2H).

### Step 3. Procedure for preparation of 7-cyclobutyl-5-(pyridin-2-ylmethyl)-7H-pyrrolo[2,3-d]pyrimidin-4-amine (4)

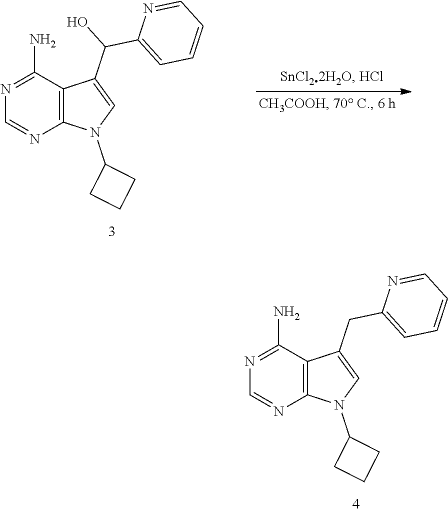 [SMILES: Nc1ncnc2c1c(C(O)c1ccccn1)cn2C1CCC1 ; Nc1ncnc2c1c(Cc1ccccn1)cn2C1CCC1]

To a solution of (4-amino-7-cyclobutyl-7H-pyrrolo[2,3-d]pyrimidin-5-yl)(pyridin-2-yl)methanol (3) (10 mg, 33.9 μmol, 1 eq) in CH₃COOH (2 mL) was added SnCl₂.2H₂O (61.1 mg, 271 μmol, 8 eq) and HCl (667 μg, 6.77 μmol, 0.65 μL, 37% purity, 0.2 eq). The mixture was stirred at 70° C. for 12 h and concentrated to afford the crude product, which was purified by prep-HPLC (HCl condition) to give 7-cyclobutyl-5-(pyridin-2-ylmethyl)-7H-pyrrolo[2,3-d]pyrimidin-4-amine (4; Compound 148) (2.8 mg, 14.8% yield) as a light yellow solid. LCMS: (M+H)⁺: 280.1, Rt: 2.517 min. ¹H NMR: (400 MHz, MeOD-d₄) δ=8.81 (d, J=5.6 Hz, 1H), 8.55 (t, J=7.6 Hz, 1H), 8.31 (s, 1H), 7.98 (t, J=6.8 Hz, 1H), 7.84 (d, J=8.0 Hz, 1H), 7.65 (s, 1H), 5.36-5.25 (m, 1H), 4.82 (s, 2H), 2.63-2.49 (m, 4H), 1.94 (tt, 7=5.2, 9.6 Hz, 2H).

### Synthesis Method P: General Procedure Represented by the Preparation of 1-cyclopropyl-3-((4-methoxypyridin-2-yl)methyl)-1H-pyrazolo[3,4-d]pyrimidin-4-amine

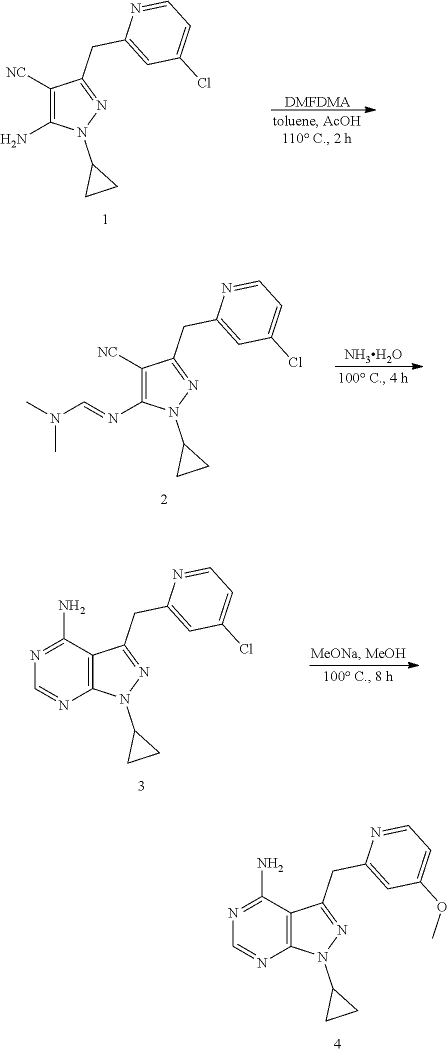

### Step 1. Procedure for preparation of (E)-N′-(3-((4-chloropyridin-2-yl)methyl)-4-cyano-1-cyclopropyl-1H-pyrazol-5-yl)-N,N-dimethylformimidamide (2)

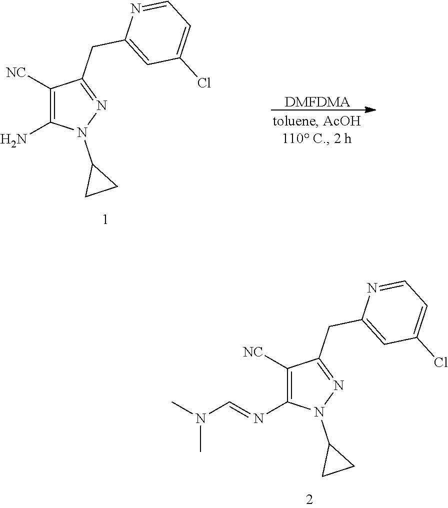

Two reactions were carried out in parallel. To a solution of 5-amino-3-((4-chloropyridin-2-yl)methyl)-1-cyclopropyl-1H-pyrazole-4-carbonitrile (1) (175 mg, 639 mmol, 1 eq) in toluene (3 mL) was added DMF-DMA (114 mg, 959 μmol, 127 μL, 1.5 eq) and AcOH (384 μg, 6.39 μmol, 0.37 μL, 0.01 eq). The mixture was stirred at 110° C. for 2 h and the two reactions were combined and concentrated under reduced pressure to give (E)-N′-(3-((4-chloropyridin-2-yl)methyl)-4-cyano-1-cyclopropyl-1H-pyrazol-5-yl)-N,N′-dimethylformimidamide (2) (400 mg, crude) as brown oil which was used in the next step without further purification.

### Step 2. Procedure for preparation of 3-((4-chloropyridin-2-yl)methyl)-1-cyclopropyl-1H-pyrazolo[3,4-d]pyrimidin-4-amine (3)

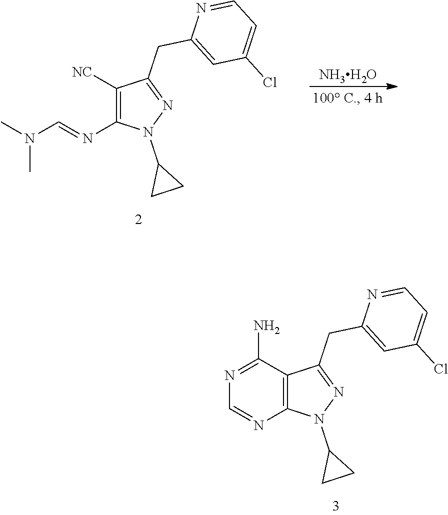 [SMILES: CN(C)/C=N/c1c(C#N)c(Cc2cc(Cl)ccn2)nn1C1CC1 ; "NH3·H2O" ; Nc1ncnc2c1c(Cc1cc(Cl)ccn1)nn2C1CC1]

A mixture of (E)-N′-(3-((4-chloropyridin-2-yl)methyl)-4-cyano-1-cyclopropyl-1H-pyrazol-5-yl)-N,N′-dimethylformimidamide (2) (400 mg, 1.22 mmol, 1 eq) in NH₃.H₂O (6 mL) was stirred at 100° C. for 4 h. The mixture was concentrated under reduced pressure to give a residue which was purified by prep-HPLC (neutral condition) to give 3-((4-chloropyridin-2-yl)methyl)-1-cyclopropyl-1H-pyrazolo[3,4-d]pyrimidin-4-amine (3) (60 mg, 200 μmol, 16.4% yield) as a yellow solid.

### Step 3. Procedure for preparation of 3-((4-chloropyridin-2-yl)methyl)-1-cyclopropyl-1H-pyrazolo[3,4-d]pyrimidin-4-amine (4)

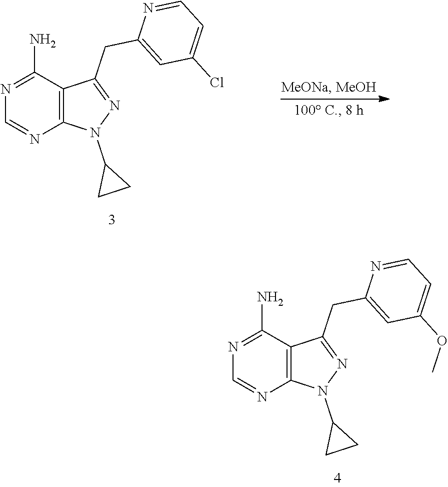 [SMILES: Nc1ncnc2c1c(Cc1cc(Cl)ccn1)nn2C1CC1 ; COc1ccnc(Cc2nn(C3CC3)c3ncnc(N)c23)c1]

The mixture of 3-((4-chloropyridin-2-yl)methyl)-1-cyclopropyl-1H-pyrazolo[3,4-d]pyrimidin-4-amine (3) (50 mg, 166.25 μmol, 1 eq) and NaOMe (44.91 mg, 831.27 μmol, eq) in MeOH (8 mL) was stirred at 100° C. for about 8 h in 30 mL sealed tube. LCMS showed the reaction was completed. The mixture was filtered and the filtrate was purified by prep-HPLC (neutral condition) to give 3-((4-chloropyridin-2-yl)methyl)-1-cyclopropyl-1H-pyrazolo[3,4-d]pyrimidin-4-amine (4; Compound 149) (7 mg, 23.6 μmol, 14.2% yield) as a white solid. LCMS: 1 (M+H)⁺: 297.1, Rt: 2.086 min. NMR: (400 MHz, METHANOL-d₄) δ=8.33 (d, J=6.0 Hz, 1H), 8.12 (s, 1H), 7.05 (d, J=2.4 Hz, 1H), 6.89 (dd, J=2.4, 6.0 Hz, 1H), 4.71 (s, 2H), 3.97 (tt, J=3.6, 7.2 Hz, 1H), 3.88 (s, 3H), 1.35-1.30 (m, 2H), 1.26-1.19 (m, 2H).

### Synthesis Method Q: General Procedure Represented by the Preparation of 1-((trans)-3-fluorocyclobutyl)-3-(3-fluorophenoxy)-1H-pyrazolo[3,4-d]pyrimidin-4-amine

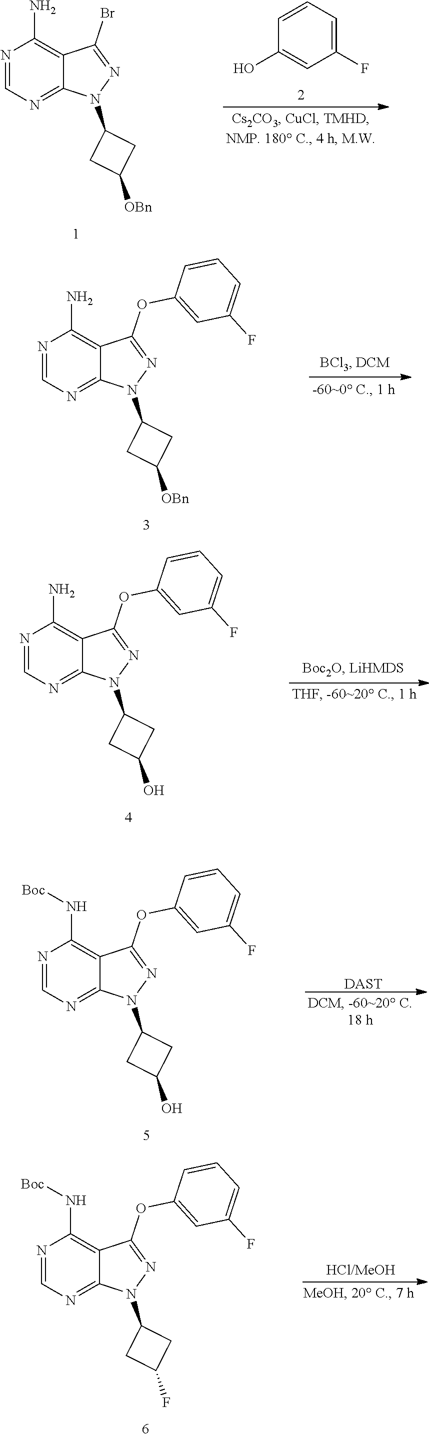

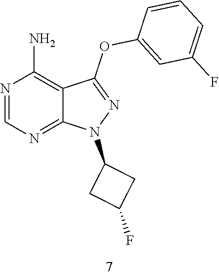 [SMILES: Nc1ncnc2c1c(Oc1cccc(F)c1)nn2[C@H]1C[C@H](F)C1]

### Step 1. Procedure for preparation of 1-((cis)-3-(benzyloxy)cyclobutyl)-3-(3-fluorophenoxy)-1H-pyrazolo[3,4-d]pyrimidin-4-amine

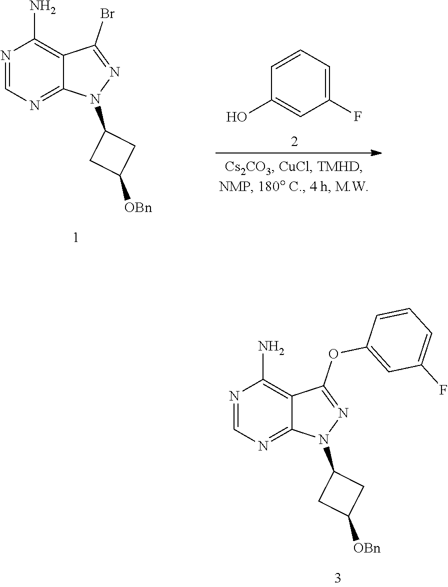 [SMILES: Nc1ncnc2c1c(Br)nn2[C@H]1C[C@@H](OCc2ccccc2)C1 ; Oc1cccc(F)c1 ; Nc1ncnc2c1c(Oc1cccc(F)c1)nn2[C@H]1C[C@@H](OCc2ccccc2)C1]

Three reactions were carried out in parallel. A mixture of 1 (0.2 g, 534.42 μmol, 1 eq), 2 (119.82 mg, 1.07 mmol, 98.21 μL, 2 eq), Cs₂CO₃ (348.25 mg, 1.07 mmol, 2 eq), TMHD (222.57 mg, 1.21 mmol, 248.69 μL, 2.26 eq) and CuCl (55.55 mg, 561.15 μmol, 13.42 μL, 1.05 eq) in NMP (2 mL) was added to a 3 mL Biotage microwave vial with a Teflon coated stirring bar under N₂. The vial was sealed and heated at 180° C. for 4 h under microwave irradiation. LCMS showed the reaction was completed. The reaction mixture was filtered and the filtrate was purified by prep-HPLC (TFA condition) to give 1-((1s,3s)-3-(benzyloxy)cyclobutyl)-3-(3-fluorophenoxy)-1H-pyrazolo[3,4-d]pyrimidin-4-amine (3) (0.1 g, 192.51 μmol, 12.01% yield, TFA) as a light yellow solid.

### Step 2. Procedure for the preparation of (cis)-3-(4-amino-3-(3-fluorophenoxy)-1H-pyrazolo[3,4-d]pyrimidin-1-yl)cyclobutan-1-ol (4)

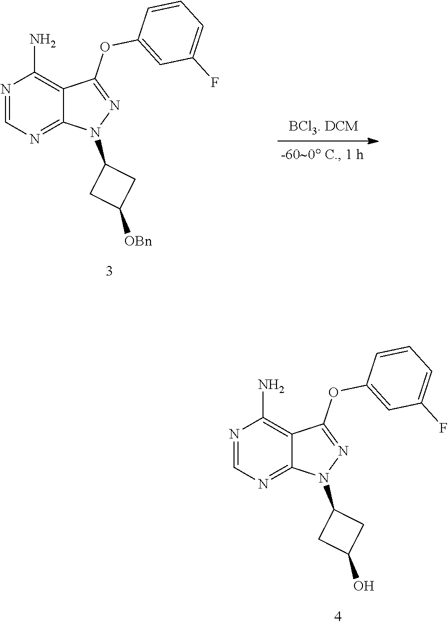 [SMILES: Nc1ncnc2c1c(Oc1cccc(F)c1)nn2[C@H]1C[C@@H](OCc2ccccc2)C1 ; Nc1ncnc2c1c(Oc1cccc(F)c1)nn2[C@H]1C[C@@H](O)C1]

To a mixture of 1-((cis)-3-(benzyloxy)cyclobutyl)-3-(3-fluorophenoxy)-1H-pyrazolo[3,4-d]pyrimidin-4-amine (3) (95 mg, 183 μmol, 1 eq. TFA) in DCM (3 mL) was added BCl₃ (1 M, 1.83 mL, 10 eq) dropwise at −60° C. The mixture was slowly warmed to 0° C. and stirred for about 1 h. The reaction mixture was quenched by addition of 2 mL of methanol at −60° C., then purified by prep-HPLC (TFA condition) to give (cis)-3-(4-amino-3-(3-fluorophenoxy)-1H-pyrazolo[3,4-d]pyrimidin-1-yl)cyclobutan-1-ol (4; Compound 150) (16.2 mg, 36.7 μmol, 20.1% yield, 97.3% purity, TFA) as a white solid. LCMS: (M+H)⁺: 316.1, Rt: 2.184 min. LC/MS method: The gradient was 1-90% B in 3.4 min, 90-100% B in 0.45 min. 100-1% B in 0.01 min. and then held at 1% B for 0.65 min (0.8 mL/min flow rate). Mobile phase A was 0.0375% CF₃CO₂H in water, mobile phase B was 0.018% CF₃CO₂H in CH₃CN. The column used for the chromatography was a 2.0×50 mm phenomenex Luna-C₁₋₈ column (5 μm particles). Detection methods are diode array (DAD) and evaporative light scattering (ELSD) detection as well as positive electrospray ionization (MS). ¹H NMR: (400 MHz, ACETONITRILE-d₃) δ=10.91 (s, 1H), 8.21 (s, 1H), 7.48-7.46 (m, 1H), 7.26-7.23 (m, 2H), 7.04 (t, J=7.6 Hz, 1H), 4.81-4.78 (m, 1H), 4.10-4.03 (m, 1H), 2.76-2.73 (m, 2H), 2.44-2.41 (m, 2H).

Trans-3-fluorocyclobutyl analogs were produced from (cis)-3-hydroxycyclobutyl compound 4 in Synthesis Method Q in an analogous manner as described in Synthesis Method L, by protecting the amino group with Boc (Boc₂O, LiHMDS), fluorinating with DAST and then deprotection of the amino group.

TABLE 15
Compounds Prepared by Method Q

| | | | LC/MS | |
|---|---|---|---|---|
| Compound | | | Observed | |
| No. | IUPAC Name | MW | (M + H) | 1H NMR (400 MHz) |
| 151 | (cis)-3-(4-amino-3-(3- | 365.1 | 366.1 | 1H NMR (400 MHz, |
| | (trifluoromethyl) | | | METHANOL-d4) δ = 8.29 |
| | phenoxy)-1H-pyrazolo | | | (s, 1H), 7.79-7.73 (m, 2H), |
| | [3,4-d]pyrimidin-1- | | | 7.67 (t, J = 8.0 Hz, 1H), |
| | yl)cyclobutan-1-ol | | | 7.61-7.57 (m, 1H), |
| | | | | 4.94-4.89 (m, 1H), |
| | | | | 4.15-4.07 (m, 1H), |
| | | | | 2.81-2.73 (m, 2H) |
| | | | | 2.53-2.50 (m, 2H), |
| 152 | (cis)-3-(4-amino-3-(3- | 331 | 332.1 | 1H NMR (400 MHz, |
| | chlorophenoxy)-1H- | | | ACETONITRILE-d3) δ = |
| | pyrazolo[3-4-d] | | | 10.58 (s, 1H), 8.22 (s, 1H), |
| | pyrimidin-1-yl) | | | 7.50-7.48 (m, 1H), |
| | cyclobutan-1-ol | | | 7.46 (d, J = 8.0 Hz, 1H), |
| | | | | 7.39 (d, J = 7.2 Hz, 1H), |
| | | | | 7.32 (d, J = 8.0 Hz, 1H), |
| | | | | 7.16 (s, 1H), 4.87-4.79 |
| | | | | (m, 1H), 4.12-4.04 (m, 1H), |
| | | | | 2.77-2.74 (m, 2H), |
| | | | | 2.45-2.42 (m, 2H) |
| 153 | (cis)-3-(4-amino-3-(3- | 315.1 | 316.1 | 1H NMR (400 MHz, |
| | fluorophenoxy)-1H- | | | (ACETONITRILE-d3) δ = |
| | pyrazolo[3,4-d] | | | 10.91 (s, 1H), 8.21 (s, 1H), |
| | pyrimidin-1-yl) | | | 7.48-7.46 (m, 1H), |
| | cyclobutan-1-ol | | | 7.26-7.23 (m, 2H), |
| | | | | 7.04 (t, J = 7.6 Hz, 1H), |
| | | | | 4.81-4.78 (m, 1H), |
| | | | | 4.10-4.03 (m, 1H), |
| | | | | 2.76-2.73 (m, 2H), |
| | | | | 2.44- 2.41 (m, 2H) |

### Synthesis Method R: Synthetic route for the preparation of (trans)-3-(4-amino-3-((5-fluoro-1H-indol-3-yl)(hydroxy)methyl)-1H-pyrazolo[3,4-d]pyrimidin-1-yl)cyclobutan-1-ol

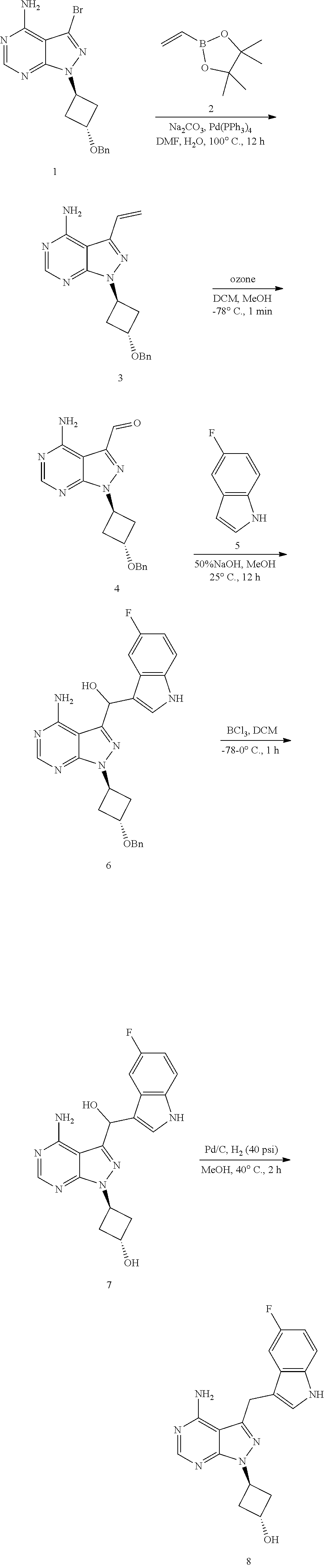

### Step 1. Procedure for preparation of 1-((trans)-3-(benzyloxy)cyclobutyl)-3-vinyl-1H-pyrazolo[3,4-d]pyrimidin-4-amine (3)

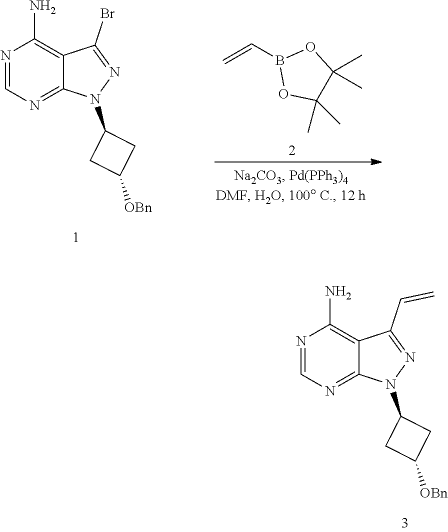 [SMILES: Nc1ncnc2c1c(Br)nn2[C@H]1C[C@H](OCc2ccccc2)C1 ; C=CB1OC(C)(C)C(C)(C)O1 ; C=Cc1nn([C@H]2C[C@H](OCc3ccccc3)C2)c2ncnc(N)c12]

Two round-bottom flasks were each charged with 1-((trans)-3-(benzyloxy)cyclobutyl)-3-bromo-1H-pyrazolo[3,4-d]pyrimidin-4-amine (1) (1.25 g, 3.34 mmol, 1 eq), 4,4,5,5-tetramethyl-2-vinyl-1,3,2-dioxaborolane (2) (1.03 g, 6.68 mmol, 1.13 mL, 2 eq), Na₂CO₃ (708 mg, 6.68 mmol, 2 eq), H₂O (5 mL) and DMF (15 mL). Each flask was filled with N₂ and evacuated (3×), and then N₂ gas was bubbled through the mixture for about 5 minutes. Pd(PPh₃)₄ (193 mg, 167 μmol, 0.05 eq) was added to both mixtures, and the flasks were maintained under N₂. The mixtures were heated to about 100° C. for about 12 h. Both batches were combined and filtered through celite and the filtrate was partitioned between ethyl acetate (50 mL) and water (100 mL), and then the aqueous phase was further extracted with ethyl acetate (3×50 mL). The combined organic layers were washed with brine (200 mL), dried over Na₂SO₄, filtered and concentrated under reduced pressure to give a residue. The residue was purified by column chromatography (SiO₂, Petroleum ether/Ethyl acetate=3/1) to afford 1-((1r,3r)-3-(benzyloxy)cyclobutyl)-3-vinyl-1H-pyrazolo[3,4-d]pyrimidin-4-amine (3) (2.1 g, 5.55 mmol, 83.1% yield, 85% purity) as a yellow solid.

### Step 2. Procedure for preparation of 4-amino-1-((trans)-3-(benzyloxy)cyclobutyl)-1H-pyrazolo[3,4-d]p yrimidine-3-carbaldehyde (4)

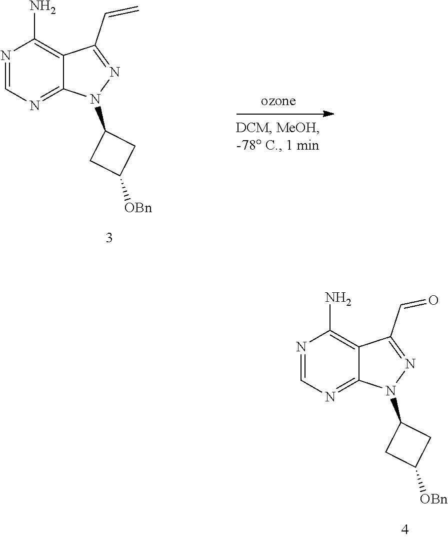 [SMILES: C=Cc1nn([C@H]2C[C@H](OCc3ccccc3)C2)c2ncnc(N)c12 ; Nc1ncnc2c1c(C=O)nn2[C@H]1C[C@H](OCc2ccccc2)C1]

Ozone was bubbled into a solution of 1-((1r,3r)-3-(benzyloxy)cyclobutyl)-3-vinyl-1H-pyrazolo[3,4-d]pyrimidin-4-amine (3) (500 mg, 1.56 mmol, 1 eq) in DCM (10 mL) and MeOH (10 mL) at −78° C. for 1 min. After excess O₃ was purged with O₂, Me₂S (4.23 g, 68.08 mmol, 5.00 mL, 43.76 eq) was added at −78° C. and warmed to 25° C. and stirred at 25° C. for 12 h. The 3 batches were combined together and the mixture was concentrated under reduced pressure to give a residue which was purified by column chromatography (SiO₂, Petroleum ether/Ethyl acetate=3/1) to give 4-amino-1-((trans)-3-(benzyloxy)cyclobutyl)-1H-pyrazolo[3,4-d]pyrimidine-3-carbaldehyde (4) (800 mg, 2.38 mmol, 50.9% yield, 96% purity) as a yellow solid. NMR: (400 MHz, DMSO-d₆) δ=10.00 (s, 1H), 8.31 (s, 1H), 8.26 (s, 1H), 7.50 (s, 1H), 7.40-7.36 (m, 4H), 7.34-7.28 (m, 1H), 5.51 (m, 1H), 4.54-4.46 (m, 3H), 2.87-2.76 (m, 2H), 2.72-2.60 (m, 3H).

### Step 3. Procedure for preparation of (4-amino-1-((trans)-3-(benzyloxy)cyclobutyl)-1H-pyrazolo[3,4-d]pyrimidin-3-yl)(5-fluoro-1H-indol-3-yl)methanol (6)

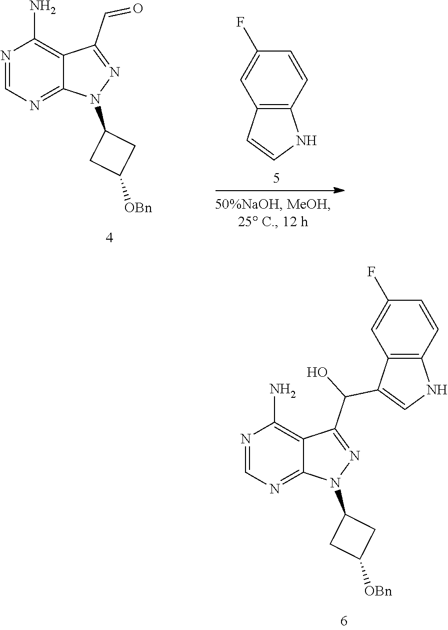 [SMILES: Nc1ncnc2c1c(C=O)nn2[C@H]1C[C@H](OCc2ccccc2)C1 ; Fc1ccc2[nH]ccc2c1 ; Nc1ncnc2c1c(C(O)c1c[nH]c3ccc(F)cc13)nn2[C@H]1C[C@H](OCc2ccccc2)C1]

To a solution of 4-amino-1-((trans)-3-(benzyloxy)cyclobutyl)-1H-pyrazolo[3,4-d]pyrimidine-3-carbaldehyde (4) (400 mg, 1.24 mmol, 1 eq) and 5-fluoro-1H-indole (5) (167 mg, 1.24 mmol, 1 eq) in MeOH (20 mL) was added NaOH (247 mg, 3.09 mmol, 50% purity, 2.5 eq) in one portion at 25° C. for 12 h. The mixture was filtered and the filtrate was concentrated under reduced pressure to give a residue that was purified by column chromatography (SiO₂, Petroleum ether/EtOAc: going from 50/1 to 0/1) to give (4-amino-1-((trans)-3-(benzyloxy)cyclobutyl)-1H-pyrazolo[3,4-d]pyrimidin-3-yl)(5-fluoro-1H-indol-3-yl)methanol (6) (260 mg, 329 μmol, 13.3% yield) as a green solid.

### Step 4. Procedure for preparation of (trans)-3-(4-amino-3-((5-fluoro-1H-indol-3-yl)(hydroxy)methyl)-1H-pyrazolo[3,4-d]pyrimidin-1-yl)cyclobutan-1-ol

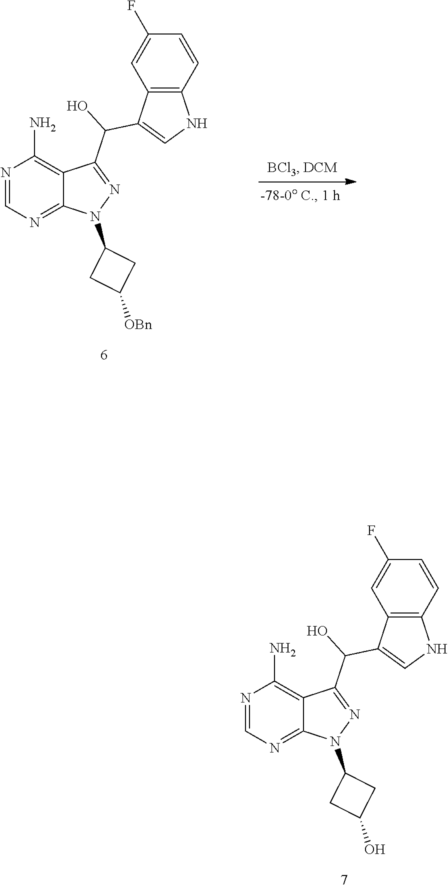 [SMILES: Nc1ncnc2c1c(C(O)c1c[nH]c3ccc(F)cc13)nn2[C@H]1C[C@H](OCc2ccccc2)C1 ; Nc1ncnc2c1c(C(O)c1c[nH]c3ccc(F)cc13)nn2[C@H]1C[C@H](O)C1]

To a solution of (4-amino-1-((trans)-3-(benzyloxy)cyclobutyl)-1H-pyrazolo[3,4-d]pyrimidin-3-yl)(5-fluoro-1H-indol-3-yl)methanol (6) (50 mg, 109 μmol, 1 eq) in DCM (2 mL) was added BCl₃ (1 M, 1.09 mL, 10 eq) drop-wise at −78° C. and then warmed to 0° C. and stirred at 0° C. for 1 h. The reaction was quenched by addition of 2 mL of saturated NaHCO₃ at 0° C., and then the resulting mixture was extracted with DCM (3×3 mL). The organic phase was washed with brine (3 mL) and dried over Na₂SO₄. Altered and concentrated under reduced pressure to a residue that was purified by prep-HPLC (neutral condition) to give (trans)-3-(4-amino-3-((5-fluoro-1H-indol-3-yl)(hydroxy)methyl)-1H-pyrazolo[3,4-d]pyrimidin-1-yl)cyclobutan-1-ol (1 mg, 2.70 μmol, 2.48% yield, 99.6% purity) as a yellow solid. LCMS: (M+H)⁺: 369.1, Rt: 2.187 min. (The gradient was 5-90% B in 3.4 min, 90-100% B in 0.45 min, 100-5% B in 0.01 min. and then held at 5% B for 0.65 min (0.8 mL/min flow rate). Mobile phase A was 10 mM NH4HCO3, mobile phase B was HPLC grade CH₃CN. The column used for the chromatography is a 2.1×50 mm Xbridge Shield RPC 18 column (5 μm particles). Detection methods are diode array (DAD) and evaporative light scattering (ELSD) detection as well as positive electrospray ionization (MS). ¹H NMR: (400 MHz, ACETONITRILE-d₃) δ=9.35 (s, 1H), 8.16 (s, 1H), 7.40-7.31 (m, 2H), 7.16 (d, J=2.0 Hz, 1H), 6.91 (dt, 7=2.4, 9.2 Hz, 1H), 6.35 (s, 1H), 5.47-5.38 (m, 1H), 4.85 (s, 1H), 4.70-4.61 (m, 1H), 3.35 (d, J=5.2 Hz, 1H), 2.87-2.71 (m, 2H), 2.50-2.40 (m, 2H).

### Step 5. Procedure for preparation of (trans)-3-(4-amino-3-((5-fluoro-1H-indol-3-yl)methyl)-1H-pyrazolo[3,4-d]pyrimidin-1-yl)cyclobutan-1-ol

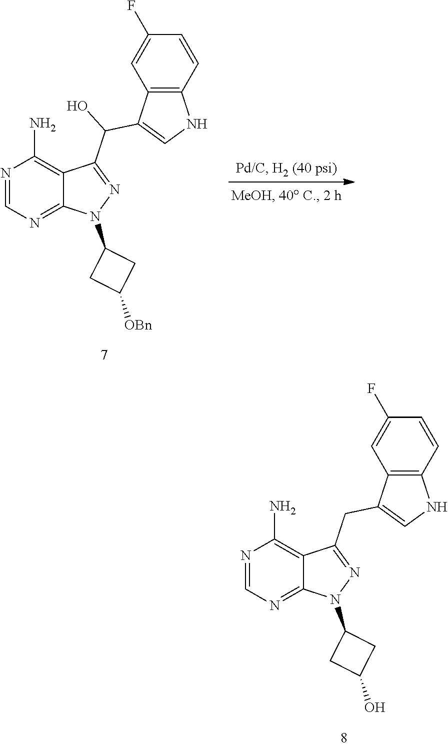 [SMILES: Nc1ncnc2c1c(C(O)c1c[nH]c3ccc(F)cc13)nn2[C@H]1C[C@H](OCc2ccccc2)C1 ; Nc1ncnc2c1c(Cc1c[nH]c3ccc(F)cc13)nn2[C@H]1C[C@H](O)C1]

To a solution of (4-amino-1-((trans)-3-(benzyloxy)cyclobutyl)-1H-pyrazolo[3,4-d]pyrimidin-3-yl)(5-fluoro-1H-indol-3-yl)methanol (7) (50 mg, 109 μmol, 1 eq) in MeOH (10 mL) was added Pd/C (100 mg) under argon atmosphere. The suspension was degassed and purged with H₂ for 3 times. The mixture was stirred under H₂ (40 Psi) at 40° C. for 2 h. The two batches of mixture were cooled to 25° C., combined and filtered through celite. The filtrate was concentrated under reduced pressure and the residue purified by prep-HPLC (neutral conditions) to give (trans)-3-(4-amino-3-((5-fluoro-1H-indol-3-yl)methyl)-1H-pyrazolo[3,4-d]pyrimidin-1-yl)cyclobutan-1-ol (8; Compound 154) (16.8 mg, 47.6 μmol, 21.8% yield, 99.8% purity) as a white solid. LCMS: (M+H)⁺: 353.1, Rt: 2.344 min. LC/MS Method: The gradient was 5-90% B in 3.4 min, 90-100% B in 0.45 min, 100-5% B in 0.01 min, and then held at 5% B for 0.65 min (0.8 mL/min flow rate. Mobile phase A was 10 mM NH₄HCO₃, mobile phase B was HPLC grade CH₃CN. The column used for the chromatography is a 2.1×50 mm Xbridge Shield RPC 18 column (5 μm particles). Detection methods are diode array (DAD) and evaporative light scattering (ELSD) detection as well as positive electrospray ionization (MS). ¹H NMR: (400 MHz, ACETONITRILE-d₃) δ=9.27 (s, 1H), 8.12 (s, 1H), 7.40-7.36 (m, 1H), 7.20 (s, 1H), 7.15 (d, J=10.4 Hz, 1H), 6.92 (t, J=9.2 Hz, 1H), 5.59 (s, 2H), 5.46-5.42 (m, 1H), 4.70-4.67 (m, 1H), 4.36 (s, 2H), 3.27 (s, 1H), 2.87-2.82 (m, 2H), 2.50-2.46 (m, 2H).

### Synthesis Method S: General Procedure Represented by the Preparation of 3-[4-amino-5-[(3-fluorophenyl)methyl]pyrrolo[2,3-d]pyrimidin-7-yl]cyclobutanol (6)

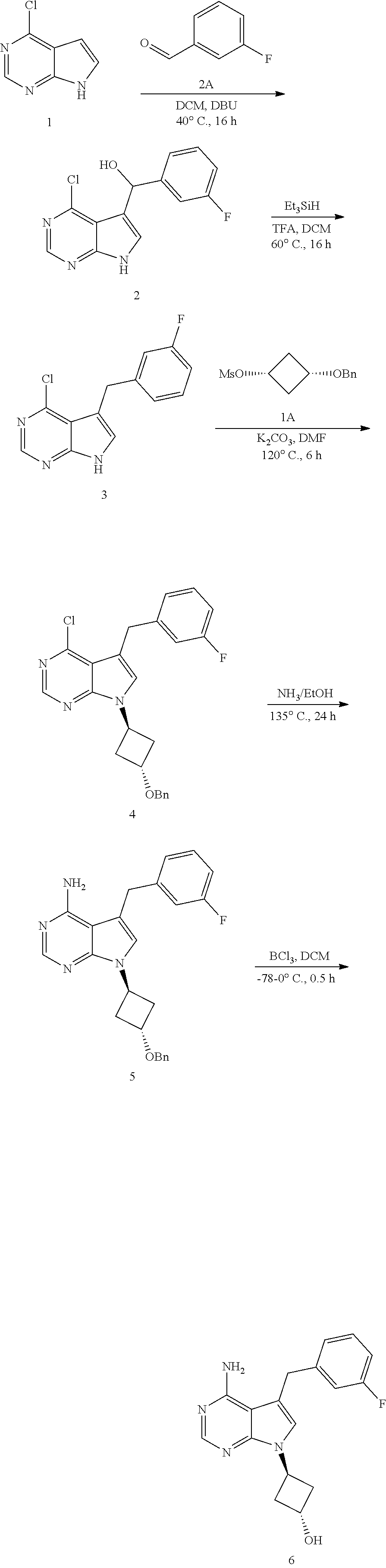

### Step 1. Procedure for preparation of (4-chloro-7H-pyrrolo[2,3-d]pyrimidin-5-yl)-(3-fluorophenyl)methanol (2)

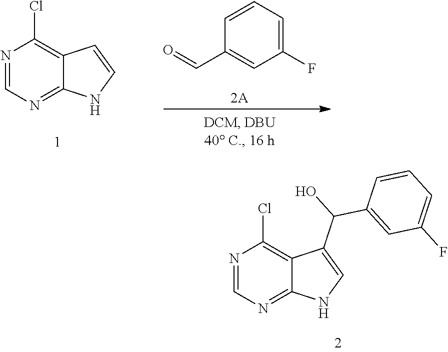 [SMILES: Clc1ncnc2[nH]ccc12 ; O=Cc1cccc(F)c1 ; OC(c1cccc(F)c1)c1c[nH]c2ncnc(Cl)c12]

DBU (793 mg, 5.20 mmol, 785 μL, 0.2 eq) was added via syringe into a stirred mixture of 4-chloro-7H-pyrrolo[2,3-d]pyrimidine (1) (4 g, 26.0 mmol, 1 eq) and 3-fluorobenzaldehyde (6.48 g, 52.1 mmol, 5.48 mL, 2 eq) (2A) in the DCM (20 mL). The mixture was stirred at 40° C. for 16 h under N₂. The reaction was cooled to 20° C., and the white solid was removed by filtration and washed with 40 mL of DCM. The filtrate was concentrated under reduced pressure using a rotary evaporator. The crude product was purified by silica gel column chromatography (DCM/MeOH=1/0 to 10/1) to afford (4-chloro-7H-pyrrolo[2,3-d]pyrimidin-5-yl)-(3-fluorophenyl)methanol (2) (1.2 g, 4.32 mmol, 16.6% yield) as a yellow solid.

### Step 2. Procedure for preparation of 4-chloro-5-[(3-fluorophenyl)methyl]-7H-pyrrolo[2,3-d]pyrimidine (3)

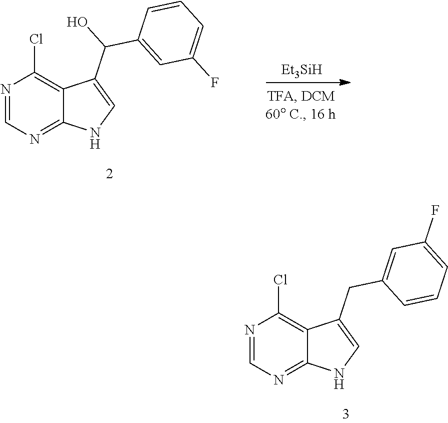 [SMILES: "Et3SiH" ; OC(c1cccc(F)c1)c1c[nH]c2ncnc(Cl)c12 ; Fc1cccc(Cc2c[nH]c3ncnc(Cl)c23)c1]

A suspension of (4-chloro-7H-pyrrolo[2,3-d]pyrimidin-5-yl)-(3-fluorophenyl)methanol (500 mg, 1.80 mmol, 1 eq) (2) in DCM (5 mL) was treated sequentially with triethylsilane (628 mg, 5.40 mmol, 863 μL, 3 eq) and TFA (411 mg, 3.60 mmol, 267 μL, 2 eq) and stirred at 60° C. for 16 h. The reaction was quenched by addition of 10 mL of water, and then extracted by diethyl ether (3×10 mL). The organic phase was concentrated and the crude product was purified by silica gel column chromatography (DCM/MeOH=1/0 to 10/1) to afford 4-chloro-5-[(3-fluorophenyl)methyl]-7H-pyrrolo[2,3-d]pyrimidine (3) (300 mg, 1.15 mmol, 63.67% yield) as a white solid.

### Step 3. Procedure for preparation of 7-(3-benzyloxycyclobutyl)-4-chloro-5-[(3-fluorophenyl)methyl]pyrrolo[2,3-d]pyrimidine (4)

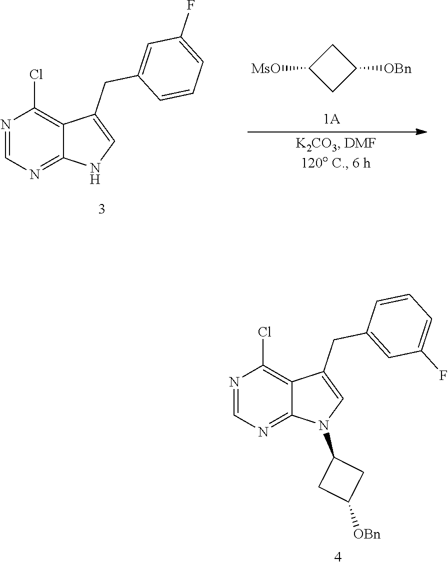 [SMILES: Fc1cccc(Cc2c[nH]c3ncnc(Cl)c23)c1 ; CS(=O)(=O)O[C@H]1C[C@@H](OCc2ccccc2)C1 ; Fc1cccc(Cc2cn([C@H]3C[C@H](OCc4ccccc4)C3)c3ncnc(Cl)c23)c1]

A mixture of 4-chloro-5-[(3-fluorophenyl)methyl]-7H-pyrrolo[2,3-d]pyrimidine (250 mg, 955 μmol, 1 eq) (3), K₂CO₃ (264 mg, 1.91 mmol, 2 eq) and (3-benzyloxycyclobutyl) methanesulfonate (416 mg, 1.62 mmol, 1.7 eq) (1A) in DMF (5 mL) was stirred at 120° C. for 6 h. The reaction was quenched by addition of 10 mL of water, and then extracted by EtOAc (3×5 mL). The crude product was purified by silica gel column chromatography (DCM/MeOH=1/0 to 10/1) to afford 7-(3-benzyloxycyclobutyl)-4-chloro-5-[(3-fluorophenyl)methyl]pyrrolo[2,3-d]pyrimidine (4) (100 mg, 237 μmol, 24.8% yield) as a yellow solid.

### Step 4. Procedure for preparation of 7-(3-benzyloxycyclobutyl)-S-[(3-fluorophenyl)methyl]pyrrolo[2,3-d]pyrimidin-4-amine (Si

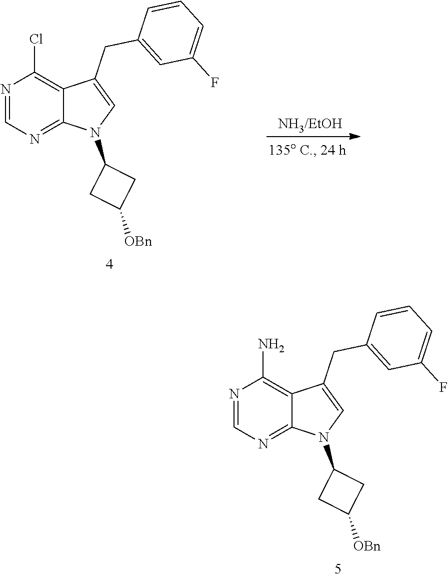 [SMILES: Fc1cccc(Cc2cn([C@H]3C[C@H](OCc4ccccc4)C3)c3ncnc(Cl)c23)c1 ; "NH3/EtOH" ; Nc1ncnc2c1c(Cc1cccc(F)c1)cn2[C@H]1C[C@H](OCc2ccccc2)C1]

A solution of 7-(3-benzyloxycyclobutyl)-4-chloro-5-[(3-fluorophenyl)methyl]-pyrrolo[2,3-d]pyrimidine (100 mg, 237 μmol, 1 eq) (4) in NH₃/EtOH (4 M, 150 mL, 2531 eq) was stirred at 135° C. for 24 h in a sealed tube. The reaction mixture was concentrated under reduced pressure using a rotary evaporator. The crude product was purified by prep-TLC (DCM/MeOH=10/1) to afford 7-(3-benzyloxycyclobutyl)-5-[(3-fluorophenyl)methyl]pyrrolo[2,3-d]pyrimidin-4-amine (5) (50 mg, 124 μmol, 52.4% yield) as a white solid.

### Step 5. Procedure for preparation of 3-[4-amino-5-[(3-fluorophenyl)methyl]pyrrolo[2,3-d]pyrimidin-7-yl]cyclobutanol (6)

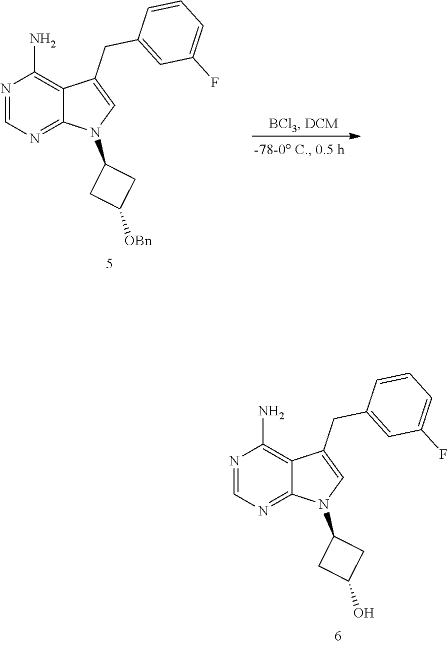

BCl₃ (1 M, 1.24 mL, 10 eq) was added dropwise to a stirred mixture of 7-(3-benzyloxycyclobutyl)-5-[(3-fluorophenyl)methyl]pyrrolo[2,3-d]pyrimidin-4-amine (5) (50 mg, 124 μmol, 1 eq) in DCM (2 mL) at −78° C. under N₂. The mixture was stirred at 0° C. for 0.5 h. The reaction was quenched by addition of 5 mL of MeOH at −78° C., and then stirred for 2 min. The mixture was basified by slowly adding ammonium hydrooxide at 0° C. to pH=8, filtered, and the filtrate was concentrated and the residue was purified by prep-HPLC (TFA condition) to give 3-[4-amino-5-[(3-fluorophenyl)methyl]pyrrolo[2,3-d]pyrimidin-7-yl]cyclobutanol (6) (15.3 mg, 35.5 μmol, 28.6% yield, 99% purity, TFA) as a white solid.

¹H NMR: (400 MHz, acetonitrile-d₃) δ=8.11 (s, 1H), 7.36-7.32 (m, 1H), 7.26 (s, 1H), 7.08 (d, J=7.6 Hz, 1H), 7.00-6.95 (m, 2H), 5.43-5.35 (m, 1H), 4.55-4.50 (m, 1H), 4.19 (s, 2H), 2.70-2.64 (m, 2H), 2.51-2.48 (m, 2H).

### Synthesis Method T: General Procedure Represented by the Preparation of 2-(4-amino-1-cyclopropyl-pyrazolo[3,4-d]pyrimidin-3-yl)oxypyridin-4-ol (3)

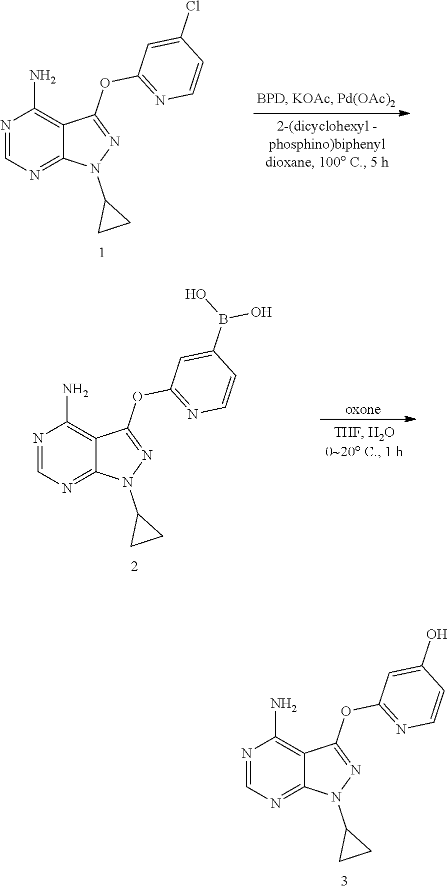 [SMILES: Nc1ncnc2c1c(Oc1cc(Cl)ccn1)nn2C1CC1 ; Nc1ncnc2c1c(Oc1cc(B(O)O)ccn1)nn2C1CC1 ; Nc1ncnc2c1c(Oc1cc(O)ccn1)nn2C1CC1]

### Step 1. Procedure for preparation of [2-(4-amino-1-cyclopropyl-pyrazolo[3,4-d]pyrimidin-3-yl)oxy-4-pyridyl]boronic acid (2)

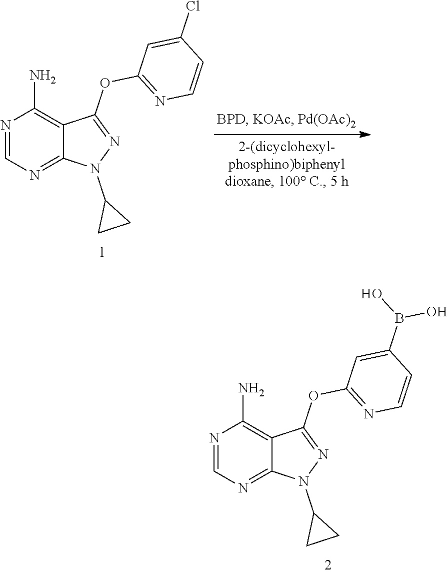 [SMILES: Nc1ncnc2c1c(Oc1cc(Cl)ccn1)nn2C1CC1 ; Nc1ncnc2c1c(Oc1cc(B(O)O)ccn1)nn2C1CC1]

Pd(OAc)₂ (37.1 mg, 165 μmol, 0.05 eq) was added into a stirred mixture of 3-[(4-chloro-2-pyridyl)oxy]-1-cyclopropyl-pyrazolo[3,4-d]pyrimidin-4-amine (1) (1 g, 3.30 mmol, 1 eq), BPD (1.68 g, 6.61 mmol, 2 eq), KOAc (973 mg, 9.91 mmol, 3 eq) and dicyclohexyl-(2-phenylphenyl)phosphane (57.9 mg, 165 μmol, 0.05 eq) in dioxane (10 mL). The mixture was stirred at 100° C. for 5 h, quenched by addition of 20 mL of water, and the yellow solid was collected by filtration, washed with three portions (5 mL each) of water and dried under reduced pressure using a rotary evaporator to give [2-(4-amino-1-cyclopropyl-pyrazolo[3,4-d]pyrimidin-3-yl)oxy-4-pyridyl]boronic acid (2) (2 g, crude) as a yellow solid.

¹H NMR: (400 MHz, DMSO-d₆) δ=8.62 (s, 1H), 8.27 (s, 1H), 8.11 (d, J=5.6 Hz, 1H), 7.50-7.47 (m, 2H), 3.74-3.68 (m, 1H), 1.07-0.99 (m, 4H)

### Step 2. Procedure for preparation of 2-(4-amino-1-cyclopropyl-pyrazolo[3,4-d]pyrimidin-3-yl)oxypyridin-4-ol (1.5 g, crude) (3)

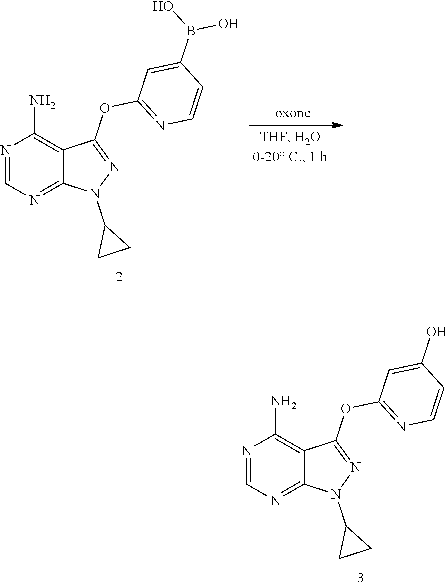 [SMILES: Nc1ncnc2c1c(Oc1cc(B(O)O)ccn1)nn2C1CC1 ; Nc1ncnc2c1c(Oc1cc(O)ccn1)nn2C1CC1]

A solution of oxone (3.27 g, 5.33 mmol, 1.05 eq) in water (20 mL) was added to a stirred mixture of [2-(4-amino-1-cyclopropyl-pyrazolo[3,4-d]pyrimidin-3-yl)oxy-4-pyridyl]boronic acid (2 g, 6.41 mmol, 1 eq) in THF (20 mL) at 0° C. The mixture was stirred at 20° C. for 1 h and quenched by addition of 20 mL of saturation sodium sulfite. The white solid was collected by filtration, washed with three portions (10 mL each) of water and dried under reduced pressure using a rotary evaporator to give 2-(4-amino-1-cyclopropyl-pyrazolo[3,4-d]pyrimidin-3-yl)oxypyridin-4-ol (1.5 g, crude) as a white solid. A 200 mg portion of the crude product was purified by prep-HPLC (TFA condition) to give 5 mg of 2-(4-amino-1-cyclopropyl-pyrazolo[3,4-d]pyrimidin-3-yl)oxypyridin-4-ol (100% LCMS purity. TFA) as a white solid.

¹H NMR: (400 MHz, methanol-d₄) δ=8.34 (s, 1H), 7.% (d, J=5.6 Hz, 1H), 6.89 (d, J=2.0 Hz, 1H), 6.73 (dd, J=2.0, 6.0 Hz, 1H), 3.85 (tt, J=3.6, 7.2 Hz, 1H), 1.30-1.20 (m, 2H), 1.20-1.09 (m, 2H).

### Synthesis Method U: General Procedure Represented by the Preparation of 1-cyclopropyl-3-[(4-cyclopropyl-2-pyridyl)oxy]pyrazolo[3,4-d]pyrimidin-4-amine

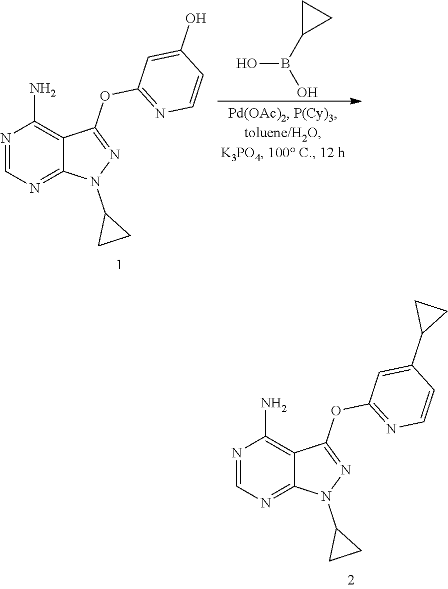 [SMILES: Nc1ncnc2c1c(Oc1cc(O)ccn1)nn2C1CC1 ; OB(O)C1CC1 ; Nc1ncnc2c1c(Oc1cc(C3CC3)ccn1)nn2C1CC1]

A mixture of 3-[(4-chloro-2-pyridyl)oxy]-1-cyclopropyl-pyrazolo[3,4-d]pyrimidin-4-amine (1) (80 mg, 264 μmol, 1 eq), cyclopropylboronic acid (45.4 mg, 529 μmol, 2 eq), Pd(OAc)₂ (1.19 mg, 5.29 μmol, 0.02 eq), K₃PO₄ (1% mg, 925 μmol, 3.5 eq), P(Cy)₃ (7.41 mg, 26.4 μmol, 8.57 μL, 0.1 eq) in H₂O (0.2 mL) and toluene (4 mL) was degassed and purged with N₂ for 3 times, and then stirred at 100° C. for 12 h under N₂ atmosphere. The mixture was filtered and the filtrate was concentrated under reduced pressure and the residue was purified by prep-HPLC (TFA condition) to give 1-cyclopropyl-3-[(4-cyclopropyl-2-pyridyl)oxy]pyrazolo[3,4-d]pyrimidin-4-amine (2) (6 mg, 18.9 μmol, 7.14% yield, 97% purity) as a white solid. LCMS: (M+H)⁺: 309.1, Rt: 4.548 min. ¹H NMR: (400 MHz, MeOD-d₄) δ=8.31 (s, 1H), 8.01 (d, J=5.2 Hz, 1H), 7.13 (s, 1H), 6.98 (d, J=4.0 Hz, 1H), 3.83-3.77 (m, 1H), 2.10-2.01 (m, 1H), 1.22-1.19 (m, 4H), 1.18-1.17 (m, 2H), 0.92-0.89 (m, 2H).

### Synthesis Method V: General Procedure Represented by the Preparation of 2-((4-amino-1-cyclopentyl-1H-pyrazolo[3,4-d]pyrimidin-3-yl)oxy)isonicotinonitrile (8)

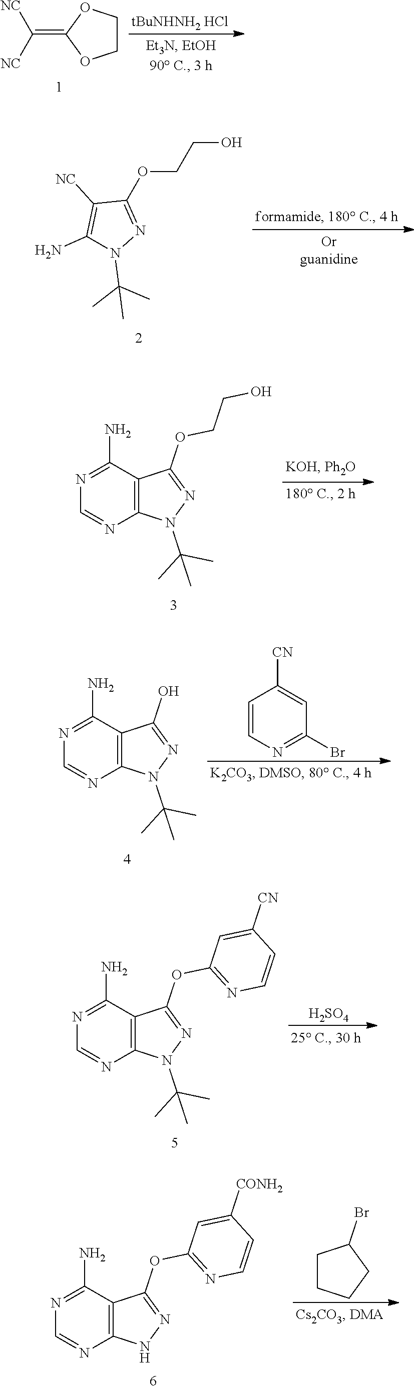

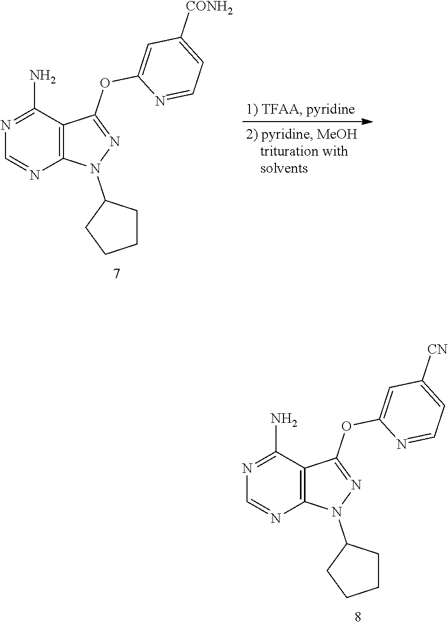 [SMILES: NC(=O)c1ccnc(Oc2nn(C3CCCC3)c3ncnc(N)c23)c1 ; N#Cc1ccnc(Oc2nn(C3CCCC3)c3ncnc(N)c23)c1]

### Step 1. Procedure for preparation of 5-amino-1-tert-butyl-3-(2-hydroxyethoxy)pyrazole-4-carbonitrile (2)

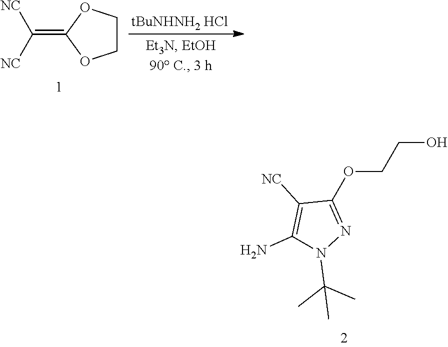 [SMILES: "tBuNHNH2 HCl" ; N#CC(C#N)=C1OCCO1 ; CC(C)(C)n1nc(OCCO)c(C#N)c1N]

To a solution of 2-(1,3-dioxolan-2-ylidene)propanedinitrile (1) (600 g, 4.41 mol, 1 eq) in EtOH (1 L) was added tert-butylhydrazine (582.28 g, 4.67 mol, 1.06 eq, HCl) and Et₃N (892.14 g, 8.82 mol, 1.23 L, 2 eq) slowly. The mixture was stirred at 90° C. for 3 hrs. LCMS showed the reaction was completed. The reaction was concentrated to afford the crude product. The residue was purified by column chromatography (SiO₂, Petroleum ether/Ethyl acetate=3/1 to 0/1) to afford 5-amino-1-tert-butyl-3-(2-hydroxyethoxy)pyrazole-4-carbonitrile (2) (550 g, 2.33 mol, 52.85% yield, 95% purity) as a light yellow solid.

LCMS: [M+H]⁺: 225.1.

¹H NMR: (400 MHz, DMSO-d₆) δ=6.20 (s, 2H), 4.81-4.83 (m, 1H), 4.07-4.09 (m, 2H), 3.63-3.67 (m, 2H), 1.45 (s, 9H).

### Step 2. Procedure for preparation of 2-(4-amino-1-tert-butyl-pyrazolo[3,4-d]pyrimidin-3-yl)oxyethanol (3)

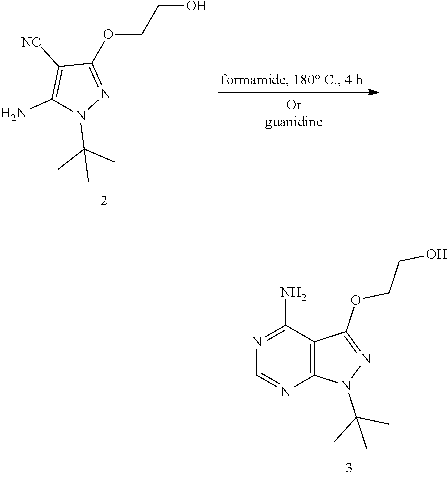 [SMILES: CC(C)(C)n1nc(OCCO)c(C#N)c1N ; CC(C)(C)n1nc(OCCO)c2c(N)ncnc21]

The solution of 5-amino-1-tert-butyl-3-(2-hydroxyethoxy)pyrazole-4-carbonitrile (2) (110 g, 490.50 mmol, 1 eq) and formamide (550 g, 12.21 mol, 486.73 mL, 24.90 eq) was stirred at 180° C. for 4 hrs. The reaction mixture was diluted with water 5 L and extracted with EtOAc 9 L (3×3 L). The combined organic layers were dried over anhydrous sodium sulfate, filtered and concentrated under reduced pressure to give a residue. The residue was purified by column chromatography (SiO₂, Petroleum ether/Ethyl acetate=1/1 to 1/2) to give 2-(4-amino-1-tert-butyl-pyrazolo[3,4-d]pyrimidin-3-yl)oxyethanol (3) (300 g, 1.19 mol, 48.68% yield) as a yellow solid.

LCMS: [M+H]⁺: 252.1

¹H NMR: (400 MHz, DMSO-d₆) δ=8.14 (s, 1H), 5.09-5.13 (m, 1H), 4.26-4.28 (m, 2H), 3.77-3.81 (m, 2H), 1.67 (s, 9H).

### Step 3. Procedure for preparation of 4-amino-1-tert-butyl-pyrazolo[3,4-d]pyrimidin-3-ol (4)

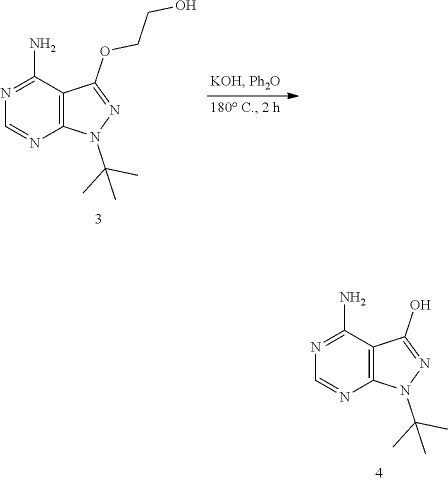 [SMILES: CC(C)(C)n1nc(OCCO)c2c(N)ncnc21 ; CC(C)(C)n1nc(O)c2c(N)ncnc21]

To a solution of 2-(4-amino-1-tert-butyl-pyrazolo[3,4-d]pyrimidin-3-yl)oxyethanol (3) (80 g, 318.36 mmol, 1 eq) in Ph₂O (500 mL) was added KOH (178.62 g, 3.18 mol, 10 eq). The mixture was stirred at 180° C. for 2 hrs. The resulting mixture was partitioned between Petroleum ether (500 mL) and water (800 mL), and then the aqueous phase was adjust to pH=7. Collect the crystalline solid by suction filtration, wash with three 800-mL portions of cold water and dry constant weight at 45° C. 4-amino-1-tert-butyl-pyrazolo[3,4-d]pyrimidin-3-ol (4) (50 g, 75.79% yield) was obtained as a white solid.

LCMS: [M+H]⁺: 208.1

¹H NMR: (400 MHz, DMSO-d₆) δ=8.04 (s, 1H), 1.60 (s, 9H).

### Step 4. Procedure for preparation of 2-(4-amino-1-tert-butyl-pyrazolo[3,4-d]pyrimidin-3-yl)oxypyridine-4-carbonitrile (5)

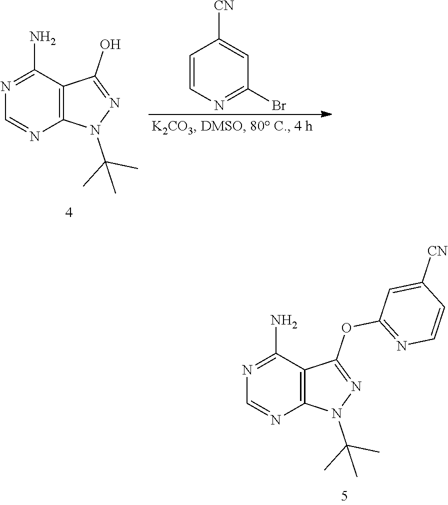 [SMILES: CC(C)(C)n1nc(O)c2c(N)ncnc21 ; N#Cc1ccnc(Br)c1 ; CC(C)(C)n1nc(Oc2cc(C#N)ccn2)c2c(N)ncnc21]

To a solution of 4-amino-1-tert-butyl-pyrazolo[3,4-d]pyrimidin-3-ol (4) (50 g, 241.28 mmol, 1 eq) and 2-bromopyridine-4-carbonitrile (52.99 g, 289.53 mmol, 1.2 eq) in DMSO (500 mL) was added K₂CO₃ (66.69 g, 482.55 mmol, 2 eq) stirred at 80° C. for 4 hrs. The resulting mixture was partitioned between ethyl acetate (900 mL) and water (1000 mL), and then the aqueous phase was further extracted with ethyl acetate (3×300 mL), then the organic phase was washed with brine (500 mL) and dried over Na₂SO₄ and concentrated. The residue was purified by column chromatography (SiO₂, Petroleum ether/Ethyl acetate=30/1 to 1/1). 2-(4-amino-1-tert-butyl-pyrazolo[3,4-d]pyrimidin-3-yl)oxypyridine-4-carbonitrile (5) (43 g, 139.01 mmol, 57.62% yield) was obtained as a white solid.

¹H NMR: (400 MHz, DMSO-d₆) δ=8.36 (d, J=5.14 Hz, 1H), 8.18 (s, 1H), 7.77 (s, 1H), 7.62 (dd, J=5.14, 1.10 Hz, 1H), 1.66 (s, 9H).

### Step 5. Procedure for preparation of 2-[(4-amino-1H-pyrazolo[3,4-d]pyrimidin-3-yl)oxy]pyridine-4-carboxamide (6)

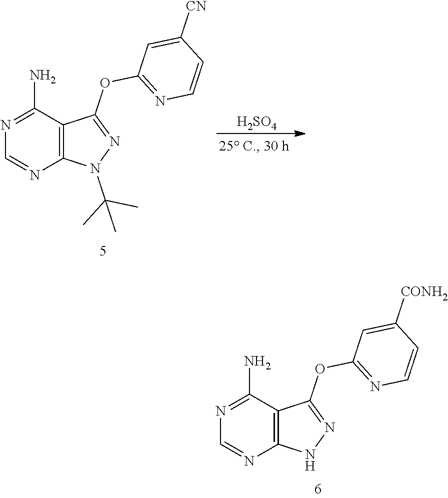

The mixture of 2-(4-amino-1-tert-butyl-pyrazolo[3,4-d]pyrimidin-3-yl) oxypyridine-4-carbonitrile (5) (43 g, 139.01 mmol, 1 eq) and H₂SO₄ (130 mL) was stirred at 25° C. for 30 h. The reaction mixture was quenched by addition water (200 mL) at 0° C., and then the mixture was basified by slowly adding NaOH saturated solution at 0° C. to pH=7. Then, the reaction mixture was filtered to give filter cake. The filter cake was washed with water 600 mL (3×200 mL), and the mixture was concentrated under reduced pressure. The crude product was used to the next step without further purification.

¹H NMR (400 MHz, DMSO-d₆): δ=8.30 (s, 1H), 8.25 (d, J=5.70 Hz, 1H), 8.17 (s, 1H), 7.80 (s, 1H), 7.56-7.59 (m, 2H).

### Step 6. Procedure for preparation of 2-((4-amino-1-cyclopentyl-1H-pyrazolo[3,4-d]pyrimidin-3-yl)oxy)isonicotinomide (7)

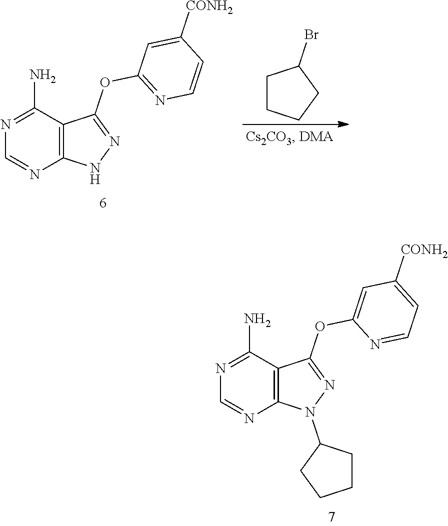 [SMILES: NC(=O)c1ccnc(Oc2n[nH]c3ncnc(N)c23)c1 ; BrC1CCCC1 ; NC(=O)c1ccnc(Oc2nn(C3CCCC3)c3ncnc(N)c23)c1]

To a solution of 2-[(4-amino-1H-pyrazolo[3,4-d]pyrimidin-3-yl)oxy]pyridine-4-carboxamide (0.2 g, 737.37 umol, 1 eq) and bromocyclopentane (219.78 mg, 1.47 mmol, 158.11 uL, 2 eq) in DMA (3 mL) was added Cs₂CO₃ (720.75 mg, 2.21 mmol, 3 eq). The mixture was stirred at 100° C. for 16 h. LCMS and HPLC showed the reaction was complete. Then the mixture was filtered and concentrated under reduced pressure to give a residue. The residue was purified by prep-HPLC (TFA condition) to give 2-(4-amino-1-cyclopentyl-pyrazolo[3,4-d]pyrimidin-3-yl)oxypyridine-4-carboxamide (0.054 g. yield 21.51%, purity 99.67%) as a white solid.

¹H NMR (400 MHz, DMSO-d₆): δ=8.29-8.31 (m, 2H), 8.23-8.25 (m, 1H), 7.80 (s, 1H), 7.57-7.58 (m, 2H), 5.12-5.19 (m, 1H), 2.03-2.06 (m, 2H), 1.77-1.88 (m, 4H), 1.61-1.63 (m, 2H).

### Step 7. Procedure for preparation of 2-((4-amino-1-cyclopentyl-1H-pyrazolo[3,4-d]pyrimidin-3-yl)oxy)isonicotinonitrile (8)

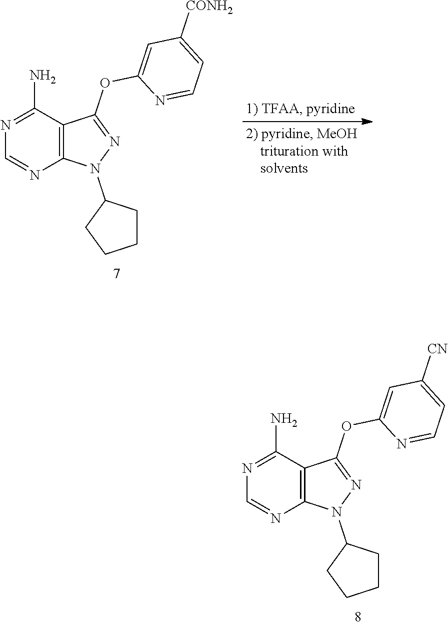 [SMILES: NC(=O)c1ccnc(Oc2nn(C3CCCC3)c3ncnc(N)c23)c1 ; N#Cc1ccnc(Oc2nn(C3CCCC3)c3ncnc(N)c23)c1]

To a solution of 2-(4-amino-1-cyclopentyl-pyrazolo[3,4-d]pyrimidin-3-yl)oxypyridine-4-carboxamide (50 mg, 147.34 umol, 1 eq) in dioxane (2 mL) was added pyridine (116.55 mg, 1.47 mmol, 118.92 uL, 10 eq) at 25° C. Then TFAA (154.73 mg, 736.70 umol, 102.47 uL, 5 eq) was added dropwise at 25° C. The mixture was stirred at 25° C. for 1 h. The reaction mixture was concentrated under reduced pressure to give a residue. Py (2 mL) was added into the residue, the mixture was stirred at 60° C. for 3 h. LCMS and HPLC showed the reaction was complete. Then the mixture was filtered and concentrated under reduced pressure to give a residue. The residue was purified by prep-HPLC (TFA condition) to give 2-(4-amino-1-cyclopentyl-pyrazolo[3,4-d]pyrimidin-3-yl)oxypyridine-4-carbonitrile (31 mg, yield 64.82%, purity 99%) as a white solid. LCMS: [M+H]⁺: 322.2.

¹H NMR (400 MHz, DMSO-d₆): δ=8.34-8.35 (d, 2H), 8.26 (s, 1H), 7.79 (s, 1H), 7.63-7.64 (m, 1H), 5.11-5.18 (m, 1H), 2.02-2.05 (m, 2H), 1.77-1.89 (m, 4H), 1.61-1.63 (m, 2H).

The following compound was prepared in a similar manner as for method V using different starting materials.

TABLE 16
Compounds Prepared by Method V

| Com- | | | LC/MS | |
|---|---|---|---|---|
| pound | | | Observed | |
| No. | IUPAC Name | MW | (M + H) | ¹H NMR (400 MHz) |
| 192 | 1-cyclopentyl-3-((4- | 364.33 | 365.1 | (DMSO-d₆) δ = 8.39 (d, J = 4.0 |
| | (trifluoromethyl)pyridin-2- | | | Hz, 1H), 8.18 (s, 1H), 7.61 (s, |
| | yl)oxy)-1H-pyrazolo[3,4- | | | 1H), 7.54 (d, J = 8.0 Hz 1H), |
| | d]pyrimidin-4-amine | | | 5.18-5.11 (m, 1H), 2.05-2.02 |
| | | | | (m, 2H), 1.89-1.88 (m, 2H), |
| | | | | 1.79-1.78 (m, 2H), 1.64-1.62 |
| | | | | (m, 2H) |
| 193 | 1-(tetrahydrofuran-3-yl)-3- | 366.30 | 367.0 | (METHANOL-d₄) δ = 8.42 (d, J = |
| | ((4- | | | 5.2 Hz, 1H), 8.33 (s, 1H), 7.73 |
| | (trifluoromethyl)pyridin-2- | | | (s, 1H), 7.54 (d, J = 5.2 Hz, 1H), |
| | yl)oxy)-1H-pyrazolo[3,4- | | | 5.60-5.55 (m, 1H), 4.16-4.10 |
| | d]pyrimidin-4-amine | | | (m, 2H), 4.09-3.93 (m, 2H), |
| | | | | 2.53-2.38 (m, 2H) |
| 194 | 1-(piperidin-4-yl)-3-((4- | 379.34 | 380.1 | (METHANOL-d₄) δ = 8.41 (d, J = |
| | (trifluoromethyl)pyridin-2- | | | 4.8 Hz, 1H), 8.33 (s, 1H), 7.68 |
| | yl)oxy)-1H-pyrazolo[3,4- | | | (s, 1H), 7.55 (d, J = 5.2 Hz, 1H), |
| | d]pyrimidin-4-amine | | | 5.14-5.08 (m, 1H), 3.59-3.54 |
| | | | | (m, 2H), 3.27-3.23 (m, 2H), |
| | | | | 2.42-2.25 (m, 4H) |
| 195 | 1-(piperidin-3-yl)-3-((4- | 379.34 | 380.1 | (METHANOL-d₄) δ = 8.40 (d, J = |
| | (trifluoromethyl)pyridin-2- | | | 4.8 Hz, 1H), 8.32 (s, 1H), 7.66 |
| | yl)oxy)-1H-pyrazolo[3,4- | | | (s, 1H), 7.54 (d, J = 4.8 Hz, 1H), |
| | d]pyrimidin-4-amine | | | 5.15-5.08 (m, 1H), 3.64-3.57 |
| | | | | (m, 2H), 3.21-3.14 (m, 2H), |
| | | | | 2.22-2.19 (m, 2H), 2.10-1.92 |
| | | | | (m, 1H) |
| 196 | tert-butyl 4-((4-amino-3- | 529.46 | 530.2 | (METHANOL-d₄) δ = 8.41 (d, J = |
| | ((4- | | | 5.2 Hz, 1H), 8.22 (s, 1H), 7.67 |
| | (trifluoromethyl)pyridin-2- | | | (s, 1H), 7.51 (d, J = 5.6 Hz, 1H), |
| | yl)oxy)-1H-pyrazolo[3,4- | | | 4.63-4.57 (m, 2H), 4.40-4.33 |
| | d]pyrimidin-1-yl)methyl)- | | | (m, 1H), 4.25 (s, 1H), 4.05- |
| | 3,3-difluoropiperidine-1- | | | 4.01 (m, 1H), 2.81-2.66 (m, |
| | carboxylate | | | 2H), 1.62-1.49 (m, 2H), 1.46 |
| | | | | (s, 9H) |
| 197 | 1-((3,3-difluoropiperidin- | 429.35 | 430.1 | (METHANOL-d₄) δ = 8.41 (d, J = |
| | 4-yl)methyl)-3-((4- | | | 5.6 Hz, 1H), 8.31 (s, 1H), 7.68 |
| | (trifluoromethyl)pyridin-2- | | | (s, 1H), 7.55 (d, J = 5.2 Hz, 1H), |
| | yl)oxy)-1H-pyrazolo[3,4- | | | 4.75-4.70 (m, 1H), 4.48-4.41 |
| | d]pyrimidin-4-amine | | | (m, 1H), 3.77-3.72 (m, 1H), |
| | | | | 3.54-3.40 (m, 2H), 3.14-3.08 |
| | | | | (m, 1H), 3.08-2.89 (m, 1H), |
| | | | | 2.01-1.83 (m, 2H) |
| 198 | 1-(tetrahydro-2H-pyran-4- | 380.32 | 381.1 | (METHANOL-d₄) δ = 8.39 (d, J = |
| | yl)-3-((4- | | | 5.2 Hz, 1H), 8.20 (s, 1H), 7.62 |
| | (trifluoromethyl)pyridin-2- | | | (s, 1H), 7.56 (d, J = 5.2 Hz, 1H), |
| | yl)oxy)-1H-pyrazolo[3,4- | | | 4.88-4.80 (m, 1H), 3.98-3.94 |
| | d]pyrimidin-4-amine | | | (m, 2H), 3.50 (t, J = 12.0 Hz, |
| | | | | 2H), 2.09-1.99 (m, 2H), 1.86- |
| | | | | 1.82 (m, 2H) |
| 199 | tert-butyl 3-(4-amino-3- | 465.43 | 466.2 | (ACETONITRILE)-d₃) δ = 8.39 |
| | ((4- | | | (d, J = 5.2 Hz, 1H), 8.28 (s, 1H), |
| | (trifluoromethyl)pyridin-2- | | | 7.56 (s, 1H), 7.49 (d, J = 5.6 Hz, |
| | yl)oxy)-1H-pyrazolo[3,4- | | | 1H), 5.99 (s, 2H), 5.44-5.39 |
| | d]pyrimidin-1- | | | (m, 1H), 3.75-3.67 (m, 1H), |
| | yl)pyrrolidine-1- | | | 3.65-3.62 (m, 1H), 3.58-3.54 |
| | carboxylate | | | (m, 1H), 3.52-3.45 (m, 1H), |
| | | | | 2.36-2.27 (m, 2H), 1.41 (s, 9H) |
| 200 | 1-cyclohexyl-3-((4- | 378.35 | 379.1 | (ACETONITRILE-d₃) δ = 8.41 |
| | (trifluoromethyl)pyridin-2- | | | (d, J = 4.8 Hz, 1 H), 8.27 (s, 1 |
| | yl)oxy)-1H-pyrazolo[3,4- | | | H), 7.59 (s, 1 H), 7.53 (d, J = |
| | d]pyrimidin-4-amine | | | 4.8 Hz, 1 H), 5.76-4.69 (m, 1 |
| | | | | H), 2.92-1.71 (m, 6 H), 1.53- |
| | | | | 1.23 (m, 4H) |
| 201 | 3-((4-chloropyridin-2- | 330.77 | 331.0 | (DMSO-d₆) δ = 8.17 (s, 1H), |
| | yl)oxy)-1-cyclopentyl-1H- | | | 8.11 (d, J = 5.6 Hz, 1H), 7.37 (s, |
| | pyrazolo[3,4-d]pyrimidin- | | | 1H), 7.32 (dd, J = 1.6, 5.6 Hz |
| | 4-amine | | | 1H), 5.17-5.09 (m, 1H), 2.05- |
| | | | | 2.01 (m, 2H), 1.91-1.87 (m, |
| | | | | 2H), 1.79-1.78 (m, 2H), 1.64- |
| | | | | 1.61 (m, 2H) |
| 202 | 3-((4-chloropyridin-2- | 332.75 | 333.0 | (DMSO-d₆) δ = 8.27 (s, 1H), |
| | yl)oxy)-1- | | | 8.14 (d, J = 5.6 Hz 1H), 7.42 (d, |
| | (tetrahydrofuran-3-yl)-1H- | | | J = 1.6 Hz, 1H), 7.35 (dd, J = |
| | pyrazolo[3,4-d]pyrimidin- | | | 2.0, 5.6 Hz, 1H), 5.46-5.41 (m, |
| | 4-amine | | | 1H), 4.08-3.94 (m, 2H), 3.85- |
| | | | | 3.82 (m, 2H), 2.41-2.24 (m, |
| | | | | 2H) |
| 203 | 2-((4-amino-1-(4,4- | 371.34 | 372.1 | (DMSO-d₆) δ = 8.36 (d, J = 4.8 |
| | difluorocyclohexyl)-1H- | | | Hz, 1 H), 8.23 (s, 1 H), 7.79 (s, |
| | pyrazolo[3,4-d]pyrimidin- | | | 1 H), 7.65 (d, J = 5.6 Hz, 1 H), |
| | 3-yl)oxy)isonicotinonitrile | | | 4.86 (s, 1 H), 2.14-1.98 (m, 8 |
| | | | | H) |
| 204 | 2-((4-amino-1- | 339.35 | 340.1 | (DMSO-d₆) δ = 8.31 (s, 1H), |
| | cyclopentyl-1H- | | | 8.29 (s, 1H), 8.24 (d, J = 5.6 Hz, |
| | pyrazolo[3,4-d]pyrimidin- | | | 1H), 7.80 (s, 1H), 7.59-7.57 |
| | 3-yl)oxy)isonicotinamide | | | (m, 2H), 5.21-5.11 (m, 1H), |
| | | | | 2.06-2.03 (m, 2H), 1.94-1.71 |
| | | | | (m, 4H), 1.69-1.56 (m, 2H) |
| 205 | 2-((4-amino-1-cyclohexyl- | 353.38 | 354.1 | (METHANOL-d₄) δ = 8.31 (s, |
| | 1H-pyrazolo[3,4- | | | 2H), 7.78 (s, 1H), 7.64 (s, 1H), |
| | d]pyrimidin-3- | | | 4.73 (s, 1H), 1.99-1.87 (m, |
| | yl)oxy)isonicotinamide | | | 6H), 1.76-1.73 (m, 1H), 1.53- |
| | | | | 1.46 (m, 2H), 1.32-1.27 (m, |
| | | | | 1H) |
| 206 | 2-((4-amino-1-(4,4- | 389.36 | 390.1 | (DMSO-d₆ + D₂O) δ = 8.27-8.25 |
| | difluorocyclohexyl)-1H- | | | (m, 2H), 7.58 (m, 2H), 4.87- |
| | pyrazolo[3,4-d]pyrimidin- | | | 4.75 (m, 1H), 2.15-2.01 (m, 8H) |
| | 3-yl)oxy)isonicotinamide | | | |
| 207 | 2-((4-amino-1-((cis)-4- | 403.36 | 404.1 | (METHANOL-d4) δ = 8.39 (d, J = |
| | (trifluoromethyl)cyclohexyl)- | | | 5.2 Hz, 1H), 8.33 (s, 1H), 7.75 |
| | 1H-pyrazolo[3,4- | | | (s, 1H), 7.56 (d, J = 5.2 Hz, 1H), |
| | d]pyrimidin-3- | | | 5.05-5.02 (m, 1H), 2.36-2.22 |
| | yl)oxy)isonicotinonitrile | | | (m, 3H), 1.99-1.95 (m, 4H), |
| | | | | 1.83-1.79 (m, 2H) |
| 208 | 2-((4-amino-1-((cis)-4- | 421.38 | 422.1 | (METHANOL-d₄) δ = 8.34-8.32 |
| | (trifluoromethyl)cyclohexyl)- | | | (m, 2H), 7.78 (s, 1H), 7.65 (d, J = |
| | 1H-pyrazolo[3,4- | | | 5.2 Hz, 1H), 5.05-4.97 (m, |
| | d]pyrimidin-3- | | | 1H), 2.35-2.24 (m, 3H), 2.00- |
| | yl)oxy)isonicotinamide | | | 1.94 (m, 4H), 1.82-1.77 (m, |
| | | | | 2H) |
| 209 | 2-((4-amino-1-((trans)-4- | 403.36 | 404.1 | (METHANOL-d₄) δ = 8.37 (d, J = |
| | (trifluoromethyl)cyclohexyl)- | | | 5.2 Hz, 1H), 8.33 (s, 1H), 7.72 |
| | 1H-pyrazolo[3,4- | | | (s, 1H), 7.56 (d, J = 5.2 Hz, 1H), |
| | d]pyrimidin-3- | | | 4.85-4.74 (m, 1H), 2.30-2.27 |
| | yl)oxy)isonicotinonitrile | | | (m, 1H), 2.13-2.10 (m, 4H), |
| | | | | 2.07-1.94 (m, 2H), 1.68-1.53 |
| | | | | (m, 2H) |
| 210 | 1-((cis)-4- | 446.35 | 447.2 | (DMSO-d₆) δ = 8.41 (d, J = 5.2 |
| | (trifluoromethyl)cyclohexyl)- | | | Hz, 1 H), 8.30 (s, 1 H), 7.68 (s, |
| | 3-((4- | | | 1 H), 7.59 (d, J = 5.6 Hz, 1 H), |
| | (trifluoromethyl)pyridin-2- | | | 4.89-4.87 (m, 1H), 2.14-2.10 |
| | yl)oxy)-1H-pyrazolo[3,4- | | | (m, 2 H), 1.92-1.74 (m, 7 H) |
| | d]pyrimidin-4-amine | | | |
| 211 | 1-((trans)-4- | 446.35 | 447.2 | (DMSO-d₆) δ = 8.39 (d, J = 5.6 |
| | (trifluoromethyl)cyclohexyl)- | | | Hz, 1 H), 8.26 (s, 1 H), 7.61 (s, |
| | 3-((4- | | | 1 H), 7.56 (d, J = 4.4 Hz, 1 H), |
| | (trifluoromethyl)pyridin-2- | | | 4.69-4.63 (m, 1H), 1.99-1.86 |
| | yl)oxy)-1H-pyrazolo[3,4- | | | (m, 7 H), 1.57-1.48 (m, 2 H) |
| | d]pyrimidin-4-amine | | | |
| 212 | tert-butyl 4-((4-amino-3- | 495.91 | 496.1 | (ACETONITRILE-d₃) δ = 8.25 |
| | ((4-chloropyridin-2- | | | (s, 1H), 8.09 (s, 1H), 7.30 (s, |
| | yl)oxy)-1H-pyrazolo[3,4- | | | 1H), 7.25 (s, 1H), 5.96 (s, 2H), |
| | d]pyrimidin-1-yl)methyl)- | | | 4.61-4.57 (m, 1H), 4.30-4.23 |
| | 3,3-difluoropiperidine-1- | | | (m, 2H), 4.00-3.99 (m, 1H), |
| | carboxylate | | | 3.04-3.03 (m, 1H), 2.74-2.61 |
| | | | | (m, 2H), 1.53-1.52 (m, 2H), |
| | | | | 1.42 (s, 9H) |
| 213 | 3-((4-chloropyridin-2- | 395.79 | 396.2 | (ACETONITRILE-d₃) δ = 8.28 |
| | yl)oxy)-1-((3,3- | | | (s, 1H), 8.13 (d, J = 5.2 Hz, 1H), |
| | difluoropiperidin-4- | | | 7.36 (s, 1H), 7.30 (d, J = 5.6 |
| | yl)methyl)-1H- | | | Hz, 1H), 4.72-4.67 (m, 1H), |
| | pyrazolo[3,4-d]pyrimidin- | | | 4.38-4.32 (m, 1H), 3.66-3.61 |
| | 4-amine | | | (m, 1H), 3.40-3.26 (m, 2H), |
| | | | | 2.96 (dd, J = 3.6, 12.8 Hz, 1H), |
| | | | | 2.96-2.78 (m, 1H), 1.94-1.76 |
| | | | | (m, 2H) |
| 214 | 3-((4-chloropyridin-2- | 344.80 | 345.1 | (CHLOROFORM-d) δ = 8.32 |
| | yl)oxy)-1-cyclohexyl-1H- | | | (s, 1H), 8.15 (d, J = 6.0 Hz, 1H), |
| | pyrazolo[3,4-d]pyrimidin- | | | 7.32 (d, J = 1.6 Hz, 1H), 7.14 |
| | 4-amine | | | (dd, J = 1.6, 6.0 Hz, 1H), 5.84 (s, |
| | | | | 2H), 4.72-4.64 (m, 1H), 2.03- |
| | | | | 1.98 (m, 2H), 1.97-1.87 (m, |
| | | | | 4H), 1.72-1.70 (m, 1H), 1.54- |
| | | | | 1.40 (m, 2H), 1.34-1.21 (m, |
| | | | | 1H) |
| 215 | 2-((4-amino-1-cyclohexyl- | 335.36 | 336.2 | (METHANOL-d₄) δ = 8.37 (d, J = |
| | 1H-pyrazolo[3,4- | | | 5.2 Hz, 1H), 8.31 (s, 1H), 7.72 |
| | d]pyrimidin-3- | | | (s, 1H), 7.55 (d, J = 5.2 Hz, 1H), |
| | yl)oxy)isonicotinonitrile | | | 4.79-4.69 (m, 1H), 2.01-1.85 |
| | | | | (m, 6H), 1.75 (d, J = 11.6 Hz, |
| | | | | 1H), 1.57-1.44 (m, 2H), 1.35- |
| | | | | 1.22 (m, 1H) |
| 216 | 3-((4-chloropyridin-2- | 346.77 | 347.0 | (METHANOL-d₄) δ = 8.33 (s, |
| | yl)oxy)-1-(tetrahydro-2H- | | | 1H), 8.14 (d, J = 5.6 Hz, 1H), |
| | pyran-3-yl)-1H- | | | 7.49 (d, J = 1.2 Hz, 1H), 7.35- |
| | pyrazolo[3,4-d]pyrimidin- | | | 7.32 (m, 1H), 4.86-4.83 (m, |
| | 4-amine | | | 1H), 4.03-4.00 (m, 1H), 3.93- |
| | | | | 3.91 (m, 1H), 3.75-3.70 (m, |
| | | | | 1H), 3.50-3.46 (m, 1H), 2.24- |
| | | | | 2.17 (m, 2H), 1.88-1.82 (m, |
| | | | | 2H) |
| 188 | 3-((4-chloropyridin-2- | 345.79 | 346.1 | (DMSO-d₆) δ = 8.75 (d, J = 9.6 |
| | yl)oxy)-1-(piperidin-4-yl)- | | | Hz, 1H), 8.38 (d, J = 11.2 Hz, |
| | 1H-pyrazolo[3,4- | | | 1H), 8.28 (s, 1H), 8.14 (d, J = |
| | d]pyrimidin-4-amine | | | 5.2 Hz, 1H), 7.43-7.35 (m, |
| | | | | 2H), 5.00-4.95 (m, 1H), 3.40 |
| | | | | (d, J = 12.8 Hz, 2H), 3.20-3.11 |
| | | | | (m, 2H), 2.24-1.99 (m, 4H) |
| 217 | 3-((2-chloropyridin-4- | 331.76 | 332.0 | (METHANOL-d₄) δ = 8.39 (d, J = |
| | yl)oxy)-1-(pyrrolidin-3- | | | 5.6 Hz, 1H), 8.37 (s, 1H), 7.58 |
| | yl)-1H-pyrazolo[3,4- | | | (d, J = 2.4 Hz, 1H), 7.50 (dd, J = |
| | d]pyrimidin-4-amine | | | 2.0, 5.6 Hz, 1H), 5.75-5.69 |
| | | | | (m, 1H), 3.82-3.80 (m, 2H), |
| | | | | 3.64-3.54 (m, 2H), 2.63-2.50 |
| | | | | (m, 2H) |
| 218 | 3-((2-chloropyridin-4- | 345.79 | 346.0 | (DMSO-d₆) δ = 8.75 (d, J = 9.6 |
| | yl)oxy)-1-(piperidin-4-yl)- | | | Hz, 1H), 8.41 (d, J = 5.6 Hz, |
| | 1H-pyrazolo[3,4- | | | 1H), 8.27 (d, J = 5.6 Hz, 1H), |
| | d]pyrimidin-4-amine | | | 7.58 (d, J = 2.0 Hz, 1H), 7.47- |
| | | | | 7.48 (m, 1H), 5.02-4.95 (m, |
| | | | | 1H), 3.43 (d, J = 12.4 Hz, 2H), |
| | | | | 3.21-3.13 (m, 2H), 2.28-2.22 |
| | | | | (m, 2H), 2.19-2.09 (m, 2H) |
| 219 | 3-((4-methoxypyridin-2- | 328.33 | 329.1 | (ACETONITRILE-d₃) δ = 8.28 |
| | yl)oxy)-1- | | | (s, 1H), 8.02 (d, J = 5.6 Hz, 1H), |
| | (tetrahydrofuran-3-yl)-1H- | | | 6.86-6.83 (m, 2H), 5.57-5.53 |
| | pyrazolo[3,4-d]pyrimidin- | | | (m, 1H), 4.10-4.05 (m, 2H), |
| | 4-amine | | | 3.97-3.89 (m, 5H), 2.49-2.42 |
| | | | | (m, 1H), 2.36-2.33 (m, 1H) |
| 220 | 3-((4-methoxypyridin-2- | 342.35 | 343.1 | (METHANOL-d₄) δ = 8.28 (s, |
| | yl)oxy)-1-(tetrahydro-2H- | | | 1H), 8.04 (d, J = 5.6 Hz, 1H), |
| | pyran-4-yl)-1H- | | | 7.29 (s, 1H), 6.87-6.84 (m, |
| | pyrazolo[3,4-d]pyrimidin- | | | 2H), 5.02-4.95 (m, 1H), 4.06- |
| | 4-amine | | | 4.02 (m, 2H), 3.94 (s, 3H), 3.61- |
| | | | | 3.55 (m, 2H), 2.20-2.11 (m, |
| | | | | 2H), 1.94-1.91 (m, 2H) |
| 221 | 2-((4-amino-1- | 307.31 | 308.1 | (METHANOL-d4) δ = 8.39 (d, J = |
| | (cyclopropylmethyl)-1H- | | | 4.4 Hz, 1H), 8.32 (s, 1H), 7.76 |
| | pyrazolo[3,4-d]pyrimidin- | | | (s, 1H), 7.57 (d, J = 4.8 Hz, 1H), |
| | 3-yl)oxy)isonicotinonitrile | | | 4.21 (d, J = 7.2 Hz, 2H), 1.33 (s, |
| | | | | 1H), 0.58 (d, J = 7.6 Hz, 2H), |
| | | | | 0.46-0.43 (m, 2H) |
| 222 | 3-((4-chloropyridin-2- | 345.79 | 346.1 | (METHANOL-d₄) δ = 8.21 (s, |
| | yl)oxy)-1-(piperidin-3-yl)- | | | 1H), 8.13 (d, J = 5.6 Hz, 1H), |
| | 1H-pyrazolo[3,4- | | | 7.46 (d, J = 1.2 Hz, 1H), 7.29 |
| | d]pyrimidin-4-amine | | | (dd, J = 1.6, 5.4 Hz, 1H), 4.76- |
| | | | | 4.69 (m, 1H), 3.20-3.16 (m, |
| | | | | 1H), 3.09-2.98 (m, 2H), 2.67- |
| | | | | 2.62 (m, 1H), 2.12-2.07 (m, |
| | | | | 2H), 1.89-1.85 (m, 1H), 1.84- |
| | | | | 1.69 (m, 1H) |
| 223 | 1-(cyclopropylmethyl)-3- | 312.33 | 313.1 | (METHANOL-d₄) δ = 8.30 (s, |
| | ((4-methoxypyridin-2- | | | 1H), 8.03 (d, J = 5.6 Hz, 1H), |
| | yl)oxy)-1H-pyrazolo[3,4- | | | 7.04 (d, J = 2.0 Hz, 1H), 6.90- |
| | d]pyrimidin-4-amine | | | 6.87 (m, 1H), 4.19 (d, J = 7.2 |
| | | | | Hz, 2H), 3.95 (s, 3H), 1.36- |
| | | | | 1.30 (m, 1H), 0.59-0.55 (m, |
| | | | | 2H), 0.47-0.44 (m, 2H) |
| 224 | tert-butyl 3-(4-amino-3- | 445.90 | 446.1 | (CHLOROFORM-d) δ = 8.35 |
| | ((4-chloropyridin-2- | | | (s, 1H), 8.16 (d, J = 5.6 Hz, 1H), |
| | yl)oxy)-1H-pyrazolo[3,4- | | | 7.32 (s, 1H), 7.18 (dd, J = 4.0, |
| | d]pyrimidin-1- | | | 1.6 Hz, 1H), 5.63 (s, 2H), 4.78- |
| | yl)piperidine-1- | | | 4.72 (m, 1H), 4.24-4.11 (m, |
| | carboxylate | | | 3H), 3.31 (s, 1H), 2.82-2.76 |
| | | | | (m, 1H), 2.16-2.10 (m, 2H), |
| | | | | 1.86 (d, J = 12.4 Hz, 1H), 1.45 |
| | | | | (s, 9H) |
| 225 | 3-((4-chloropyridin-2- | 316.75 | 317.1 | (METHANOL-d₄) δ = 8.26 (s, |
| | yl)oxy)-1- | | | 1H), 8.15 (d, J = 5.2 Hz, 1H), |
| | (cyclopropylmethyl)-1H- | | | 7.51 (d, J = 1.6 Hz, 1H), 7.32 |
| | pyrazolo[3,4-d]pyrimidin- | | | (dd, J = 1.6, 3.6 Hz, 1H), 4.17 |
| | 4-amine | | | (d, J = 7.2 Hz, 2H), 1.36-1.30 |
| | | | | (m, 1H), 0.59-0.55 (m, 2H), |
| | | | | 0.45-0.43 (m, 2H) |
| 226 | 1-(2-methylcyclohexyl)-3- | 392.38 | 393.2 | (DMSO-d₆) δ = 8.38 (d, J = 5.2 |
| | ((4- | | | Hz, 1 H), 8.16 (s, 1 H), 7.59 (s, |
| | (trifluoromethyl)pyridin-2- | | | 1 H), 7.53 (d, J = 5.2 Hz, 1 H), |
| | yl)oxy)-1H-pyrazolo[3,4- | | | 4.87-4.82 (m, 1 H), 2.18 (s, 1 |
| | d]pyrimidin-4-amine | | | H), 2.07-2.00 (m, 1 H), 1.84 (s, |
| | | | | 1 H), 1.76-1.39 (m, 6 H), 0.66 |
| | | | | (d, J = 6.8 Hz, 3 H) |
| 227 | 1-(4- | 421.42 | 422.2 | (METHANOL-d₄) δ = 8.39 (d, J = |
| | (dimethylamino)cyclohexyl)- | | | 4.4 Hz, 1H), 8.21 (s, 1H), 7.65 |
| | 3-((4- | | | (s, 1H), 7.50 (s, 1H), 4.72-4.68 |
| | (trifluoromethyl)pyridin-2- | | | (m, 1H), 3.10-3.06 (m, 1H), |
| | yl)oxy)-1H-pyrazolo[3,4- | | | 2.73 (s, 6H), 2.22-2.02 (m, |
| | d]pyrimidin-4-amine | | | 6H), 1.74-7.70 (m, 2H) |
| 228 | methyl 4-(4-amino-3-((4- | 436.39 | 437.1 | (DMSO-d₆) δ = 8.38 (d, J = 5.2 |
| | (trifluoromethyl)pyridin-2- | | | Hz, 1H), 8.20 (s, 1H), 7.60 (s, |
| | yl)oxy)-1H-pyrazolo[3,4- | | | 1H), 7.55 (d, J = 5.2 Hz, 1H), |
| | d]pyrimidin-1- | | | 4.73-4.55 (m, 1H), 3.67-3.59 |
| | yl)cyclohexanecarboxylate | | | (m, 3H), 2.77-2.66 (m, 1H), |
| | | | | 2.20-2.00 (m, 2H), 1.99-1.47 |
| | | | | (m, 6H) |
| 229 | 1-(4,4- | 406.40 | 407.1 | (DMSO-d₆) δ = 8.39 (d, J = 5.2 |
| | dimethylcyclohexyl)-3- | | | Hz, 1H), 8.18 (s, 1H), 7.61 (s, |
| | ((4- | | | 1H), 7.55 (d, J = 5.2 Hz, 1H), |
| | (trifluoromethyl)pyridin-2- | | | 4.53 (t, J = 11.6 Hz, 1H), 2.08- |
| | yl)oxy)-1H-pyrazolo[3,4- | | | 1.93 (m, 2H), 1.71 (d, J = 10.8 |
| | d]pyrimidin-4-amine | | | Hz, 2H), 1.53-1.34 (m, 4H), |
| | | | | 0.96 (d, J = 3.2 Hz, 6H) |
| 230 | 3-(4-amino-3-((4- | 380.32 | 381.1 | (METHANOL-d₄) δ = 8.42 (d, J = |
| | (trifluoromethyl)pyridin-2- | | | 5.2 Hz, 1H), 8.32 (s, 1H), 7.71 |
| | yl)oxy)-1H-pyrazolo[3,4- | | | (s, 1H), 7.53 (d, J = 5.2 Hz, 1H), |
| | d]pyrimidin-1- | | | 5.33-5.25 (m, 1H), 4.35-4.29 |
| | yl)cyclopentanol | | | (m, 1H), 2.55-2.46 (m, 1H), |
| | | | | 2.28-2.11 (m, 2H), 2.05 (td, J = |
| | | | | 6.8, 13.6 Hz, 1H), 2.00-1.92 |
| | | | | (m, 1H), 1.91-1.82 (m, 1H) |
| 231 | (cis)-4-(4-amino-3-((4- | 394.35 | 395.1 | (METHANOL-d₄) δ = 8.41 (d, J = |
| | (trifluoromethyl)pyridin-2- | | | 5.2 Hz, 1H), 8.31 (s, 1H), 7.69 |
| | yl)oxy)-1H-pyrazolo[3,4- | | | (s, 1H), 7.53 (d, J = 5.2 Hz, 1H), |
| | d]pyrimidin-1- | | | 4.78 (tt, J = 4.0, 11.6 Hz, 1H), |
| | yl)cyclohexanol | | | 4.05-3.97 (m, 1H), 2.42-2.29 |
| | | | | (m, 2H), 1.98-1.89 (m, 2H), |
| | | | | 1.81-1.68 (m, 4H) |
| 232 | 1-(4-aminocyclohexyl)-3- | 393.37 | 394.1 | (METHANOL-d4) δ = 8.39 (d, |
| | ((4- | | | J = 5.2 Hz, 1H), 8.35 (s, 1H), |
| | (trifluoromethyl)pyridin-2- | | | 7.65 (s, 1H), 7.53 (d, J = 5.2 Hz, |
| | yl)oxy)-1H-pyrazolo[3,4- | | | 1H), 5.07-4.95 (m, 1H), 3.42- |
| | d]pyrimidin-4-amine | | | 3.38 (m, 1H), 2.36-2.21 (m, |
| | | | | 2H), 2.13-1.92 (m, 6H) |
| 233 | 1-((1S,2R)-2- | 396.34 | 397.1 | (METHANOL-d₄) δ = 8.39 (d, J = |
| | fluorocyclohexyl)-3-((4- | | | 5.2 Hz, 1H), 8.21 (s, 1H), 7.71 |
| | (trifluoromethyl)pyridin-2- | | | (s, 1H), 7.48 (d, J = 5.2 Hz, 1H), |
| | yl)oxy)-1H-pyrazolo[3,4- | | | 5.04-5.02 (m, 0.5H), 4.93-4.90 |
| | d]pyrimidin-4-amine | | | (m, 0.5H), 4.86-4.75 (m, 1H), |
| | | | | 2.53-2.41 (m, 1H), 2.12- |
| | | | | 2.08 (m, 1H), 2.02-1.53 (m, |
| | | | | 6H) |
| 234 | 1-(2- | 391.35 | 392.1 | (MEOH) δ = 8.41-8.40 (d, 1H), |
| | azabicyclo[2.2.1]heptan-6- | | | 8.34 (s, 1H), 7.67 (s, 1H), 7.56- |
| | yl)-3-((4- | | | 7.54 (s, 1H), 5.32-5.28 (m, |
| | (trifluoromethyl)pyridin-2- | | | 1H), 4.21 (s, 1H), 3.31-3.20 |
| | yl)oxy)-1H-pyrazolo[3,4- | | | (m, 1H), 3.12-2.90 (m, 1H), |
| | d]pyrimidin-4-amine | | | 2.44-2.43 (m, 2H), 2.42-2.27 |
| | | | | (m, 1H), 1.80-1.77 (d, 2H) |
| 235 | (trans)-4-(4-amino-3-((4- | 394.35 | 395.1 | (METHANOL-d₄) δ = 8.40 (d, J = |
| | (trifluoromethyl)pyridin-2- | | | 5.2 Hz, 1H), 8.31 (s, 1H), 7.68 |
| | yl)oxy)-1H-pyrazolo[3,4- | | | (s, 1H), 7.53 (d, J = 5.2 Hz, 1H), |
| | d]pyrimidin-1- | | | 4.80-4.70 (m, 1H), 3.63 (tt, J = |
| | yl)cyclohexanol | | | 4.4, 10.8 Hz, 1H), 2.13-1.95 |
| | | | | (m, 6H), 1.56-1.44 (m, 2H) |
| 236 | 1-(2-methoxycyclohexyl)- | 408.38 | 409.1 | (METHANOL-d₄) δ = 8.40 (d, J = |
| | 3-((4- | | | 5.2 Hz, 1H), 8.34 (s, 1H), 7.67 |
| | (trifluoromethyl)pyridin-2- | | | (s, 1H), 7.53 (d, J = 5.2 Hz, 1H), |
| | yl)oxy)-1H-pyrazolo[3,4- | | | 4.66-3.60 (m, 1H), 3.61-3.55 |
| | d]pyrimidin-4-amine | | | (m, 1H), 3.10 (s, 3H), 2.34- |
| | | | | 2.26 (m, 1H), 2.04-1.91 (m, |
| | | | | 2H), 1.88-1.81 (m, 2H), 1.48- |
| | | | | 1.27 (m, 3H) |
| 237 | 1-((trans)-4- | 408.38 | 409.1 | (METHANOL-d₄) δ = 8.41 (d, J = |
| | methoxycyclohexyl)-3-((4- | | | 5.2 Hz, 1H), 8.34 (s, 1H), 7.68 |
| | (trifluoromethyl)pyridin-2- | | | (s, 1H), 7.54 (d, J = 5.2 Hz, 1H), |
| | yl)oxy)-1H-pyrazolo[3,4- | | | 4.82-4.76 (m, 1H), 3.37 (s, |
| | d]pyrimidin-4-amine | | | 3H), 3.34-3.26 (m, 1H), 2.22 |
| | | | | (d, J = 10.8 Hz, 2H), 2.08-1.96 |
| | | | | (m, 4H), 1.47-1.40 (m, 2H) |
| 238 | 1-((cis)-4- | 408.38 | 409.1 | (METHANOL-d₄) δ = 8.43 (td, J = |
| | methoxycyclohexyl)-3-((4- | | | 5.2 Hz, 1H), 8.33 (s, 1H), 7.72 |
| | (trifluoromethyl)pyridin-2- | | | (s, 1H), 7.56 (d, J = 5.2 Hz, 1H), |
| | yl)oxy)-1H-pyrazolo[3,4- | | | 4.87-4.79 (m, 1H), 3.54 (s, |
| | d]pyrimidin-4-amine | | | 1H), 3.34 (s, 3H), 2.27-2.23 |
| | | | | (m, 2H), 2.13 (d, J = 15.6 Hz, |
| | | | | 2H), 1.79 (dd, J = 3.2, 9.6 Hz, |
| | | | | 2H), 1.76-1.67 (m, 2H) |
| 239 | 1-((cis)-4- | 393.37 | 394.1 | (DMSO-d₆) δ = 8.40 (d, J = 5.2 |
| | aminocyclohexyl)-3-((4- | | | Hz, 1H), 8.34 (s, 1H), 7.93 (s, |
| | (trifluoromethyl)pyridin-2- | | | 3H), 7.63 (s, 1H), 7.57 (d, J = |
| | yl)oxy)-1H-pyrazolo[3,4- | | | 5.2 Hz, 1H), 4.81-4.77 (m, |
| | d]pyrimidin-4-amine | | | 1H), 3.33 (d, J = 4.4 Hz, 1H), |
| | | | | 2.17-2.11 (m, 2H), 1.87 (s, 6H) |
| 240 | tert-butyl ((1S,3R)-3-(4- | 479.46 | 480.1 | (METHANOL-d₄) δ = 8.43 (d, J = |
| | amino-3-((4- | | | 5.2 Hz, 1H), 8.33 (s, 1H), 7.68 |
| | (trifluoromethyl)pyridin-2- | | | (s, 1H), 7.55 (d, J = 5.6 Hz, 1H), |
| | yl)oxy)-1H-pyrazolo[3,4- | | | 5.40-5.32 (m, 1H), 4.06-4.02 |
| | d]pyrimidin-1- | | | (m, 1H), 2.55-2.51 (m, 1H), |
| | yl)cyclopentyl)carbamate | | | 2.20-1.97 (m, 4H), 1.81-1.75 |
| | | | | (m, 1H), 1.38 (s, 9H) |
| 241 | tert-butyl 6-(4-amino-3- | 491.47 | 492.1 | (DMSO-d₆) δ = 8.41-8.39 (d, J = |
| | ((4- | | | 5.2 Hz, 1H), 8.33-8.31 (m, |
| | (trifluoromethyl)pyridin-2- | | | 1H), 7.66 (s, 1H), 7.59-7.57 (d, |
| | yl)oxy)-1H-pyrazolo[3,4- | | | J = 5.2 Hz, 1H), 4.90 (m, 1H), |
| | d]pyrimidin-1-yl)-2- | | | 4.01 (s, 1H), 3.13-2.98 (m, |
| | azabicyclo[2.2.1]heptane- | | | 1H), 2.95 (m, 1H), 2.67-2.65 |
| | 2-carboxylate | | | (m, 1H), 2.22 (m, J = 14.0 Hz, |
| | | | | 1H), 2.11-2.02 (m, 2H), 1.99- |
| | | | | 1.96 (m, 1H), 1.58-1.42 (m, |
| | | | | 9H) |
| 242 | ((cis)-4-(4-amino-3-((4- | 408.38 | 409.1 | (METHANOL-d₄) δ = 8.41 (d, J = |
| | (trifluoromethyl)pyridin-2- | | | 5.2 Hz, 1H), 8.32 (s, 1H), 7.72 |
| | yl)oxy)-1H-pyrazolo[3,4- | | | (s, 1H), 7.54 (d, J = 5.6 Hz, 1H), |
| | d]pyrimidin-1- | | | 4.87-4.85 (m, 1H), 3.55 (d, J = |
| | yl)cyclohexyl)methanol | | | 6.4 Hz, 2H), 2.12-2.08 (m, |
| | | | | 2H), 1.84-1.81 (m, 5H), 1.73- |
| | | | | 1.68 (m, 2H) |
| 243 | ((trans)-4-(4-amino-3-((4- | 408.38 | 409.1 | (METHANOL-d₄) δ = 8.40 (d, J = |
| | (trifluoromethyl)pyridin-2- | | | 4.8 Hz, 1H), 8.32 (s, 1H), 7.69 |
| | yl)oxy)-1H-pyrazolo[3,4- | | | (s, 1H), 7.53 (d, J = 5.2 Hz, 1H), |
| | d]pyrimidin-1- | | | 4.78-4.71 (m, 1H), 3.42 (d, J = |
| | yl)cyclohexyl)methanol | | | 6.0 Hz, 2H), 2.05-1.95 (m, |
| | | | | 6H), 1.54-1.53 (m, 1H), 1.26- |
| | | | | 1.20 (m, 2H) |
| 244 | 1-((1- | 418.30 | 419.1 | (DMSO-d₆) δ = 8.41-8.40 (m, |
| | (trifluoromethyl)cyclopropyl) | | | 1H), 8.22 (s, 1H), 7.61 (1, 1H), |
| | methyl)-3-((4- | | | 7.56-7.55 (d, 1H), 4.50 (s, 2H), |
| | (trifluoromethyl)pyridin-2- | | | 1.12 (s, 2H), 1.03-1.00 (m, 2H) |
| | yl)oxy)-1H-pyrazolo[3,4- | | | |
| | d]pyrimidin-4-amine | | | |
| 190 | 1-(5,5-difluoropiperidin-3- | 415.32 | 416.1 | (METHANOL-d₄) δ = 8.40 (d, J = |
| | yl)-3-((4- | | | 4.8 Hz, 1H), 8.31 (s, 1H), 7.66 |
| | (trifluoromethyl)pyridin-2- | | | (s, 1H), 7.54 (d, J = 5.2 Hz, 1H), |
| | yl)oxy)-1H-pyrazolo[3,4- | | | 5.36-5.28 (m, 1H), 3.77-3.60 |
| | d]pyrimidin-4-amine | | | (m, 4H), 2.87-2.76 (m, 2H) |
| 186 | 1-(cyclopropylmethyl)-3- | 350.30 | 351.1 | (ACETONITRILE-d₃) δ = 8.40 |
| | ((4- | | | (d, J = 5.6 Hz, 1H), 8.25 (s, 1H), |
| | (trifluoromethyl)pyridin-2- | | | 7.59 (s, 1H), 7.51 (d, J = 5.2 Hz, |
| | yl)oxy)-1H-pyrazolo[3,4- | | | 1H), 4.18 (d, J = 7.2 Hz, 2H), |
| | d]pyrimidin-4-amine | | | 1.31-1.23 (m, 1H), 0.57-0.52 |
| | | | | (m, 2H), 0.43-0.40 (m, 2H) |
| 245 | 2-(4-amino-3-((4- | 354.36 | 355.1 | (CHLOROFORM-d) δ = 8.30 |
| | methoxypyridin-2-yl)oxy)- | | | (s, 1H), 8.05 (d, J = 5.6 Hz, 1H), |
| | 1H-pyrazolo[3,4- | | | 6.76 (d, J = 2.0 Hz, 1H), 6.70- |
| | d]pyrimidin-1- | | | 6.67 (m, 1H), 5.82 (s, 2H), 5.42- |
| | yl)cyclohexanone | | | 5.37 (m, 1H), 3.87 (s, 3H), |
| | | | | 2.67-2.61 (m, 2H), 2.51-2.47 |
| | | | | (m, 2H), 2.16-2.15 (m, 2H), |
| | | | | 1.92-1.85 (m, 2H) |

### Synthesis Method W: General Procedure Represented by the Preparation of (1R,2R)-2-[4-amino-3-[[4-(trifluoromethyl)-2-pyridyl]oxy]pyrazolo[3,4-d]pyrimidin-1-yl]cyclohexanol and (1S,2S)-2-[4-amino-3-[[4-(trifluoromethyl)-2-pyridyl]oxy]pyrazolo[3,4-d]pyrimidin-1-yl]cyclohexanol

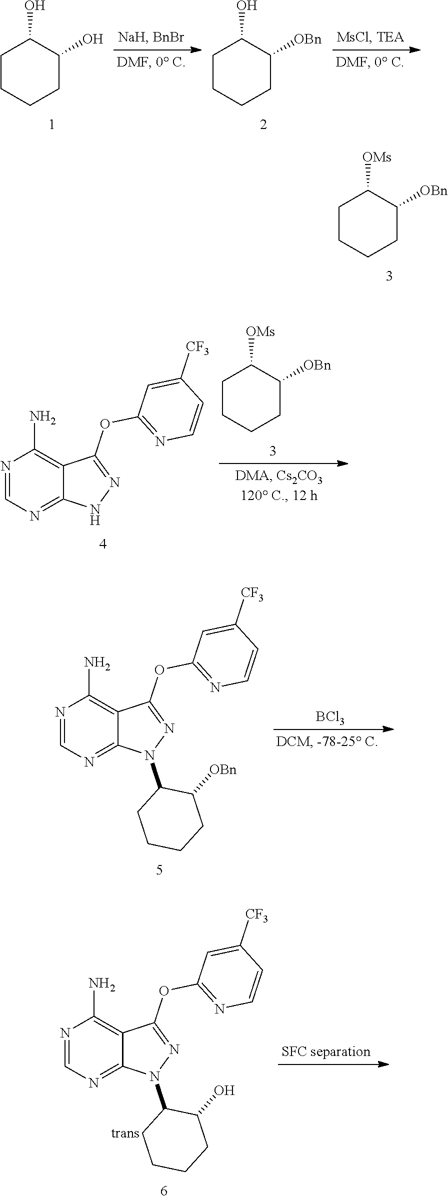

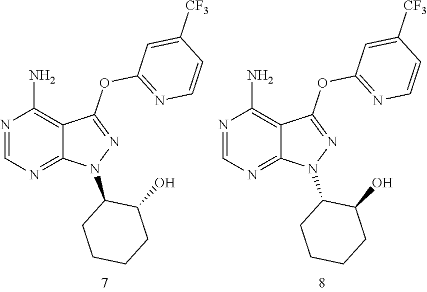

### Step 1. Procedure far preparation of racemic (cis)-2-benzyloxycyclohexanol (2)

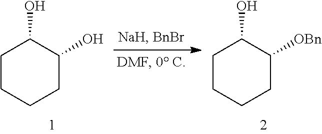 [SMILES: O[C@@H]1CCCC[C@@H]1O ; O[C@H]1CCCC[C@H]1OCc1ccccc1]

(1 S,2R)-cyclohexane-1,2-diol (1) (12 g, 103.31 mmol, 1 eq) was dissolved into DMF (250 mL) under N₂. NaH (4.55 g, 113.64 mmol, 60% purity, 1.1 eq) was added at 0° C. in one portion and the mixture was stirred at 0° C. for 30 min. BnBr (16.79 g, 98.14 mmol, 11.66 mL, 0.95 eq) was added dropwise. The mixture was allowed to warm at 25° C. and stirred for 12 h. The resulting mixture was partitioned between EtOAc (300 mL) and water (300 mL), and then the aqueous phase was further extracted with EtOAc (3×100 mL), then the organic phase was washed with brine (3×200 mL) and dried over Na₂SO₄. The residue was purified by column chromatography (SiO₂, petroleum ether/ethyl acetate=10/1 to 2/1) to afford (1S,2R)-2-benzyloxycyclohexanol (2) (7.1 g, 34.42 mmol, 33.32% yield) as colorless oil.

¹H NMR: (400 MHz, chloroform-d) δ=7.28-7.40 (m, 4H), 4.61-4.67 (m, 1H), 4.51-4.56 (m, 1H), 3.85-3.91 (m, 1H), 3.53 (dt, J=8.4, 3.2 Hz, 1H), 2.31-2.36 (m, 1H), 1.79-1.91 (m, 2H), 1.49-1.71 (m, 4H), 1.26-1.37 (m, 2H).

### Step 2. Procedure for preparation of racemic [(cis)-2-benzyloxycyclohexyl]methanesulfonate (3)

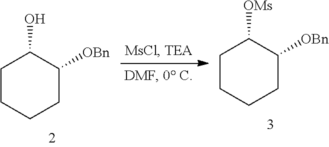 [SMILES: O[C@H]1CCCC[C@H]1OCc1ccccc1 ; CS(=O)(=O)O[C@H]1CCCC[C@H]1OCc1ccccc1]

To a solution of (1S,2R)-2-benzyloxycyclohexanol (2) (7.1 g, 34.42 mmol, 1 eq), Et₃N (6.97 g, 68.84 mmol, 9.58 mL, 2 eq) in DCM (100 mL) was added MsCl (6.31 g, 55.07 mmol, 4.26 mL, 1.6 eq) dropwise at 0° C. over a period of 5 mins under N₂. During which the temperature was maintained below 0° C. The reaction mixture was warmed to 25° C. over a period of 5 mins and stirred at 25° C. for 2 hr. The reaction mixture was quenched by addition water 100 mL at 0° C., and then extracted with CH₂Cl₂ (3×100 mL). The combined organic layers were dried over anhydrous sodium sulfate filtered and concentrated under reduced pressure to give [(1S,2R)-2-benzyloxycyclohexyl]methanesulfonate (3) (8 g, 28.13 mmol, 81.73% yield) as brown oil without further purification.

¹H NMR: (400 MHz, chloroform-d) δ=7.41-7.28 (m, 5H), 5.04-4.97 (m, 1H), 4.67-4.58 (m, 2H), 3.60-3.51 (m, 1H), 2.21-2.10 (m, 1H), 1.85-1.56 (m, 5H), 1.53-1.22 (m, 3H)

### Step 3. Procedure for preparation of racemic 1-[(trans)-2-benzyloxycyclohexyl]-3-[[4-(trifluoromethyl)-2-pyridyl]oxy]pyrazolo[3,4-d]pyrimidin-4-amine (5)

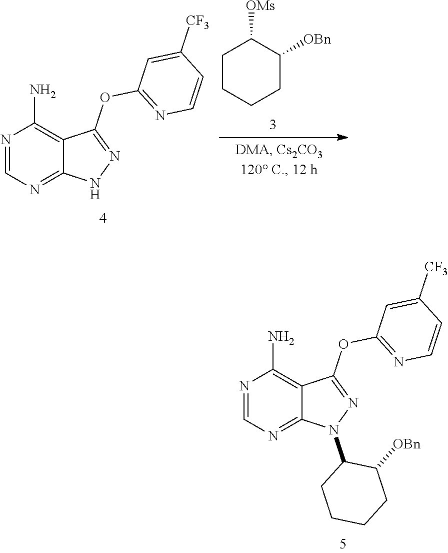

To a solution of 3-[[4-(trifluoromethyl)-2-pyridyl]oxy]-1H-pyrazolo[3,4-d]pyrimidin-4-amine (4) (1 g, 3.38 mmol, 1 eq) and racemic [(cis)-2-benzyloxycyclohexyl]methanesulfonate (3) (1.44 g, 5.06 mmol, 1.5 eq) in DMA (1 mL) was added Cs₂CO₃ (2.20 g, 6.75 mmol, 2 eq) and stirred at 120° C. for 12 h. The mixture was filtered. Then the filtrate was diluted with EtOAc 10 mL and water 5 mL. The combined organic layers were washed with water 30 mL (3×10 mL), dried over anhydrous sodium sulfate, Altered and concentrated under reduced pressure to give racemic 1-[(trans)-2-benzyloxycyclohexyl]-3-[[4-(trifluoromethyl)-2-pyridyl]oxy]pyrazolo[3,4-d]pyrimidin-4-amine (5) (200 mg, 412.82 umol, 12.23% yield) as a white solid.

### Step 4. Procedure for preparation of racemic (trans)-2-[4-amino-3-[[4-(trifluoromethyl)-2-pyridyl]oxy]pyrazolo[3,4-d]pyrimidin-1-yl]cyclohexanol (6)

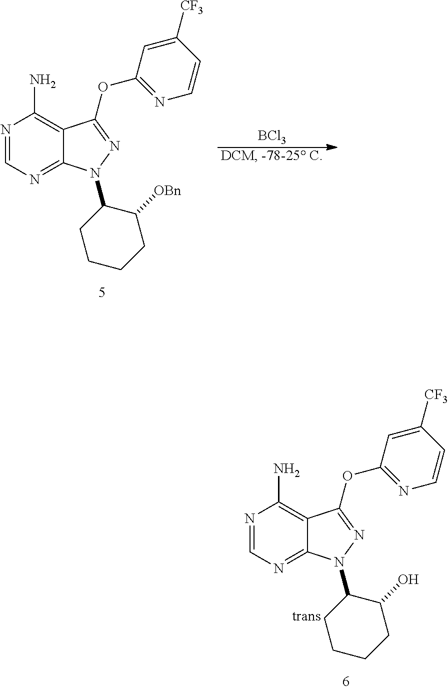

To a mixture of 1-(2-benzyloxycyclohexyl)-3-[[4-(trifluoromethyl)-2-pyridyl]oxy]Pyrazolo[3,4-d]pyrimidin-4-amine (5) (0.9 g, 1.86 mmol, 1 eq) in DCM (10 mL) was added BCl₃ (1 M, 9.29 mL, 5 eq) dropwise keeping the temperature below −78° C. under N₂. Then the mixture was stirred at −78° C. to 25° C. for 2.5 h under N₂. The reaction mixture was quenched by addition MeOH 2 mL at −78° C., filtered and concentrated under reduced pressure to give a residue. The residue was purified by prep-HPLC (neutral condition) to give racemic (trans)-2-[4-amino-3-[[4-(trifluoromethyl)-2-pyridyl]oxy]pyrazolo[3,4-d]pyrimidin-1-yl]cyclohexanol (6) (0.6 g, 1.52 mmol, 81.63% yield, 99.67% purity) as a white solid.

¹H NMR: (400 MHz, DMSO-d₆) δ=8.41 (d, J=5.2 Hz, 1H), 8.16 (s, 1H), 7.60 (s, 1H), 7.55 (d, J=5.2 Hz, 1H), 4.67 (d, J=5.2 Hz, 1H), 4.43-4.29 (m, 1H), 3.87-3.72 (m, 1H), 2.02-1.62 (m, 5H), 1.44-1.20 (m, 3H).

### Step 5. Procedure for preparation of (1R,2R)-2-[4-amino-3-[[4-(trifluoromethyl)-2-pyridyl]oxy]pyrazolo[3,4-d]pyrimidin-1-yl]cyclohexanol (7) and (1S,2S)-2-[4-amino-3-[[4-(trifluromethyl)-2-pyridyl]oxy]pyrazolo[3,4-d]pyrimidin-1-yl]cyclohexanol (8)

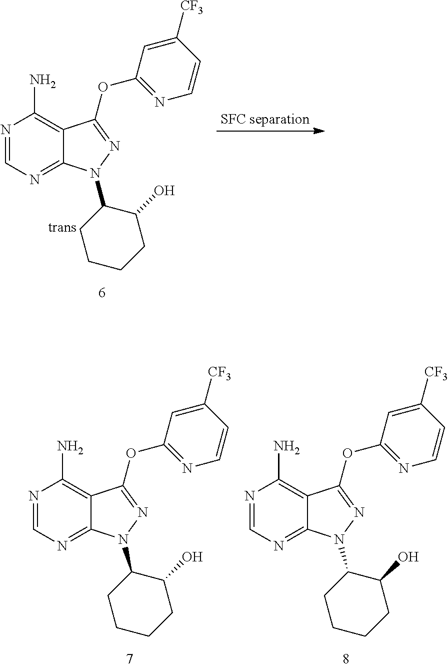

Racemic (trans)-2-[4-amino-3-[[4-(trifluoromethyl)-2-pyridyl]oxy]pyrazolo[3,4-d]pyrimidin-1-yl]cyclohexanol (0.6 g, 1.52 mmol, 81.63% yield, 99.67% purity) was further separated by SFC (Instrument: Waters prep-SFC 80Q; Column: Chiralpak AD-H, 250*25 mm i.d. 5u; Mobile phase: A for CO₂ and B for IPA (0.1% NH₃.H₂O); Gradient: B %=30%; Flow rate: 60 g/min; Column temperature: 40° C.; System back pressure: 100 bar) to give (1R,2R))-2-[-4-amino-3-[[4-(trifluoromethyl)-2-pyridyl]oxy]pyrazolo[3,4-d]pyrimidin-1-yl]cyclohexanol (7) (29.6 mg, 74.23 umol, 4.00% yield, 98.9% purity) and (1 S,2S)-2-[4-amino-3-[[4-(trifluoromethyl)-2-pyridyl]oxy]pyrazolo[3,4-d]pyrimidin-1-yl]cyclohexanol (8) (31.1 mg, 78.86 umol, 4.25% yield, 100% purity).

¹H NMR (7): (400 MHz, DMSO-d₆) δ=8.41-8.40 (d, J=5.2 Hz, 1H), 8.16 (s, 1H), 7.60 (s, 1H), 7.55-7.54 (d, J=5.2 Hz, 1H), 4.66 (s, 1H), 4.39-4.32 (m, 1H), 3.79 (m, 1H), 1.98-1.70 (m, 5H), 1.36-1.23 (m, 3H).

¹H NMR (8): (400 MHz, DMSO-d₆) δ=8.41-8.40 (m, 1H), 8.16 (s, 1H), 7.60 (s, 1H), 7.56-7.54 (m, 2H), 4.67 (s, 1H), 4.36-4.32 (m, 1H), 3.79 (m, 1H), 2.02-1.70 (m, 5H), 1.35-1.23 (m, 3H)

The following compounds were prepared in a similar manner as described in method W using different starting materials.

TABLE 17
Compounds Prepared by Method W

| | | | LC/MS | |
|---|---|---|---|---|
| Compound | | | Observed | |
| No. | IUPAC Name | MW | (M + H) | ¹H NMR (400 MHz) |
| 191 | (trans)-2-(4-amino-3-((4- | 394.35 | 395.1 | (DMSO-d₆) δ = 8.41 (d, J = |
| trans | (trifluoromethyl)pyridin- | | | 5.2 Hz, 1H), 8.16 (s, 1H), 7.60 |
| (rac) | 2-yl)oxy)-1H- | | | (s, 1H), 7.55 (d, J = 5.2 Hz, |
| | pyrazolo[3,4- | | | 1H), 4.67 (d, J = 5.2 Hz, 1H), |
| | d]pyrimidin-1- | | | 4.43-4.29 (m, 1H), 3.87- |
| | yl)cyclohexanol | | | 3.72 (m, 1H), 2.02-1.62 (m, |
| | (racemic) | | | 5H), 1.44-1.20 (m, 3H). |
| 191 | (1S,2S)-2-(4-amino-3- | 394.35 | 395.1 | (DMSO-d₆) δ = 8.41-8.40 (m, |
| (S,S) | ((4- | | | 1H), 8.16 (s, 1H), 7.60 (s, 1H), |
| | (trifluoromethyl)pyridin- | | | 7.56-7.54 (m, 2H), 4.67 (s, |
| | 2-yl)oxy)-1H- | | | 1H), 4.36-4.32 (m, 1H), 3.79 |
| | pyrazolo[3,4- | | | (m, 1H), 2.02-1.70 (m, 5H), |
| | d]pyrimidin-1- | | | 1.35-1.23 (m, 3H) |
| | yl)cyclohexanol | | | |
| 191 | (1R,2R)-2-(4-amino-3- | 394.35 | 395.1 | (DMSO-d₆) δ = 8.41-8.40 (d, |
| (R,R) | ((4- | | | J = 5.2 Hz, 1H), 8.16 (s, 1H), |
| | (trifluoromethyl)pyridin- | | | 7.60 (s, 1H), 7.55-7.54 (d, J = |
| | 2-yl)oxy)-1H- | | | 5.2 Hz, 1H), 4.66 (s, 1H), |
| | pyrazolo[3,4- | | | 4.39-4.32 (m, 1H), 3.79 (m, |
| | d]pyrimidin-1- | | | 1H), 1.98-1.70 (m, 5H), 1.36- |
| | yl)cyclohexanol | | | 1.23 (m, 3H) |
| 191 | (cis)-2-(4-amino-3-((4- | 394.35 | 395.1 | (METHANOL-d₄) δ = 8.43- |
| cis | (trifluoromethyl)pyridin- | | | 8.42 (d, J = 5.2 Hz, 1H), 8.31 |
| (rac) | 2-yl)oxy)-1H- | | | (s, 1H), 7.76 (s, 1H), 7.56- |
| | pyrazolo[3,4- | | | 7.55 (d, J = 5.2 Hz, 1H), 4.89- |
| | d]pyrimidin-1- | | | 4.82 (m, 1H), 4.24-4.23 (m, |
| | yl)cyclohexanol | | | 1H), 2.46-2.41 (m, 1H), 1.97 |
| | (racemic) | | | 1.93 (m, 2H), 1.93-1.87 (m, |
| | | | | 1H), 1.74-1.72 (m, 2H), 1.53- |
| | | | | 1.50 (m, 2H) |

### Synthesis Method X: General Procedure Represented by the Preparation of 2-((4-amino-1-(4,4-difluorocyclohexyl)-1H-pyrazolo[3,4-d]pyrimidin-3-yl)oxy)pyridin-4-ol (187)

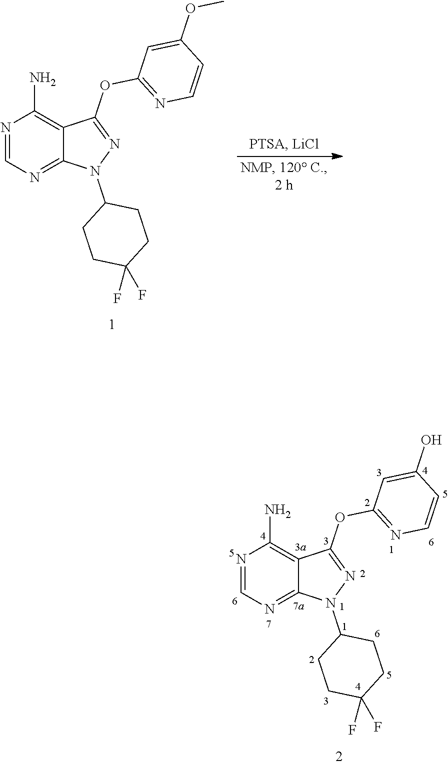 [SMILES: COc1ccnc(Oc2nn(C3CCC(F)(F)CC3)c3ncnc(N)c23)c1 ; Nc1ncnc2c1c(Oc1cc(O)ccn1)nn2C1CCC(F)(F)CC1]

To a solution of 1-(4,4-difluorocyclohexyl)-3-[(4-methoxy-2-pyridyl)oxy]pyrazolo[3,4-d]pyrimidin-4-amine (1) (90 mg, 239.13 umol, 1 eq) in NMP (2 mL) and was added LiCl (101.38 mg, 2.39 mmol, 48.97 uL, 10 eq) and PTSA (411.79 mg, 2.39 mmol, 10 eq). The mixture was stirred at 120° C. for 2 h. The mixture was purified by prep-HPLC (TFA condition) to afford 2-[4-amino-1-(4,4-difluorocyclohexyl)pyrazolo[3,4-d]pyrimidin-3-yl]oxypyridin-4-ol (2) (50 mg, 136.61 umol, 57.13% yield, 99% purity) as a white solid.

¹H NMR: (400 MHz, MeOD-d₄) δ=8.29 (d, J=2.0 Hz, 1H), 7.86 (d, J=5.6 Hz, 1H), 6.63 (dd, J=1.8, 5.6 Hz, 1H), 6.52 (d, J=2.0 Hz, 1H), 4.86 (s, 1H), 2.19-1.93 (m, 8H)

### Synthesis Method AA: General Procedure Represented by the Preparation of 3-(3-chlorophenoxy)-1-(1H-pyrrol-3-ylmethyl)pyrazolo[3,4-d]pyrimidin-4-amine (6)

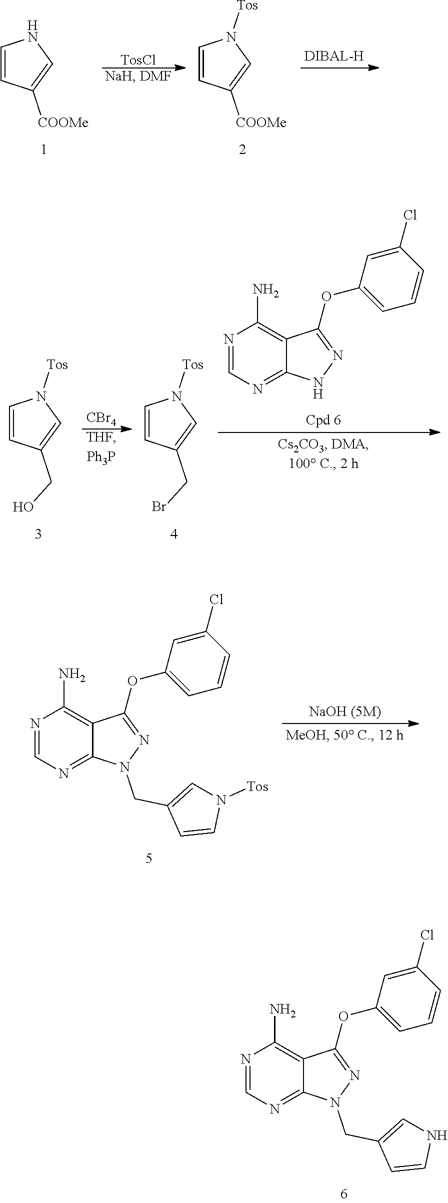

### Step 1. Procedure for preparation of methyl 2-[1-(p-tolylsulfonyl) pyrrol-3-yl]acetate (2)

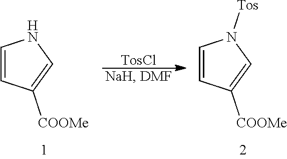

To a stirred solution of TosCl (8.38 g, 43.96 mmol, 1.1 eq) in THF (50 mL) at 0° C. was added NaH (1.76 g, 43.96 mmol, 60% purity, 1.1 eq) in portions. After 30 min, methyl 1H-pyrrole-3-carboxylate (1) (5 g, 39.% mmol, 1 eq) was added. After 30 min, the reaction mixture was warmed to 20° C. and stirred for 15 h. Water (30 mL) was added and the mixture was extracted with EtOAc (40 mL). The organic layer was washed with 5 percent aqueous NaHCO₃ then dried over MgSO₄. The crude product was purified by silica gel column chromatography (PE/EA=1/0 to 0/1) to give methyl 2-[1-(p-tolylsulfonyl)pyrrol-3-yl]acetate (2) (6 g, 20.45 mmol, 51.19% yield) as a red solid.

¹H NMR: (400 MHz, chloroform-d) δ=7.40-7.38 (d, 2H), 7.35-7.34 (d, 1H), 6.94-6.92 (d, 2H), 6.79-6.78 (s, 1H), 6.16-6.15 (d, 2H), 3.31-3.29 (s, 3H), 1.93 (s, 3H)

### Step 2. Procedure for preparation of [3-(hydroxymethyl)pyrrol-1-yl]methyl 4-methylbenzenesulfonate (3)

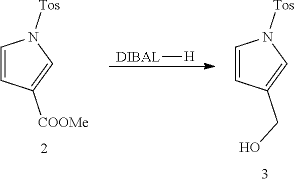

To a solution of methyl 1-(p-tolylsulfonyloxymethyl)pyrrole-3-carboxylate methyl 1-(p-tolylsulfonyloxymethyl)pyrrole-3-carboxylate (2) (3 g, 9.70 mmol, 1 eq) in toluene (30 mL) was added DIBAL-H (1 M, 19.40 mL, 2 eq) at 0° C. under an argon atmosphere. The mixture was stirred at 0° C. for 2 hr. The reaction mixture was diluted with water (30 mL) and extracted with EtOAc (3×30 mL). The combined organic layer's were dried over anhydrous sodium sulfate, filtered and concentrated under reduced pressure to give a residue. The residue was purified by column chromatography (SiO₂, petroleum ether/ethyl acetate=10/1 to 1/1) to afford [3-(hydroxymethyl)pyrrol-1-yl]methyl 4-methylbenzenesulfonate (3) (2 g, 7.11 mmol, 73.30% yield) as brown oil.

¹H NMR: (400 MHz, chloroform-d) δ=7.79-7.73 (m, 2H), 7.30 (d, J=8.8 Hz, 2H), 7.16-7.11 (m, 2H), 6.31 (dd, J=1.6, 3.2 Hz, 1H), 4.51 (s, 2H), 2.41 (s, 3H)

### Step 3. Procedure for preparation of 3-(bromomethyl)-1-(p-tolylsulfonyl)pyrrole (4)

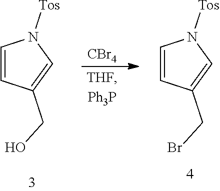

To a stirred solution of [1-(p-tolylsulfonyl)pyrrol-3-yl]methanol (3) (1 g, 3.98 mmol, 1 eq) and CBr₄ (2.64 g, 7.% mmol, 2 eq) in DCM (10 mL) at 0° C. was added PPh₃ (2.09 g, 7.96 mmol, 2 eq) was stirred at 25° C. for 1 hr. The reaction mixture was diluted with water 10 mL and extracted with DCM (3×30 mL). The combined organic layer's were dried over anhydrous sodium sulfate, filtered and concentrated under reduced pressure to give a residue. The residue was purified by column chromatography (SiO₂, petroleum ether/ethyl acetate=5/1 to 3/1) to afford 3-(bromomethyl)-1-(p-tolylsulfonyl)pyrrole (4) (1 g, 3.18 mmol, 79.98% yield) as a brown solid.

¹H NMR: (400 MHz, chloroform-d) δ=7.76 (d, J=8.4 Hz, 2H), 7.31 (d, J=8.4 Hz, 2H), 7.18 (s, 1H), 7.15-7.10 (m, 1H), 6.33 (dd, J=1.6, 3.2 Hz, 1H), 4.34 (s, 2H), 2.42 (s, 3H)

### Step 4. Procedure for preparation of 3-(3-chlorophenoxy)-1-[[1-(p-tolylsulfonyl)pyrrol-3-yl]methyl]pyrazolo[3,4-d]pyrimidin-4-amine (5)

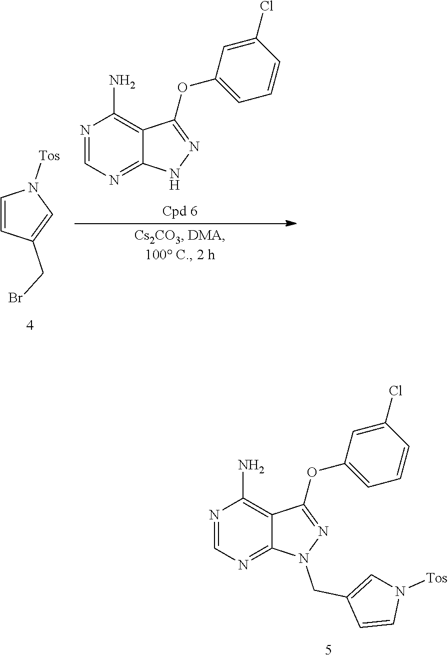

The mixture of 3-(3-chlorophenoxy)-1H-pyrazolo[3,4-d]pyrimidin-4-amine (Cpd 6) (100 mg, 382.17 umol, 1 eq), 3-(bromomethyl)-1-(p-tolylsulfonyl)pyrrole (4) (156.10 mg, 496.82 umol, 1.3 eq), KI (63.44 mg, 382.17 umol, 1 eq) and Cs₂CO₃ (161.87 mg, 496.82 umol, 1.3 eq) in DMA (1 mL) was stirred 100° C. for 2 h under N₂. The reaction mixture was filtered and concentrated under reduced pressure to give a residue. The residue was purified by prep-HPLC (neutral condition) to afford 3-(3-chlorophenoxy)-1-[[1-(p-tolylsulfonyl)pyrrol-3-yl]methyl]pyrazolo[3,4-d]pyrimidin-4-amine (5) (20 mg, 40.41 umol, 10.57% yield, 100% purity) as a white solid.

¹H NMR: (400 MHz, methanol-d₄) δ=8.30 (s, 1H), 7.74 (d, J=8.4 Hz, 2H), 7.44 (t, J=2.2 Hz, 1H), 7.40 (d, J=8.4 Hz, 1H), 7.36-7.26 (m, 4H), 7.22 (s, 1H), 7.14 (t, J=2.8 Hz, 1H), 5.25 (s, 2H), 2.39 (s, 3H).

### Step 5. Procedure for preparation of 3-(3-chlorophenoxy)-1-(1H-pyrrol-3-ylmethyl)pyrazolo[3,4-d]pyrimidin-4-amine (6)

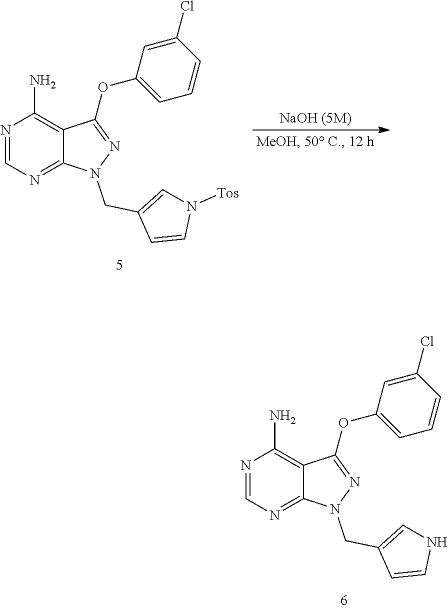

The mixture of 3-(3-chlorophenoxy)-1-[[1-(p-tolylsulfonyl)pyrrol-3-yl]methyl]pyrazolo [3,4-d]pyrimidin-4-amine (5) (20 mg, 40.41 umol, 1 eq) and NaOH (10 M, 1 mL, 247.48 eq) in MeOH (1 mL) was stirred at 50° C. for 12 h under N₂. The reaction mixture was Altered and concentrated under reduced pressure to give a residue. The residue was purified by prep-HPLC (neutral condition) to afford 3-(3-chlorophenoxy)-1-(1H-pyrrol-3-ylmethyl)pyrazolo[3,4-d]pyrimidin-4-amine (6 mg, 17.26 umol, 42.70% yield, 98% purity) as a white solid.

¹H NMR: (400 MHz, DMSO-d₆) δ=8.20 (s, 1H), 7.48 (m, J=2.4 Hz, 1H), 7.42-7.40 (m, 1H), 7.35-7.33 (m, 1H), 7.25-7.22 (m, 1H), 6.68 (s, 1H), 6.61 (s, J=2.4 Hz, 1H), 5.93 (d, J=1.6 Hz, 1H), 5.17 (s, 2H).

### Synthesis Method AB: General Procedure Represented by the Preparation of 1-(4,4-difluorocyclohexyl)-3-[[4-(trifluoromethyl)-2-pyridyl]oxy]pyrazolo[3,4-d]pyrimidin-4-amine (4)

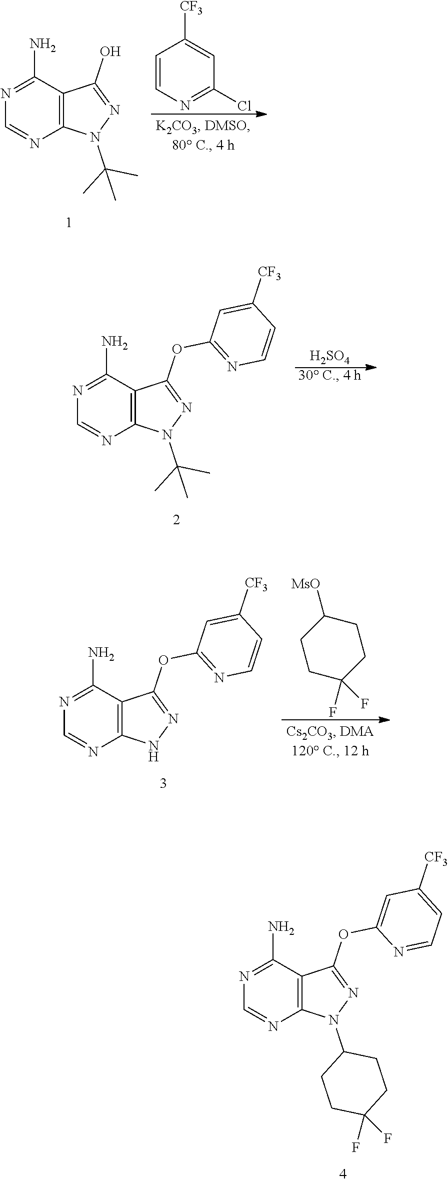

### Step 1. Procedure for preparation of 1-tert-butyl-3-[[4-(trifluoromethyl)-2-pyridyl]oxy]pyrazolo[3,4-d]pyrimidin-4-amine (2)

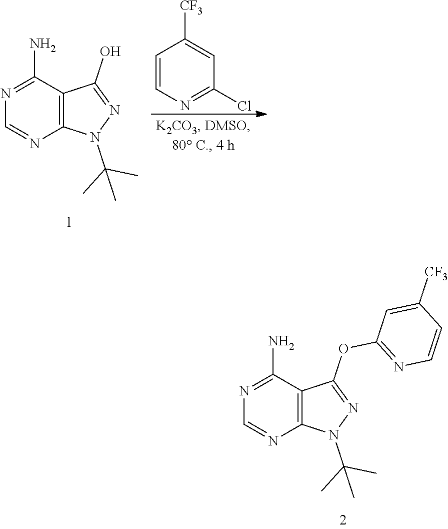

To a solution of 4-amino-1-tert-butyl-pyrazolo[3,4-d]pyrimidin-3-ol (1) (20 g, 96.51 mmol, eq) and 2-chloro-4-(trifluoromethyl)pyridine (35.04 g, 193.02 mmol, 2 eq) in DMSO (300 mL) was added K₂CO₃ (26.68 g, 193.02 mmol, 2 eq). The mixture was stirred at 80° C. for 4 hrs. LC-MS showed the reaction was completed. The mixture solution was filtered. Then the filtrate was diluted with EtOAc (2 L) and water (1 L). The mixture was separated and the aqueous phase was extracted with ethyl acetate (2×500 mL). The combined organic layers were dried over anhydrous sodium sulfate, filtered and concentrated under reduced pressure to give a residue. The residue was purified by re-crystallization from MTBE (300 mL) at 25° C. to give 1-tert-butyl-3-[[4-(trifluoromethyl)-2-pyridyl]oxy]pyrazolo[3,4-d]pyrimidin-4-amine (2) (31 g, 86.23 mmol, 44.67% yield, 98% purity) as a white solid.

¹H NMR: (400 MHz, DMSO-d₆) δ=8.39 (d, J=5.2 Hz, 1H), 8.18 (s, 1H), 7.60 (s, 1H), 7.54 (d, J=5.2, 1H), 1.67 (s, 9H).

### Step 2. Procedure for preparation of 3-[[4-(trifluoromethyl)-2-pyridyl]oxy]-1H-pyrazolo[3,4-d]pyrimidin-4-amine (3)

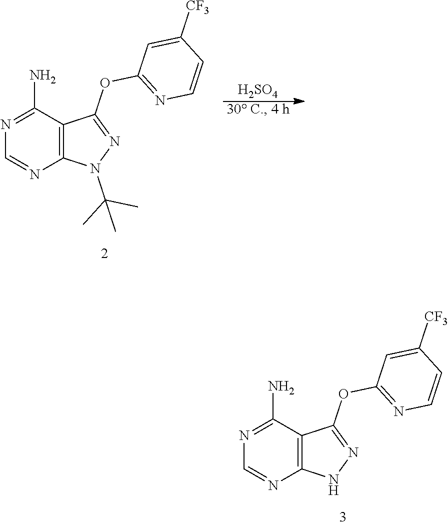

The mixture of 1-tert-butyl-3-[[4-(trifluoromethyl)-2-pyridyl]oxy]pyrazolo[3,4-d]pyrimidin-4-amine (2) (46 g, 130.57 mmol, 1 eq) and H₂SO₄ (200 mL) was stirred at 30° C. for 4 hrs. LC-MS showed the reaction was completed. The reaction mixture was quenched by adding water (1 L) at 0° C., and then the mixture was basified by adding a saturated solution of NaOH at 0° C. to pH=7. Then the reaction mixture was filtered to give filter cake. The filter cake was washed with water (3×1 L). Then the solid was dried under reduced pressure to give 3-[[4-(trifluoromethyl)-2-pyridyl]oxy]-1H-pyrazolo[3,4-d]pyrimidin-4-amine (3) (37 g, 124.91 mmol, 95.67% yield) as a white solid without further purification.

¹H NMR: (400 MHz, DMSO-d₆) δ=13.06 (s, 1H), 8.38 (d, J=5.2 Hz, 1H), 8.17 (s, 1H), 7.61 (s, 1H), 7.55 (d, J=5.2, 1H).

### Step 3. Procedure for preparation of 1-(4,4-difluorocyclohexyl)-3-[[4-(trifluoromethyl)-2-pyridyl]oxy]pyrazolo[3,4-d]pyrimidin-4-amine (4)

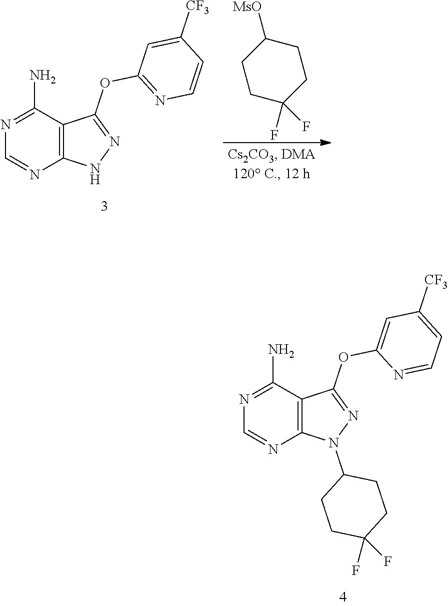

The mixture of (4,4-difluorocyclohexyl) methanesulfonate (79.56 mg, 1.1 eq), and 3-[[4-(trifluoromethyl)-2-pyridyl]oxy]-1H-pyrazolo[3,4-d]pyrimidin-4-amine (3) (100 mg, 337.60 umol, 1 eq), Cs₂CO₃ (329.99 mg, 1.01 mmol, 3 eq) in DMA (2 mL) was stirred at 120° C. for 12 hrs. LC-MS showed the reaction was completed. The mixture was filtered. The filtrate was purified by prep-HPLC (HCl condition) to give 1-(4,4-difluorocyclohexyl)-3-[[4-(trifluoromethyl)-2-pyridyl]oxy]pyrazolo[3,4-d]pyrimidin-4-amine (4) (21.1 mg, 45.89 umol, 13.59% yield, 98.04% purity) as a white solid.

LCMS: (M+H)⁺: 415.1, Rt: 2.601 min.

LC/MS (The gradient was 10-100% B in 3.4 min with a hold at 100% B for 0.45 min, 100-10% B in 0.01 min, and then held at 10% B for 0.65 min (0.8 mL/min flow rate). Mobile phase A was 0.0375% CF3CO2H in water, mobile phase B was 0.018% CF3CO2H in CH3CN. The column used for the chromatography was a 2.0×50 mm phenomenex Luna-C18 column (5 μm particles). Detection methods are diode array (DAD) and evaporative light scattering (ELSD) detection as well as positive electrospray ionization (MS).)

¹H NMR: (400 MHz, DMSO-d₆) δ=8.49-8.38 (d, J=5.2 Hz, 1H), 8.21-8.19 (m, 1H), 7.61 (s, 1H), 7.56-7.54 (d, J=5.2 Hz, 1H), 4.86-4.82 (m, 1H), 2.13-2.10 (m, 6H), 2.05-1.95 (m, 2H)

### Synthesis Method AC: General Procedure Represented by the Preparation of 1-((1s,4s)-4-(methylamino)cyclohexyl)-3-((4-(trifluoromethyl)pyridin-2-yl)oxy)-1H-pyrazolo[3,4-d]pyrimidin-4-amine and 1-((1r,4r)-4-(methylamino)cyclohexyl)-3-((4-(trifluoromethyl)pyridin-2-yl)oxy)-1H-pyrazolo[3,4-d]pyrimidin-4-amine

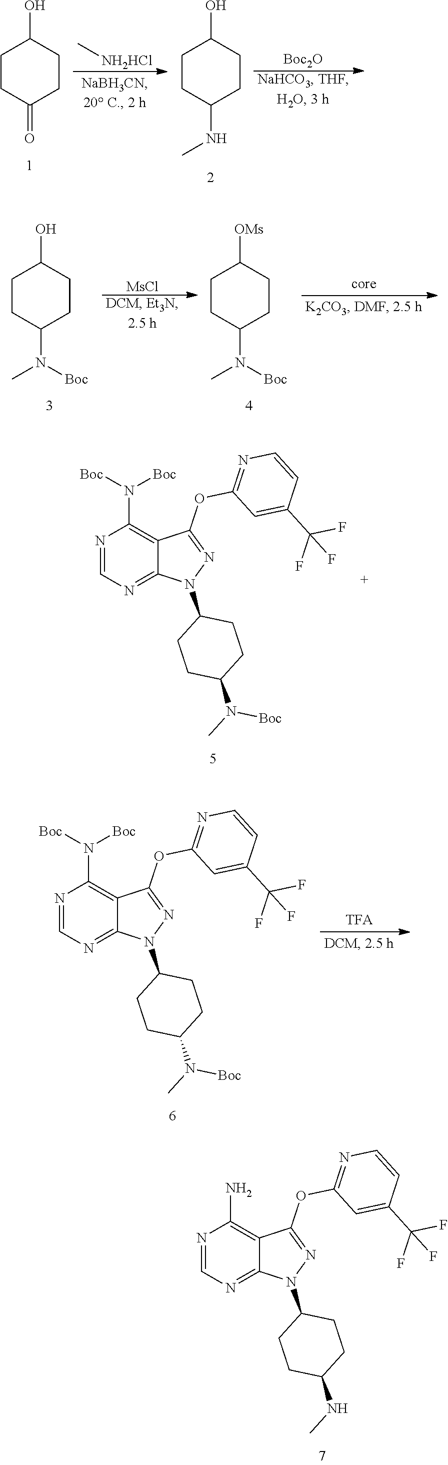

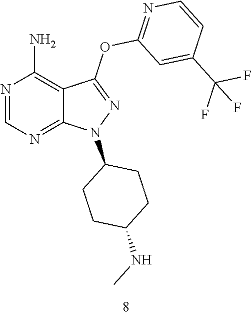 [SMILES: CN[C@H]1CC[C@H](n2nc(Oc3cc(C(F)(F)F)ccn3)c3c(N)ncnc32)CC1]

### Step 1. Procedure for preparation of 4-(methylamino)cyclohexanol (2)

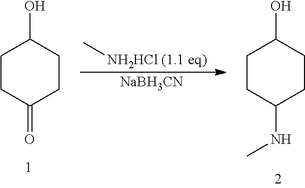

A mixture of 4-hydroxycyclohexanone (1) (5 g, 43.81 mmol, 1 eq), NaBH₃CN (4.13 g, 65.71 mmol, 1.5 eq) and methanamine (1.63 g, 52.57 mmol, 25 mL, 1.2 eq) in MeOH (10 mL) was degassed and purged with N₂ for 3 times, and then the mixture was stirred at 20° C. for 2 h under N₂ atmosphere. TLC indicated starting material was consumed, and one major new spot with larger polarity was detected. The reaction mixture was concentrated to give 4-(methylamino)cyclohexanol (2) (2 g, 15.48 mmol, yield 35.34%) as yellow oil, which was used to next step directly.

### Step 2. Procedure for preparation of tert-butyl (4-hydroxycyclohexyl)(methyl)carbamate (3)

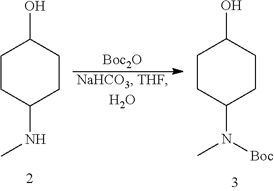

A mixture of 4-(methylamino)cyclohexanol (2) (3.36 g, 26 mmol, 1 eq) and Boc₂O (5.67 g, 26.00 mmol, 5.97 mL, 1 eq) in THF (100 mL) and water (50 mL) was added NaHCO₃ (2.18 g, 26.00 mmol, 1 eq), and the mixture was stirred at 20° C. for 3 h. TLC indicated starting material was consumed, and one major new spot with lower polarity was detected. The reaction mixture was concentrated and extracted with ethyl acetate (30 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=100/1 to 0:1) to afford tert-butyl N-(4-hydroxycyclohexyl)-N-methyl-carbamate (3) (1.2 g, 5.23 mmol, yield 20.13%) as yellow oil.

¹H NMR (400 MHz, CHLOROFORM-d) δ=4.01 (br s, 1H), 2.79-2.66 (m, 3H), 1.93-1.52 (m, 7H), 1.48-1.40 (m, 12H)

### Step 3. Procedure for preparation of 4-((tert-butoxycarbonyl)(methyl)amino)cyclohexyl methanesulfonate (4)

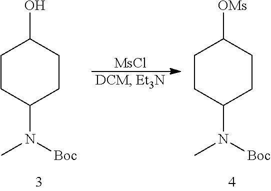

A mixture of tert-butyl N-(4-hydroxycyclohexyl)-N-methyl-carbamate (3) (0.5 g, 2.18 mmol, 1 eq) in DCM (5 mL) was added MsCl (299.72 mg, 2.62 mmol, 1.2 eq) and TEA (264.76 mg, 2.62 mmol, 1.2 eq) at 0° C. The mixture was stirred at 20° C. for 2.5 h. TLC indicated starting material was consumed and one major new spot with lower polarity was detected. The reaction mixture was diluted with dichloromethane (30 mL) and aqueous sodium hydrogen carbonate (30 mL). After partitioning, the organic phase was collected. The mixture was concentrated under reduced pressure to afford [4-[tert-butoxycarbonyl(methyl)amino]cyclohexyl] methanesulfonate (4) (0.6 g, 1.95 mmol, yield 89.52%) as yellow oil, which was used to next step directly.

### Step 4. Procedure for preparation of tert-butyl tert-butoxycarbonyl(1-((1s,4s)-4-((tert-butoxycarbonyl)(methyl)amino)cyclohexyl)-3-((4-(trifluoromethyl)pyridin-2-yl)oxy)-JH-pyrazolo[3,4-d]pyrimidin-4-yl)carbamate (5) & tert-butyl tert-butoxycarbonyl(1-((1r,4r)-4-((tert-butoxycarbonyl)(methyl)amino)cyclohexyl)-3-((4-(trifluoromethyl)pyridin-2-yl)oxy)-1H-pyrazolo[3,4-d]pyrimidin-4-yl)carbamate (6)

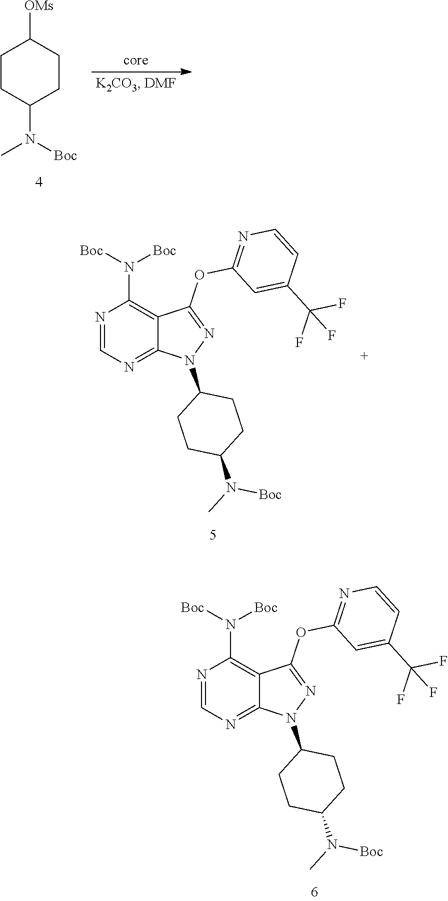 [SMILES: CN(C(=O)OC(C)(C)C)C1CCC(OS(C)(=O)=O)CC1 ; CN(C(=O)OC(C)(C)C)[C@H]1CC[C@@H](n2nc(Oc3cc(C(F)(F)F)ccn3)c3c(N(C(=O)OC(C)(C)C)C(=O)OC(C)(C)C)ncnc32)CC1 ; CN(C(=O)OC(C)(C)C)[C@H]1CC[C@H](n2nc(Oc3cc(C(F)(F)F)ccn3)c3c(N(C(=O)OC(C)(C)C)C(=O)OC(C)(C)C)ncnc32)CC1]

A mixture of [4-[tert-butoxycarbonyl(methyl)amino]cyclohexyl] methanesulfonate (4) (495.38 mg, 1.61 mmol, 2 eq), tert-butyl N-tert-butoxycarbonyl-N-[3-[[4-(trifluoromethyl)-2-pyridyl]oxy]-1H-pyrazolo[3,4-d]pyrimidin-4-yl]carbamate (core) (400 mg, 805.74 umol, eq) and K₂CO₃ (167.04 mg, 1.21 mmol, 1.5 eq) in DMF (5 mL) was degassed and purged with N₂ for 3 times, and then the mixture was stirred at 60° C. for 2.5 h under N₂ atmosphere. LCMS showed ˜50% of Core remaining and ˜40% of product with desired MS was detected. The reaction mixture was filtered and filtrate was collected. The crude product was purified by prep-HPLC (TFA condition) to afford tert-butyl N-[4-[4-[bis(tert-butoxycarbonyl)amino]-3-[[4-(trifluoromethyl)-2-pyridyl]oxy]pyrazolo[3,4-d]pyrimidin-1-yl]cyclohexyl]-N-methyl-carbamate (5) (80 mg, 113.04 umol, yield 14.03%) and tert-butyl N-[4-[4-[bis(tert-butoxycarbonyl)amino]-3-[[4-(trifluoromethyl)-2-pyridyl]oxy]pyrazolo[3,4-d]pyrimidin-1-yl]cyclohexyl]-N-methyl-carbamate (6) (80 mg, 113.04 umol, 14.03% yield) as a white solid.

Prep-HPLC method:
Instrument: Shimadzu LC-8A preparative HPLC

### Column: Nano-micro Kromasil C₁₈ 80*25 mm 3 um

Mobile phase: [water (0.1% TFA)-ACN]

### Gradient: B %: 63%-93%, 7 min

Flow rate: 40 mL/min

### Wavelength: 220&254 nm

### Step 5. Procedure for preparation of 1-((1s,4s)-4-(methylamino)cyclohexyl)-3-((4-(trifluoromethyl)pyridin-2-yl)oxy)-1H-pyrazolo[3,4-d]pyrimidin-4-amine (7)

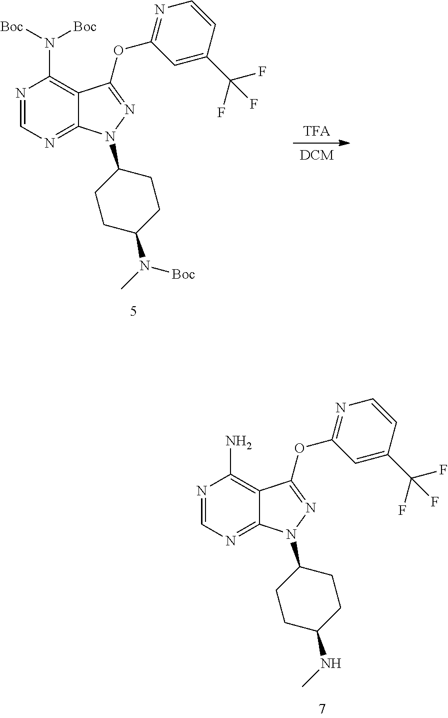

To a solution of tert-butyl tert-butoxycarbonyl(1-((1s,4s)-4-((tert-butoxycarbonyl)(methyl)amino)cyclohexyl)-3-((4-(trifluoromethyl)pyridin-2-yl)oxy)-1H-pyrazolo[3,4-d]pyrimidin-4-yl)carbamate (5) (80 mg, 113.04 umol, 1 eq) in DCM (3 mL) was added TFA (18.01 mmol, 1.33 mL, 159.31 eq). The mixture was stirred at 20° C. for 2.5 h. LC-MS showed the reaction was completed. The reaction mixture was concentrated and purified by pre-HPLC (TFA condition) to afford 1-[4-(methylamino)cyclohexyl]-3-[[4-(trifluoromethyl)-2-pyridyl]oxy]pyrazolo[3,4-d]pyrimidin-4-amine (7) (23 mg, 43.63 umol, yield 38.59%, 98.9% purity, TFA) as a white solid.

¹H NMR (7) (400 MHz, METHANOL-d₄)

δ=8.39 (d, J=5.2 Hz, 1H), 8.33-8.29 (m, 1H), 7.63 (s, 1H), 7.53 (d, J=5.2 Hz, 1H), 5.01 (br s, 1H), 3.29-3.24 (m, 1H), 2.69 (s, 3H), 2.34-2.21 (m, 2H), 2.16-1.97 (m, 6H)

### Step 6. Procedure for preparation of 1-((1r,4r)-4-(methylamino)cyclohexyl)-3-((4-(trifluoromethyl)pyridin-2-yl)oxy)-1H-pyrazolo[3,4-d]pyrimidin-4-amine (8)

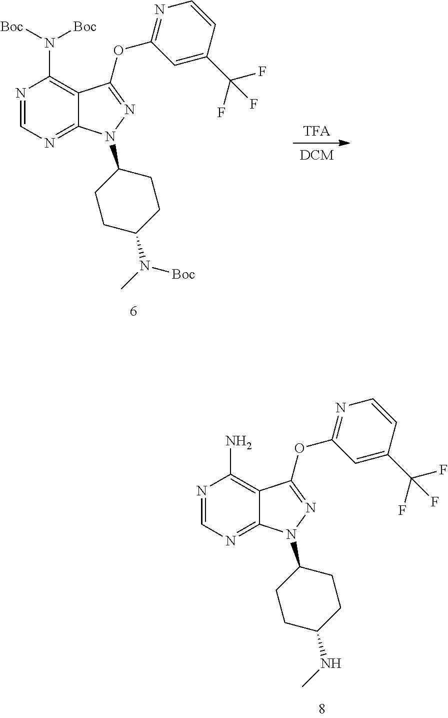

To a solution of tert-butyl tert-butoxycarbonyl(1-((1r,4r)-4-((tert-butoxycarbonyl)(methyl)amino)cyclohexyl)-3-((4-(trifluoromethyl)pyridin-2-yl)oxy)-1H-pyrazolo[3,4-d]pyrimidin-4-yl)carbamate (6) (80 mg, 113.04 umol, 1 eq) in DCM (3 mL) was added TFA (18.01 mmol, 1.33 mL, 159.31 eq). The mixture was stirred at 20° C. for 2.5 h. LC-MS showed the reaction was completed. The reaction mixture was concentrated and purified by pre-HPLC (TFA condition) to afford 1-((1r, 4r)-4-(methylamino)cyclohexyl)-3-((4-(trifluoromethyl)pyridin-2-yl)oxy)-1H-pyrazolo[3,4-d]pyrimidin-4-amine (8) (15 mg, 28.45 umol, yield 25.17%, 100% purity, TFA) as a white solid.

¹H NMR (8) (400 MHz, METHANOL-d₄)

δ=8.40 (d, J=5.2 Hz, 1H), 8.32-8.28 (m, 1H), 7.67 (s, 1H), 7.53 (d, J=5.2 Hz, 1H), 4.81-4.75 (m, 1H), 3.21-3.09 (m, 1H), 2.73 (s, 3H), 2.29 (br d, J=12.0 Hz, 2H), 2.22-2.00 (m, 4H), 1.68-1.62 (m, 2H)

### Synthesis Method AD: General Procedure Represented by the Preparation of 5-((4-amino-3-((4-(trifluoromethyl)pyridin-2-yl)oxy)-1H-pyrazolo[3,4-d]pyrimidin-1-yl)methyl)-1,2-oxaborolan-2-ol

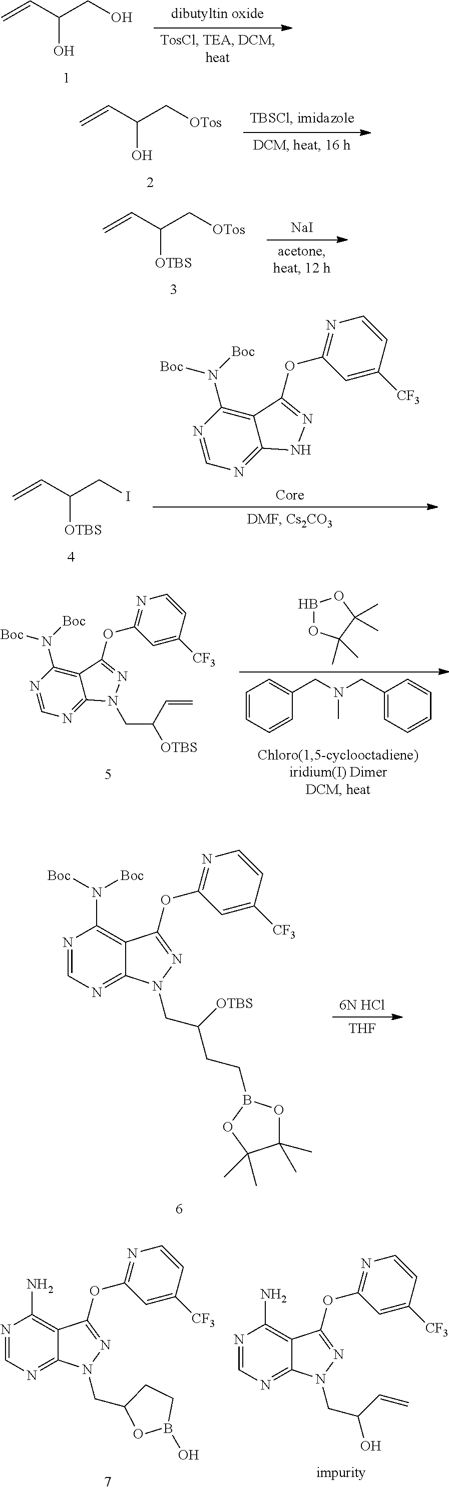

### Step 1. Procedure for preparation of 2-hydroxybut-3-en-1-yl 4-methylbenzenesulfonate (2)

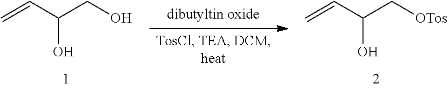

To a mixture of but-3-ene-1,2-diol (1) (3 g, 34.05 mmol, 1 eq) and dibutyl(oxo)tin (847.64 mg, 3.41 mmol, 0.1 eq) in dichloromethane (30 mL) was added TosCl (6.49 g, 34.05 mmol, eq) and triethylamine (3.45 g, 34.05 mmol, 1 eq) at 25° C. The reaction was stirred for 16 h at 25° C. TLC (Petroleum ether: Ethyl acetate=3:1, R_{f}=0.65) indicated Reactant was consumed completely, and one major new spot with lower polarity was detected. The reaction mixture was partitioned between water (100 mL) and dichloromethane (100 mL). The organic phase was washed with brine (100 mL), dried over Na₂SO₄, filtered and concentrated under reduced pressure to give a residue. The residue was purified by column chromatography (SiO₂, Petroleum ether: Ethyl acetate=5:1 to 3:1) to give 2-hydroxybut-3-enyl 4-methylbenzenesulfonate (2) (4.5 g, 18.57 mmol, yield 54.54%) as colorless oil.

### Step 2. Procedure for preparation of 2-((tert-butyldimethylsilyl)oxy)but-3-en-1-yl 4-methylbenzenesulfonate (3)

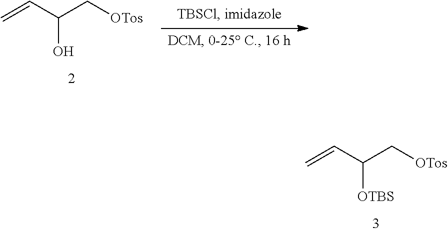

To a solution of 2-hydroxybut-3-enyl 4-methylbenzenesulfonate (2) (4 g, 16.51 mmol, 1 eq) in dichloromethane (40 mL) was added imidazole (6.74 g, 99.05 mmol, 6 eq) and TBSCl (9.95 g, 66.04 mmol, 8.09 mL, 4 eq) at 0° C. The reaction mixture was stirred for 16 h at 25° C. TLC (petroleum ether: ethyl acetate=3:1, Rf=0.76) indicated Reactant was consumed completely, and one major new spot with lower polarity was detected. The reaction system was filtered. DCM (200 mL) was added into the filtrate and washed with water (200 mL), hydrochloric acid (1 M, 200 mL×3). Finally, the organic phase was combined, dried over Na₂SO₄ and concentrated under reduced pressure to give a crude product. The residue was purified by column chromatography (SiO₂, Petroleum ether: Ethyl acetate=3:1 to 1:1) to give 2-((tert-butyldimethylsilyl)oxy)but-3-en-1-yl 4-methylbenzenesulfonate (3) (3.33 g, 9.35 mmol, yield 56.63%) as colorless oil.

### Step 3. Procedure for preparation of tert-butyl((1-iodobut-3-en-2-yl)oxy)dimethylsilane (4)

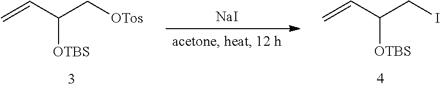

To a solution of 2-[tert-butyl(dimethyl)silyl]oxybut-3-enyl 4-methylbenzenesulfonate (3) (4 g, 11.22 mmol, 1 eq) in acetone (40 mL) was added NaI (16.82 g, 112.19 mmol, 10 eq). The reaction mixture was stirred for 12 h at 60° C. TLC (Petroleum ether: Ethyl acetate 5:1, Rf=0.7) indicated Reactant was consumed completely, and one major new spot with lower polarity was detected. The reaction system was decompressed to concentrate the solvent, and then ethyl acetate (200 mL) and water (200 mL) were added to retain the organic phase. The organic phase was washed with water (200 mL×3), dried over Na₂SO₄ and concentrated to afford a crude product. The residue was purified by column chromatography (SiO₂, Petroleum ether/Ethyl acetate=1:0 to 5:1) to give tert-butyl ((1-iodobut-3-en-2-yl) oxy) dimethylsilane (4) (1 g, 3.20 mmol, yield 28.55%) as colorless oil.

### Step 4. Procedure for preparation of tert-butyl N-tert-butoxycarbonyl-N-(1-(2-((tert-butyldimethylsilyl)oxy)but-3-en-1-yl)-3-((4-(trifluoromethyl)pyridin-2-yl)oxy)-1H-pyrazolo[3,4-d]pyrimidin-4-yl)carbamate (5)

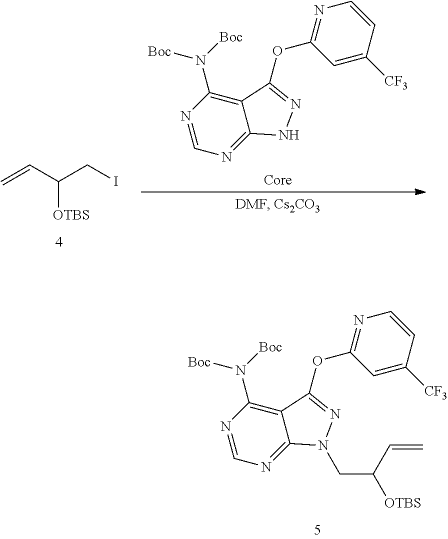

To a solution of tert-butyl ((l-iodobut-3-en-2-yl) oxy) dimethylsilane (4) (500 mg, 1.60 mmol, 1.3 eq) and tert-butyl N-tert-butoxycarbonyl-N-[3-[[4-(trifluoromethyl)-2-pyridyl]oxy]-1H-pyrazolo[3,4-d]pyrimidin-4-yl]carbamate (611.47 mg, 1.23 mmol, 1 eq) (Core) in DMF (2 mL) was added Cs₂CO₃ (1.00 g, 3.08 mmol, 2.5 eq). The mixture was stirred at 60° C. for 12 h. LCMS showed 23% of Core remaining and 51% of desired compound was detected. The reaction mixture was concentrated under reduced pressure to remove solvent. The residue was purified by column chromatography (SiO₂, Petroleum ether/Ethyl acetate=10/1 to 5/1) to give tert-butyl N-tert-butoxycarbonyl-N-(1-(2-((tert-butyldimethylsilyl)oxy)but-3-en-1-yl)-3-((4-(trifluoromethyl)pyridin-2-yl)oxy)-1H-pyrazolo[3,4-d]pyrimidin-4-yl)carbamate (5) (700 mg, 1.03 mmol, yield 41.74%) as a white solid.

### Step 5. Procedure for preparation of tert-butyl N-tert-butoxycarbonyl-N-(1-(2-((tert-butyldimethylsilyl)oxy)-4-(4,4,5,5-tetramethyl-1,3,2-dioxaborolan-2-yl)butyl)-3-((4-(trifluoromethyl)pyridin-2-yl)oxy)-1H-pyrazolo[3,4-d]pyrimidin-4-yl)carbamate (6)

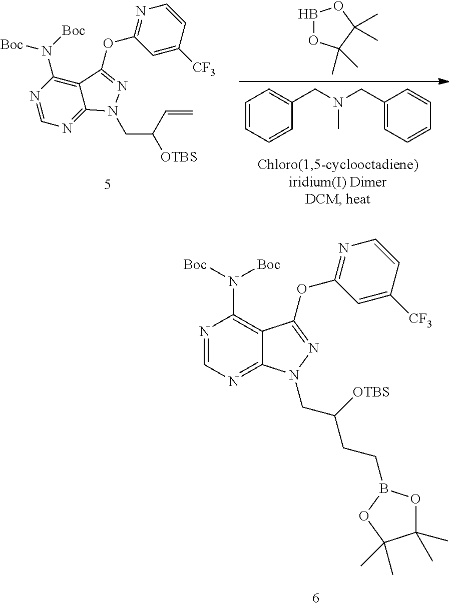

A mixture of tert-butyl N-tert-butoxycarbonyl-N-(1-(2-((tert-butyldimethylsilyl)oxy)but-3-en-1-yl)-3-((4-(trifluoromethyl)pyridin-2-yl)oxy)-1H-pyrazolo[3,4-d]pyrimidin-4-yl)carbamate (5) (0.1 g, 146.89 umol, 1 eq), N-benzyl-N-methyl-1-phenyl-methanamine (31.04 mg, 146.89 umol, 1 eq) in dichloromethane (5 mL) was added 4,4,5,5-tetramethyl-1,3,2-dioxaborolane (18.80 mg, 146.89 umol, 1 eq), Chloro(1,5-cyclooctadiene)iridium(I)dimer (98.67 mg, 146.89 umol, 1 eq) and purged with N₂ for 3 times, and then the mixture was stirred at 20° C. for 12 h under N₂ atmosphere. LCMS showed starting material was consumed and the desired product was detected. The reaction mixture was concentrated under reduced pressure to remove solvent. The residue was diluted with water (10 mL) and extracted with ethyl acetate (5 mL×3). The combined organic layers were dried over Na₂SO₄, filtered and concentrated under reduced pressure to give a residue. The crude product was purified by prep-HPLC (TFA condition, Column: Welch Ultimate AQ-C18 150*30 mm*5 um; mobile phase: [water (0.1% TFA)-ACN]; B %: 90%-98%, 12 min) to give tert-butyl N-tert-butoxycarbonyl-N-(1-(2-((tert-butyldimethylsilyl)oxy)-4-(4,4,5,5-tetramethyl-1,3,2-dioxaborolan-2-yl)butyl)-3-((4-(trifluoromethyl)pyridin-2-yl)oxy)-1H-pyrazolo[3,4-d]pyrimidin-4-yl)carbamate (6) (100 mg, contained (5)) as a white solid.

### Step 6. Procedure for preparation of 5-((4-amino-3-((4-(trifluoromethyl)pyridin-2-yl)ox)-1H-pyrazolo[3,4-d]pyrimidin-1-yl)methyl)-1,2-oxaborolan-2-ol (7)

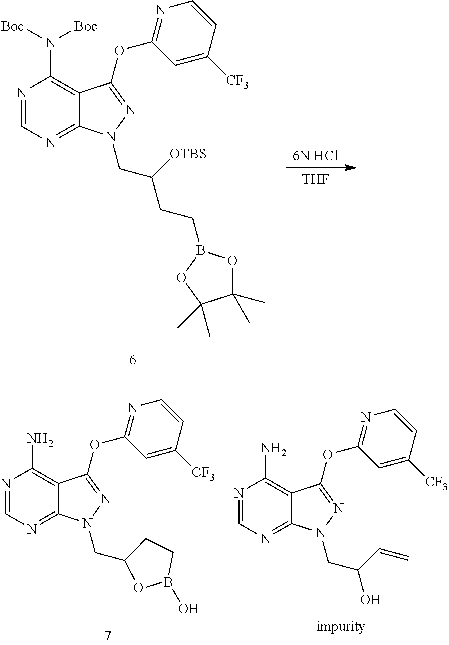 [SMILES: CC(C)(C)OC(=O)N(C(=O)OC(C)(C)C)c1ncnc2c1c(Oc1cc(C(F)(F)F)ccn1)nn2CC(CCB1OC(C)(C)C(C)(C)O1)O[Si](C)(C)C(C)(C)C ; "6N HCl" ; "THF" ; Nc1ncnc2c1c(Oc1cc(C(F)(F)F)ccn1)nn2CC1CCB(O)O1 ; C=CC(O)Cn1nc(Oc2cc(C(F)(F)F)ccn2)c2c(N)ncnc21]

A mixture of tert-butyl N-tert-butoxycarbonyl-N-(1-(2-((tert-butyldimethylsilyl)oxy)-4-(4,4,5,5-tetramethyl-1,3,2-dioxaborolan-2-yl)butyl)-3-((4-(trifluoromethyl)pyridin-2-yl)oxy)-1H-pyrazolo[3,4-d]pyrimidin-4-yl)carbamate (6) (100 mg, 123.65 umol, 1 eq) in THF (2 mL) was added HCl (1 mL, 6 moL/L) and the mixture was stirred at 20° C. for 12 h. LCMS showed the starting material was consumed and desired product was detected. The reaction mixture was extracted with ethyl acetate (30 mL×2). The combined organic layers were dried over Na₂SO₄, Altered and concentrated under reduced pressure to give a residue. The residue was purified by prep-HPLC (TFA condition, column: Nano-micro Kromasil C₁₈ 100*40 mm 10 um; mobile phase: [water (0.1% TFA)-ACN]; B %: 10%-40%, 7 min) to give 5-((4-amino-3-((4-(trifluoromethyl)pyridin-2-yl)oxy)-1H-pyrazolo[3,4-d]pyrimidin-1-yl)methyl)-1,2-oxaborolan-2-ol (7) (60 mg, 152.24 umol, yield 61.56%) as a white solid.

¹H NMR (7) (400 MHz, METHANOL-d₄) δ=8.41 (s, 1H), 8.30 (s, 1H), 7.71 (s, 1H), 7.53 (s, 1H), 4.41-4.30 (m, 3H), 1.87 (m, 1H), 1.67 (m, 1H), 0.93-0.86 (m, 2H)

¹H NMR (impurity) (400 MHz, METHANOL-d₄) δ=8.42 (d, J=4.8 Hz, 1H), 8.29 (s, 1H), 7.71 (s, 1H), 7.55 (d, J=4.8 Hz, 1H), 5.96-5.88 (m, 1H), 5.29 (d, 7=17.2 Hz, 1H) 5.15 (d. J=10.8 Hz, 1H) 4.58-4.56 (m, 1H), 4.38-4.28 (m, 2H)

### Synthesis Method AE: General Procedure Represented by the Preparation of (3-((4-amino-3-((4-(trifluoromethyl)pyridin-2-yl)oxy)-1H-pyrazolo[3,4-d]pyrimidin-1-yl)methyl)cyclobutyl)boronic acid

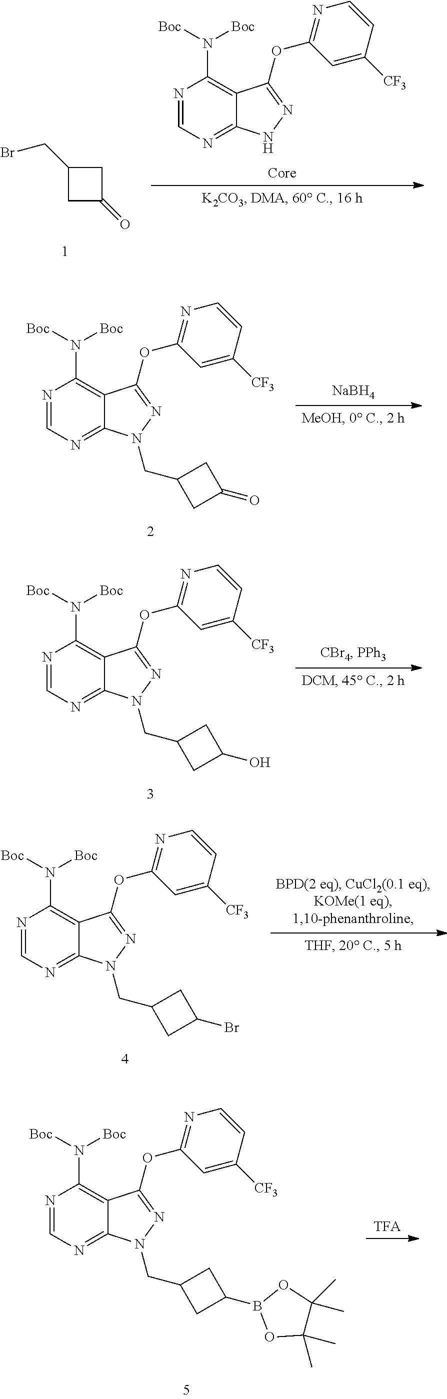

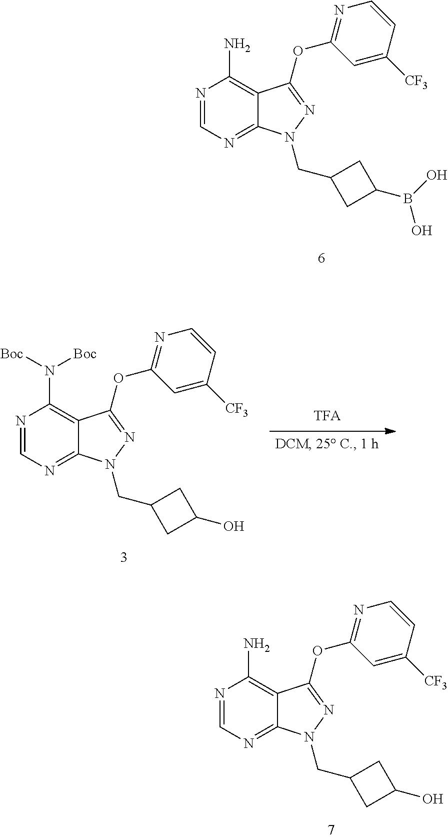 [SMILES: Nc1ncnc2c1c(Oc1cc(C(F)(F)F)ccn1)nn2CC1CC(B(O)O)C1 ; CC(C)(C)OC(=O)N(C(=O)OC(C)(C)C)c1ncnc2c1c(Oc1cc(C(F)(F)F)ccn1)nn2CC1CC(O)C1 ; "DCM, 25° C., 1 h" ; "TFA" ; Nc1ncnc2c1c(Oc1cc(C(F)(F)F)ccn1)nn2CC1CC(O)C1]

### Step 1. Procedure for preparation of tert-butyl N-tert-butoxycarbonyl-N-[1-[(3-oxocyclobutyl)methyl]-3-[[4-(trifluoromethyl)-2-pyridyl]oxy]pyrazolo[3,4-d]pyrimidin-4-yl]carbamate (2)

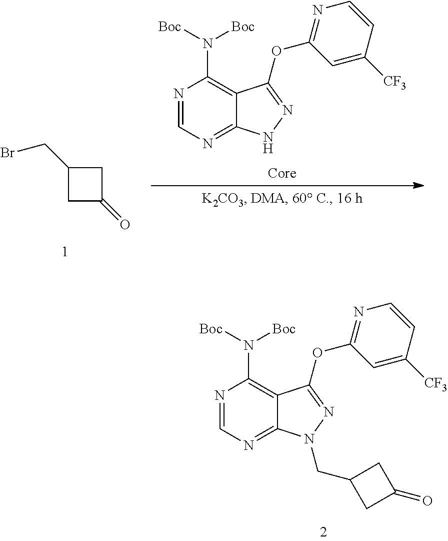

To a mixture of tert-butyl N-tert-butoxycarbonyl-N-[3-[[4-(trifluoromethyl)-2-pyridyl]oxy]-1H-pyrazolo[3,4-d]pyrimidin-4-yl]carbamate (Core) (500 mg, 1.01 mmol, 1 eq) and K₂CO₃ (278.40 mg, 2.01 mmol, 2 eq) in dimethylacetamide (10 mL) was added 3-(bromomethyl)cyclobutanone (1) (246.27 mg, 1.51 mmol, 1.5 eq) under N₂ at 25° C. The reaction was stirred for 16 h at 60° C. under N₂. LCMS showed the reactant was consumed and desired compound was detected. The resulting mixture was filtered to remove the insoluble matter. The filtrate was purified by prep-HPLC (neutral condition) to give tert-butyl N-tert-butoxycarbonyl-N-[1-[(3-oxocyclobutyl)methyl]-3-[[4-(trifluoromethyl)-2-pyridyl]oxy]pyrazolo[3,4-d]pyrimidin-4-yl]carbamate (2) (2.5 g. yield 71.51%) as yellow oil.

### Step 2. Procedure for preparation of tert-butyl N-tert-butoxycarbonyl-N-[1-[(3-hydroxycyclobutyl)methyl]-3-[[4-(trifluoromethyl)-2-pyridyl]oxy]pyrazolo[3,4-d]pyrimidin-4-yl]carbamate (3)

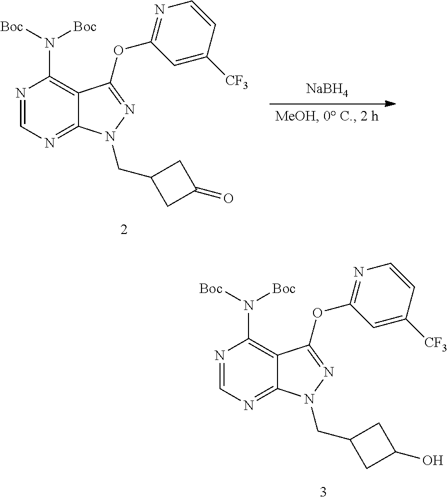 [SMILES: CC(C)(C)OC(=O)N(C(=O)OC(C)(C)C)c1ncnc2c1c(Oc1cc(C(F)(F)F)ccn1)nn2CC1CC(=O)C1 ; "MeOH, 0° C., 2 h" ; "NaBH4" ; CC(C)(C)OC(=O)N(C(=O)OC(C)(C)C)c1ncnc2c1c(Oc1cc(C(F)(F)F)ccn1)nn2CC1CC(O)C1]

To a solution of tert-butyl N-tert-butoxycarbonyl-N-[1-[(3-oxocyclobutyl)methyl]-3-[[4-(trifluoromethyl)-2-pyridyl]oxy]pyrazolo[3,4-d]pyrimidin-4-yl]carbamate (2) (2.5 g, 4.32 mmol, 1 eq) in MeOH (30 mL) was added NaBH₄ (245.22 mg, 6.48 mmol, 1.5 eq) at 0° C. The reaction mixture was stirred for 2 h at 0° C. LCMS showed the reactant was consumed and the desired compound was detected. The resulting mixture was acidified by adding HCl (5%) to pH=7, then was concentrated in vacuum to give the residue. The residue was purified by prep-HPLC (basic condition) to give tert-butyl N-tert-butoxycarbonyl-N-[1-[(3-hydroxycyclobutyl)methyl]-3-[[4-(trifluoromethyl)-2-pyridyl]oxy]pyrazolo[3,4-d]pyrimidin-4-yl]carbamate (3) (1.7 g, yield 67.76%) as a white solid.

### Step 3. Procedure for preparation of tert-butyl N-tert-butoxycarbonyl-N-(1-((3-bromocyclobutyl)methyl)-3-((4-(trifluoromethyl)pyridin-2-yl)oxy)-1H-pyrazolo[3,4-d]pyrimidin-4-yl)carbamate (4)

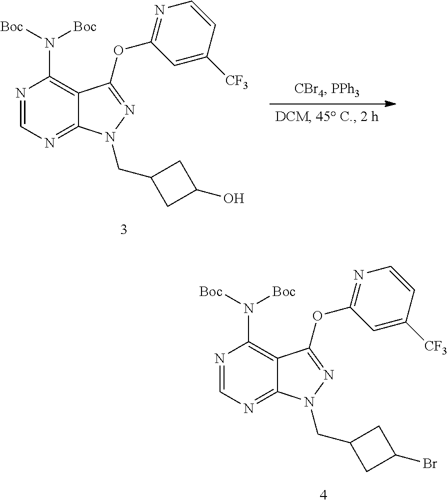

To a solution of tert-butyl N-tert-butoxycarbonyl-N-[1-[(3-hydroxycyclobutyl)methyl]-3-[[4-(trifluoromethyl)-2-pyridyl]oxy]pyrazolo[3,4-d]pyrimidin-4-yl]carbamate (3) (400 mg, 689.00 umol, 1 eq) in DCM (10 mL) was added PPh₃ (410.22 mg, 1.56 mmol, 2.27 eq), CBr₄ (571.22 mg, 1.72 mmol, 2.5 eq) at 25° C. The reaction was stirred for 2 h at 45° C. LCMS showed the reactant was consumed and the desired compound was detected. The resulting mixture was concentrated to give the residue, which was purified by prep-HPLC (TFA condition) to give tert-butyl N-[1-[(3-bromocyclobutyl)methyl]-3-[[4-(trifluoromethyl)-2-pyridyl]oxy]pyrazolo[3,4-d]pyrimidin-4-yl]-N-tert-butoxycarbonyl-carbamate (4) (160 mg, yield 35.93%) as red oil.

¹H NMR (400 MHz, CHLOROFORM-d) δ=8.88 (s, 1H), 8.30 (d, J=5.2 Hz, 1H), 7.33 (s, 1H), 7.31 (d, J=5.2 Hz, 1H), 4.56-4.51 (m, 3H), 3.31-3.19 (m, 1H), 2.69-2.55 (m, 4H), 1.39 (s, 18H)

### Step 4. Procedure for preparation of tert-butyl N-tert-butoxycarbonyl-N-(1-((3-(4,4,5,5-tetramethyl-1,3,2-dioxaborolan-2-yl)cyclobutyl)methyl)-3-((4-(trifluoromethyl)pyridin-2-yl)oxy)-1H-pyrazolo[3,4-d]pyrimidin-4-yl)carbamate(5)

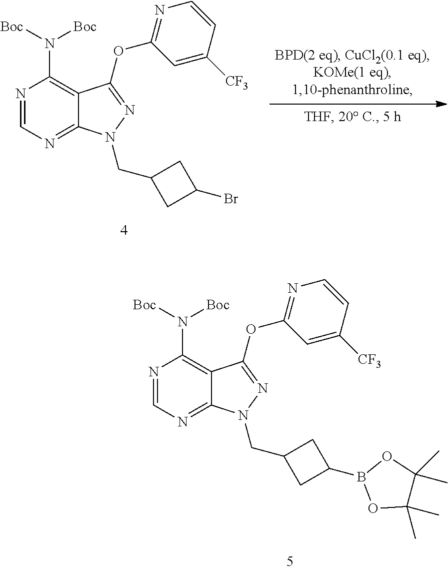 [SMILES: CC(C)(C)OC(=O)N(C(=O)OC(C)(C)C)c1ncnc2c1c(Oc1cc(C(F)(F)F)ccn1)nn2CC1CC(Br)C1 ; "BPD(2 eq), CuCl2(0.1 eq),KOMe(1 eq),1,10-phenanthroline," ; "THF, 20° C., 5 h" ; CC(C)(C)OC(=O)N(C(=O)OC(C)(C)C)c1ncnc2c1c(Oc1cc(C(F)(F)F)ccn1)nn2CC1CC(B2OC(C)(C)C(C)(C)O2)C1]

To a solution of tert-butyl N-[1-[(3-bromocyclobutyl)methyl]-3-[[4-(trifluoromethyl)-2-pyridyl]oxy]pyrazolo[3,4-d]pyrimidin-4-yl]-N-tert-butoxycarbonyl-carbamate (4) (300 mg, eq) in THF (2 mL) was added BPD (236 mg, 2 eq) and CuCl₂ (19 mg, 0.3 eq) and methoxypotassium (65 mg, 2 eq) and 1, 10-phenanthroline (42 mg, 0.5 eq). The mixture was stirred at 20° C. for 5 h. TLC indicated 5% of (4) remaining and one major new spot with larger polarity is detected. The reaction mixture was concentrated under reduced pressure to remove solvent. The residue was diluted with water (10 mL) and extracted with ethyl acetate (10 mL×3). The combined organic layers were washed with brine (5 mL×2), dried over Na₂SO₄, filtered and concentrated under reduced pressure to give a residue. The crude product is purified by Pre-TLC (petroleum ether: ethyl acetate 5:1) to tert-butyl N-tert-butoxycarbonyl-N-(1-((3-(4,4,5,5-tetramethyl-1,3,2-dioxaborolan-2-yl)cyclobutyl)methyl)-3-((4-(trifluoromethyl)pyridin-2-yl)oxy)-1H-pyrazolo[3,4-d]pyrimidin-4-yl)carbamate (5) (60 mg, 18.64% yield) was obtained as a white solid, which was used to next step directly.

### Step 5. Procedure for preparation of (3-((4-amino-3-((4-(trifluoromethyl)pyridin-2-yl)oxy)-1H-pyrazolo[3,4-d]pyrimidin-1-yl)methyl)cyclobutyl)boronic acid(6)

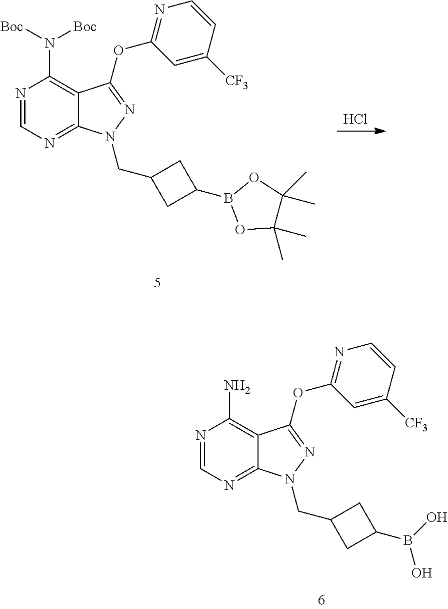 [SMILES: CC(C)(C)OC(=O)N(C(=O)OC(C)(C)C)c1ncnc2c1c(Oc1cc(C(F)(F)F)ccn1)nn2CC1CC(B2OC(C)(C)C(C)(C)O2)C1 ; Cl ; Nc1ncnc2c1c(Oc1cc(C(F)(F)F)ccn1)nn2CC1CC(B(O)O)C1]

To a solution of tert-butyl N-tert-butoxycarbonyl-N-(1-((3-(4,4,5,5-tetramethyl-1,3,2-dioxaborolan-2-yl)cyclobutyl)methyl)-3-((4-(trifluoromethyl)pyridin-2-yl)oxy)-1H-pyrazolo[3,4-d]pyrimidin-4-yl)carbamate (5) (60 mg, 86.89 umol, 1 eq) in THF (3 mL) was added HCl (1.5 mL, 12 mol/L). The reaction mixture was stirred at 20° C. for 16 h. LC-MS showed reaction was completed. The reaction mixture was concentrated. The residue was purified by prep-HPLC (TFA condition) to give (3-((4-amino-3-((4-(trifluoromethyl)pyridin-2-yl)oxy)-1H-pyrazolo[3,4-d]pyrimidin-1-yl)methyl)cyclobutyl)boronic acid (6) (3 mg, yield 8.6%, 98.6% purity) as a white solid.

¹HNMR (400 MHz, METHANOL-d₄) δ=8.41 (s, 1H), 8.30 (s, 1H), 7.71 (s, 1H), 7.53 (s, 1H), 4.41-4.38 (d, J=12 Hz, 1H), 4.30-4.28 (d, J=8 Hz, 1H), 2.84-2.81 (m, 1H), 2.11-1.89 (m, 5H) Scheme 2:

### Step 1. Procedure for preparation of 3-[[4-amino-3-[[4-(trifluoromethyl)-2-pyridyl]oxy]pyrazolo[3,4-d]pyrimidin-1-yl]methyl]cyclobutanol (7)

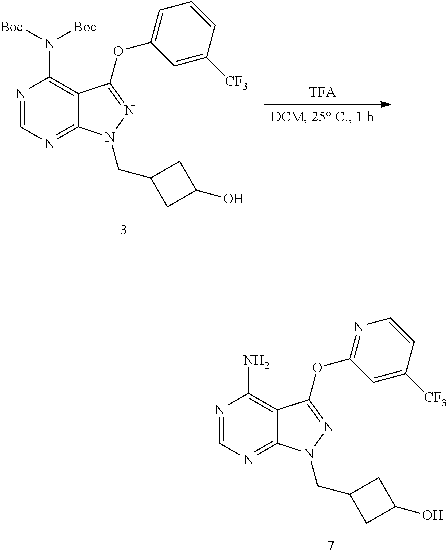 [SMILES: CC(C)(C)OC(=O)N(C(=O)OC(C)(C)C)c1ncnc2c1c(Oc1cccc(C(F)(F)F)c1)nn2CC1CC(O)C1 ; "DCM, 25° C., 1 h" ; "TFA" ; Nc1ncnc2c1c(Oc1cc(C(F)(F)F)ccn1)nn2CC1CC(O)C1]

To a solution of tert-butyl N-tert-butoxycarbonyl-N-[1-[(3-hydroxycyclobutyl)methyl]-3-[[4-(trifluoromethyl)-2-pyridyl]oxy]pyrazolo[3,4-d]pyrimidin-4-yl]carbamate (3) (80 mg, 137.80 umol, 1 eq) in DCM (4 mL) was added TFA (154.00 mg, 1.35 mmol, 0.1 mL, 9.80 eq) at 25° C. The reaction mixture was stirred for 1 h at 25° C. LCMS showed the reactant was consumed and the desired product was detected. The resulting mixture was concentrated to give the residue which was purified by prep-HPLC (HCl condition) to give 3-[[4-amino-3-[[4-(trifluoromethyl)-2-pyridyl]oxy]pyrazolo[3,4-d]pyrimidin-1-yl]methyl]cyclobutanol (7) (21 mg, yield 36.56%) as a white solid.

LCMS: (M+Hr: 381.2, Rt: 1.416 min.

¹H NMR (400 MHz, METHANOL-d₄) δ=8.43 (d, J=5.2 Hz, 1H), 8.37 (s, 1H), 7.73 (s, 1H), 7.57 (d, J=4.8 Hz, 1H), 4.39 (d, J=6.0 Hz, 2H), 4.09-4.00 (m, 1H), 2.40-2.26 (m, 3H), 1.77-1.67 (m, 2H)

### Synthetic Method AF: General Procedure Represented by the Preparation of 1-(3, 3-difluoro-4-piperidyl)-3-[[4-(trifluoromethyl)-2-pyridyl]oxy]pyrazolo[3,4-d]pyrimidin-4-amine

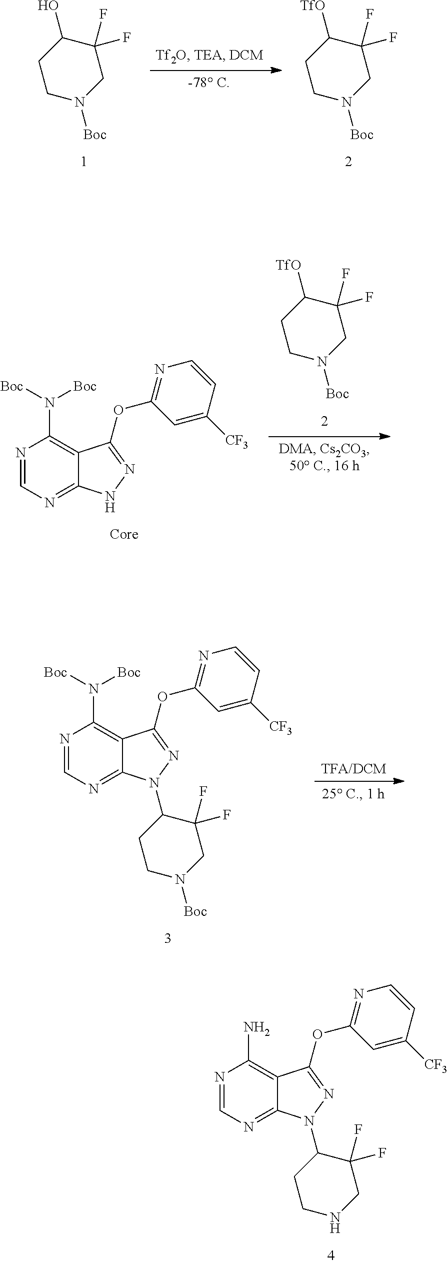

### Step 1. Procedure for preparation of tert-butyl 3,3-difluoro-4-(trifluoromethylsulfonyloxy)piperidine-1-carboxylate (2)

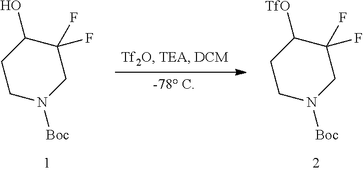 [SMILES: CC(C)(C)OC(=O)N1CCC(O)C(F)(F)C1 ; CC(C)(C)OC(=O)N1CCC(OS(=O)(=O)C(F)(F)F)C(F)(F)C1]

To a solution of tert-butyl 3,3-difluoro-4-hydroxy-piperidine-1-carboxylate (1) (500 mg, 2.11 mmol) and triethylamine (319.89 mg, 3.16 mmol) in dichloromethane (3 mL) was added a solution of Tf₂O (713.54 mg, 2.53 mmol) in dichloromethane (3 mL) dropwise at −78° C. under N₂. The mixture was stirred at −78° C. for 1 h. TLC (ethyl acetate: petroleum ether=2:1, R_{f}=0.4, I₂) showed the reaction was completed. The mixture was filtered and concentrated to give crude tert-butyl 3,3-difluoro-4-(trifluoromethylsulfonyloxy)piperidine-1-carboxylate (2) (700 mg, crude) as a dark green oil, which was used to next step directly.

### Step 2. Procedure for preparation of tert-butyl 4-[4-[bis(tert-butoxycarbamoyl)amino]-3-[[4-(trifluromethyl)-2-pyridyl]oxy]pyrazolo[3,4-d]pyrimidin-1-yl]-3,3-difluoro-piperidine-1-carboxylate (3)

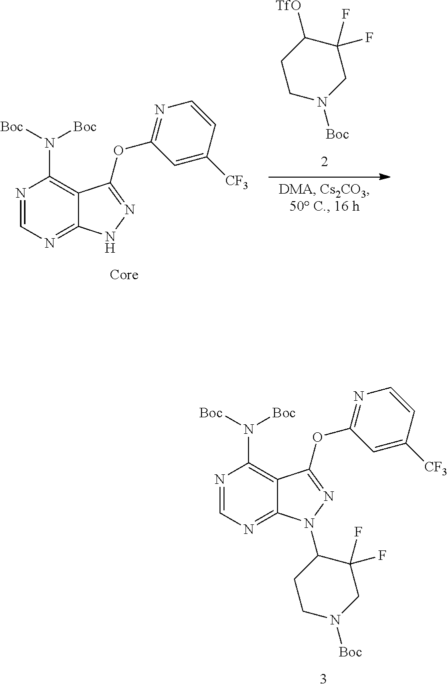 [SMILES: CC(C)(C)OC(=O)N(C(=O)OC(C)(C)C)c1ncnc2[nH]nc(Oc3cc(C(F)(F)F)ccn3)c12 ; CC(C)(C)OC(=O)N1CCC(OS(=O)(=O)C(F)(F)F)C(F)(F)C1 ; "DMA, Cs2CO3,50° C., 16 h" ; CC(C)(C)OC(=O)N1CCC(n2nc(Oc3cc(C(F)(F)F)ccn3)c3c(N(C(=O)OC(C)(C)C)C(=O)OC(C)(C)C)ncnc32)C(F)(F)C1]

To a solution of tert-butyl N-tert-butoxycarbonyl-N-[3-[[4-(trifluoromethyl)-2-pyridyl]oxy]-1H-pyrazolo[3,4-d]pyrimidin-4-yl]carbamate (Core) (500 mg, 1.01 mmol) and Cs₂CO₃ (656.31 mg, 2.01 mmol) in dimethylacetamide (2 mL) was added a solution of tert-butyl 3,3-difluoro-4-(trifluoromethylsulfonyloxy)piperidine-1-carboxylate (2) (743.91 mg, 2.01 mmol) in DMA (0.5 mL) under N₂. Then the mixture was stirred at 50° C. for 16 h. LCMS showed the starting material remaining and desired product was detected. The mixture was filtered and purified by prep-HPLC (basic condition) to give tert-butyl 4-[4-[bis(tert-butoxycarbonyl)amino]-3-[[4-(trifluoromethyl)-2-pyridyl]oxy]pyrazolo[3,4-d]pyrimidin-1-yl]-3,3-difluoro-piperidine-1-carboxylate (3) (120 mg, yield 17%) as a yellow solid.

PrepHPLC method:
Instrument: Shimadzu LC-8A preparative HPLC

### Column: Waters Xbridge Prep OBD C₁₈ 150*30 10u

Mobile phase: A for H₂O (10 mM NH4HCO3) and B for ACN
Gradient: B from 55% to 95% in 20 min
Flow rate: 40 mL/min

### Wavelength: 220&254 nm

### LCMS: (M+H)⁺: 716.3, Rt: 1.440 min.

### Step 3. Procedure for preparation of 1-(3,3-difluoro-4-piperidyl)-3-[[4-(trifluoromethyl)-2-pyridyl]oxy]pyrazolo[3,4-d]pyrimidin-4-amine (4)

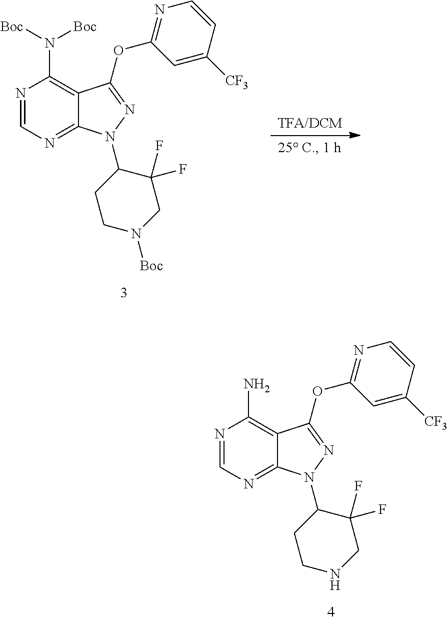 [SMILES: CC(C)(C)OC(=O)N1CCC(n2nc(Oc3cc(C(F)(F)F)ccn3)c3c(N(C(=O)OC(C)(C)C)C(=O)OC(C)(C)C)ncnc32)C(F)(F)C1 ; "25° C., 1 h" ; "TFA/DCM" ; Nc1ncnc2c1c(Oc1cc(C(F)(F)F)ccn1)nn2C1CCNCC1(F)F]

To a solution of tert-butyl 4-[4-[bis(tert-butoxycarbonyl)amino]-3-[[4-(trifluoromethyl)-2-pyridyl]oxy]pyrazolo[3,4-d]pyrimidin-1-yl]-3,3-difluoro-piperidine-1-carboxylate (3) (120 mg) in CH₂Cl₂ (1.5 mL) was added TFA (0.5 mL). The mixture was stirred at 25° C. for 1 hr. LCMS showed the starting material remaining and desired product was detected. The reaction mixture was concentrated under reduced pressure to give a residue, which was purified by prep-HPLC (TFA condition) to give 1-(3, 3-difluoro-4-piperidyl)-3-[[4-(trifluoromethyl)-2-pyridyl]oxy]pyrazolo[3,4-d]pyrimidin-4-amine (4) (35 mg, yield 60.31%) as a yellow solid.

¹H NMR (400 MHz, DMSCM6) δ=10.19-8.89 (m, 2H), 8.42 (d, J=5.2 Hz, 1H), 8.29 (s, 1H), 7.65 (s, 1H), 7.59 (d, J=5.6 Hz, 1H), 5.62-5.50 (m, 1H), 3.99-3.68 (m, 2H), 3.47 (br d, J=12.4 Hz, 1H), 3.38-3.27 (m, 1H), 2.72-2.59 (m, 1H), 2.36-2.23 (m, 1H) F NMR (400 MHz, DMSO-d6) δ=−63.357, δ=−74.373, δ=107.029, δ=107.690, δ=112.964, δ=113.625

### Synthetic Method AG: General Procedure Represented by the Preparation of 1-(1-(trifluoromethyl)cyclobutyl)-3-((4-(trifluoromethyl)pyridin-2-yl)oxy)-1H-pyrazolo[3,4-d]pyrimidin-4-amine

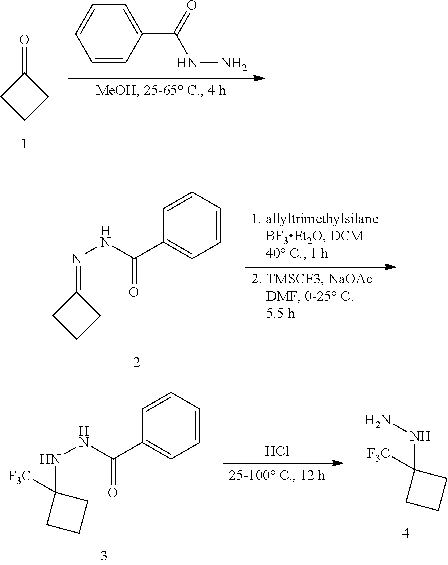

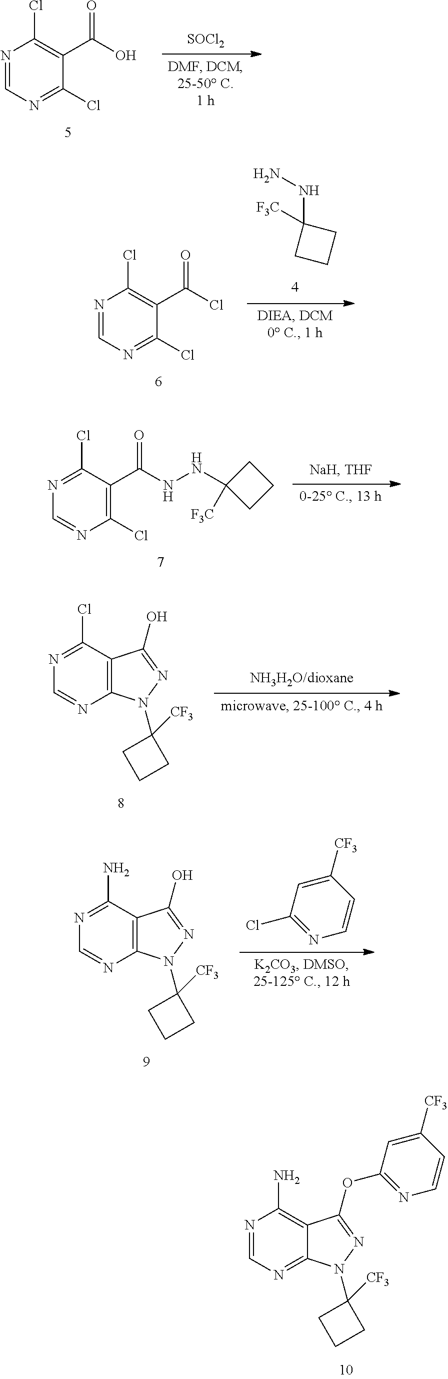

Step 1. Procedure for preparation of N′-cyclobutylidenebenzohydrazide (2)

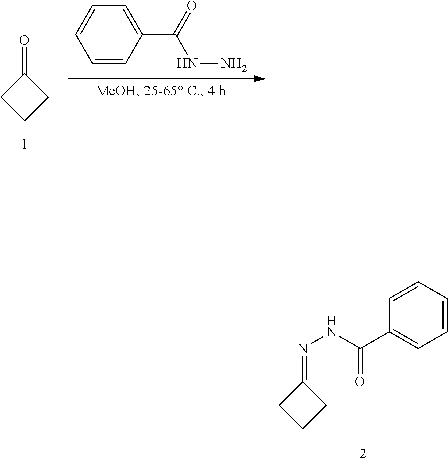

To a solution of benzohydrazide (209.79 g, 2.98 mol, 1 eq) in anhydrous MeOH (1.5 L) was added cyclobutanone (1) (108 g, 2.98 mol, 1 eq) at 25° C. The reaction mixture was stirred for 4 h at 65° C. TLC (Petroleum ether: Ethyl acetate=0:1) showed the reactant was consumed and the desired product was detected. The mixture was cooled to 25° C. and filtered. The filter cake was washed with methyl alcohol and dried under vacuum to give N-(cyclobutylidencamino)benzamide (2) (380 g, 2.02 mol, yield 65.51%) as a white solid, which was used to next step without further purification. ¹H NMR (400 MHz, DMSO-d₆) δ=10.60 (s, 1H), 7.79 (br d, J=7.2 Hz, 2H), 7.58-7.51 (m, 1H), 7.51-7.43 (m, 2H), 2.95 (td, J=8.0, 16.0 Hz, 4H), 1.92 (quin, J=8.0 Hz, 2H)

### Step 2. Procedure for preparation of N′-[1-(trifluoromethyl)cyclobutyl]benzohydrazide (3)

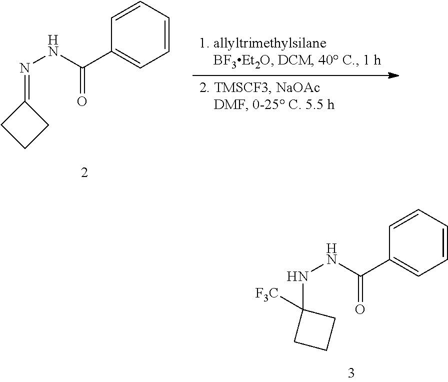

Allyltrimethylsilane (173.01 g, 1.51 mol, 1.5 eq) and BF₃.Et₂O (214.90 g, 1.51 mol, 1.5 eq) were successively added to a suspension of N-(cyclobutylideneamino)benzamide (2) (190 g, 1.01 mol, 1 eq) in DCM (2 L). The mixture was stirred at 40° C. for 1 h. To the mixture was added TMSCF₃ (287.07 g, 2.02 mol, 2 eq), NaOAc (165.61 g, 2.02 mol, 2 eq) and DMF (2 L) at 0° C. and stirred for another 0.5 h. The mixture was stirred for 5 h at 25° C. LC-MS showed 0% of Reactant 1 remaining. Several new peaks were shown on LC-MS and 40% of desired product was detected. TLC (Petroleum ether: Ethyl acetate=3:1) indicated the reactant was consumed completely and one major new spot with lower polarity was detected. The reaction mixture was quenched by addition aq. Na₂CO₃ (1.2 L) at 0° C. The organic layer was dried over Na₂SO₄, filtered and concentrated under reduced pressure to give a residue. The residue was purified by flash silica gel chromatography (petroleum ether: ethyl acetate=1:0 to 0:1) to afford N′-[1-(trifluoromethyl)cyclobutyl]benzohydrazide (3) (110 g, 425.96 mmol, yield 21.10%) as a yellow solid.

¹H NMR (400 MHz, DMSO-d₆) δ=10.04 (d, J=6.4 Hz, 1H), 7.87-7.81 (m, 2H), 7.58-7.52 (m, 1H), 7.51-7.44 (m, 2H), 5.74 (d, J=6.4 Hz, 1H), 2.30-2.15 (m, 4H), 2.04-1.90 (m, 1H), 1.86-1.73 (m, 1H)

### Step 3. Procedure for preparation of [1-(trifluoromethyl)cyclobutyl]hydrazine (4)

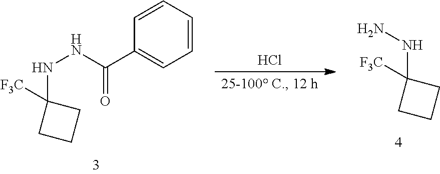

A mixture of N′-[1-(trifluoromethyl)cyclobutyl]benzohydrazide (3) (20 g, 77.45 mmol, 1 eq) and hydrogen chloride (6 M, 199.94 mL, 15.49 eq) was stirred for 12 h at 100° C. TLC (Petroleum ether: Ethyl acetate=3:1) indicated the reactant was consumed completely and one new spot was formed. The mixture was extracted with petroleum ether/ethyl acetate (3/1, 1 L). The aqueous phase was dried under vacuum to give [1-(trifluoromethyl)cyclobutyl]hydrazine (4) (12 g, 62.96 mmol, yield 81.29%, HCl) as a yellow solid.

¹H NMR (400 MHz, METHANOL-d₄) δ=2.45-2.36 (m, 2H), 2.30-2.20 (m, 2H), 2.17-2.05 (m, 1H), 2.05-1.91 (m, 1H)

### Step 4. Procedure for preparation of 4,6-dichloropyrimidine-5-carbonyl chloride (6)

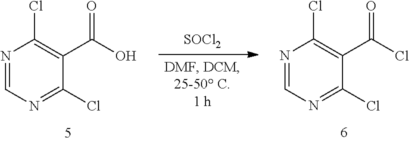 [SMILES: O=C(O)c1c(Cl)ncnc1Cl ; "SOCl2" ; "DMF, DCM,25-50° C.1 h" ; O=C(Cl)c1c(Cl)ncnc1Cl]

To a solution of 4,6-dichloropyrimidine-5-carboxylic acid (5) (5 g, 25.91 mmol, 1 eq) in DCM (100 mL) was added SOCl₂ (15.41 g, 129.54 mmol, 5 eq), DMF (18.94 mg, 259.08 umol, 0.01 eq) at 25° C. The reaction mixture was stirred for 1 h at 50° C. A sample was quenched by MeOH. TLC (Ethyl acetate:Methanol=5:1) indicated the reactant was consumed, and one major new spot with lower polarity was detected. The resulting mixture was concentrated under reduced pressure to give 4,6-dichloropyrimidine-5-carbonyl chloride (6) (5 g, crude) as yellow oil, which was used to next step without further purification.

### Step 5. Procedure for preparation of 4,6-dichloro-N′-[1-(trifluoromethyl)cyclobutyl]pyrimidine-5-carbohydrazide (7)

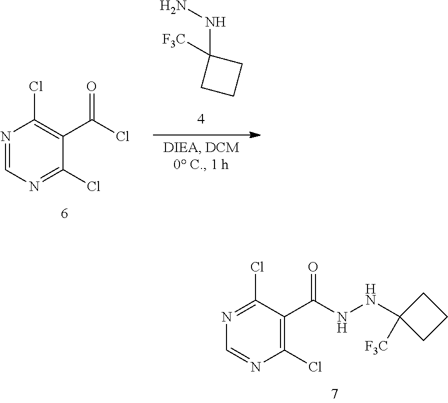 [SMILES: O=C(Cl)c1c(Cl)ncnc1Cl ; NNC1(C(F)(F)F)CCC1 ; "DIEA, DCM0° C., 1 h" ; O=C(NNC1(C(F)(F)F)CCC1)c1c(Cl)ncnc1Cl]

To a solution of [1-(trifluoromethyl)cyclobutyl]hydrazine (4) (7.21 g, 37.84 mmol, 1.6 eq, HCl) and DIEA (24.45 g, 189.19 mmol, 32.95 mL, 8 eq) in DCM (50 mL) was added the solution of 4,6-dichloropyrimidine-5-carbonyl chloride (6) (5 g, 23.65 mmol, 1 eq) in DCM (50 mL) dropwise at 0° C. The mixture was stirred at 0° C. for 1 h. LC-MS showed the reactant was consumed and the desired product was detected. The resulting mixture was concentrated under reduced pressure to give a residue. The residue was purified by column chromatography (SiO₂, Petroleum ether/Ethyl acetate=10/1 to 3:1) to afford 4,6-dichloro-N′-[1-(trifluoromethyl)cyclobutyl]pyrimidine-5-carbohydrazide (7) (3.1 g, 8.95 mmol, yield 37.84%) as a yellow solid.

### Step 6. Procedure for preparation of 4-chloro-1-[1-(trifluoromethyl)cyclobutyl]pyrazolo[3,4-d]pyrimidin-3-ol (8)

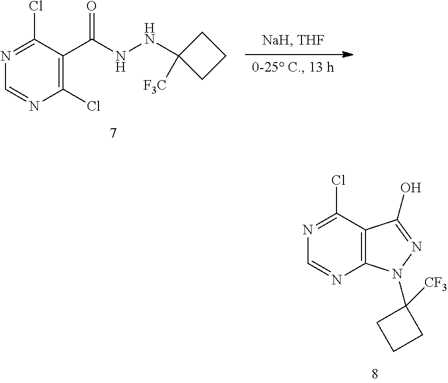

To a solution of NaH (208.00 mg, 5.20 mmol, 60% purity, 4.28 eq) in THF (8 mL) was added the solution of 4,6-dichloro-N′-[1-(trifluoromethyl)cyclobutyl]pyrimidine-5-carbohydrazide (7) (400 mg, 1.22 mmol, 1 eq) in THF (8 mL) at 0° C. and stirred for 1 h. The reaction mixture was stirred for 12 h at 25° C. A sample of the reaction mixture was quenched with aq.NH₄Cl. LC-MS showed the reactant was consumed and the desired product was detected. The two reactions were combined for purification. The combined reaction mixture was quenched with aq.NH₄Cl to pH=7. The aqueous phase was extracted with ethyl acetate (100 mL×3). The combined organic phase was washed with brine (100 mL×3), dried over anhydrous Na₂SO₄, filtered and concentrated in vacuum. The residue was purified by prep-HPLC (neutral condition) to afford 4-chloro-1-[1-(trifluoromethyl)cyclobutyl]pyrazolo[3,4-d]pyrimidin-3-ol (8) (160 mg, 546.74 umol, yield 19.99%) as a yellow solid.

### Step 7. Procedure for preparation of 4-amino-1-[1-(trifluoromethyl)cyclobutyl]pyrazolo[3,4-d]pyrimidin-3-ol (9)

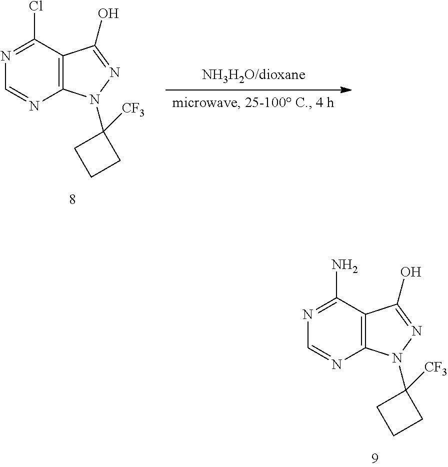 [SMILES: Oc1nn(C2(C(F)(F)F)CCC2)c2ncnc(Cl)c12 ; "NH3H2O/dioxane" ; "microwave, 25-100° C., 4 h" ; Nc1ncnc2c1c(O)nn2C1(C(F)(F)F)CCC1]

To a solution of 4-chloro-1-[1-(trifluoromethyl)cyclobutyl]pyrazolo[3,4-d]pyrimidin-3-ol (8) (60 mg, 205.03 umol, 1 eq) in dioxane (4 mL) was added NH₃.H₂O (382.21 mg, 2.73 mmol, 420.01 uL, 25% purity, 13.30 eq) at 25° C. The reaction mixture was stirred in a microwave apparatus for 4 h at 100° C. LC-MS showed the reactant was consumed and the desired product was detected. The resulting mixture was diluted with acetonitrile and water, after lyophilization, 50 mg (crude) of 4-amino-1-[1-(trifluoromethyl)cyclobutyl]pyrazolo[3,4-d]pyrimidin-3-ol (9) as a white solid.

### Step 8. Procedure for preparation of 1-[1-(trifluoromethyl)cyclobutyl]-3-[[4-(trifluoromethyl)-2-pyridyl]oxy]pyrazolo[3,4-d]pyrimidin-4-amine (10)

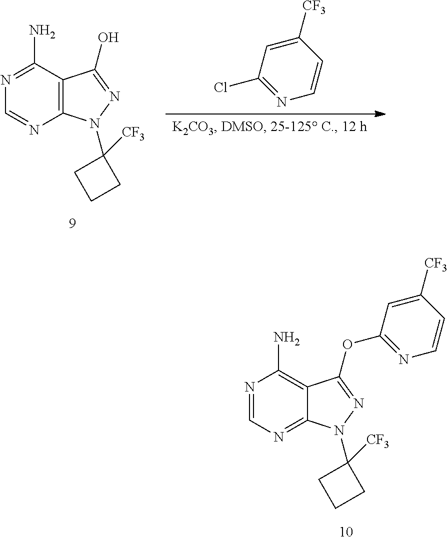

To a solution of 4-amino-1-[1-(trifluoromethyl)cyclobutyl]pyrazolo[3,4-d]pyrimidin-3-ol (9) (40 mg, 146.41 umol, 1 eq) in DMSO (0.5 mL) was added 2-chloro-4-(trifluoromethyl)pyridine (39.87 mg, 219.61 umol, 1.5 eq) and K₂CO₃ (40.47 mg, 292.81 umol, 2 eq) at 25° C. under N₂. The solution was stirred at 125° C. for 12 h. LC-MS showed the reactant was consumed and the desired product was detected. The resulting mixture was purified by prep-HPLC (TFA condition) to give 1-[1-(trifluoromethyl)cyclobutyl]-3-[[4-(trifluoromethyl)-2-pyridyl]oxy]pyrazolo[3,4-d]pyrimidin-4-amine (10) (12 mg, 28.69 umol, 15.68% yield) as an off-white solid.

LCMS: (M+H)⁺:419.0, Rt: 3.075 min.

¹H NMR (400 MHz, METHANOL-d₄) δ=8.44 (d, J=5.2 Hz, 1H), 8.30 (s, 1H), 7.72 (s, 1H), 7.55 (d, J=5.2 Hz, 1H), 3.18-3.09 (m, 2H), 2.93-2.84 (m, 2H), 2.26-2.05 (m, 2H)

### Synthetic Method AH: General Procedure Represented by the Preparation of 2-[4-amino-3-[[4-(trifluoromethyl)-2-pyridyl]oxy]pyrazolo[3,4-d]pyrimidin-1-yl]-4,4-difluoro-cyclohexanol

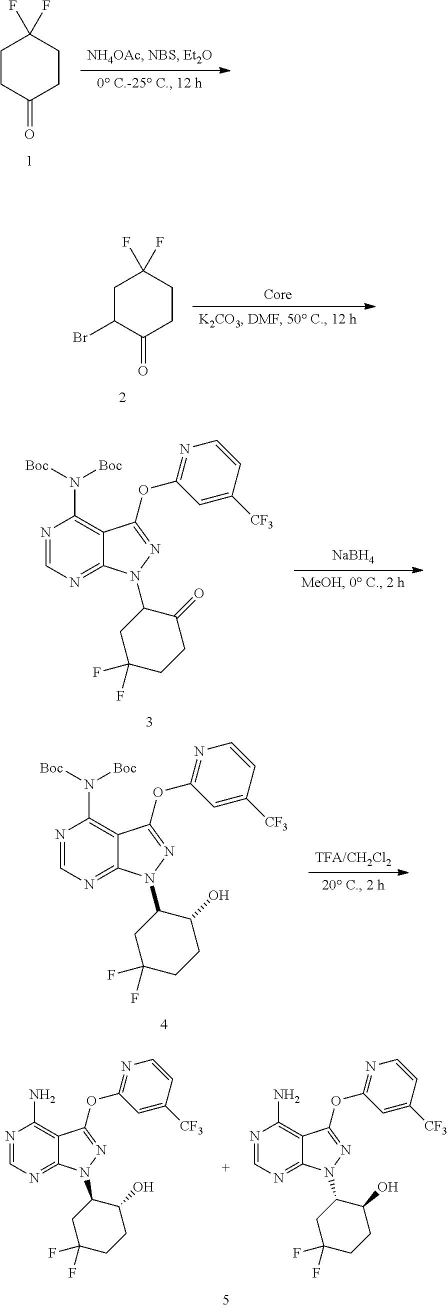

### Step 1. Procedure for preparation of 2-bromo-4,4-difluoro-cyclohexanone (2)

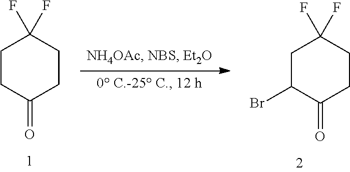

To a solution of 4,4-difluorocyclohexanone (1) (5 g, 37.28 mmol, 1 eq) in diethyl ether (50 mL) was added NH₄OAc (287.36 mg, 3.73 mmol, 0.1 eq) and NBS (6.97 g, 39.14 mmol, 1.05 eq) at 0° C. After addition, the mixture was stirred at 25° C. for 12 h. GCMS showed starting material was consumed and 60% of product with desired MS was detected. The reaction mixture was added ethyl acetate (100 mL) and H₂O (100 mL), extracted with ethyl acetate (100 mL×2), dried over Na₂SO₄, filtered and concentrated under reduced pressure to give a residue, which was used to next step directly without purification.

### Step 2. Procedure for preparation of tert-butyl N-tert-butoxycarbonyl-N-[1-(5,5-difluoro-2-oxo-cyclohexyl)-3-[[4-(trifluoromethyl)-2-pyridyl]oxy]pyrazolo[3,4-d]pyrimidin-4-yl]carbamate (3)

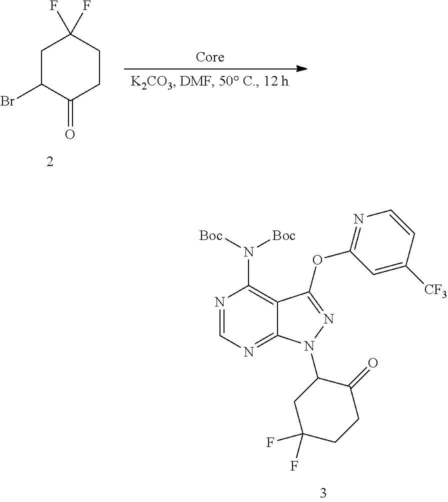 [SMILES: O=C1CCC(F)(F)CC1Br ; "Core" ; "K2CO3, DMF, 50° C., 12 h" ; CC(C)(C)OC(=O)N(C(=O)OC(C)(C)C)c1ncnc2c1c(Oc1cc(C(F)(F)F)ccn1)nn2C1CC(F)(F)CCC1=O]

To a solution of tert-butyl N-tert-butoxycarbonyl-N-[3-[[4-(trifluoromethyl)-2-pyridyl]oxy]-1H-pyrazolo[3,4-d]pyrimidin-4-yl]carbamate (Core) (896.34 mg, 1.81 mmol, eq) and 2-bromo-4,4-difluoro-cyclohexanone (2) (500 mg, 2.35 mmol, 1.3 eq) in DMF (8 mL) was added K₂CO₃ (249.54 mg, 1.81 mmol, 1 eq). The mixture was stirred at 50° C. for 12 h. LC-MS showed starting material was consumed completely and one main peak with desired mass was detected. Another additional vial was set up as described above. Two reaction mixtures were combined. The reaction mixture was added ethyl acetate (100 mL) and H₂O (150 mL), extracted with ethyl acetate (30 mL×3), dried over Na₂SO₄, filtered and concentrated under reduced pressure to give a residue. The residue was purified by prep-HPLC (TFA condition) to afford tert-butyl N-tert-butoxycarbonyl-N-[1-(5,5-difluoro-2-oxo-cyclohexyl)-3-[[4-(trifluoromethyl)-2-pyridyl]oxy]pyrazolo[3,4-d]pyrimidin-4-yl]carbamate (3) (200 mg, 318.19 umol, 17.62% yield) as a white solid.

¹H NMR (400 MHz, DMSO-d₆) δ=8.99 (s, 1H), 8.39 (d, J=5.2 Hz, 1H), 7.65 (d, J=5.2 Hz, 1H), 7.57 (s, 1H), 5.88 (dd, J=6.4, 13.0 Hz, 1H), 3.39-3.20 (m, 1H), 3.15-3.05 (m, 1H), 3.04-2.92 (m, 1H), 2.65-2.55 (m, 1H), 1.33 (s, 18H)

### Step 3. Procedure for preparation of tert-butyl N-tert-butoxycarbonyl-N-[1-(5,5-difluoro-2-hydroxy-cyclohexyl)-3-[[4-(trifluoromethyl)-2-pyridyl]oxy]pyrazolo[3,4-d]pyrimidin-4-yl]carbamate (4)

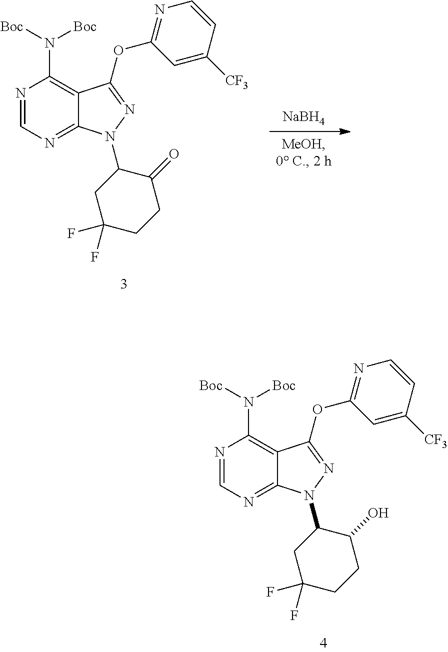 [SMILES: CC(C)(C)OC(=O)N(C(=O)OC(C)(C)C)c1ncnc2c1c(Oc1cc(C(F)(F)F)ccn1)nn2C1CC(F)(F)CCC1=O ; "MeOH,0° C., 2 h" ; "NaBH4" ; CC(C)(C)OC(=O)N(C(=O)OC(C)(C)C)c1ncnc2c1c(Oc1cc(C(F)(F)F)ccn1)nn2[C@@H]1CC(F)(F)CC[C@H]1O]

To a solution of tert-butyl N-tert-butoxycarbonyl-N-[1-(5,5-difluoro-2-oxo-cyclohexyl)-3-[[4-(trifluoromethyl)-2-pyridyl]oxy]pyrazolo[3,4-d]pyrimidin-4-yl]carbamate (3) (30 mg, 47.73 umol, 1 eq) in MeOH (3 mL) was added NaBH₄ (2.71 mg, 71.59 umol, 1.5 eq). The mixture was stirred at 0° C. for 2 h. LC-MS showed 18% of starting material remaining and 70% of desired compound was detected. Three additional vials were set up as described above. All four reaction mixtures were combined. The resulting mixture was acidified by adding HCl (1 M) to pH=7 and then concentrated in vacuum to give the residue. The residue was purified by prep-HPLC (basic condition) to afford tert-butyl N-tert-butoxycarbonyl-N-[1-(5,5-difluoro-2-hydroxy-cyclohexyl)-3-[[4-(trifluoromethyl)-2-pyridyl]oxy]pyrazolo[3,4-d]pyrimidin-4-yl]carbamate (4) (40 mg, 63.44 umol, 33.23% yield) as a white solid.

¹H NMR (400 MHz, DMSO-d₆) δ=8.97 (s, 1H), 8.39 (d, J=5.2 Hz, 1H), 7.65-7.60 (m, 2H), 4.84-4.76 (m, 1H), 4.05-3.96 (m, 1H), 2.20-1.98 (m, 3H), 1.70-1.47 (m, 1H), 1.33 (s, 18H)

### Step 4. Procedure for preparation of 2-[4-amino-3-[[4-(trifluoromethyl)-2-pyridyl]oxy]pyrazolo[3,4-d]pyrimidin-1-yl]-4,4-difluoro-cyclohexanol (5)

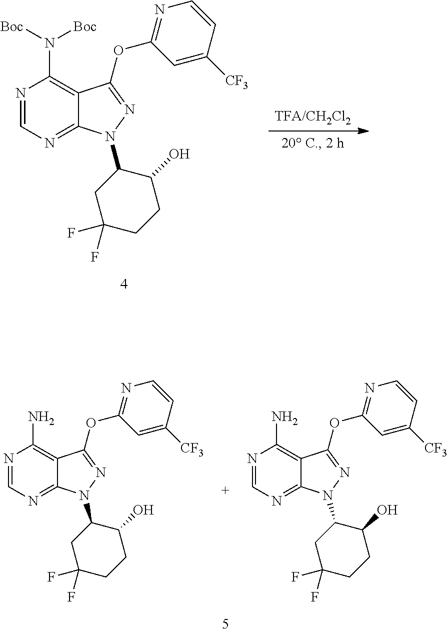 [SMILES: CC(C)(C)OC(=O)N(C(=O)OC(C)(C)C)c1ncnc2c1c(Oc1cc(C(F)(F)F)ccn1)nn2[C@@H]1CC(F)(F)CC[C@H]1O ; "20° C., 2 h" ; "TFA/CH2Cl2" ; Nc1ncnc2c1c(Oc1cc(C(F)(F)F)ccn1)nn2[C@@H]1CC(F)(F)CC[C@H]1O ; Nc1ncnc2c1c(Oc1cc(C(F)(F)F)ccn1)nn2[C@H]1CC(F)(F)CC[C@@H]1O]

To a solution of tert-butyl N-tert-butoxycarbonyl-N-(1-(5,5-difluoro-2-hydroxy-cyclohexyl)-3-[[4-(trifluoromethyl)-2-pyridyl)oxy]pyrazolo[3,4-d]pyrimidin-4-yl]carbamate (4) (40 mg, 63.44 umol, 1 eq) in CH₂Cl₂ (6 mL) was added TFA (0.3 mL). The mixture was stirred at 20° C. for 2 h. LCMS showed the reaction was completed. The reaction mixture was concentrated under reduced pressure to remove DCM. The residue was purified by prep-HPLC (TFA condition) to afford 2-[4-amino-3-[[4-(trifluoromethyl)-2-pyridyl]oxy]pyrazolo[3,4-d]pyrimidin-1-yl]-4,4-difluoro-cyclohexanol (5) (18 mg, 41.83 umol, yield 65.94%) as a white solid.

LCMS: (M+H)⁺: 431.0, Rt: 2.632 min.

¹H NMR (400 MHz, DMSO-d₆) δ=8.42 (d, J=5.2 Hz, 1H), 8.26 (s, 1H), 7.64 (s, 1H), 7.58 (d, J=5.2 Hz, 1H), 4.65-4.58 (m, 1H), 3.98-3.92 (m, 2H), 2.44-2.38 (m, 1H), 2.14-1.93 (m, 3H), 1.63-1.50 (m, 1H)

### Synthetic Method AI: General Procedure Represented by the Preparation of [2-(4-amino-1-cyclopropyl-pyrazolo[3,4-d]pyrimidin-3-yl)oxy-4-pyridyl]acetate

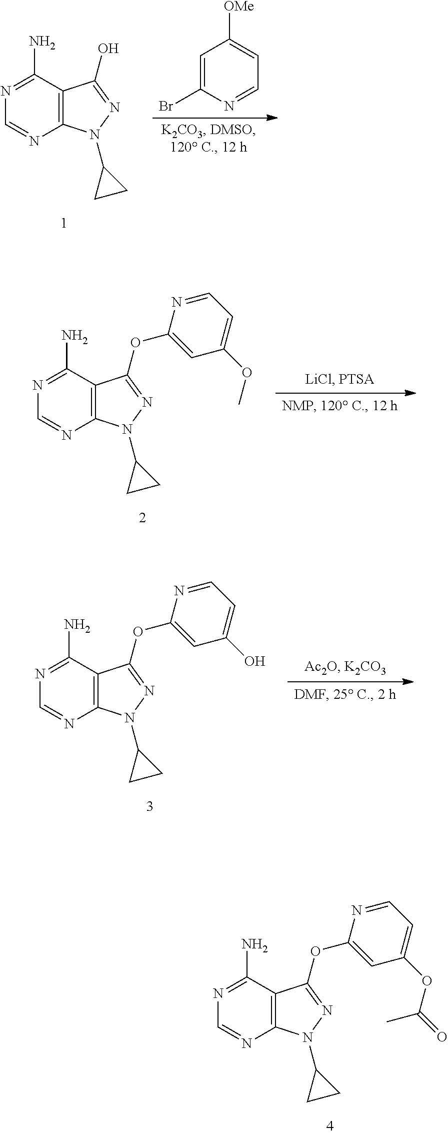

### Step 1. Procedure for preparation of 1-cyclopropyl-3-[(4-methoxy-2-pyridyl)oxy]pyrazolo[3,4-d]pyrimidin-4-amine (2)

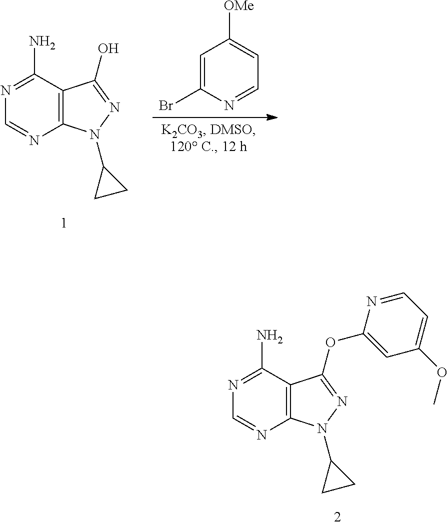

A mixture of 4-amino-1-cyclopropyl-pyrazolo[3,4-d]pyrimidin-3-ol (1) (100 mg, 523.04 umol, 1 eq) and 2-bromo-4-methoxypyridine (118.01 mg, 627.65 umol, 1.2 eq) in DMSO (3 mL) was added K₂CO₃ (144.57 mg, 1.05 mmol, 2 eq). The mixture was stirred at 120° C. for 12 h. LCMS showed the reaction was completed. Nineteen additional vials were set up as described above. All 20 reaction mixtures were combined for workup and purification. The mixture was filtered and the filtrate was concentrated under reduced pressure to give a residue. The residue was purified by prep-HPLC (basic condition) to give 1-cyclopropyl-3-[(4-methoxy-2-pyridyl)oxy]pyrazolo[3,4-d]pyrimidin-4-amine (2) (67 mg, 221.4 umol, yield 2.11%) as a white solid.

### Step 2. Procedure for preparation of 2-(4-amino-1-cyclopropyl-pyrazolo[3,4-d]pyrimidin-3-yl)oxypyridin-4-ol (3)

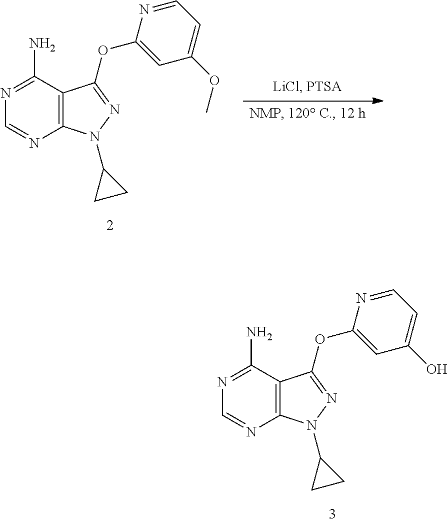

To a solution of 1-cyclopropyl-3-[(4-methoxy-2-pyridyl)oxy]pyrazolo[3,4-d]pyrimidin-4-amine (2) (78 mg, 261.48 umol, 1 eq) in NMP (3 mL) was added LiCl (110.85 mg, 2.61 mmol, 53.55 uL, 10 eq) and PTSA (450.28 mg, 2.61 mmol, 10 eq). The mixture was stirred at 120° C. for 12 h. LCMS showed the reaction was completed. The mixture was filtered and the filtrate was concentrated under reduced pressure to give a residue. The residue was purified by prep-HPLC (TFA condition) to give 2-(4-amino-1-cyclopropyl-pyrazolo[3,4-d]pyrimidin-3-yl)oxypyridin-4-ol (3) (35 mg, 118.20 umol, yield 45.20%) as a white solid.

### Step 3. Procedure for preparation of [2-(4-amino-1-cyclopropyl-pyrazolo[3,4-d]pyrimidin-3-yl)oxy-4-pyridyl]acetate (4)

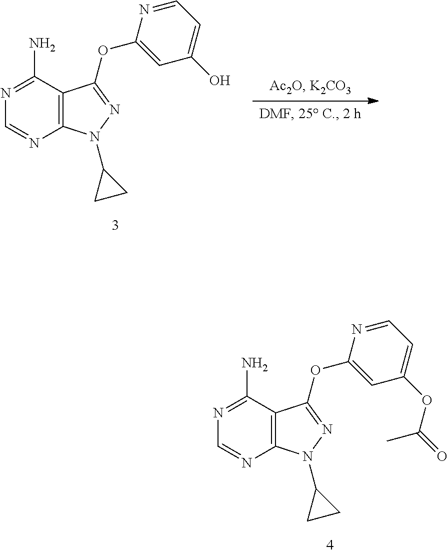

To a solution of 2-(4-amino-1-cyclopropyl-pyrazolo[3,4-d]pyrimidin-3-yl)oxypyridin-4-ol (3) (35 mg, 123.12 umol, 1 eq) in DMF (3 mL) was added K₂CO₃ (68.06 mg, 492.48 umol, 4 eq) and acetic anhydride (125.69 mg, 1.23 mmol, 10 eq). The mixture was stirred at 25° C. for 1 h. LCMS showed the reaction was completed. The mixture was filtered and the filtrate was concentrated under reduced pressure to give a residue. The residue was purified by prep-HPLC (TFA condition) to give [2-(4-amino-1-cyclopropyl-pyrazolo[3,4-d]pyrimidin-3-yl)oxy-4-pyridyl] acetate (4) (20 mg, 38.43 umol, yield 31.21%) as a white solid.

LCMS: (M+H)⁺:327.1, Rt: 1.376 min.

¹H NMR: (400 MHz, CHLOROFORM-d) δ=8.21 (t, J=2.8 Hz, 2H), 7.15 (d, J=2.0 Hz, 1H), 7.05 (dd, J=2.0, 5.6 Hz, 1H), 6.91-6.76 (m, 1H), 3.83 (tt, J=3.6, 7.6 Hz, 1H), 2.37 (s, 3H), 1.35-1.25 (m, 2H), 1.21-1.12 (m, 2H)

### Synthesis Method AJ: General Procedure Represented by the Preparation of 1-(2-fluorocyclohexen-1-yl)-3-[[4-(trifluoromethyl)-2-pyridyl]oxy]pyrazolo[3,4-d]pyrimidin-4-amine and 1-(2-fluorocyclohex-2-en-1-yl)-3-((4-(trifluoromethyl)pyridin-2-yl)oxy)-1H-pyrazolo[3,4-d]pyrimidin-4-amine

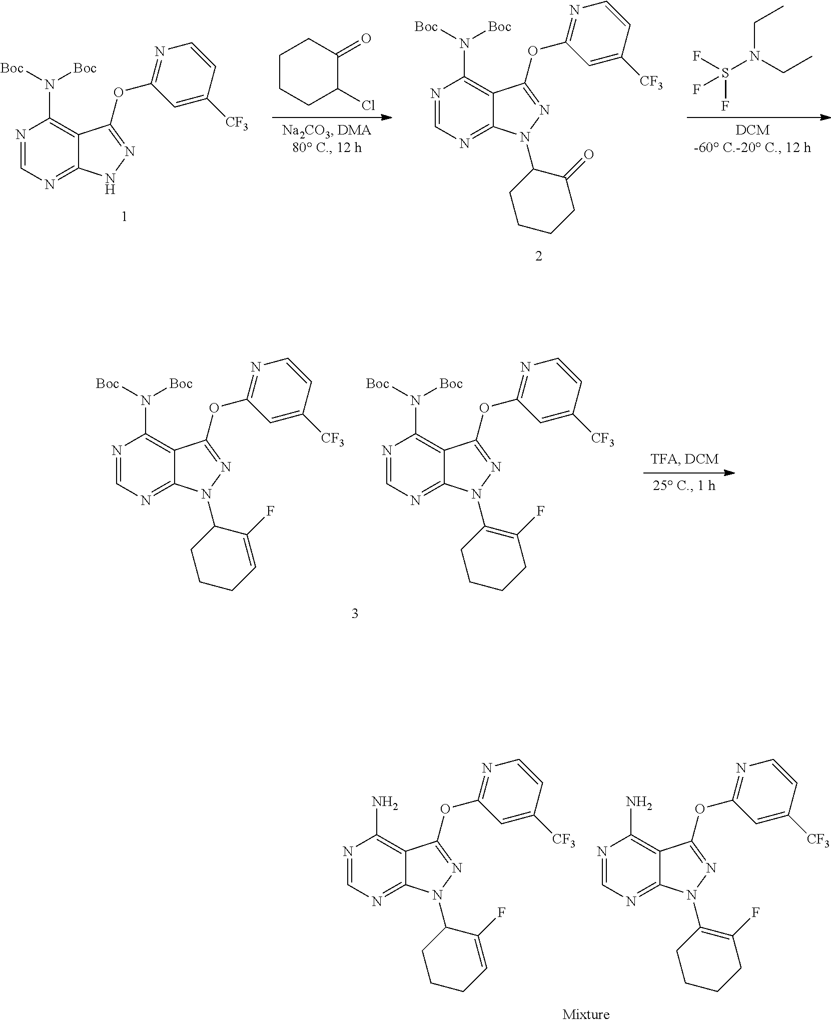

### Step 1. Procedure for preparation of tert-butyl N-tert-butoxycarbonyl-N-[1-(2-oxocyclohexyl)-3-[[4-(trifluoromethyl)-2-pyridyl]oxy]pyrazolo[3,4-d]pyrimidin-4-yl]carbamate (2)

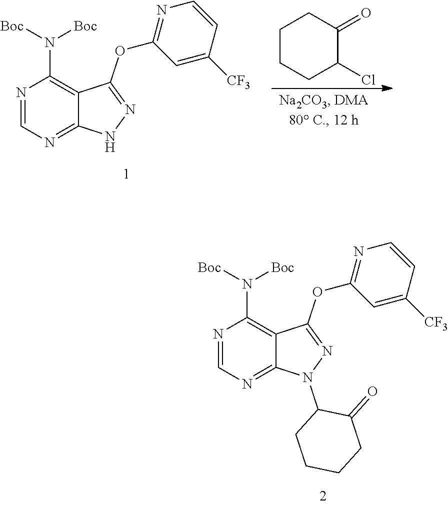 [SMILES: CC(C)(C)OC(=O)N(C(=O)OC(C)(C)C)c1ncnc2[nH]nc(Oc3cc(C(F)(F)F)ccn3)c12 ; O=C1CCCCC1Cl ; CC(C)(C)OC(=O)N(C(=O)OC(C)(C)C)c1ncnc2c1c(Oc1cc(C(F)(F)F)ccn1)nn2C1CCCCC1=O]

To a solution of tert-butyl N-tert-butoxycarbonyl-N-[3-[[4-(trifluoromethyl)-2-pyridyl]oxy]-1H-pyrazolo[3,4-d]pyrimidin-4-yl]carbamate (1) (1 g, 2.01 mmol, 1 eq) in DMA (10 mL) was added Na₂CO₃ (427.00 mg, 4.03 mmol, 2 eq) and 2-chlorocyclohexanone (400.62 mg, 3.02 mmol, 345.36 uL, 1.5 eq). The mixture was stirred at SOX for 12 h. LCMS showed the reaction was completed. The residue was diluted with H₂O (100 mL) and extracted with ethyl acetate (50 mL). The combined organic layers were washed with brine (50 mL×2), dried over Na₂SO₄, filtered and concentrated under reduced pressure to give a residue. The residue was purified by column chromatography (SiO₂, petroleum ether/Ethylacetate=10/1 to 1/1) to afford tert-butyl N-tert-butoxycarbonyl-N-[1-(2-oxocyclohexyl)-3-[[4-(trifluoromethyl)-2-pyridyl]oxy]pyrazolo[3,4-d]pyrimidin-4-yl]carbamate (2) (400 mg, 675.03 umol, 33.51% yield) as a white solid.

### Step 2. Procedure for preparation of tert-butyl N-tert-butoxycarbonyl-N-[1-(2-fluorocyclohexen-1-yl)-3-[[4-(trifluoromethyl)-2-pyridyl]oxy]pyrazolo[3,4-d]pyrimidin-4-yl]carbamate and tert-butyl N-tert-butoxycarbonyl-N-[1-(2-fluorocyclohex-2-en-1-yl)-3-[[4-(trifluoromethyl)-2-pyridyl]oxy]pyrazolo[3,4-d]pyrimidin-4-yl]carbamate (3)

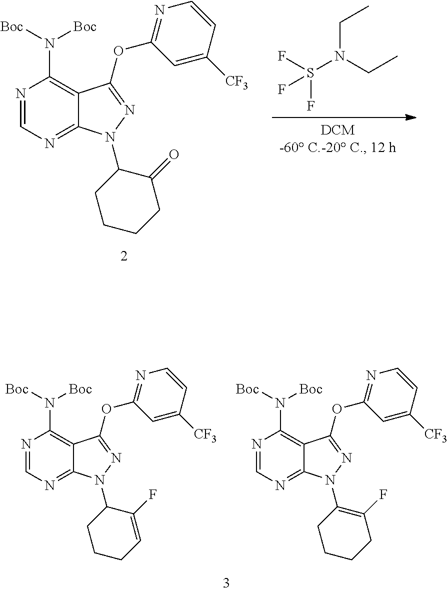

A mixture of tert-butyl N-tert-butoxycarbonyl-N-[1-(2-oxocyclohexyl)-3-[[4-(trifluoromethyl)-2-pyridyl]oxy]pyrazolo[3,4-d]pyrimidin-4-yl]carbamate (2) (100 mg, 168.76 umol, 1 eq) in DCM (5 mL) was degassed and purged with N₂ for 3 times. DAST (136.01 mg, 843.79 umol, 111.48 uL, 5 eq) was added at −60° C., and then the mixture was stirred at 20° C. for 12 h under N₂ atmosphere. LCMS showed the reaction was completed. Three additional vials were set up as described above. All four reaction mixtures were combined. The reaction mixture was partitioned between DCM (80 mL) and H₂O (60 mL). The organic phase was separated, washed with brine (30 mL×3), dried over Na₂SO₄, filtered and concentrated under reduced pressure to give a residue. The residue was purified by prep-HPLC (TFA condition) to afford tert-butyl N-tert-butoxycarbonyl-N-[1-(2-fluorocyclohexen-1-yl)-3-[[4-(trifluoromethyl)-2-pyridyl]oxy]pyrazolo[3,4-d]pyrimidin-4-yl]carbamate and tert-butyl N-tert-butoxycarbonyl-N-[1-(2-fluorocyclohex-2-en-1-yl)-3-[[4-(trifluoromethyl)-2-pyridyl]oxy]pyrazolo[3,4-d]pyrimidin-4-yl]carbamate (3) (50 mg, 84.10 umol, 12.46% yield) as a white solid.

### Step 3. Procedure for preparation of 1-(2-fluorocyclohexen-1-yl)-3-[[4-(trifluoromethyl)-2-pyridyl]oxy]pyrazolo[3,4-d]pyrimidin-4-amine and 1-(2-fluorocyclohex-2-en-1-yl)-3-((4-(trifluoromethyl)pyridin-2-yl)oxy)-1H-pyrazolo[3,4-d]pyrimidin-4-amine (Mixture)

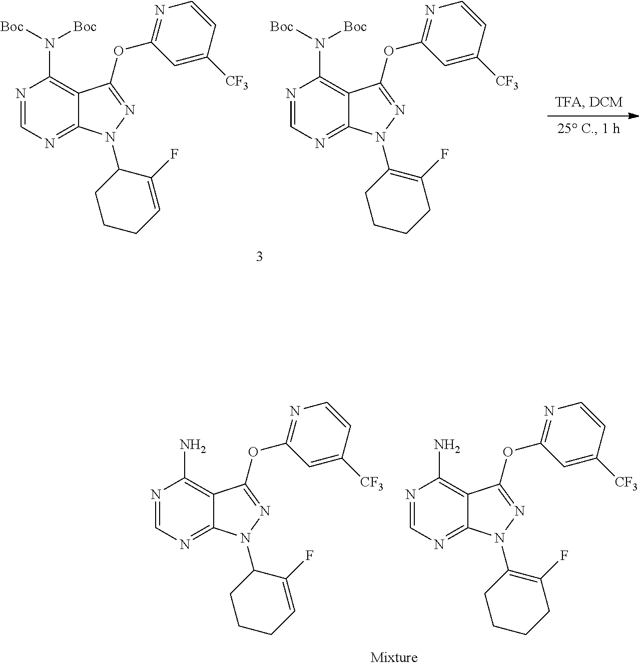

To a solution of tert-butyl N-tert-butoxycarbonyl-N-[1-(2-fluorocyclohexen-1-yl)-3-[[4-(trifluoromethyl)-2-pyridyl]oxy]pyrazolo[3,4-d]pyrimidin-4-yl]carbamate and tert-butyl N-tert-butoxycarbonyl-N-[1-(2-fluorocyclohex-2-en-1-yl)-3-[[4-(trifluoromethyl)-2-pyridyl]oxy]pyrazolo[3,4-d]pyrimidin-4-yl]carbamate (3) (50 mg, 84.10 umol, 1 eq) in DCM (0.9 mL) was added TFA (0.3 mL). The mixture was stirred at 25° C. for 1 h. LCMS showed the reaction was completed. The reaction mixture was concentrated under reduced pressure to remove DCM. The residue was purified by prep-HPLC (TFA condition) to give 1-(2-fluorocyclohexen-1-yl)-3-[[4-(trifluoromethyl)-2-pyridyl]oxy]pyrazolo[3,4-d]pyrimidin-4-amine and 1-(2-fluorocyclohex-2-en-1-yl)-3-((4-(trifluoromethyl)pyridin-2-yl)oxy)-1H-pyrazolo[3,4-d]pyrimidin-4-amine (Mixture) (30 mg, 59.01 umol, yield 70.18%, TFA) as a white solid.

LCMS: (M+H)⁺: 395.1, Rt: 1.710 min.

¹H NMR (400 MHz, DMSO-d₆) δ=8.46-8.38 (m, 1H), 8.33-8.26 (m, 1H), 7.69-7.63 (m, 1H), 7.61-7.56 (m, 1H), 5.70-5.52 (m, 2H), 2.43 (br s, 1H), 2.21-2.00 (m, 3H), 1.81-1.60 (m, 2H)

### Synthesis Method AK: General Procedure Represented by the Preparation of [2-[4-amino-1-(4,4-difluorocyclohexyl)pyrazolo[3,4-d]pyrimidin-3-yl]oxy-4-pyridyl]acetate (2)

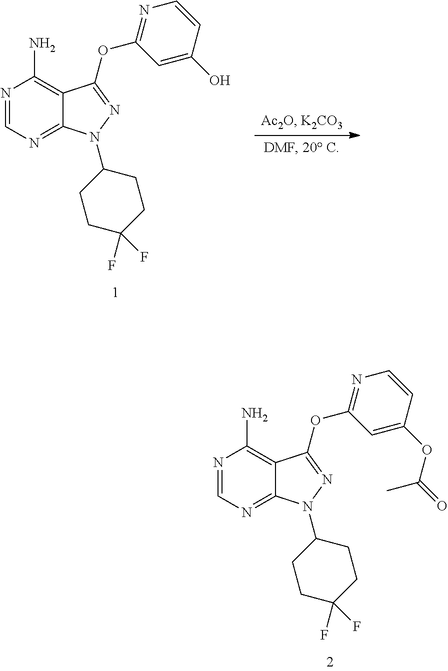

To a solution of 2-[4-amino-1-(4,4-difluorocyclohexyl)pyrazolo[3,4-d]pyrimidin-3-yl]oxypyridin-4-ol (1) (10 mg, 27.60 umol, 1 eq) in DMF (0.5 mL) was added Ac₂O (2.82 mg, 27.60 umol, 2.58 uL, 1 eq) and K₂CO₃ (15.26 mg, 110.40 umol, 4 eq). The mixture was stirred at 20° C. for 1 h. LCMS showed starting material was consumed completely and one main peak with desired mass was detected. The mixture was filtered and the filtrate was concentrated under reduced pressure to give a residue. The residue was purified by prep-HPLC (TFA condition) to afford [2-[4-amino-1-(4,4-difluorocyclohexyl)pyrazolo[3,4-d]pyrimidin-3-yl]oxy-4-pyridyl] acetate (2) (15 mg, 37.09 umol, 33.60% yield) as a white solid.

LCMS: (M+H)⁺: 405.2, Rt: 1.566 min.

¹H NMR (400 MHz, DMSO-d₆) δ=8.30 (d, J=2.2 Hz, 1H), 8.19-8.15 (m, 1H), 7.24-6.97 (m, 2H), 4.94-4.80 (m, 1H), 2.32 (s, 3H), 2.22-1.87 (m, 8H)

### Synthetic Method AL: General procedure for the preparation of 2-((4-amino-1-((1S,2S)-2-hydroxycyclohexyl)-1H-pyrazolo[3,4-d]pyrimidin-3-yl)oxy)pyridin-4-ol

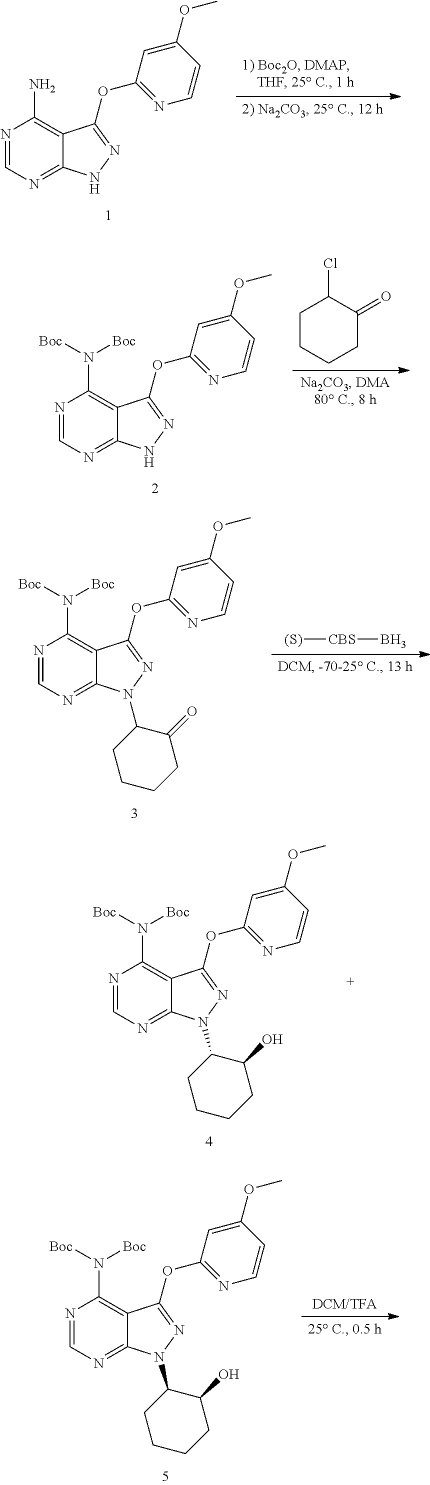

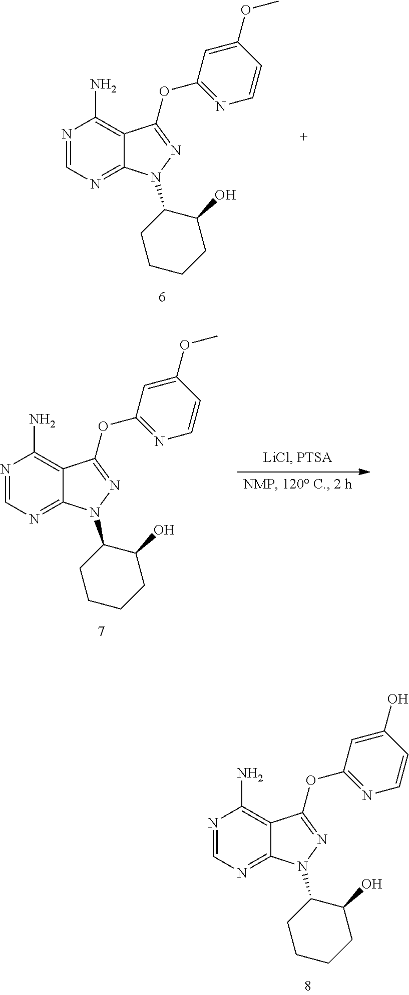

### Step 1. Procedure for preparation of tert-butyl N-tert-butoxycarbonyl-N-[3-[(4-methoxy-2-pyridyl)oxy]-1H-pyrazolo[3,4-d]pyrimidin-4-yl]carbamate (2)

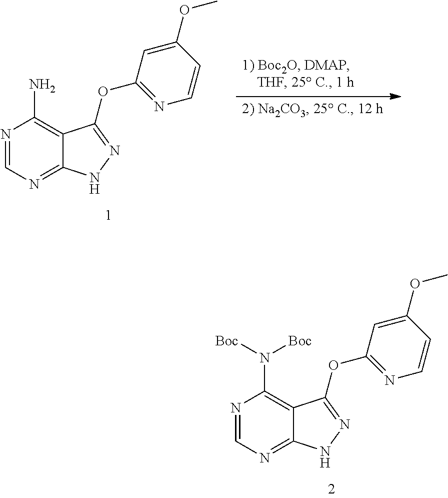

To a solution of 3-[(4-methoxy-2-pyridyl)oxy]-1H-pyrazolo[3,4-d]pyrimidin-4-amine (1) (450 mg, 1.74 mmol, 1 eq) in THF (5 mL) was added Boc₂O (1.52 g, 6.97 mmol, 1.60 mL, eq) and DMAP (2.13 mg, 17.43 umol, 0.01 eq). The mixture was stirred at 25° C. for 1 h. Na₂CO₃ (1 g) was added to the reaction and stirred at 25° C. for 12 h. The crystalline solid was collected by suction filtration, and then the organic phase was concentrated to afford the residue. The residue was purified by column chromatography (SiO₂, Petroleum ether/Ethyl acetate=50/1 to 1/1) to afford tert-butyl N-tert-butoxycarbonyl-N-[3-[(4-methoxy-2-pyridyl)oxy]-1H-pyrazolo[3,4-d]pyrimidin-4-yl]carbamate (2) (500 mg, 1.09 mmol, yield 62.58%) as a white solid.

¹H NMR: (400 MHz, MeOD-d₄) δ=8.86 (s, 1H), 7.88 (s, 1H), 6.78 (dd, J=6.0 Hz, 2.0 Hz, 1H), 6.62 (s, 1H), 3.93 (s, 3H), 1.35 (s, 18H).

### Step 2. Procedure for preparation of tert-butyl N-tert-butoxycarbonyl-N-[3-[(4-methoxy-2-pyridyl)oxy]-1-(2-oxocyclohexyl)pyrazolo[3,4-d]pyrimidin-4-yl]carbamate (3)

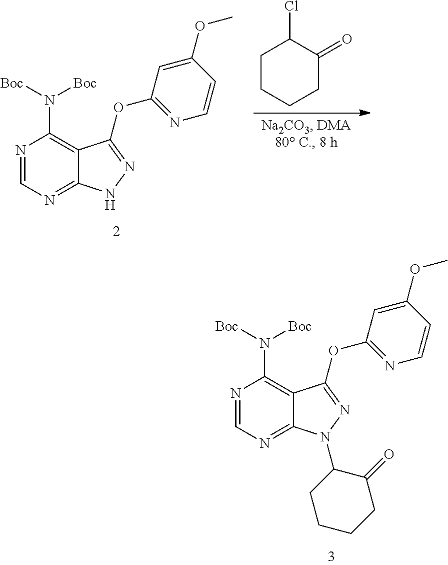

To a solution of tert-butyl N-tert-butoxycarbonyl-N-[3-[(4-methoxy-2-pyridyl)oxy]-1H-pyrazolo[3,4-d]pyrimidin-4-yl]carbamate (2) (250 mg, 545.30 umol, 1 eq) in DMA (3 mL) was added Na₂CO₃ (115.59 mg, 1.09 mmol, 2 eq) and 2-chlorocyclohexanone (108.45 mg, 817.94 umol, 93.49 uL, 1.5 eq). The mixture was stirred at 80° C. for 8 h. The resulting mixture was partitioned between ethyl acetate (90 mL) and water (100 mL), and then the aqueous phase was further extracted with ethyl acetate (30 mL×3). Then the organic phase was washed with brine (30 mL) and dried over Na₂SO₄ and concentrated under reduced pressure to give a residue. The residue was purified by column chromatography (SiO₂, Petroleum ether/Ethyl acetate=60/1 to 1/1) to afford tert-butyl N-tert-butoxycarbonyl-N-[3-[(4-methoxy-2-pyridyl)oxy]-1-(2-oxocyclohexyl)pyrazolo[3,4-d]pyrimidin-4-yl]carbamate (3) (240 mg, 432.75 umol, yield 79.36%) as a colorless oil.

¹H NMR: (400 MHz, MeOD-d₄) δ=8.84 (s, 1H), 7.90 (s, 1H), 6.80-6.76 (m, 1H), 6.61 (s, 1H), 5.71-5.66 (m, 1H), 4.61-4.56 (m, 1H), 3.92 (s, 3H), 3.04 (s, 9H), 2.91 (s, 9H), 2.68-2.63 (m, 7H).

### Step 3. Procedure for preparation of tert-butyl N-tert-butoxycarbonyl-N-[1-[(1S,2S)-2-hydroxycyclohexyl]-3-[(4-methoxy-2-pyridyl)oxy]pyrazolo[3,4-d]pyrimidin-4-yl]carbamate (4) and tert-butyl N-tert-butoxycarbonyl-N-[1-[(1R,2S)-2-hydroxycyclohexyl]-3-[(4-methoxy-2-pyridyl)oxy]pyrazolo[3,4-d]pyrimidin-4-yl]carbamate (5)

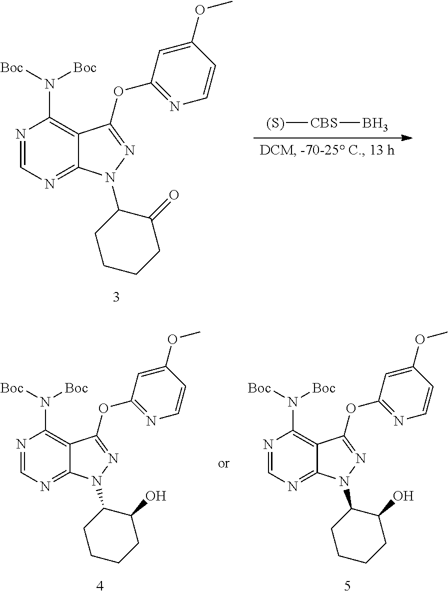

To a solution of (3aS)-1-methyl-3,3-diphenyl-3a,4,5,6-tetrahydropyrrolo[1,2-c][1,3,2]oxazaborole (1 M, 2.07 mL, 5 eq) in DCM (6 mL) was added BH₃.Me₂S (10 M, 45.62 uL, 1.1 eq) at 25° C. and stirred for 1 h. Then the solution was added to a mixture of tert-butyl N-tert-butoxycarbonyl-N-[3-[(4-methoxy-2-pyridyl)oxy]-1-(2-oxocyclohexyl)pyrazolo[3,4-d]pyrimidin-4-yl]carbamate (3) (230 mg, 414.72 umol, 1 eq) and (3aS)-1-methyl-3,3-diphenyl-3a,4,5,6-tetrahydropyrrolo[1,2-c][1,3,2]oxazaborole (1 M, 829.44 uL, 2 eq) in DCM (3 mL) at −70° C. The mixture was stirred at 25° C. for 12 h. The mixture was then quenched by MeOH (2 mL) and concentrated under reduced pressure to give a residue. The residue was purified by prep-TLC (SiO₂, Petroleum ether/Ethyl acetate=1/1) to afford tert-butyl N-tert-butoxycarbonyl-N-[1-[(1R,2S)-2-hydroxycyclohexyl]-3-[(4-methoxy-2-pyridyl)oxy]pyrazolo[3,4-d]pyrimidin-4-yl]carbamate (5) (30 mg, 53.90 umol, yield 13%) and tert-butyl N-tert-butoxycarbonyl-N-[1-[(1S,2S)-2-hydroxycyclohexyl]-3-[(4-methoxy-2-pyridyl)oxy]pyrazolo[3,4-d]pyrimidin-4-yl]carbamate (4) (50 mg, 89.83 umol, yield 21.66%) as yellow oil.

### Step 4. Procedure for preparation of (1S,2S)-2-[4-amino-3-[(4-methoxy-2-pyridyl)oxy]pyrazolo[3,4-d]pyrimidin-1-yl]cyclohexanol (6)

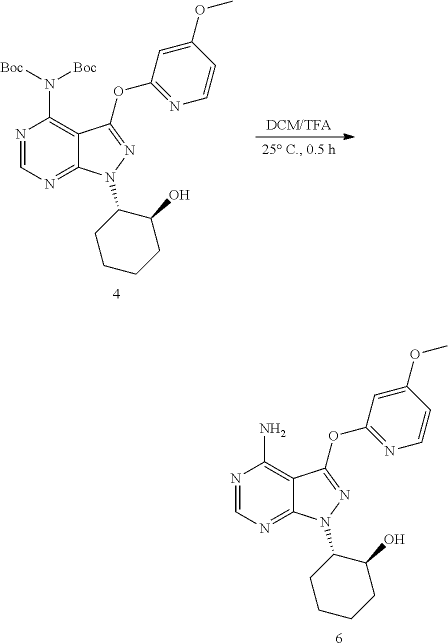

A mixture of tert-butyl N-tert-butoxycarbonyl-N-[1-[(1S,2S)-2-hydroxycyclohexyl]-3-[(4-methoxy-2-pyridyl)oxy]pyrazolo[3,4-d]pyrimidin-4-yl]carbamate (4) (50 mg, 89.83 umol, 1 eq) in DCM (1 mL) and TFA (0.1 mL) was stirred at 25° C. for 0.5 h. The mixture was concentrated under reduced pressure to give a residue. The residue was purified by prep-HPLC (TFA condition) to afford (1S,2S)-2-[4-amino-3-[(4-methoxy-2-pyridyl)oxy]pyrazolo[3,4-d]pyrimidin-1-yl]cyclohexanol (6) (9.4 mg, 26.38 umol, yield 29.36%, 100% purity) as a white solid.

¹H NMR (6): (400 MHz, MeOD-d₄) δ=8.31 (s, 1H), 8.04 (d, J=5.6 Hz, 1H), 7.04 (s, 1H), 6.91-6.88 (m, 1H), 4.58-4.51 (m, 1H), 3.95 (s, 3H), 3.94-3.88 (m, 1H), 2.14-2.10 (m, 1H), 1.99-1.36 (m, 2H), 1.84 (s, 2H), 1.50-1.39 (m, 3H).

¹H NMR (7): (400 MHz, MeOD-d₄) δ=8.30 (s, 1H), 8.03 (d, J=6.0 Hz, 1H), 7.08 (s, 1H), 6.89-6.86 (m, 1H), 4.22 (s, 1H), 3.95 (s, 3H), 2.48-2.40 (m, 1H), 1.97-1.93 (m, 2H), 1.87-1.83 (m, 1H), 1.74-1.71 (m, 2H), 1.53-1.50 (m, 2H).

### Step 5. Procedure for preparation of 2-[4-amino-1-[(1S,2S)-2-hydroxycyclohexyl]pyrazolo[3,4-d]pyrimidin-3-yl]oxypyridin-4-ol (8)

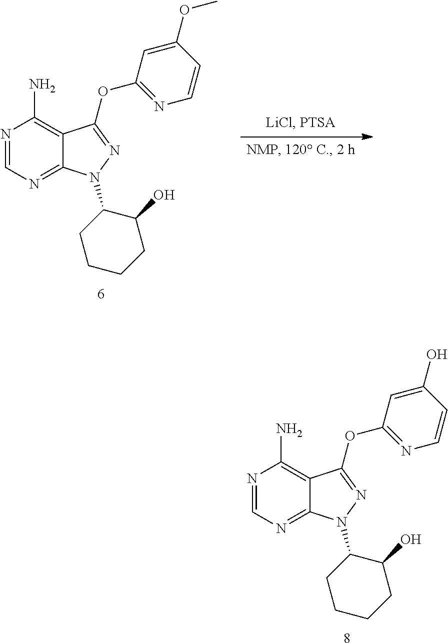

To a solution of (1S,2S)-2-[4-amino-3-[(4-methoxy-2-pyridyl)oxy]pyrazolo[3,4-d]pyrimidin-1-yl]cyclohexanol (6) (7 mg, 19.64 umol, 1 eq) in NMP (1 mL) was added LiCl (8.33 mg, 196.42 umol, 10 eq) and PTSA (33.82 mg, 196.42 umol, 10 eq). The mixture was stirred at 120° C. for 2 h. The mixture was purified by pre-HPLC (TFA condition) to give 2-[4-amino-1-[(1S,2S)-2-hydroxycyclohexyl]pyrazolo[3,4-d]pyrimidin-3-yl]oxypyridin-4-ol (8) (1.6 mg, 4.67 umol, yield 23.8%, 100% purity, TFA) as a white solid.

¹H NMR: (400 MHz, MeOD-d4) δ=8.30 (s, 1H), 8.03 (d, J=6.0 Hz, 1H), 7.08 (s, 1H), 6.89-6.86 (m, 1H), 4.22 (s, 1H), 3.95 (s, 3H), 2.48-2.40 (m, 1H), 1.97-1.93 (m, 2H), 1.87-1.83 (m, 1H), 1.74-1.71 (m, 2H), 1.53-1.50 (m, 2H).

### Synthetic Method AM: General Procedure Represented by the Preparation of 1-(2,2-difluorocyclohexyl)-3-[[4-(trifluoromethyl)-2-pyridyl]oxy]pyrazolo[3,4-d]pyrimidin-4-amine

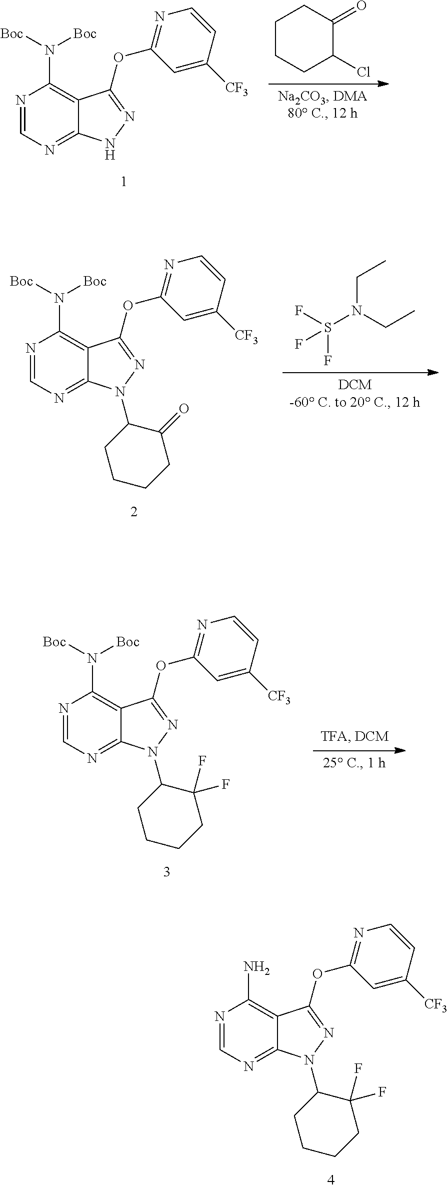

### Step 1. Procedure for preparation of tert-butyl N-tert-butoxycarbonyl-N-[1-(2-oxocyclohexyl)-3-[[4-(trifluoromethyl)-2-pyridyl]oxy]pyrazolo[3,4-d]pyrimidin-4-yl]carbamate (2

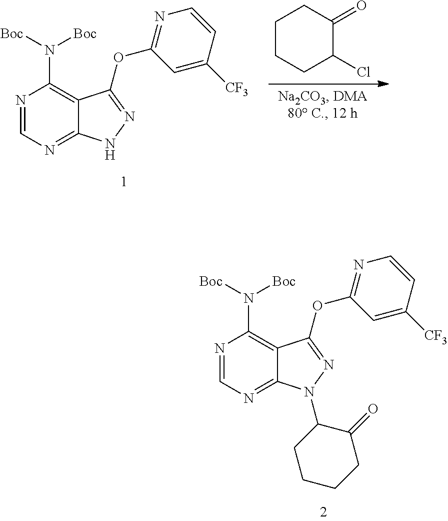

To a solution of tert-butyl N-tert-butoxycarbonyl-N-[3-[[4-(trifluoromethyl)-2-pyridyl]oxy]-1H-pyrazolo[3,4-d]pyrimidin-4-yl]carbamate (1) (1 g, 2.01 mmol, 1 eq) in DMA (10 mL) was added Na₂CO₃ (427.00 mg, 4.03 mmol, 2 eq) and 2-chlorocyclohexanone (400.62 mg, 3.02 mmol, 345.36 uL, 1.5 eq). The mixture was stirred at 80° C. for 12 h. LCMS showed the reaction was completed. The residue was diluted with H₂O (100 mL) and extracted with ethyl acetate (50 mL). The combined organic layers were washed with brine (50 mL×2), dried over Na₂SO₄, filtered and concentrated under reduced pressure to give a residue. The residue was purified by column chromatography (SiO₂, Petroleum ether/Ethylacctate=10/1 to 1/1) to give tert-butyl N-tert-butoxycarbonyl-N-[1-(2-oxocyclohexyl)-3-[[4-(trifluoromethyl)-2-pyridyl]oxy]pyrazolo[3,4-d]pyrimidin-4-yl]carbamate (2) (400 mg, 675.03 umol, yield 33.51%) as a white solid.

¹H NMR (400 MHz, CHLOROFORM-d) δ=8.84 (s, 1H), 8.30 (d, J=5.2 Hz, 1H), 7.33 (s, 1H), 7.29 (hr s, 1H), 5.51 (dd, J=6.0, 13.0 Hz, 1H), 2.76-2.63 (m, 2H), 2.60-2.48 (m, 2H), 2.24-2.14 (m, 2H), 1.98-1.80 (m, 2H), 1.39 (s, 18H)

### Step 2. Procedure for preparation of tert-butyl N-tert-butoxycarbonyl-N-[1-(2,2-difluorocyclohexyl)-3-[[4-(trifluoromethyl)-2-pyridyl]oxy]pyrazolo[3,4-d]pyrimidin-4-yl]carbamate (3)

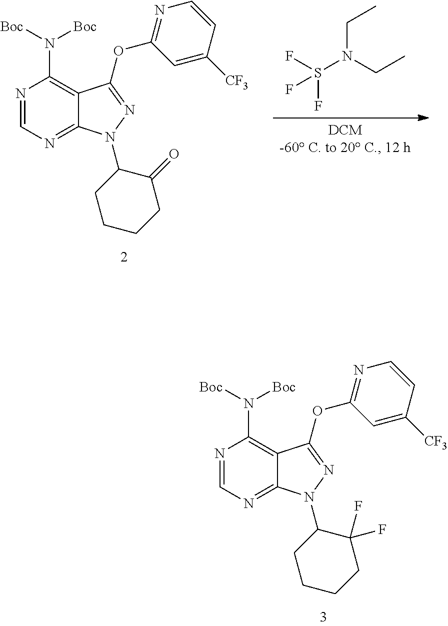

A mixture of tert-butyl N-tert-butoxycarbonyl-N-[1-(2-oxocyclohexyl)-3-[[4-(trifluoromethyl)-2-pyridyl]oxy]pyrazolo[3,4-d]pyrimidin-4-yl]carbamate (2) (100 mg, 168.76 umol, 1 eq) in DCM (5 mL) was degassed and purged with N₂ for 3 times, DAST (136.01 mg, 843.79 umol, 111.48 uL, 5 eq) was added at −60° C., and then the mixture was stirred at −60° C. to 20° C. for 12 h under N₂ atmosphere. LCMS showed the reaction was completed. Three additional vials were set up as described above. All four reaction mixtures were combined. The reaction mixture was partitioned between DCM (80 mL) and H₂O (60 mL). The organic phase was separated, washed with brine (30 mL×3), dried over Na₂SO₄, filtered and concentrated under reduced pressure to give a residue. The residue was purified by prep-HPLC (TFA condition) to afford tert-butyl N-tert-butoxycarbonyl-N-[1-(2,2-difluorocyclohexyl)-3-[[4-(trifluoromethyl)-2-pyridyl]oxy]pyrazolo[3,4-d]pyrimidin-4-yl]carbamate (3) (140 mg, 227.80 umol, 33.75% yield) as a white solid.

¹H NMR (400 MHz, DMSO-d₆) δ=9.01 (s, 1H), 8.37 (d, 7=5.2 Hz, 1H), 7.64 (d, J=5.2 Hz, 1H), 7.60 (s, 1H), 5.40-5.28 (m, 1H), 2.25-1.45 (m, 8H), 1.31 (s, 18H)

### Step 3. Procedure for preparation of 1-(2,2-difluorocyclohexyl)-3-[[4-(trifluoromethyl)-2-pyridyl]oxy]pyrazolo[3,4-d]pyrimidin-4-amine (A391)

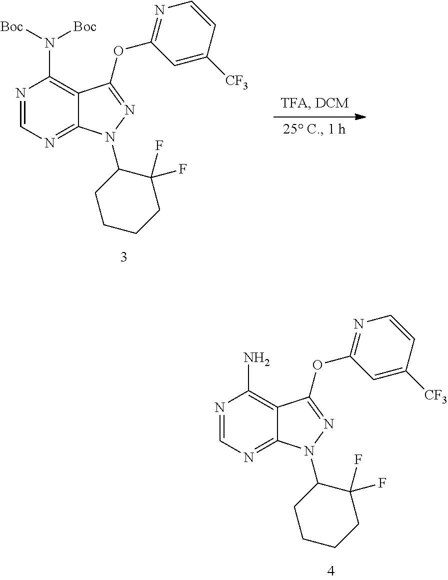

To a solution of tert-butyl N-tert-butoxycarbonyl-N-[1-(2,2-difluorocyclohexyl)-3-[[4-(trifluoromethyl)-2-pyridyl]oxy]pyrazolo[3,4-d]pyrimidin-4-yl]carbamate (3) (140 mg, 227.80 umol, 1 eq) in DCM (0.9 mL) was added TFA (0.3 mL). The mixture was stirred at 25° C. for 1 h. LCMS showed the reaction was completed. The reaction mixture was concentrated under reduced pressure to remove DCM. The residue was purified by prep-HPLC (TFA condition) to give 1-(2,2-difluorocyclohexyl)-3-[[4-(trifluoromethyl)-2-pyridyl]oxy]pyrazolo[3,4-d]pyrimidin-4-amine (4) (70 mg, 132.49 umol, 58.16% yield, TFA) as a white solid.

LCMS: (M+H)⁺: 415.1, Rt: 1.716 min.

¹H NMR (400 MHz, DMSO-d*) δ=8.41 (d, J=5.2 Hz, 1H), 8.29 (d, J=1.6 Hz, 1H), 7.65 (s, 1H), 7.58 (d, J=5.2 Hz, 1H), 5.18-5.04 (m, 1H), 2.30-2.12 (m, 2H), 2.07-1.75 (m, 4H), 1.64-1.46 (m, 2H)

### Synthetic Method AN: General Procedure Represented by the Preparation of 1-(2,2,2-trifluoroethyl)-3-((4-(trifluoromethyl)pyridin-2-yl)oxy)-1H-pyrazolo[3,4-d]pyrimidin-4-amine

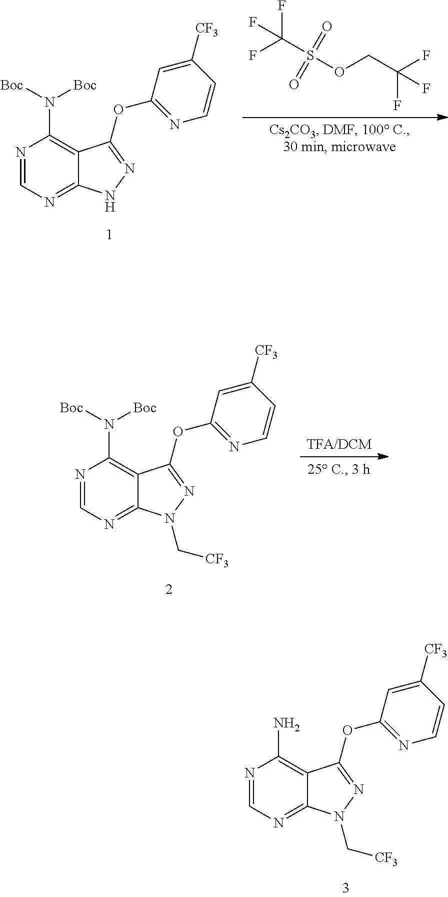

### Step 1. Procedure for preparation of tert-butyl N-tert-butoxycarbonyl-N-[1-(2,2,2-trifluoroethyl)-3-[[4-(trifluoromethyl)-2-pyridyl]oxy]pyrazolo[3,4-d]pyrimidin-4-yl]carbamate (2)

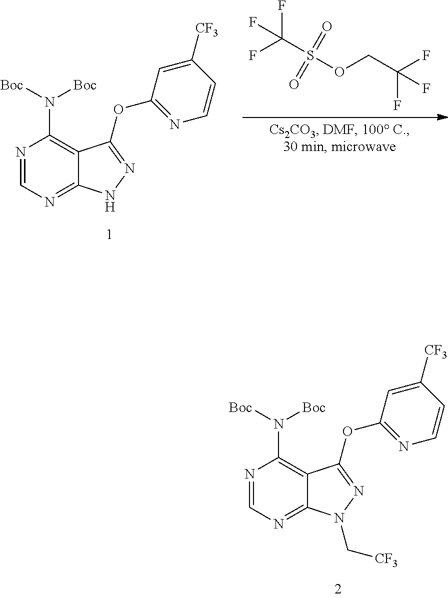 [SMILES: CC(C)(C)OC(=O)N(C(=O)OC(C)(C)C)c1ncnc2[nH]nc(Oc3cc(C(F)(F)F)ccn3)c12 ; O=S(=O)(OCC(F)(F)F)C(F)(F)F ; "Cs2CO3, DMF, 100° C.,30 min, microwave" ; CC(C)(C)OC(=O)N(C(=O)OC(C)(C)C)c1ncnc2c1c(Oc1cc(C(F)(F)F)ccn1)nn2CC(F)(F)F]

2,2,2-trifluoroethyl trifluoromethanesulfonate (49.09 mg, 211.51 umol, 1.05 eq), tert-butyl N-tert-butoxycarbonyl-N-[3-[[4-(trifluoromethyl)-2-pyridyl]oxy]-1H-pyrazolo[3,4-d]pyrimidin-4-yl]carbamate (1) (100 mg, 201.43 umol, 1 eq) and Cs₂CO₃ (131.26 mg, 402.87 umol, 2 eq) were taken up into a microwave tube in DMF (3 mL). The sealed tube was heated at 100° C. for 30 min under microwave. The mixture was added into water (50 mL). The aqueous layer was extracted with ethyl acetate (50 mL×3). The combined organic layers were washed with brine (50 mL), dried over Na₂SO₄, concentrated and purified by pre-TLC to give tert-butyl N-tert-butoxycarbonyl-N-[1-(2,2,2-trifluoroethyl)-3-[[4-(trifluoromethyl)-2-pyridyl]oxy]pyrazolo[3,4-d]pyrimidin-4-yl]carbamate (2) (100 mg) as an off-white solid. ¹H NMR (400 MHz, DMSO-d₆) δ=10.39 (s, 1H), 8.94-8.58 (m, 1H), 8.36 (d, J=5.2 Hz., 1H), 7.83-7.37 (m, 2H), 5.32 (q, J=9.0 Hz, 2H), 1.31-1.24 (m, 9H).

### Step 2. Procedure for preparation of I-(2,2,2-trifluoroethyl)-3-[[4-(trifluoromethyl)-2-pyridyl]oxy]pyrazolo[3,4-d]pyrimidin-4-amine

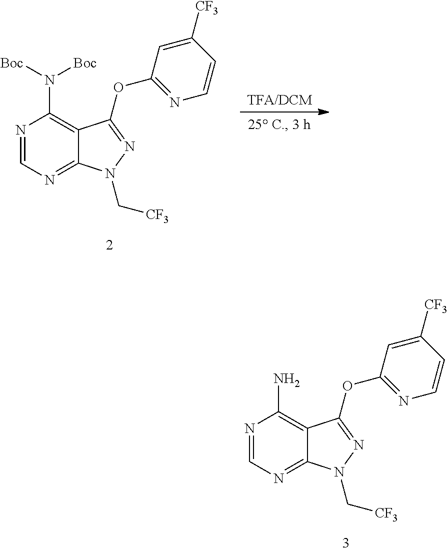 [SMILES: CC(C)(C)OC(=O)N(C(=O)OC(C)(C)C)c1ncnc2c1c(Oc1cc(C(F)(F)F)ccn1)nn2CC(F)(F)F ; "25° C., 3 h" ; "TFA/DCM" ; Nc1ncnc2c1c(Oc1cc(C(F)(F)F)ccn1)nn2CC(F)(F)F]

To a solution of tert-butyl N-tert-butoxycarbonyl-N-[1-(2,2,2-trifluoroethyl)-3-[[4-(trifluoromethyl)-2-pyridyl]oxy]pyrazolo[3,4-d]pyrimidin-4-yl]carbamate (2) (100 mg, 201.43 umol,) in DCM (1.5 mL) was added TFA (0.5 mL). The mixture was stirred at 25° C. for 3 h. The reaction mixture was concentrated and purified by pre-HPLC to give 1-(2,2,2-trifluoroethyl)-3-[[4-(trifluoromethyl)-2-pyridyl]oxy]pyrazolo[3,4-d]pyrimidin-4-amine (3) (30 mg, 79.32 umol, yield 39.38%) as a white solid.

¹H NMR (400 MHz, DMSO-d₆) δ=8.39 (d, J=5.2 Hz, 1H), 8.26 (s, 1H), 8.12-7.68 (m, 1H), 7.65 (s, 1H), 7.57 (d, 7=5.2 Hz, 1H), 7.47-6.75 (m, 1H), 5.13 (q, J=9.0 Hz, 2H).

### Synthetic Method AO: General Procedure Represented by the Preparation of 2-[4-amino-3-[[4-(trifluoromethyl)-2-pyridyl]oxy]pyrazolo[3,4-d]pyrimidin-1-yl]cyclohexanone

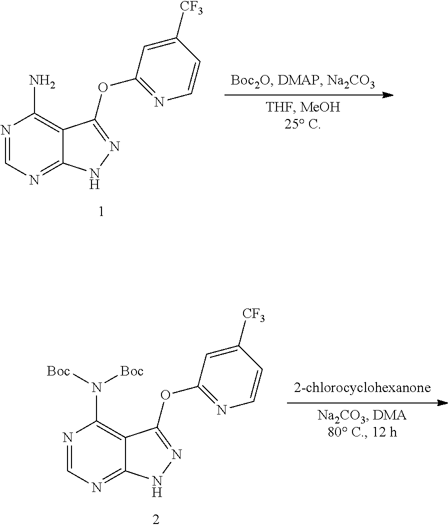

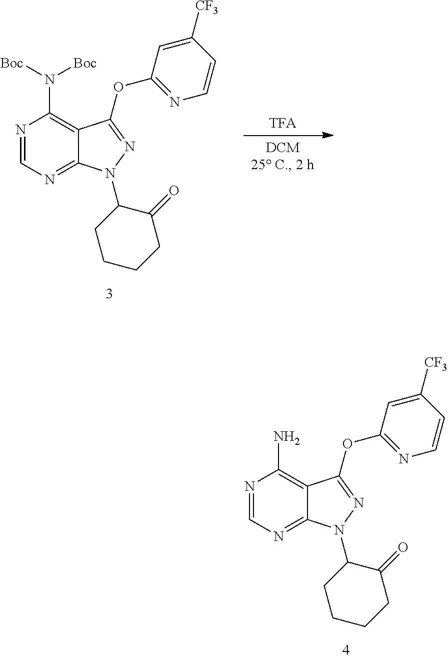 [SMILES: CC(C)(C)OC(=O)N(C(=O)OC(C)(C)C)c1ncnc2c1c(Oc1cc(C(F)(F)F)ccn1)nn2C1CCCCC1=O ; "DCM25° C., 2 h" ; "TFA" ; Nc1ncnc2c1c(Oc1cc(C(F)(F)F)ccn1)nn2C1CCCCC1=O]

### Step 1. Procedure for preparation of tert-butyl N-tert-butoxycarbonyl-N-[3-[[4-(trifluoromethyl)-2-pyridyl]oxy]-1H-pyrazolo[3,4-d]pyrimidin-4-yl]carbamate

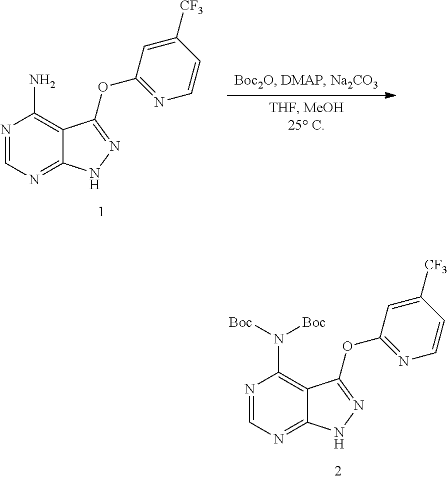

To a solution of 3-[[4-(trifluoromethyl)-2-pyridyl]oxy]-1H-pyrazolo[3,4-d]pyrimidin-4-amine (5 g, 16.88 mmol, 1 eq) (1) and DMAP (20.62 mg, 168.80 umol, 0.01 eq) in THF (50 mL) was added Boc₂O (14.74 g, 67.52 mmol, 15.51 mL, 4 eq) dropwise at 25° C. and stirred at 25° C. for 3 h. The mixture was concentrated under reduced pressure to give a residue. The residue was dissolved in methanol (50 mL) and NaHCO₃ (4.54 g, 54.02 mmol, 2.10 mL, 3.2 eq) was added in one portion. The reaction mixture was stirred at 25° C. for 12 h. LC-MS showed the reactant was consumed and the desired product was detected. The resulting mixture was partitioned between ethyl acetate (20 mL) and water (50 mL), and then the aqueous phase was further extracted with ethyl acetate (20 mL×3). The combined organic layers were washed with brine (30 mL), dried over Na₂SO₄, filtered and concentrated under reduced pressure to give a residue. The residue was purified by column chromatography (petroleum ether: ethyl acetate=1:1 to 0:1) to give compound tert-butyl N-tert-butoxycarbonyl-N-[3-[[4-(trifluoromethyl)-2-pyridyl]oxy]-1H-pyrazolo[3,4-d]pyrimidin-4-yl]carbamate (2) (5 g, 10.07 mmol, 59.67% yield) as yellow oil.

### Step 2. Procedure for preparation of tert-butyl N-tert-butoxycarbonyl-N-[1-(2-oxocyclohexyl)-3-[[4-(trifluoromethyl)-2-pyridyl]oxy]pyrazolo[3,4-d]pyrimidin-4-yl]carbamate

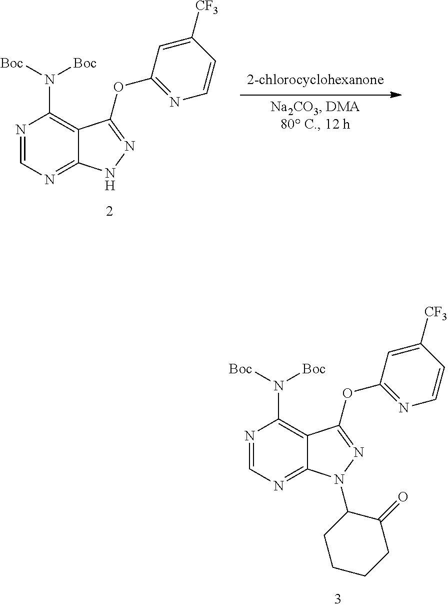 [SMILES: CC(C)(C)OC(=O)N(C(=O)OC(C)(C)C)c1ncnc2[nH]nc(Oc3cc(C(F)(F)F)ccn3)c12 ; "2-chlorocyclohexanone" ; "Na2CO3, DMA80° C., 12 h" ; CC(C)(C)OC(=O)N(C(=O)OC(C)(C)C)c1ncnc2c1c(Oc1cc(C(F)(F)F)ccn1)nn2C1CCCCC1=O]

A mixture of tert-butyl N-tert-butoxycarbonyl-N-[3-[[4-(trifluoromethyl)-2-pyridyl]oxy]-1H-pyrazolo[3,4-d]pyrimidin-4-yl]carbamate (2) (2 g, 4.03 mmol, 1 eq), 2-chlorocyclohexanone (801.23 mg, 6.04 mmol, 690.72 uL, 1.5 eq), Na₂CO₃ (853.99 mg, 8.06 mmol, 2 eq) in DMA (20 mL) was stirred at 80° C. for 12 hr. LC-MS showed the reactant was consumed and the desired product was detected. The reaction mixture was partitioned between H₂O (20 mL) and EA (10 mL). The organic phase was separated, washed with brine (10 mL×2), dried over Na₂SO₄, filtered and concentrated under reduced pressure to give a residue. The residue was purified by column chromatography (petroleum ether: Ethyl acetate=1:1 to 0:1). Compound tert-butyl N-tert-butoxycarbonyl-N-[1-(2-oxocyclohexyl)-3-[[4-(trifluoromethyl)-2-pyridyl]oxy]pyrazolo[3,4-d]pyrimidin-4-yl]carbamate (3) (2 g, 3.34 mmol, 82.94% yield, 99% purity) was obtained as yellow oil.

### Step 3. Procedure for preparation of 2-[4-amino-3-[[4-(trifluoromethyl)-2-pyridyl]oxy]pyrazolo[3,4-d]pyrimidin-1-yl]cyclohexanone

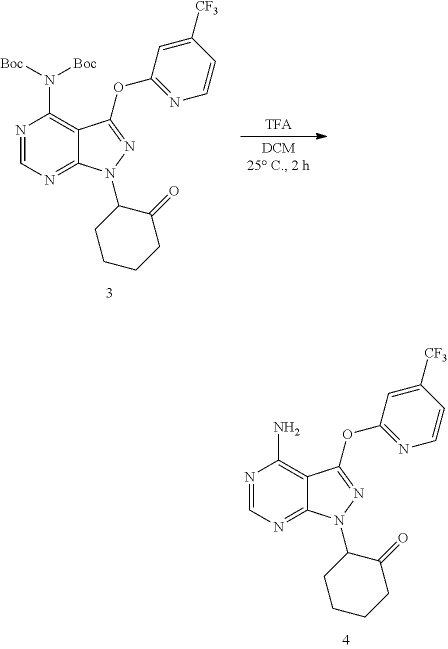 [SMILES: CC(C)(C)OC(=O)N(C(=O)OC(C)(C)C)c1ncnc2c1c(Oc1cc(C(F)(F)F)ccn1)nn2C1CCCCC1=O ; "DCM25° C., 2 h" ; Nc1ncnc2c1c(Oc1cc(C(F)(F)F)ccn1)nn2C1CCCCC1=O ; "TFA"]

A mixture of tert-butyl N-tert-butoxycarbonyl-N-[1-(2-oxocyclohexyl)-3-[[4-(trifluoromethyl)-2-pyridyl]oxy]pyrazolo[3,4-d]pyrimidin-4-yl]carbamate (3) (400 mg, 675.03 umol, 1 eq), TFA (3.08 g, 27.01 mmol, 2 mL, 40.02 eq) in DCM (10 mL) was stirred at 25° C. for 2 hr. LC-MS showed the reactant was consumed and the desired product was detected. The mixture was quenched with aq.Na₂CO₃ to pH=7 and extracted with DCM (4 mL×2). The combined organic layers were dried over Na₂SO₄, filtered and concentrated under reduced pressure to give a residue. The residue was purified by prep-HPLC (basic condition) to give Compound 2-[4-amino-3-[[4-(trifluoromethyl)-2-pyridyl]oxy]pyrazolo[3,4-d]pyrimidin-1-yl]cyclohexanone (4)(200 mg, 479.18 umol, 70.99% yield, 94% purity) was obtained as a white solid.

¹H NMR (ET8094-1130-P1Y, 400 MHz, CDCl₃)

δ=8.45-8.43 (d, J=4.8 Hz 1H), 8.33 (s, 1H), 7.60 (s, 1H), 7.38-7.36 (d, J=5.2 Hz 1H), 5.59 (brs, 2H), 5.43-5.38 (m, 1H), 2.65-2.62 (m, 2H), 2.52-2.49 (m, 2H), 2.16-2.13 (m, 2H), 1.93-1.76 (m, 2H)

### Synthetic Method AP: General Procedure Represented by the Preparation of 1-[4-amino-3-[[4-(trifluoromethyl)-2-pyridyl]oxy]pyrazolo[3,4-d]pyrimidin-1-yl]cyclobutanecarboxylic acid

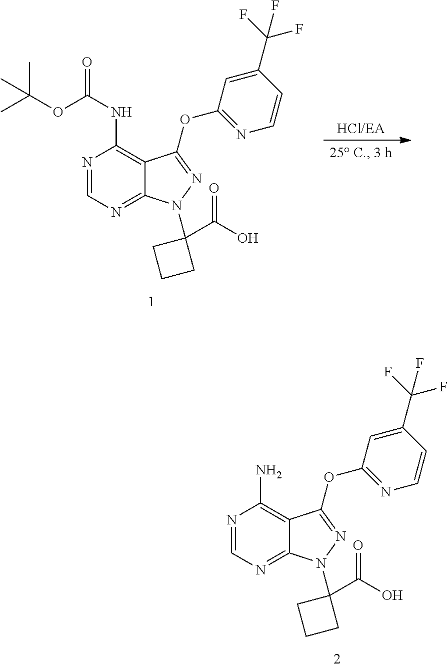 [SMILES: CC(C)(C)OC(=O)Nc1ncnc2c1c(Oc1cc(C(F)(F)F)ccn1)nn2C1(C(=O)O)CCC1 ; "25° C., 3 h" ; "HCl/EA" ; Nc1ncnc2c1c(Oc1cc(C(F)(F)F)ccn1)nn2C1(C(=O)O)CCC1]

### Step 1. Procedure for preparation of 1-[4-amino-3-[[4-(trifluoromethyl)-2-pyridyl]oxy]pyrazolo[3,4-d]pyrimidin-1-yl]cyclobutanecarboxylic acid

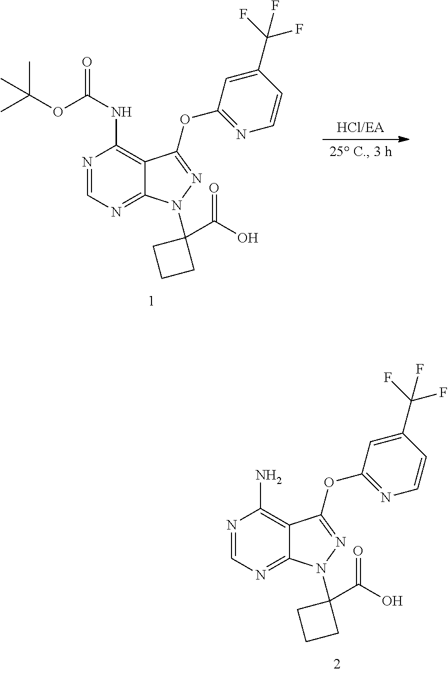 [SMILES: CC(C)(C)OC(=O)Nc1ncnc2c1c(Oc1cc(C(F)(F)F)ccn1)nn2C1(C(=O)O)CCC1 ; "25° C., 3 h" ; "HCl/EA" ; Nc1ncnc2c1c(Oc1cc(C(F)(F)F)ccn1)nn2C1(C(=O)O)CCC1]

A mixture of 1-[4-(tert-butoxycarbonylamino)-3-[[4-(trifluoromethyl)-2-pyridyl]oxy]pyrazolo[3,4-d]pyrimidin-1-yl]cyclobutanecarboxylic acid (1) (50 mg, 101.13 umol, 1 eq) in HCl/EtOAc (4 M, 3 mL, 118.66 eq) was degassed and then the mixture was stirred at 25° C. for 3 hr. LC-MS showed the reactant was consumed and the desired product was detected. The mixture was concentrated to give a residue. The residue was purified by prep-HPLC (TFA condition) to give 1-[4-amino-3-[[4-(trifluoromethyl)-2-pyridyl]oxy]pyrazolo[3,4-d]pyrimidin-1-yl]cyclobutanecarboxylic acid (2) (16.25 mg, 41.11 umol, 40.65% yield, 99.742% purity) as a white solid.

1H NMR (400 MHz, METHANOL-d4)

δ=8.46 (d, J=5.3 Hz, 1H), 8.28 (s, 1H), 7.73 (s, 1H), 7.56 (d, J=5.0 Hz, 1H), 3.18-3.03 (m, 2H), 2.96 (ddd, J=4.8, 8.9, 13.4 Hz, 2H), 2.45-2.30 (m, 1H), 2.20-2.06 (m, 1H)

### Synthetic Method AQ: General Procedure Represented by the Preparation of ethyl 1-[4-amino-3-[[4-(trifluoromethyl)-2-pyridyl]oxy]pyrazolo[3,4-d]pyrimidin-1-yl]cyclobutanecarboxylate

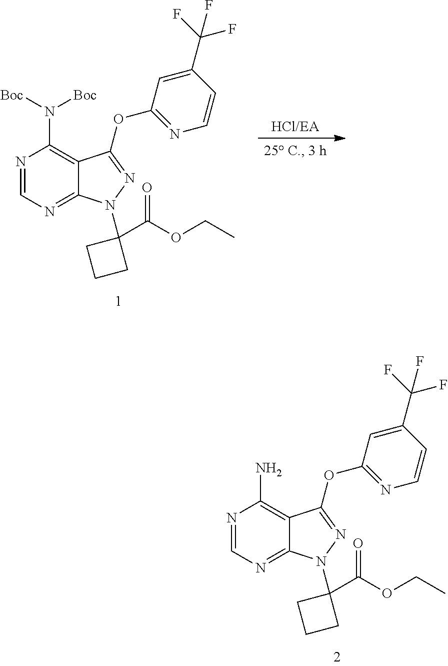 [SMILES: CCOC(=O)C1(n2nc(Oc3cc(C(F)(F)F)ccn3)c3c(N(C(=O)OC(C)(C)C)C(=O)OC(C)(C)C)ncnc32)CCC1 ; "25° C., 3 h" ; "HCl/EA" ; CCOC(=O)C1(n2nc(Oc3cc(C(F)(F)F)ccn3)c3c(N)ncnc32)CCC1]

### Step 1. Procedure for preparation of ethyl 1-[4-amino-3-[[4-(trifluoromethyl)-2-pyridyl]oxy]pyrazolo[3,4-d]pyrimidin-1-yl]cyclobutanecarboxylate

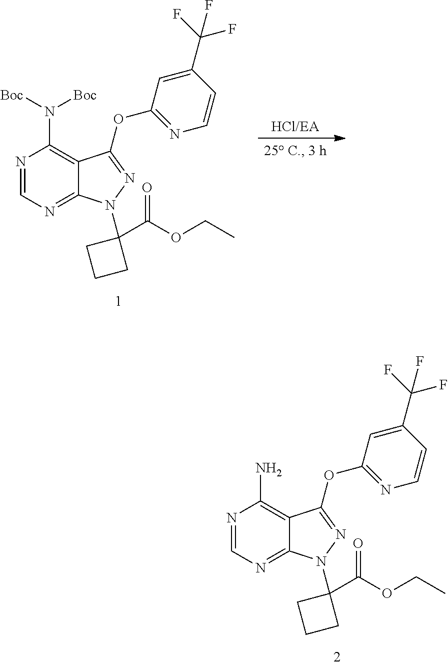 [SMILES: CCOC(=O)C1(n2nc(Oc3cc(C(F)(F)F)ccn3)c3c(N(C(=O)OC(C)(C)C)C(=O)OC(C)(C)C)ncnc32)CCC1 ; "25° C., 3 h" ; "HCl/EA" ; CCOC(=O)C1(n2nc(Oc3cc(C(F)(F)F)ccn3)c3c(N)ncnc32)CCC1]

A mixture of ethyl 1-[4-[bis(tert-butoxycarbonyl)amino]-3-[[4-(trifluoromethyl)-2-pyridyl]oxy]pyrazolo[3,4-d]pyrimidin-1-yl]cyclobutanecarboxylate (1) (100 mg, 160.62 umol, 1 eq) in HCl/EtOAc (4 M, 6.00 mL, 149.42 eq) was stirred at 25° C. for 3 hr. The mixture was concentrated to give a residue. The residue was purified by prep-HPLC (TFA condition) to give Compound ethyl 1-[4-amino-3-[[4-(trifluoromethyl)-2-pyridyl]oxy]pyrazolo[3,4-d]pyrimidin-1-yl]cyclobutanecarboxylate (2) (56.4 mg, 132.87 umol, 82.72% yield, 99.5% purity) as a white solid.

¹H NMR (400 MHz, METHANOL-da) δ=8.46 (d, J=5.3 Hz, 1H), 8.29 (s, 1H), 7.72 (s, 1H), 7.59-7.54 (m, 1H), 4.21 (q, 7=7.1 Hz, 2H), 3.16-3.04 (m, 2H), 2.99-2.89 (m, 2H), 2.33 (quind, J=8.7, 11.2 Hz, 1H), 2.19-2.07 (m, 1H)

### Synthetic Method AR: General Procedure Represented by the Preparation of 2-[4-amino-1-(3-fluorocyclobutyl)pyrazolo[3,4-d]pyrimidin-3-yl]oxypyridin-4-ol

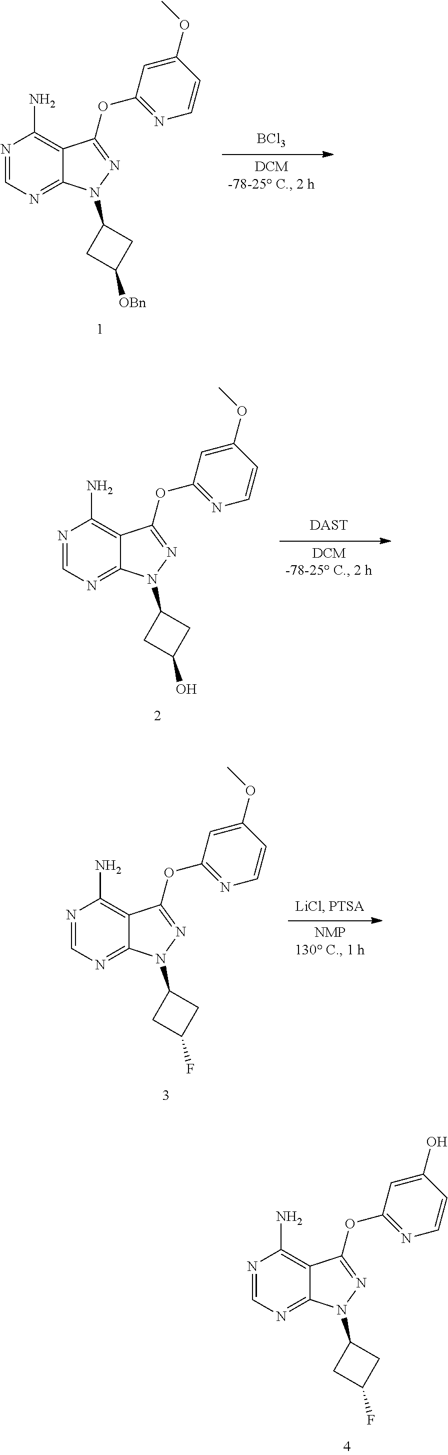

### Step 1. Procedure for preparation of 3-[4-amino-3-[(4-methoxy-2-pyridyl)oxy]pyrazolo[3,4-d]pyrimidin-1-yl]cyclobutanol

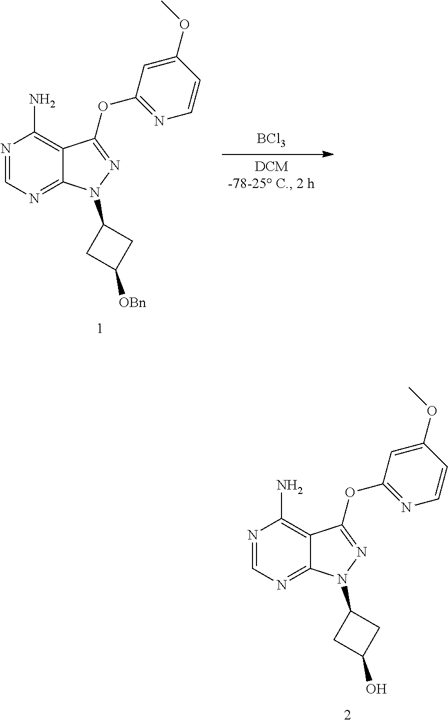 [SMILES: COc1ccnc(Oc2nn([C@H]3C[C@@H](OCc4ccccc4)C3)c3ncnc(N)c23)c1 ; "BCl3" ; "DCM-78-25° C., 2 h" ; COc1ccnc(Oc2nn([C@H]3C[C@@H](O)C3)c3ncnc(N)c23)c1]

To a solution of 1-(3-benzyloxycyclobutyl)-3-[(4-methoxy-2-pyridyl)oxy]pyrazolo[3,4-d]pyrimidin-4-amine (1) (360 mg, 860.32 umol, 1 eq) in DCM (dichloromethane) (5 mL) was added BCl₃ (1 M, 4.30 mL, 5 eq) under N₂ at −78° C. The suspension was degassed under vacuum and purged with N₂ several times. The mixture was stirred under N₂ at 25° C. for 2 hours. LC-MS showed the reactant was consumed and the desired product was detected. The mixture was added methanol at −78° C., then the solution was concentrated to afford the crude product. The crude product was used to the next step without further purification. 3-[4-amino-3-[(4-methoxy-2-pyridyl)oxy]pyrazolo[3,4-d]pyrimidin-1-yl]cyclobutanol (60 mg, 182.75 umol, 21.24% yield) (2) was obtained as a yellow oil.

### Step 2. Procedure for preparation of 1-(3-fluorocyclobutyl)-3-[(4-methoxy-2-pyridyl)oxy]pyrazolo[3,4-d]pyrimidin-4-amine

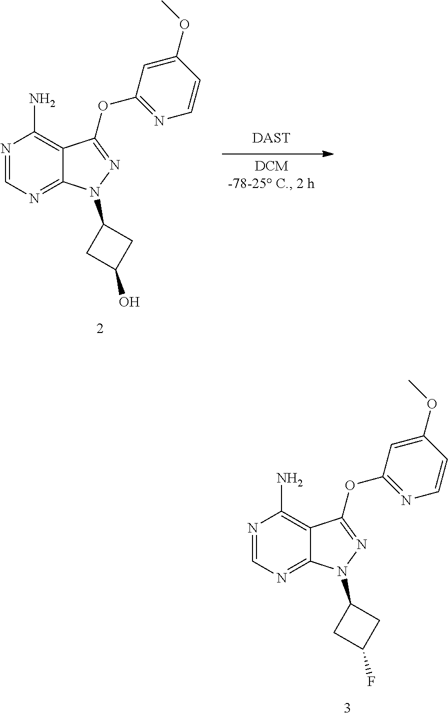

To a mixture of 3-[4-amino-3-[(4-methoxy-2-pyridyl)oxy]pyrazolo[3,4-d]pyrimidin-1-yl]cyclobutanol (2) (50 mg, 152.29 umol, 1 eq) in DCM (dichloromethane) (5 mL) was degassed and purged with N₂ for 3 times, and was added DAST (122.74 mg, 761.44 umol, 100.60 uL, 5 eq) at −78° C., then the mixture was stirred at 25° C. for 2 hr under N₂ atmosphere. LC-MS showed the reactant was consumed and the desired product was detected. The reaction was concentrated to afford the residue. The residue was purified by prep-HPLC (TFA condition) to give 1-(3-fluorocyclobutyl)-3-[(4-methoxy-2-pyridyl)oxy]pyrazolo[3,4-d]pyrimidin-4-amine (3) (35 mg, 100.78 umol, 66.17% yield, 95.11% purity) as a white solid.

¹H NMR (ET14917-655-p1a, 400 MHz, MeOD)

δ=8.32 (s, 1H), 8.05-8.03 (d, J=6 Hz 1H), 7.04-7.03 (d, J=2 Hz 1H), 6.91-6.89 (dd, J=8 Hz, J=2 Hz 1H), 5.64-5.46 (m, 1H), 5.45-5.44 (m, 1H), 5.31-5.29 (m, 1H), 3.96 (s, 1H), 3.31-2.79 (m, 4H)

### Step 3. Procedure for preparation of 2-[4-amino-1-(3-fluorocyclobutyl)pyrazolo[3,4-d]pyrimidin-3-yl]oxypyridin-4-ol

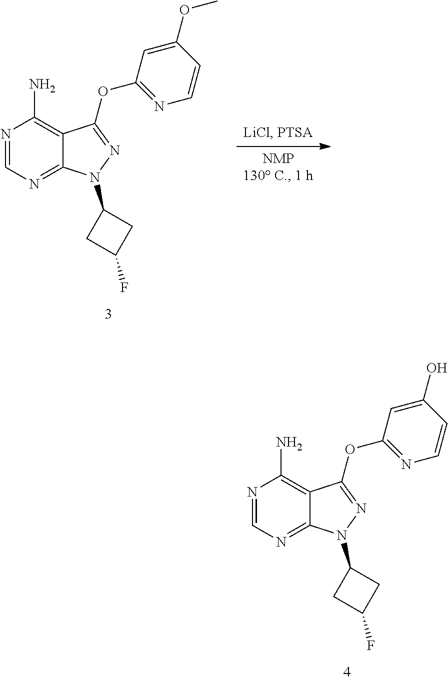

To a solution of 1-(3-fluorocyclobutyl)-3-[(4-methoxy-2-pyridyl)oxy]pyrazolo[3,4-d]pyrimidin-4-amine (3) (25 mg, 75.69 umol, 1 eq) in NMP (2 mL) was added LiCl (32.09 mg, 756.85 umol, 15.50 uL, 10 eq) and PTSA (130.33 mg, 756.85 umol, 10 eq). The mixture was stirred at 130° C. for 1 hr. LC-MS showed the reactant was consumed and the desired product was detected. The reaction was added MeCN. The residue was purified by prep-HPLC (TFA condition) to give 2-[4-amino-1-(3-fluorocyclobutyl)pyrazolo[3,4-d]pyrimidin-3-yl]oxypyridin-4-ol (4) (10.8 mg, 34.15 umol, 45.12% yield, 100% purity) as a white solid.

NMR (ET14917-656-p1a, 400 MHz, MeOD)

δ=8.32 (s, 1H), 7.98-7.97 (d, J=5.6 Hz 1H), 6.94-6.93 (d, J=2 Hz 1H) 6.76-6.74 (dd, J=8 Hz, J=2 Hz 1H), 5.64-5.49 (m, 1H), 5.48-5.46 (m, 1H), 5.33-5.32 (m, 1H), 3.31-2.81 (m, 4H)

### Synthetic Method AS: General Procedure Represented by the Preparation of (1R,2S)-2-[4-amino-3-[(4-methoxy-2-pyridyl)oxy]pyrazolo[3,4-d]pyrimidin-1-yl]cyclohexanol

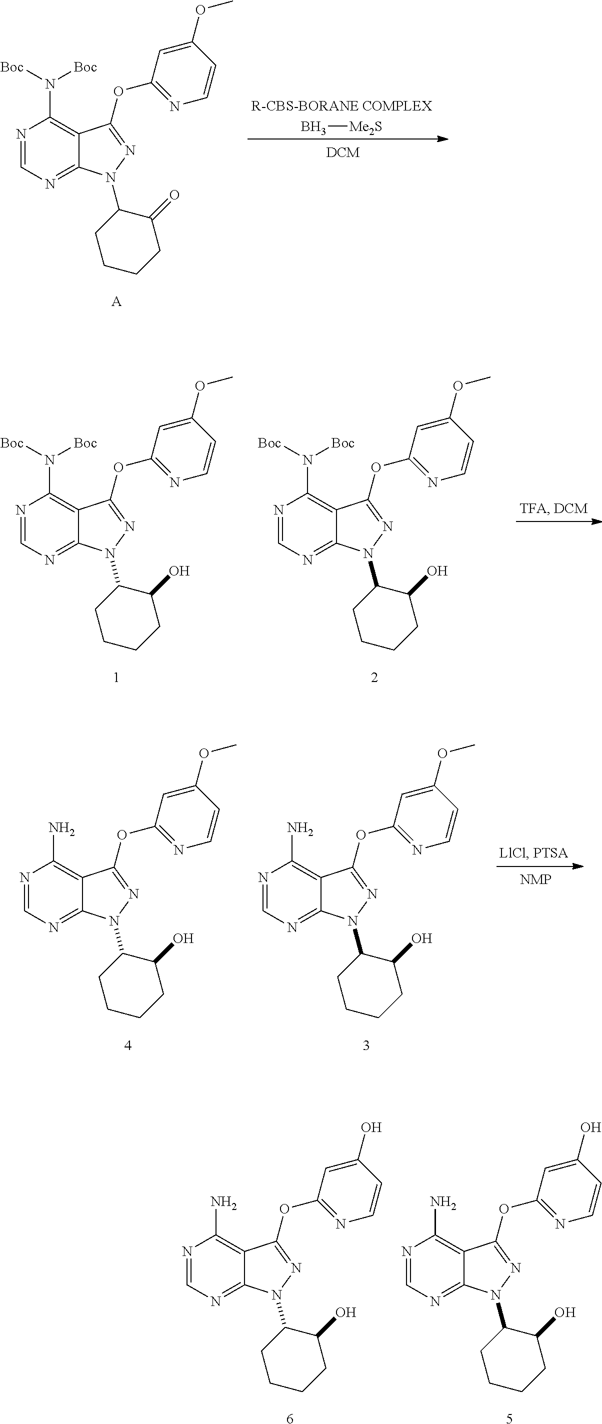

Step 1. Procedure for preparation of trans-Compound 1 and cis-Compound 2

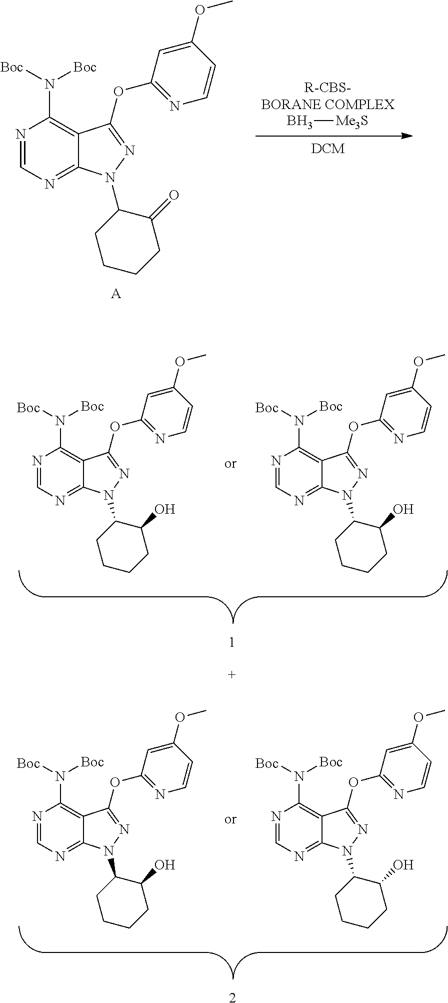

To a solution of r-cbs-borane complex (1 M, 2.88 mL, 5 eq) in DCM (3 mL) was added BH₃-Me₂S (10 M, 63.47 uL, 1.1 eq) at 25° C. and stirred at 25° C. for 1 h. Then the solution was added to a mixture of tert-butyl N-tert-butoxycarbonyl-N-[3-[(4-methoxy-2-pyridyl)oxy]-1-(2-oxocyclohexyl)pyrazolo[3,4-d]pyrimidin-4-yl]carbamate (A) (320 mg, 577.00 umol, 1 eq) and r-cbs-borane complex (1 M, 1.15 mL, 2 eq) in DCM (6 mL) at −70° C. The mixture was stirred at −10° C. for 12 h. LC-MS showed the reactant was consumed and the desired product was detected. The mixture was quenched by methanol (5 mL) and then concentrated under reduced pressure to give a residue. The residue was purified by prep-TLC (SiO₂, Petroleum ether/Ethyl acetate=1:1) to give trans-Compound (1) (90 mg, 161.69 umol, 28.02% yield) and cis-Compound (2) (60 mg, 107.80 umol, 18.68% yield) was obtained as yellow oil.

### Step 2. Procedure for preparation of (1R,2S)-2-(4-amino-3-((4-methoxypyridin-2-yl)oxy)-1H-pyrazolo[3,4-d]pyrimidin-1-yl)cyclohexanol or (1S,2R)-2-(4-amino-3-((4-methoxypyridin-2-yl)oxy)-1H-pyrazolo[3,4-d]pyrimidin-1-yl)cyclohexanol

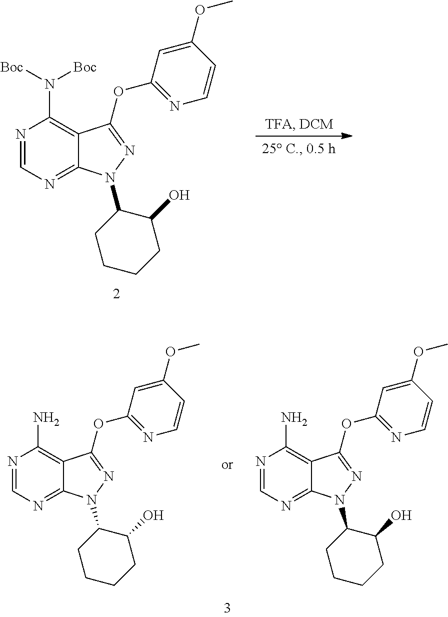

A mixture of cis-Compound (2) (50.00 mg, 89.83 umol, 1 eq) in DCM (1 mL) and TFA (0.1 mL) was stirred at 25 C for 0.5 hr. LC-MS showed the reactant was consumed and the desired product was detected. The mixture was adjusted to pH=7 by sat Na₂CO₃ aq and extracted with DCM (2 mL×2). The combined organic layers were dried over Na₂SO₄, filtered and concentrated under reduced pressure to give a residue. The residue was purified by re-crystallization from dioxane (1 mL) at 100° C. to give the mixture of (1R,2S)-2-(4-amino-3-((4-methoxypyridin-2-yl)oxy)-1H-pyrazolo[3,4-d]pyrimidin-1-yl)cyclohexanol or (1S,2R)-2-(4-amino-3-((4-methoxypyridin-2-yl)oxy)-1H-pyrazolo[3,4-d]pyrimidin-1-yl)cyclohexanol (30 mg, 82.50 umol, 91.84% yield, 98% purity) was obtained as a white solid.

¹H NMR (ET8094-1190-P1L, 400 MHz, DMSO)

δ=8.16 (s, 1H), 7.96-7.95 (d, J=5.6 Hz 1H), 6.81-6.78 (m, 2H), 4.61-4.56 (m, 2H), 4.13 (s, 1H), 3.87 (s, 3H), 2.50-2.26 (m, 1H), 1.79-1.67 (m, 2H), 1.63 (m, 1H), 1.60 (m, 1H), 1.42-1.36 (m, 2H), 1.24 (s, 1H)

### Synthetic Method AT: General Procedure Represented by the Preparation of (1R,2R)-2-(4-amino-3-((4-methoxypyridin-2-yl)oxy)-1H-pyrazolo[3,4-d]pyrimidin-1-yl)cyclohexanol or (1 S,2S)-2-(4-amino-3-((4-methoxypyridin-2-yl)oxy)-1H-pyrazolo[3,4-d]pyrimidin-1-yl)cyclohexanol

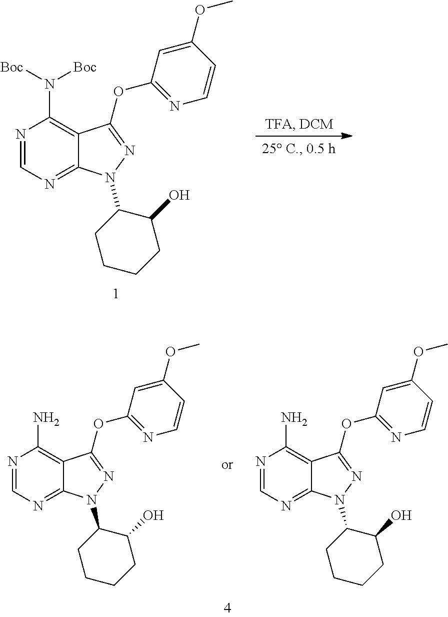

### Step 1. Procedure for preparation of (1R,2R)-2-(4-amino-3-((4-methoxypyridin-2-yl)oxy)-1H-pyrazolo[3,4-d]pyrimidin-1-yl)cyclohexanol or (1S,2S)-2-(4-amino-3-((4-methoxypyridin-2-yl)oxy)-1H-pyrazolo[3,4-d]pyrimidin-1-yl)cyclohexanol

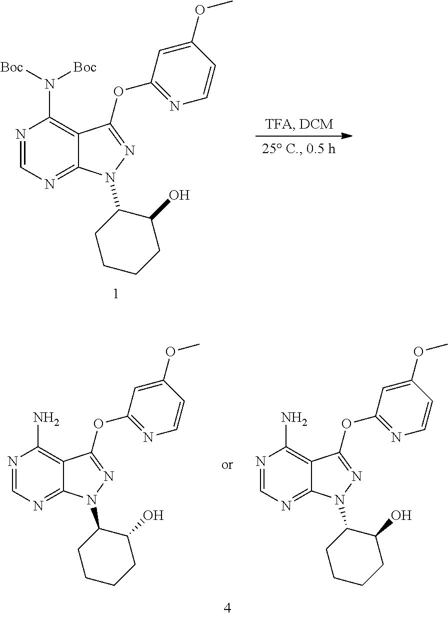

A mixture of trans-compound (1) (90.00 mg, 161.69 umol, 1 eq) in DCM (1 mL) and TFA (0.1 mL) was stirred at 25° C. for 0.5 hr. LC-MS showed the reactant was consumed and the desired product was detected. The mixture was adjusted to pH=7 by sat Na₂CO₃ aq and extracted with DCM (2 mL×2). The combined organic layers were dried over Na₂SO₄, filtered and concentrated under reduced pressure to give a residue. The crude product was purified by re-crystallization from dioxane (1 mL) at 100° C. to give Compound (1R,2R)-2-(4-amino-3-((4-methoxypyridin-2-yl)oxy)-1H-pyrazolo[3,4-d]pyrimidin-1-yl)cyclohexanol or (1S,2S)-2-(4-amino-3-((4-methoxypyridin-2-yl)oxy)-1H-pyrazolo[3,4-d]pyrimidin-1-yl)cyclohexanol

(50 mg, 137.49 umol, 85.03% yield, 98% purity) was obtained as a white solid.

¹H NMR (ET8094-1191-P1L, 400 MHz, DMSO)

δ=8.14 (s, 1H), 7.97-7.% (d, J=6 Hz 1H), 6.81-6.79 (dd, J=2 Hz, J=8 Hz 1H), 6.76-6.75 (d, J=2 Hz 1H), 4.65 (s, 1H), 4.63-4.30 (m, 1H), 3.87 (m, 3H), 3.79 (m, 1H), 1.95 (m, 1H), 1.80 (m, 2H), 1.70 (m, 2H), 1.36-1.24 (m, 3H)

### Synthetic Method AU: General Procedure Represented by the Preparation of 2-((4-amino-1-((1S,2R)-2-hydroxycyclohexyl)-1H-pyrazolo[3,4-d]pyrimidin-3-yl)oxy)pyridin-4-ol and 2-((4-amino-1-((1R,2S)-2-hydroxycyclohexyl)-1H-pyrazolo[3,4-d]pyrimidin-3-yl)oxy)pyridin-4-ol

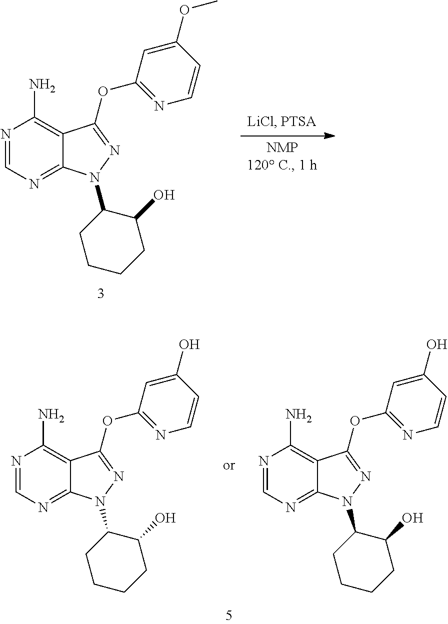

### Step 1. Procedure for preparation of 2-((4-amino-1-((1S,2R)-2-hydroxycyclohexyl)-1H-pyrazolo[3,4-d]pyrimidin-3-yl)oxy)pyridin-4-ol or 2-((4-amino-1-((1R,2S)-2-hydroxycyclohexyl)-1H-pyrazolo[3,4-d]pyrimidin-3-yl)oxy)pyridin-4-ol

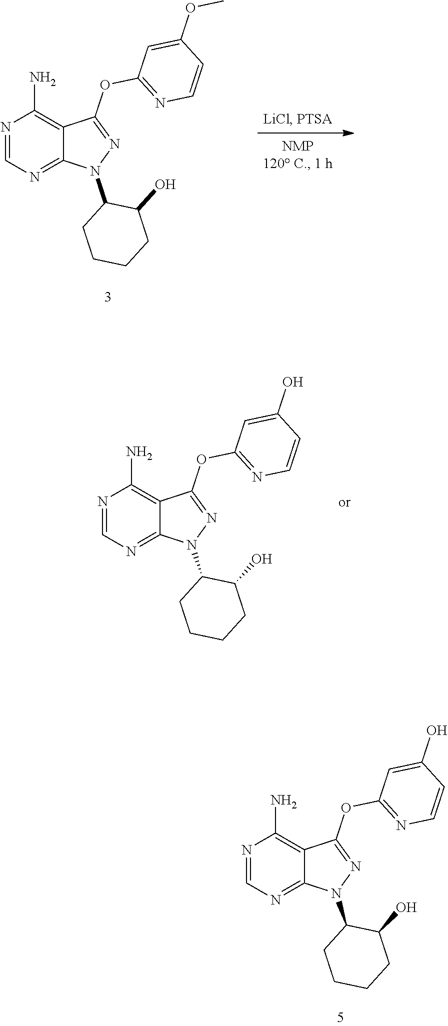

A mixture of cis-compound 3 (30 mg, 84.18 umol, 1 eq), LiCl (35.68 mg, 841.80 umol, 17.24 uL, 10 eq), PTSA (144.96 mg, 841.80 umol, 10 eq) in NMP (1 mL) was stirred at 120° C. for 1 hr. LC-MS showed the reactant was consumed and the desired product was detected. The mixture was filtered and the filtrate was purified by prep-HPLC (neutral condition). The mixture of 2-((4-amino-1-((1S,2R)-2-hydroxycyclohexyl)-1H-pyrazolo[3,4-d]pyrimidin-3-yl)oxy)pyridin-4-ol or 2-((4-amino-1-((1R,2S)-2-hydroxycyclohexyl)-1H-pyrazolo[3,4-d]pyrimidin-3-yl)oxy)pyridin-4-ol (5.8 mg, 16.89 umol, 20.07% yield, 99.71% purity) was obtained as a white solid.

¹H NMR (ET8094-1206-P1L, 400 MHz, DMSO)

δ=8.15 (s, 1H), 7.85-7.83 (d, J=6 Hz 1H), 6.61-6.59 (dd, J=2 Hz, J=7.6 Hz 1H), 6.48-6.47 (d, J=1.2 Hz 1H), 4.63-4.62 (d, J=3.6 Hz, 1H), 4.58-4.55 (dd, J=7.6 Hz, 1H), 4.13 (s, 1H), 2.29-2.23 (m, 1H), 1.79-1.77 (m, 2H), 1.66-1.57 (m, 3H), 1.39-1.36 (m, 2H)

### Synthetic Method AV: General Procedure Represented by the Preparation of 2-((4-amino-1-((1R,2R)-2-hydroxycyclohexyl)-1H-pyrazolo[3,4-d]pyrimidin-3-yl)oxy)pyridin-4-ol or 2-((4-amino-1-((1S,2S)-2-hydroxycyclohexyl)-1H-pyrazolo[3,4-d]pyrimidin-3-yl)oxy)pyridin-4-ol

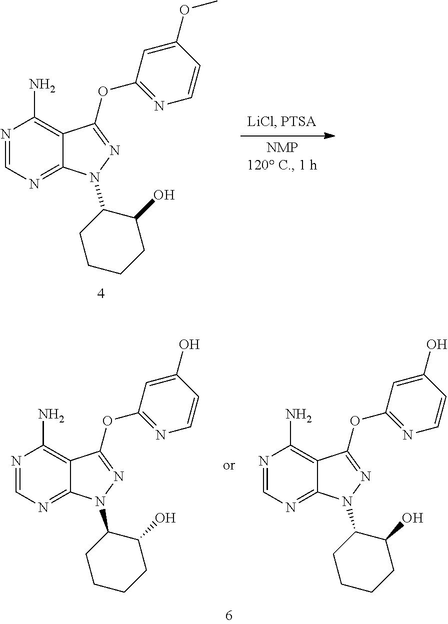

### Step 1. Procedure for preparation of 2-((4-amino-1-((1R,2R)-2-hydroxycyclohexyl)-JH-pyrazolo[3,4-d]pyrimidin-3-yl)oxy)pyridin-4-ol or 2-((4-amino-1-((1S,2S)-2-hydroxycyclohexyl)-1H-pyrazolo[3,4-d]pyrimidin-3-yl)oxy)pyridin-4-ol

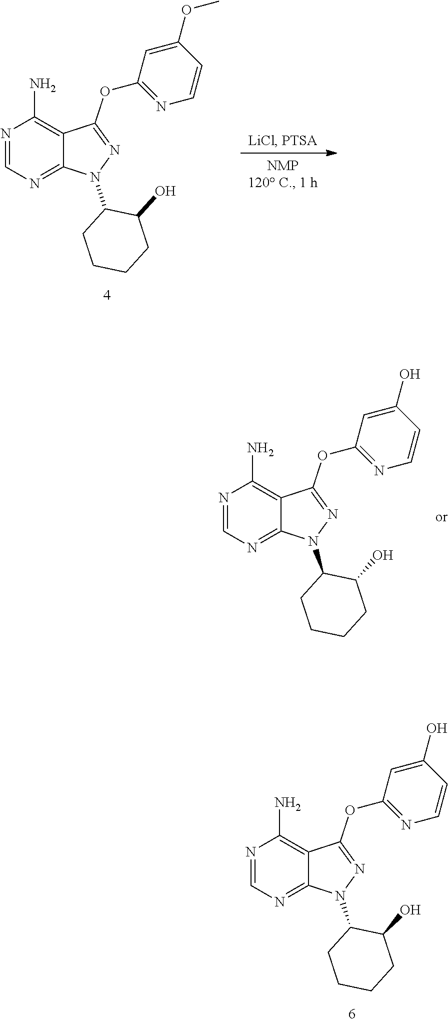

A mixture of trans-compound 4 (30 mg, 84.18 umol, 1 eq), LiCl (35.68 mg, 841.80 umol, 17.24 uL, 10 eq), PTSA (144.96 mg, 841.80 umol, 10 eq) in NMP (1 mL) was stirred at 120° C. for 1 hr. LC-MS showed the reactant was consumed and the desired product was detected. The mixture was filtered and the filtrate was purified by prep-HPLC (neutral condition). The mixture of 2-((4-amino-1-((1R,2R)-2-hydroxycyclohexyl)-1H-pyrazolo[3,4-d]pyrimidin-3-yl)oxy)pyridin-4-ol or 2-((4-amino-1-((1S,2S)-2-hydroxycyclohexyl)-1H-pyrazolo[3,4-d]pyrimidin-3-yl)oxy)pyridin-4-ol (6.2 mg, 18.05 umol, 21.45% yield, 99.69% purity) was obtained as a white solid.

¹H NMR (ET8094-1207-P1L, 400 MHz, DMSO)

δ=8.14 (s, 1H), 7.86-7.85 (d, J=5.6 Hz 1H), 6.61-6.59 (dd, J=2 Hz, J=3.6 Hz 1H), 6.48-6.47 (d, J=1.6 Hz 1H), 4.67-4.66 (d, J=5.2 Hz 1H), 4.36-4.29 (m, 1H), 3.79 (s, 1H), 1.95 (m, 1H), 1.79-1.70 (m, 4H), 1.68-1.24 (m, 3H)

Additional Compounds of the Invention: The following compounds may be prepared according to the methods described herein.

TABLE 18
Further Compounds of the Invention

| | | | LC/MS | |
|---|---|---|---|---|
| Compound | | | Observed | |
| No. | IUPAC Name | MW | (M + H) | ¹H NMR (400 MHz) |
| 156 | 3-(3-chlorophenoxy)-1- | 333.74 | 334 | (Methanol-d₄) δ = 8.18 (s, 1H), |
| | [(trans)-3-fluorocyclobutyl]- | | | 7.47 (t, J = 2.0 Hz, 1H), |
| | 1H-pyrazolo[3,4-d]pyrimidin- | | | 7.44-7.39 (m, 1H), |
| | 4-amine | | | 7.36-7.32 (m, 1H), |
| | | | | 7.24 (dd, J = 1.2, 8.0 Hz, 1H), |
| | | | | 5.53-5.45 (m, 1H), |
| | | | | 5.40-5.38 (m, 0.5H), |
| | | | | 5.28-5.23 (m, 0.5H), |
| | | | | 2.89-2.70 (m, 4H) |
| 157 | 3-[(5-fluoroindol-3-yl) | 353.35 | 355.1 | (Acetonitrile-d₃) δ = 9.29 |
| | methyl]-1-[(trans)-3- | | | (s, 1H), 8.14 (s, 1H), |
| | fluorocyclobutyl]-1H- | | | 7.38 (dd, J = 4.4, 8.8 Hz, |
| | pyrazolo[3,4-d] | | | 1H), 7.20 (d, J = 2.4 Hz, 1H), |
| | pyrimidin-4-amine | | | 7.15 (dd, J = 2.8, 7.6 Hz, 1H), |
| | | | | 6.92 (dt, J = 2.4, 9.2 Hz, 1H), |
| | | | | 5.71-5.45 (m, 4H), |
| | | | | 4.36 (s, 2H), |
| | | | | 3.00-2.76 (m, 4H) |
| 158 | 3-[(4-chloropyridin-2-yl) | 334.74 | 335.1 | (DMSO-d₆) δ = 8.19 (s, 1H), |
| | oxy]-1-[(trans)-3- | | | 8.12 (d, J = 5.6 Hz, 1H), |
| | fluorocyclobutyl]-1H- | | | 7.41 (d, J = 1.6 Hz, 1H), |
| | pyrazolo[3,4-d] | | | 7.34 (dd, J = 1.6, 5.6 Hz, 1H), |
| | pyrimidin-4-amine | | | 5.50-5.45 (m, 1.5H), |
| | | | | 5.35-5.31 (m, 0.5H), |
| | | | | 2.82-2.67 (m, 4H) |
| 159 | 1-cyclopropyl-3-{[4- | 336.27 | 337 | (Methanol-d₄) δ = 8.41 |
| | (trifluoromethyl)pyridin- | | | (d, J = 5.2 Hz, 1H), 8.34 |
| | 2-yl]oxy}- | | | (s, 1H), 7.68 (s, 1H), |
| | 1H-pyrazolo[3,4-d] | | | 7.54 (d, J = 5.2 Hz, 1H), |
| | pyrimidin-4-amine | | | 3.88-3.83 (m, 1H), |
| | | | | 1.26-1.20 (m, 2H), |
| | | | | 1.14-1.13 (m, 2H) |
| 160 | 2-({4-amino-1- | 284.27 | 285.1 | (Methanol-d₄) δ = 8.34 |
| | cyclopropyl-1H- | | | (s, 1H), 7.96 (d, J = 5.6 |
| | pyrazolo[3,4-d] | | | Hz, 1H), 6.89 (d, J = 2.0 |
| | pyrimidin-3-yl)oxy) | | | Hz, 1H), 6.74-6.72 (m, |
| | pyridin-4-ol | | | 1H), 3.88-3.82 (m, 1H), |
| | | | | 1.28-1.25 (m, 2H), |
| | | | | 1.16-1.12 (m, 2H) |
| 161 | 3-[(3-fluorophenyl)methyl]- | 315.32 | 316.2 | (Methanol-d₄) δ = 8.31 |
| | 1-[(trans)-3-fluorocyclobutyl]- | | | (s, 1H), 7.37-7.32 (m, 1H), |
| | 1H-pyrazolo[3,4-d]pyrimidin- | | | 7.09 (d, J = 7.6 Hz, 1H), |
| | 4-amine | | | 7.05-6.98 (m, 2H), |
| | | | | 5.68-5.62 (m, 1H), |
| | | | | 5.55-5.53 (m, 0.5H), |
| | | | | 5.40-5.39 (m, 0.5H), |
| | | | | 4.48 (s, 2H), |
| | | | | 3.00-2.85 (m, 4H) |
| 162 | 3-[(4-methoxypyridin-2- | 330.32 | 331 | (Methanol-d₄) δ = 8.32 (s, 1H), |
| | yl)oxy]-1-[trans)-3- | | | 8.04 (d, J = 6.0 Hz ,1H), |
| | fluorocydobutyl]-1H- | | | 7.04 (d, J = 2.0 Hz, 1H), |
| | pyrazolo[3,4-d] | | | 6.91-6.89 (m, 1H), |
| | pyrimidin-4-amine | | | 5.64-5.60 (m, 1H), |
| | | | | 5.46-5.44 (m, 0.5H), |
| | | | | 5.31-5.29 (m, 0.5H), |
| | | | | 3.96 (s, 3H), |
| | | | | 2.93-2.79 (m, 4H) |
| 163 | (cis)-3-{4-amino-3-[(4- | 332.75 | 333 | (DMSO-d₆ + D₂O) δ = 8.27 |
| | chloropyridin-2-yl)oxy]- | | | (s, 1H), 8.11 (d, J = 5.6 Hz, |
| | 1H-pyrazolo[3,4-d] | | | 1H), 7.43 (d, J = 1.6 Hz, 1H) |
| | pyrimidin-1-yl} | | | 7.34 (dd, J = 1.6, 5.6 Hz, 1H), |
| | cyclobutan-1-ol | | | 4.83-4.75 (m, 1H), |
| | | | | 4.03-3.95 (m, 1H), |
| | | | | 2.71-2.67 (m, 2H), |
| | | | | 2.41-2.33 (m, 2H) |
| 164 | (trans)-3-{4-amino-5-[(3- | 312.34 | 313.1 | (Acetonitrile-d₃) δ = 8.11 |
| | fluorophenyl)methyl]- | | | (s, 1H), 7.36-7.32 (m, 1H), |
| | 7H-pyrrolo[2,3-d] | | | 7.26 (s, 1H), |
| | pyrimidin-7-yl} | | | 7.08 (d, J = 7.6 Hz, 1H), |
| | cyclobutan-1-ol | | | 7.0-6.94 (m, 2H), |
| | | | | 5.44-5.34 (m, 1H), |
| | | | | 4.55-4.49 (m, 1H), |
| | | | | 4.19 (s, 2H), |
| | | | | 2.73-2.63 (m, 2H), |
| | | | | 2.53-2.44 (m, 2H) |
| 165 | 1-cyclopropyl-3-[(4- | 308.34 | 309.1 | (Methanol-d₄) δ = 8.31 (s, 1H), |
| | cyclopropylpyridin-2-yl) | | | 8.01 (d, J = 5.2 Hz, 1H), |
| | oxy]-1H-pyrazolo[3,4-d] | | | 7.13 (s, 1H), |
| | pyrimidin-4-amine | | | 6.98 (d, J = 4.0 Hz, 1H), |
| | | | | 3.83-3.77 (m, 1H), |
| | | | | 2.10-2.01 (m, 1H), |
| | | | | 1.22-1.19 (m, 4H), |
| | | | | 1.18- 1.17 (m, 2H), |
| | | | | 0.92-0.89 (m, 2H) |
| 166 | 1-[(trans)-3- | 367.30 | 368.1 | (Methanol-d₄) δ = 8.18 |
| | fluorocyclobutyl]-3-[3- | | | (s, 1H), 7.80 (s, 1H), |
| | (triflueromethypphenoxy]- | | | 7.72-7.68 (m, 1H), |
| | 1H-pyrazolo[3,4-d]pyrimidin- | | | 7.66-7.61 (m, 1H), |
| | 4-amine | | | 7.56-7.51 (m, 1H), |
| | | | | 5.49 (t, J = 7.2 Hz, 1H), |
| | | | | 5.42-5.36 (m, 0.5H), |
| | | | | 5.27-5.20 (m, 0.5H), |
| | | | | 2.90-2.68 (m, 4H) |
| 167 | 3-(3-fluorephenoxy)-1- | 317.29 | 318.1 | (Methanol-d₄) δ = 8.18 |
| | [(trans)-3-fluorocyclobutyl]- | | | (s, 1H), 7.47-7.40 (m, |
| | 1H-pyrazolo[3,4-d] | | | 1H), 7.24-7.19 (m, 2H), |
| | pyrimidin-4-amine | | | 7.01-6.95 (m, 1H), |
| | | | | 5.53-5.45 (m, 1H), |
| | | | | 5.40-5.39 (m, 0.5H), |
| | | | | 5.26-5.24 (m, 0.5H), |
| | | | | 2.86-2.70 (m, 4H) |
| 168 | (cis)-3-{4-amino-3-[(4- | 328.33 | 329.1 | (Methanol-d₄) δ = 8.17 (s, 1H), |
| | methoxypyridin-2-yl) | | | 8.01 (d, J = 6.0 Hz, 1H), |
| | oxy]-1H-pyrazolo | | | 7.05 (d, J = 2.0 Hz, 1H), |
| | [3,4-d]pyrimidin- | | | 6.84 (dd, J = 2.0, 6.0 Hz, 1H), |
| | 1-yl}cyclobutan-1-ol | | | 4.82-4.79 (m, 1H), |
| | | | | 4.17-4.09 (m, 1H), |
| | | | | 3.94 (s, 3H), |
| | | | | 2.83-2.79 (m, 2H), |
| | | | | 2.60-2.57 (m, 2H) |
| 169 | 1-cyclopropyl-3-{[4- | 352.27 | 353.1 | (Methanol-d₄) δ = 8.25-8.22 |
| | (trifluoromethoxy) | | | (m, 2H), 7.32 (s, 1H), |
| | pyridin-2-yl]oxy}- | | | 7.16 (d , J = 5.6 Hz, H), |
| | 1H-pyrazolo[3,4-d] | | | 3.71-3.64 (m, 1H), |
| | pyrimidin-4-amine | | | 1.20-1.17 (m, 2H), |
| | | | | 1.12-1.08 (m, 2H) |
| 170 | (cis)-3-(4-amino-3- | 366.30 | 367.1 | (Methanol-d₄) δ = 8.42 |
| | {[4-(trifluoromethyl) | | | (d, J = 5.2 Hz, 1H), 8.32 |
| | pyridin-2-yl]oxy}- | | | (s, 1H), 7.70 (s, 1H), |
| | 1H-pyrazolo[3,4-d] | | | 7.55 (d, J = 5.6 Hz, 1H) |
| | pyrimidin-1-yl) | | | 5.00-4.92 (m, 1H), |
| | cyclobutan-1-ol | | | 4.18-4.11 (m, 1H), |
| | | | | 2.86-2.81 (m, 2H), |
| | | | | 2.58-2.55 (m, 2H) |
| 171 | 3-(3-chlorophenoxy)-1- | 333.75 | 334 | (Methanol-d₄) δ = 8.18 (s, 1H), |
| | [(trans)-3-fluorocyclobutyl]- | | | 7.47 (d, J = 2.0 Hz, 1H), |
| | 1H-pyrazolo[3,4-d] | | | 7.42-7.40 (m, 1H), |
| | pyrimidin-4-amine | | | 7.37 (d, J = 15.6 Hz, 1H), |
| | | | | 7.23 (d, J = 8.4 Hz, 1H), |
| | | | | 5.51-5.47 (m, 1H), |
| | | | | 5.40-5.38 (m, 0.5H), |
| | | | | 5.26-5.24 (m, 0.5H), |
| | | | | 2.88-2.71 (m, 4H) |
| 172 | 3-({4-amino-1-[(trans)-3- | 322.34 | 323.1 | (Acetonitrile-d₃) δ = 8.23 |
| | fluorocyclobutyl]-1H- | | | (s, 1H), 7.65 (d, J = 7.6 Hz, |
| | pyrazolo[3,4-d] | | | 1H), 7.62-7.57 (m, 2H), |
| | pyrimidin-3-yl} | | | 7.55-7.47 (m, 1H), |
| | methyl)benzonitrile | | | 5.62-5.51 (m, 1.5H), |
| | | | | 5.45-5.35 (m, 0.5H), |
| | | | | 4.43 (s, 2H), |
| | | | | 3.01-2.76 (m, 4H) |
| 173 | 3-({4-amino-1-[(trans)-3- | 324.31 | 325.1 | (Methanol-d₄) δ = 8.18 |
| | fluorocyclobutyl]-1H- | | | (s, 1H), 7.83 (s, 1H), |
| | pyrazolo[3,4-d]pyrimidin- | | | 7.76 (dd, J = 2.4, 7.6 Hz, 1H), |
| | 3-yl}oxy)benzonitrile | | | 7.65-7.60 (m, 2H), |
| | | | | 5.51-5.47 (m, 1H), |
| | | | | 5.39-5.37 (m, 0.5H), |
| | | | | 5.25-5.22 (m, 0.5H), |
| | | | | 2.88-2.71 (m, 4H) |
| 174 | 2-({4-amino-1-[(trans)-3- | 343.32 | 344.1 | (Methanol-d₄) δ = 8.34 |
| | fluorocyclobutyl]-1H- | | | (d, J = 5.6Hz, 1H), |
| | pyrazolo[3,4-d] | | | 8.30 (s, 1H), 7.82 (s, 1H), |
| | pyrimidin-3-yl} | | | 7.66 (dd, J = 1.2. 5.2 Hz, 1H), |
| | oxy)pyridine-4- | | | 5.66-5.58 (m, 1H), |
| | carboxamide | | | 5.47-5.41 (m, 0.5H), |
| | | | | 5.33-5.27 (m, 0.5H), |
| | | | | 2.98-2.80 (m, 4H) |
| 175 | 3-[(4-methoxypyridin- | 330.32 | 331.1 | (Acetonitrile-d₃) δ = 8.22 (s, 1H), |
| | 2-yl)oxy]-1-[(trans)-3- | | | 7.95 (d, J = 6.4 Hz, 1H), |
| | fluorocyclobutyl]-1H- | | | 6.77-6.76 (m, 2H), |
| | pyrazolo[3,4-d] | | | 5.94 (br s, 2H), |
| | pyrimidin-4-amine | | | 5.55-5.48 (m, 1.5H), |
| | | | | 5.43-5.34 (m, 0.5H), , |
| | | | | 3.90 (s, 3H) |
| | | | | 2.91-2.77 (m, 4H) |
| 176 | 2-({4-amino-1-[(trans)-3- | 314.30 | 315 | (Methanol-d₄) δ = 8.31 (s, 1H), |
| | hydroxycyclobutyl]- | | | 7.97 (d, J = 5.6 Hz, 1H), |
| | 1H-pyrazolo[3,4-d] | | | 6.94 (d, J = 2.0 Hz, 1H), |
| | pyrimidin-3-yl)oxy) | | | 6.75 (dd, J = 5.6, 2.0 Hz, 1H), |
| | pyridin-4-ol | | | 5.58-5.51 (m, 1H), |
| | | | | 4.64-4.60 (m, 1H), |
| | | | | 2.86-2.81 (m, 2H), |
| | | | | 2.54-2.49 (m, 2H) |
| 177 | 2-({4-amino-1-[(trans)- | 316.29 | 317 | (Methanol-d₄) δ = 8.32 |
| | 3-fluorocyclobutyl]- | | | (s, 1H), 7.98 (d, J = 5.6 |
| | 1H-pyrazolo[3,4-d] | | | Hz, 1H), 6.94 (d, J = 2.0 |
| | pyrimidin-3-yl} | | | Hz, 1H), 6,75 (dd, J = |
| | oxy)pyridin-4-ol | | | 2.0, 6.0 Hz, 1H), 5.64- |
| | | | | 5.61 (m, 1H), 5.49-5.46 |
| | | | | (m, 1H), 5.33-5.32 (m, 1H), |
| | | | | 2.98-2.81 (m, 4H) |
| 178 | 3-((4-amino-1-((trans)- | 322.3 | 323.1 | (Methanol-d₄) δ = 8.28 |
| | 3-hydroxycyclobutyl)- | | | (s, 1H), 7.87 (d, J = 0.8 |
| | 1H-pyrazolo[3,4-d] | | | Hz, 1H), 7.82-7.78 (m, 1H), |
| | pyrimidin-3-yl)oxy) | | | 7.67-7.63 (m, 2H), |
| | benzonitrile | | | 5.51-5.43 (m, 1H), |
| | | | | 4.58-4.52 (m., 1H), |
| | | | | 2.80-2.72 (m, 2H), |
| | | | | 2.48 (ddd, J = 4.4, 8.8, |
| | | | | 13.2 Hz, 2H) |
| 179 | (trans)-3-(4-amino-3-(3- | 365.31 | 366.1 | (Methanol-d₄) δ = 8.28 |
| | (trifluoromethyl)phenoxy)- | | | (s, 1H), 7.84 (s, 1H), |
| | 1H-pyrazolo[3,4-d] | | | 7.77-7.72 (m, 1H), |
| | pyrimidin-1-yl) | | | 7.67 (t, J = 8.0 Hz, 1H), |
| | cyclobutan-1-ol | | | 7.61-7.57 (m, 1H), |
| | | | | 5.51-5.43 (m, 1H), |
| | | | | 4.59-4.52 (m, 1H), |
| | | | | 2.80-2.70 (m, 2H), |
| | | | | 2.48 (ddd, J = 4.4, 8.8, |
| | | | | 13.2 Hz, 2H) |
| 180 | 1-(4-amino-3-((4- | 394.1 | 395.1 | (Methanol-d₄) δ = 8.44 |
| | (trifluoromethyl) | | | (d, J = 5.2 Hz, 1H), |
| | pyridin-2-yl)oxy)- | | | 8.26 (s, 1H), 7.71 (s, 1H), |
| | 1H-pyrazolo[3,4-d] | | | 7.54 (d, J = 5.2 Hz, 1H), |
| | pyrimidin-1-yl) | | | 3.15-3.01 (m, 2H), |
| | cyclobutane-1- | | | 2.94 (m, 2H), |
| | carboxylic acid | | | 2.40-2.29 (m, 1H), |
| | | | | 2.16-2.04 (m, 1H) |
| 181 | ethyl 1-(4-amino-3-((4- | 422.13 | 423.1 | (Methanol-d₄) δ = 8.44 |
| | (trifluoromethyl)pyridin- | | | (d, J = 5.2 Hz, 1H), |
| | 2-yl)oxy)-1H- | | | 8.27 (s, 1H), 7.70 (s, 1H), |
| | pyrazolo[3,4-d] | | | 7.56-7.53 (m, 1H), |
| | pyrimidin-1-yl) | | | 4.19 (q, J = 7.2 Hz, 2H), |
| | cyclobutane- | | | 3.12-3.03 (m, 2H), |
| | 1-carboxylate | | | 2.96-2.88 (m, 2H), |
| | | | | 2.31 (m, 1H), |
| | | | | 2.17-2.04 (m, 1H), |
| | | | | 1.20 (t, J = 7.2 Hz, 3H) |
| 182 | 1-(1-(trifluoromethyl) | | | |
| | cyclobutyl)-3-((4- | | | |
| | (trifluoromethyl) | | | |
| | pyridin-2-yl)oxy)- | | | |
| | 1H-pyrazolo[3,4-d] | | | |
| | pyrimidin-4-amine | | | |
| 183 | 1-(1-trifluoromethyl) | | | |
| | cyclopropyl)-3-((4- | | | |
| | (trifluoromethyl) | | | |
| | pyridin-2-yl)oxy)- | | | |
| | 1H-pyrazolo[3,4-d] | | | |
| | pyrimidin-4-amine | | | |
| 184 | (trans)-3-{4-amino-3- | 351.36 | | (ACETONITRILE-d₃) δ = 9.27 |
| | [(5-fluoroindol-3-yl) | | | (s, 1H), 8.12 (s, 1H), |
| | methyl]-1H-pyrazolo | | | 7.40-7.36 (m, 1H), |
| | [3,4-d]pyrimidin-1- | | | 7.20 (s, 1H), |
| | yl)cyclobutan-1-ol | | | 7.15 (d, J = 10.4 Hz, 1H), |
| | | | | 6.92 (t, J = 9.2 Hz, 1H), |
| | | | | 5.59 (s, 1H), |
| | | | | 5.46-5.42 (m, 1H), |
| | | | | 4.70-4.67 (m, 1H), |
| | | | | 4.36 (s, 2H), 3.27 (s, 1H), |
| | | | | 2.87-2.82 (m, 2H), |
| | | | | 2.50-2.46 (m, 2H) |
| 246 | 1-(4,4-difluorocyclohexyl)- | 376.36 | 377 | (DMSO-d₆) δ = 8.32 |
| | 3-((4-methoxypyridin-2-yl) | | | (d, J = 6.0 Hz, 1H), |
| | oxy)-1H-pyrazolo[3,4-d] | | | 7.96 (d, J= 5.6 Hz, 1H), |
| | pyrimidin-4-amine | | | 6.83-6.80 (m, 2H), |
| | | | | 4.87 (s, 1H), 3.88 (s, 3H), |
| | | | | 2.15-1.99 (m, 8H) |

The following compounds may also be prepared according to the methods of the present disclosure:

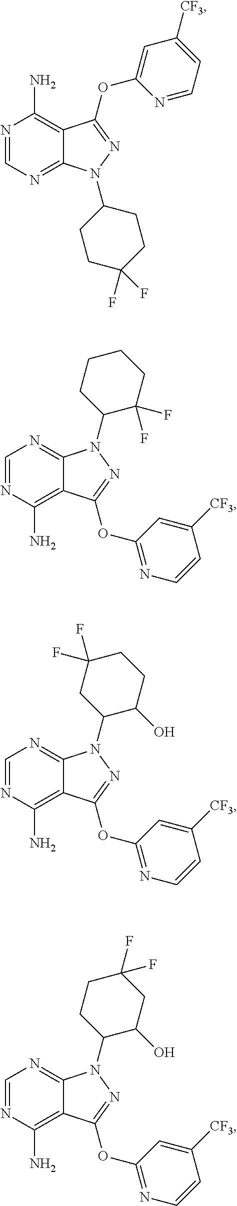 [SMILES: Nc1ncnc2c1c(Oc1cc(C(F)(F)F)ccn1)nn2C1CCC(F)(F)CC1 ; Nc1ncnc2c1c(Oc1cc(C(F)(F)F)ccn1)nn2C1CCCCC1(F)F ; Nc1ncnc2c1c(Oc1cc(C(F)(F)F)ccn1)nn2C1CC(F)(F)CCC1O ; Nc1ncnc2c1c(Oc1cc(C(F)(F)F)ccn1)nn2C1CCC(F)(F)CC1O]

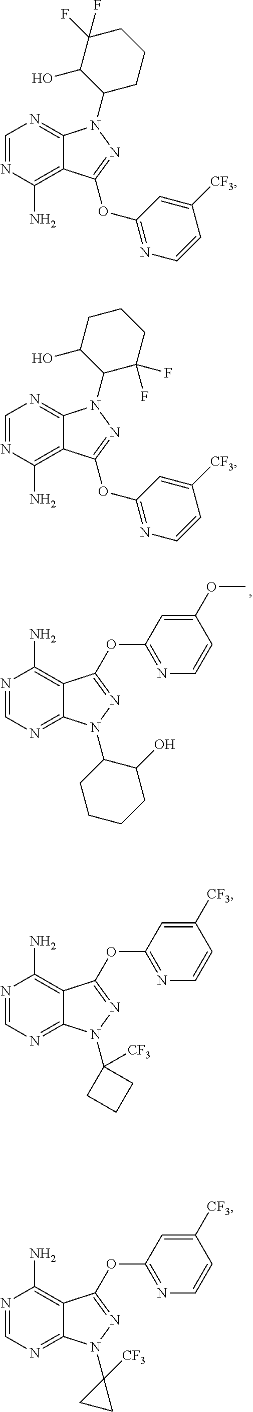 [SMILES: Nc1ncnc2c1c(Oc1cc(C(F)(F)F)ccn1)nn2C1CCCC(F)(F)C1O ; Nc1ncnc2c1c(Oc1cc(C(F)(F)F)ccn1)nn2C1C(O)CCCC1(F)F ; COc1ccnc(Oc2nn(C3CCCCC3O)c3ncnc(N)c23)c1 ; Nc1ncnc2c1c(Oc1cc(C(F)(F)F)ccn1)nn2C1(C(F)(F)F)CCC1 ; Nc1ncnc2c1c(Oc1cc(C(F)(F)F)ccn1)nn2C1(C(F)(F)F)CC1]

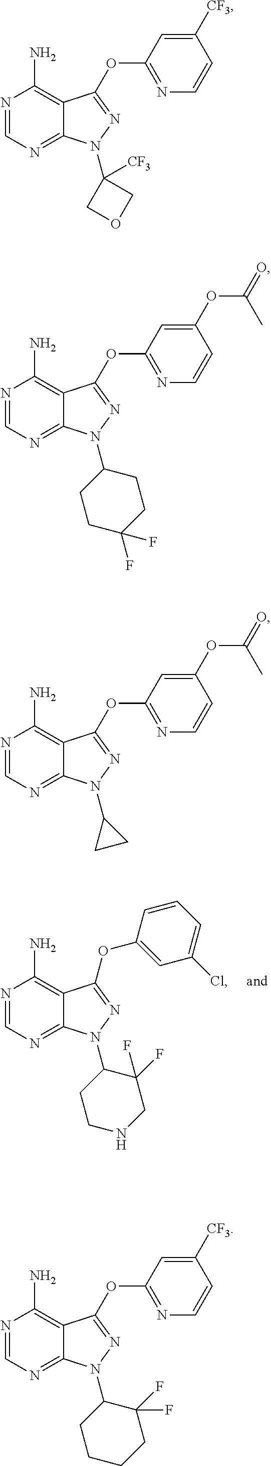 [SMILES: Nc1ncnc2c1c(Oc1cc(C(F)(F)F)ccn1)nn2C1(C(F)(F)F)COC1 ; CC(=O)Oc1ccnc(Oc2nn(C3CCC(F)(F)CC3)c3ncnc(N)c23)c1 ; CC(=O)Oc1ccnc(Oc2nn(C3CC3)c3ncnc(N)c23)c1 ; Nc1ncnc2c1c(Oc1cccc(Cl)c1)nn2C1CCNCC1(F)F ; "and" ; Nc1ncnc2c1c(Oc1cc(C(F)(F)F)ccn1)nn2C1CCCCC1(F)F]

### Example 3

Certain of the compounds prepared as described above were assayed to determine their IC₅₀ for inhibition of T. gondii CDPK1 (tgCDPK1). At least three independent replicates of the assay were conducted for each compound tested. The results are presented in Table 19 below. Compounds described herein that are selective for tgCDPK1 are expected to be selective for CDPK1 derived from the genera Plasmodium and Cryptosporidium as well.

TABLE 19
Potency of Exemplary Compounds against T. gondii CDPK1

| | | tgCDPK1 IC₅₀ |
|---|---|---|
| No. | Compound Name | (nM) |
| 12 | 3-(4-chlorobenzyl)-1-cyclopropyl- | 1830 |
| | 1H-pyrazolo[3,4-d]pyrimidin-4- | |
| | amine | |
| 23 | 3-(3-chlorobenzyl)-1-cyclopropyl- | 17.2 |
| | 1H-pyrazolo[3,4-d]pyrimidin-4- | |
| | amine | |
| 9 | 3-([1,1′-biphenyl]-3-ylmethyl)-1- | 26.3 |
| | cyclopropyl-1H-pyrazolo[3,4- | |
| | d]pyrimidin-4-amine | |
| 10 | 1-cyclopropyl-3-(3-(pyrimidin-5- | 373 |
| | yl)benzyl)-1H-pyrazolo[3,4- | |
| | d]pyrimidin-4-amine | |
| 11 | 1-cyclopropyl-3-(3-(pyridin-4- | 1170 |
| | yl)benzyl)-1H-pyrazolo[3,4- | |
| | d]pyrimidin-4-amine | |
| 4 | 3-(3-chloro-5-fluorobenzyl)-1- | 91.7 |
| | cyclopropyl-1H-pyrazolo[3,4- | |
| | d]pyrimidin-4-amine | |
| 5 | 1-cyclopropyl-3-(3-fluorobenzyl)- | 25.3 |
| | 1H-pyrazolo[3,4-d]pyrimdin-4- | |
| | amine | |
| 6 | 3-(3-chlorobenzyl)-1-cyclobutyl-1H- | 8.70 |
| | pyrazolo[3,4-d]pyrimidin-4-amine | |
| 16 | (4-amino-1-cyclopropyl-1H- | 1750 |
| | pyrazolo[3,4-d]pyrimidin-3- | |
| | yl)(phenyl)methanol | |
| 17 | 3-benzyl-1-cyclopropyl-1H- | 25.4 |
| | pyrazolo[3,4-d]pyrimidin-4-amine | |
| 18 | (4-amino-1-cyclopropyl-1H- | 4870 |
| | pyrazolo[3,4-d]pyrimidin-3- | |
| | yl)(pyridin-3-yl)methanol | |
| 107 | 3-((4-chloropyridin-2-yl)oxy)-1- | 6.16 |
| | cyclobutyl-1H-pyrazolo[3,4- | |
| | d]pyrimidin-4-amine | |
| 135 | 3-(3-chlorobenzyl)-1-((cis)-3- | 7.05 |
| | fluorocyclobutyl)-1H-pyrazolo[3,4- | |
| | d]pyrimidin-4-amine | |
| 121 | 3-(3-chlorophenoxy)-1-cyclobutyl- | 7.41 |
| | 1H-pyrazolo[3,4-d]pyrimidin-4- | |
| | amine | |
| 42 | 3-((4-chloropyridin-2-yl)oxy)-1- | 8.37 |
| | cyclopropyl-1H-pyrazolo[3,4- | |
| | d]pyrimidin-4-amine | |
| 124 | (trans)-3-(4-amino-3-(3- | 8.87 |
| | chlorobenzyl)-1H-pyrazolo[3,4- | |
| | d]pyrimidin-1-yl)cyclobutan-1-ol | |
| 109 | 1-cyclobutyl-3-((4- | 9.79 |
| | (trifluoromethyl)pyridin-2-yl)oxy)- | |
| | 1H-pyrazolo[3,4-d]pyrimidin-4- | |
| | amine | |
| 111 | 2-((4-amino-1-cyclobutyl-1H- | 10.2 |
| | pyrazolo[3,4-d]pyrimidin-3- | |
| | yl)oxy)isonicotinonitrile | |
| 131 | (cis)-3-(4-amino-3-(3-bromobenzyl)- | 11.4 |
| | 1H-pyrazolo[3,4-d]pyrimidin-1- | |
| | yl)cyclobutan-1-ol | |
| 154 | (trans)-3-(4-amino-3-(3- | 11.8 |
| | chlorophenoxy)-1H-pyrazolo[3,4- | |
| | d]pyrimidin-1-yl)cyclobutan-1-ol | |
| 112 | 1-cyclobutyl-3-((4-methoxypyridin- | 14.6 |
| | 2-yl)oxy)-1H-pyrazolo[3,4- | |
| | d]pyrimidin-4-amine | |
| 130 | (cis)-3-(4-amino-3-(3-chlorobenzyl)- | 15.1 |
| | 1H-pyrazolo[3,4-d]pyrimidin-1- | |
| | yl)cyclobutan-1-ol | |
| 116 | (trans)-3-(4-amino-3-((4- | 15.4 |
| | chloropyridin-2-yl)oxy)-1H- | |
| | pyrazolo[3,4-d]pyrimidin-1- | |
| | yl)cyclobutan-1-ol | |
| 77 | 1-cyclopropyl-3-((4- | 16.5 |
| | (trifluoromethyl)pyridin-2-yl)oxy)- | |
| | 1H-pyrazolo[3,4-d]pyrimidin-4- | |
| | amine | |
| 127 | (trans)-3-(4-amino-3-(3- | 18.4 |
| | bromobenzyl)-1H-pyrazolo[3,4- | |
| | d]pyrimidin-1-yl)cyclobutan-1-ol | |
| 54 | 1-cyclopropyl-3-((4-phenylpyridin-2- | 18.9 |
| | yl)oxy)-1H-pyrazolo[3,4- | |
| | d]pyrimidin-4-amine | |
| 83 | 1-cyclopropyl-3-((4-methylpyridin-2- | 20.6 |
| | yl)oxy)-1H-pyrazolo[3,4- | |
| | d]pyrimidin-4-amine | |
| 78 | 2-((4-amino-1-cyclopropyl-1H- | 21.7 |
| | pyrazolo[3,4-d]pyrimidin-3- | |
| | yl)oxy)isonicotinonitrile | |
| 88 | 1-cyclopropyl-3-((4-methoxypyridin- | 25.4 |
| | 2-yl)oxy)-1H-pyrazolo[3,4- | |
| | d]pyrimidin-4-amine | |
| 113 | (trans)-3-(4-amino-3-((4- | 26.5 |
| | (trifluoromethyl)pyridin-2-yl)oxy)- | |
| | 1H-pyrazolo[3,4-d]pyrimidin-1- | |
| | yl)cyclobutan-1-ol | |
| 152 | (cis)-3-(4-amino-3-(3- | 27.3 |
| | chlorophenoxy)-1H-pyrazolo[3,4- | |
| | d]pyrimidin-1-yl)cyclobutan-1-ol | |
| 128 | (trans)-3-(4-amino-3-(3- | 28.3 |
| | fluorobenzyl)-1H-pyrazolo[3,4- | |
| | d]pyrimidin-1-yl)cyclobutan-1-ol | |
| 132 | (cis)-3-(4-amino-3-(3-fluorobenzyl)- | 28.4 |
| | 1H-pyrazolo[3,4-d]pyrimidin-1- | |
| | yl)cyclobutan-1-ol | |
| 86 | 2-((4-amino-1-cyclopropyl-1H- | 29.6 |
| | pyrazolo[3,4-d]pyrimidin-3- | |
| | yl)oxy)pyridin-4-yl)methanol | |
| 151 | (cis)-3-(4-amino-3-(3- | 31.3 |
| | (trifluoromethyl)phenoxy)-1H- | |
| | pyrazolo[3,4-d]pyrimidin-1- | |
| | yl)cyclobutan-1-ol | |
| 153 | (cis)-3-(4-amino-3-(3- | 33.8 |
| | fluorophenoxy)-1H-pyrazolo[3,4- | |
| | d]pyrimidin-1-yl)cyclobutan-1-ol | |
| 125 | (trans)-3-(4-amino-3-(3- | 36.0 |
| | (trifluoromethyl)benzyl)-1H- | |
| | pyrazolo[3,4-d]pyrimidin-1- | |
| | yl)cyclobutan-1-ol | |
| 38 | 1-cyclopropyl-3-(pyridin-2-yloxy)- | 37.6 |
| | 1H-pyrazolo[3,4-d]pyrimidin-4- | |
| | amine | |
| 155 | (trans)-3-(4-amino-3-(3- | 38.0 |
| | fluorophenoxy)-1H-pyrazolo[3,4- | |
| | d]pyrimidin-1-yl)cyclobutan-1-ol | |
| 156 | 3-(3-chlorophenoxy)-1-[(1r,3r)-3- | 10.0 |
| | fluorocyclobutyl]-1H-pyrazolo[3,4- | |
| | d]pyrimidin-4-amine | |
| 157 | 3-[(5-fluoroindol-3-yl)methyl]-1- | 11.0 |
| | [(trans)-3-fluorocyclobutyl]-1H- | |
| | pyrazolo[3,4-d]pyrimidin-4-amine | |
| 158 | 3-[(4-chloropyridin-2-yl)oxy]-1- | 11.4 |
| | [(trans)-3-fluorocyclobutyl]-1H- | |
| | pyrazolo[3,4-d]pyrimidin-4-amine | |
| 159 | 1-cyclopropyl-3-{[4- | 13.0 |
| | (trifluoromethyl)pyridin-2-yl]oxy}- | |
| | 1H-pyrazolo[3,4-d]pyrimidin-4- | |
| | amine | |
| 160 | 2-({4-amino-1-cyclopropyl-1H- | 13.6 |
| | pyrazolo[3,4-d]pyrimidin-3- | |
| | yl}oxy)pyridin-4-ol | |
| 161 | 3-[(3-fluorophenyl)methyl]-1- | 14.2 |
| | [(trans)-3-fluorocyclobutyl]-1H- | |
| | pyrazolo[3,4-d]pyrimidin-4-amine | |
| 162 | 3-[(4-methoxypyridin-2-yl)oxy]-1- | 17.4 |
| | [(trans)-3-fluorocyclobutyl]-1H- | |
| | pyrazolo[3,4-d]pyrimidin-4-amine | |
| 163 | (cis)-3-{4-amino-3-[(4-chloropyridin- | 18.0 |
| | 2-yl)oxy]-1H-pyrazolo[3,4- | |
| | d]pyrimidin-1-yl}cyclobutan-1-ol | |
| 164 | (trans)-3-{4-amino-5-[(3- | 28.3 |
| | fluorophenyl)methyl]-7H- | |
| | pyrrolo[2,3-d]pyrimidin-7- | |
| | yl}cyclobutan-1-ol | |
| 165 | 1-cyclopropyl-3-[(4- | 28.9 |
| | cyclopropylpyridin-2-yl)oxy]-1H- | |
| | pyrazolo[3,4-d]pyrimidin-4-amine | |
| 166 | 1-[(trans)-3-fluorocyclobutyl]-3-[3- | 28.9 |
| | (trifluoromethyl)phenoxy]-1H- | |
| | pyrazolo[3,4-d]pyrimidin-4-amine | |
| 167 | 3-(3-fluorophenoxy)-1-[(trans)-3- | 36.4 |
| | fluorocyclobutyl]-1H-pyrazolo[3,4- | |
| | d]pyrimidin-4-amine | |
| 168 | (cis)-3-{4-amino-3-[(4- | 37.1 |
| | methoxypyridin-2-yl)oxy]-1H- | |
| | pyrazolo[3,4-d]pyrimidin-1- | |
| | yl}cyclobutan-1-ol | |
| 169 | 1-cyclopropyl-3-{[4- | 37.7 |
| | (trifluoromethoxy)pyridin-2-yl]oxy}- | |
| | 1H-pyrazolo[3,4-d]pyrimidin-4- | |
| | amine | |
| 170 | (cis)-3-(4-amino-3-{[4- | 38.6 |
| | (trifluoromethyl)pyridin-2-yl]oxy}- | |
| | 1H-pyrazolo[3,4-d]pyrimidin-1- | |
| | yl)cyclobutan-1-ol | |
| 171 | 3-(3-chlorophenoxy)-1-[(trans)-3- | 41.1 |
| | fluorocyclobutyl]-1H-pyrazolo[3,4- | |
| | d]pyrimidin-4-amine | |
| 172 | 3-({4-amino-1-[(trans)-3- | 45.9 |
| | fluorocyclobutyl]-1H-pyrazolo[3,4- | |
| | d]pyrimidin-3-yl}methyl)benzonitrile | |
| 173 | 3-({4-amino-1-[(trans)-3- | 49.8 |
| | fluorocyclobutyl]-1H-pyrazolo[3,4- | |
| | d]pyrimidin-3-yl}oxy)benzonitrile | |
| 174 | 2-({4-amino-1-[(trans)-3- | 69.7 |
| | fluorocyclobutyl]-1H-pyrazolo[3,4- | |
| | d]pyrimidin-3-yl}oxy)pyridine-4- | |
| | carboxamide | |
| 175 | 3-[(4-methoxypyridin-2-yl)oxy]-1- | 17.4 |
| | [(trans)-3-fluorocyclobutyl]-1H- | |
| | pyrazolo[3,4-d]pyrimidin-4-amine | |
| 178 | 3-((4-amino-1-((trans)-3- | 79.6 |
| | hydroxycyclobutyl)-1H-pyrazolo[3,4- | |
| | d]pyrimidin-3-yl)oxy)benzonitrile | |
| 179 | (trans)-3-(4-amino-3-(3- | 47.9 |
| | (trifluoromethyl)phenoxy)-1H- | |
| | pyrazolo[3,4-d]pyrimidin-1- | |
| | yl)cyclobutan-1-ol | |
| 184 | (trans)-3-{4-amino-3-[(5-fluoroindol- | 10.2 |
| | 3-yl)methyl]-1H-pyrazolo[3,4- | |
| | d]pyrimidin-1-yl}cyclobutan-1-ol | |
| 185 | 1-(4,4-difluorocyclohexyl)-3-((4- | 38.4 |
| | (trifluoromethyl)pyridin-2-yl)oxy)- | |
| | 1H-pyrazolo[3,4-d]pyrimidin-4- | |
| | amine | |
| 186 | 1-(cyclopropylmethyl)-3-((4- | 22.4 |
| | (trifluoromethyl)pyridin-2-yl)oxy)- | |
| | 1H-pyrazolo[3,4-d]pyrimidin-4- | |
| | amine | |
| 187 | 2-((4-amino-1-(4,4- | 10 |
| | difluorocyclohexyl)-1H- | |
| | pyrazolo[3,4-d]pyrimidin-3- | |
| | yl)oxy)pyridin-4-ol | |
| 188 | 3-((4-chloropyridin-2-yl)oxy)-1- | 29.7 |
| | (piperidin-4-yl)-1H-pyrazolo[3,4- | |
| | d]pyrimidin-4-amine | |
| 189 | 3-((4-chloropyridin-2-yl)oxy)-1- | ~24 |
| | (piperidin-3-yl)-1H-pyrazolo[3,4- | |
| | d]pyrimidin-4-amine | |
| 190 | 1-(5,5-difluoropiperidin-3-yl)-3-((4- | ~54 |
| | (trifluoromethyl)pyridin-2-yl)oxy)- | |
| | 1H-pyrazolo[3,4-d]pyrimidin-4- | |
| | amine | |
| 191 | (trans)-2-(4-amino-3-((4- | 25 |
| trans | (trifluoromethyl)pyridin-2-yl)oxy)- | |
| (rac) | 1H-pyrazolo[3,4-d]pyrimidin-1- | |
| | yl)cyclohexanol, racemic | |
| 191 | (1R,2R)-2-(4-amino-3-((4- | 8.4 |
| (R,R) | (trifluoromethyl)pyridin-2-yl)oxy)- | |
| | 1H-pyrazolo[3,4-d]pyrimidin-1- | |
| | yl)cyclohexanol | |
| 191 | (1S,2S)-2-(4-amino-3-((4- | 125 |
| (S,S) | (trifluoromethyl)pyridin-2-yl)oxy)- | |
| | 1H-pyrazolo[3,4-d]pyrimidin-1- | |
| | yl)cyclohexanol | |
| 191 | (cis)-2-(4-amino-3-((4- | 44.0 |
| cis | (trifluoromethyl)pyridin-2-yl)oxy)- | |
| (rac) | 1H-pyrazolo[3,4-d]pyrimidin-1- | |
| | yl)cyclohexanol (racemic) | |
| 247 | 3-(3-chlorophenoxy)-1-(1H-pyrrol-3- | 19.4 |
| | ylmethyl)pyrazolo[3,4-d]pyrimidin- | |
| | 4-amine | |
| 244 | 1-((1- | 31.0 |
| | (trifluoromethyl)cyclopropyl)methyl)- | |
| | 3-((4-(trifluoromethyl)pyridin-2- | |
| | yl)oxy)-1H-pyrazolo[3,4- | |
| | d]pyrimidin-4-amine | |
| 248 | 1-((3R,5R)-5-aminotetrahydro-2H- | 60.7 |
| | pyran-3-yl)-3-((4- | |
| | (trifluoromethyl)pyridin-2-yl)oxy)- | |
| | 1H-pyrazolo[3,4-d]pyrimidin-4- | |
| | amine | |
| 249 | 1-((3R,5S)-5-aminotetrahydro-2H- | 55.0 |
| | pyran-3-yl)-3-((4- | |
| | (trifluoromethyl)pyridin-2-yl)oxy)- | |
| | 1H-pyrazolo[3,4-d]pyrimidin-4- | |
| | amine | |
| 243 | ((trans)-4-(4-amino-3-((4- | 10.3 |
| | (trifluoromethyl)pyridin-2-yl)oxy)- | |
| | 1H-pyrazolo[3,4-d]pyrimidin-1- | |
| | yl)cyclohexyl)methanol | |
| 242 | ((cis)-4-(4-amino-3-((4- | 15.1 |
| | (trifluoromethyl)pyridin-2-yl)oxy)- | |
| | 1H-pyrazolo[3,4-d]pyrimidin-1- | |
| | yl)cyclohexyl)methanol | |
| 250 | (1-((4-amino-3-(3-chlorophenoxy)- | 34.9 |
| | 1H-pyrazolo[3,4-d]pyrimidin-1- | |
| | yl)methyl)cyclopropyl)methanol | |
| 240 | tert-butyl ((1S,3R)-3-(4-amino-3-((4- | 12.1 |
| | (trifluoromethyl)pyridin-2-yl)oxy)- | |
| | 1H-pyrazolo[3,4-d]pyrimidin-1- | |
| | yl)cyclopentyl)carbamate | |
| 251 | 1-((1R,3S)-3-aminocyclopentyl)-3- | 42.1 |
| | ((4-(trifluoromethyl)pyridin-2- | |
| | yl)oxy)-1H-pyrazolo[3,4- | |
| | d]pyrimidin-4-amine | |
| 239 | 1-((cis)-4-aminocyclohexyl)-3-((4- | 23.1 |
| | (trifluoromethyl)pyridin-2-yl)oxy)- | |
| | 1H-pyrazolo[3,4-d]pyrimidin-4- | |
| | amine | |
| 238 | 1-((cis)-4-methoxycyclohexyl)-3-((4- | 44.7 |
| | (trifluoromethyl)pyridin-2-yl)oxy)- | |
| | 1H-pyrazolo[3,4-d]pyrimidin-4- | |
| | amine | |
| 237 | 1-((trans)-4-methoxycyclohexyl)-3- | 38.1 |
| | ((4-(trifluoromethyl)pyridin-2- | |
| | yl)oxy)-1H-pyrazolo[3,4- | |
| | d]pyrimidin-4-amine | |
| 252 | 3-(3-chlorophenoxy)-1H- | 77.3 |
| | pyrazolo[3,4-d]pyrimidin-4-amine | |
| 236 | 1-(2-methoxycyclohexyl)-3-((4- | 85.6 |
| | (trifluoromethyl)pyridin-2-yl)oxy)- | |
| | 1H-pyrazolo[3,4-d]pyrimidin-4- | |
| | amine | |
| 235 | (trans)-4-(4-amino-3-((4- | 15.9 |
| | (trifluoromethyl)pyridin-2-yl)oxy)- | |
| | 1H-pyrazolo[3,4-d]pyrimidin-1- | |
| | yl)cyclohexanol | |
| 234 | 1-(2-azabicyclo[2.2.1]heptan-6-yl)-3- | 76.7 |
| | ((4-(trifluoromethyl)pyridin-2- | |
| | yl)oxy)-1H-pyrazolo[3,4- | |
| | d]pyrimidin-4-amine | |
| 233 | 1-((1S,2R)-2-fluorocyclohexyl)-3- | 34.4 |
| | ((4-(trifluoromethyl)pyridin-2- | |
| | yl)oxy)-1H-pyrazolo[3,4- | |
| | d]pyrimidin-4-amine | |
| 232 | 1-(4-aminocyclohexyl)-3-((4- | 24.3 |
| | (trifluoromethyl)pyridin-2-yl)oxy)- | |
| | 1H-pyrazolo[3,4-d]pyrimidin-4- | |
| | amine | |
| 231 | (cis)-4-(4-amino-3-((4- | 18.6 |
| | (trifluoromethyl)pyridin-2-yl)oxy)- | |
| | 1H-pyrazolo[3,4-d]pyrimidin-1- | |
| | yl)cyclohexanol | |
| 230 | 3-(4-amino-3-((4- | 24.2 |
| | (trifluoromethyl)pyridin-2-yl)oxy)- | |
| | 1H-pyrazolo[3,4-d]pyrimidin-1- | |
| | yl)cyclopentanol | |
| 253 | 1-(tert-butyl)-3-(3-chlorophenoxy)- | 10 |
| | 1H-pyrazolo[3,4-d]pyrimidin-4- | |
| | amine | |
| 254 | 3-((1H-indol-3-yl)methyl)-1-(tert- | 9 |
| | butyl)-1H-pyrazolo[3,4-d]pyrimidin- | |
| | 4-amine | |
| 255 | 3-((4-amino-1-(tert-butyl)-1H- | 13.4 |
| | pyrazolo[3,4-d]pyrimidin-3- | |
| | yl)methyl)benzonitrile | |
| 256 | 1-(tert-butyl)-3-(m-tolyloxy)-1H- | 13.6 |
| | pyrazol[3,4-d]pyrimidin-4-amine | |
| 257 | 1-cyclopentyl-3-((4- | 23.4 |
| | (trifluoromethyl)pyridin-2-yl)oxy)- | |
| | 1H-pyrazolo[3,4-d]pyrimidin-4- | |
| | amine | |
| 193 | 1-(tetrahydrofuran-3-yl)-3-((4- | 47.7 |
| | (trifluoromethyl)pyridin-2-yl)oxy)- | |
| | 1H-pyrazolo[3,4-d]pyrimidin-4- | |
| | amine | |
| 194 | 1-(piperidin-4-yl)-3-((4- | 67.8 |
| | (trifluoromethyl)pyridin-2-yl)oxy)- | |
| | 1H-pyrazolo[3,4-d]pyrimidin-4- | |
| | amine | |
| 195 | 1-(piperidin-3-yl)-3-((4- | 56.8 |
| | (trifluoromethyl)pyridin-2-yl)oxy)- | |
| | 1H-pyrazolo[3,4-d]pyrimidin-4- | |
| | amine | |
| 196 | tert-butyl 4-((4-amino-3-((4- | 64.8 |
| | (trifluoromethyl)pyridin-2-yl)oxy)- | |
| | 1H-pyrazolo[3,4-d]pyrimidin-1- | |
| | yl)methyl)-3,3-difluoropiperidine-1- | |
| | carboxylate | |
| 197 | 1-((3,3-difluoropiperidin-4- | 13.8 |
| | yl)methyl)-3-((4- | |
| | (trifluoromethyl)pyridin-2-yl)oxy)- | |
| | 1H-pyrazolo[3,4-d]pyrimidin-4- | |
| | amine | |
| 198 | 1-(tetrahydro-2H-pyran-4-yl)-3-((4- | 29.4 |
| | (trifluoromethyl)pyridin-2-yl)oxy)- | |
| | 1H-pyrazolo[3,4-d]pyrimidin-4- | |
| | amine | |
| 258 | 1-(tert-butyl)-3-((4- | 13.9 |
| | (trifluoromethyl)pyridin-2-yl)oxy)- | |
| | 1H-pyrazolo[3,4-d]pyrimidin-4- | |
| | amine | |
| 259 | 2-((4-amino-1-(tert-butyl)-1H- | 18.1 |
| | pyrazolo[3,4-d]pyrimidin-3- | |
| | yl)oxy)isonicotinonitrile | |
| 260 | 2-((4-amino-1-(tert-butyl)-1H- | 40.5 |
| | pyrazolo[3,4-d]pyrimidin-3- | |
| | yl)oxy)isonicotinamide | |
| 199 | tert-butyl 3-(4-amino-3-((4- | 38.9 |
| | (trifluoromethyl)pyridin-2-yl)oxy)- | |
| | 1H-pyrazolo[3,4-d]pyrimidin-1- | |
| | yl)pyrrolidine-1-carboxylate | |
| 200 | 1-cyclohexyl-3-((4- | 14.7 |
| | (trifluoromethyl)pyridin-2-yl)oxy)- | |
| | 1H-pyrazolo[3,4-d]pyrimidin-4- | |
| | amine | |
| 201 | 3-((4-chloropyridin-2-yl)oxy)-1- | 16.7 |
| | cyclopentyl-1H-pyrazolo[3,4- | |
| | d]pyrimidin-4-amine | |
| 202 | 3-((4-chloropyridin-2-yl)oxy)-1- | 55.7 |
| | (tetrahydrofuran-3-yl)-1H- | |
| | pyrazolo[3,4-d]pyrimidin-4-amine | |
| 261 | 3-((4-chloropyridin-2-yl)oxy)-1-(4,4- | 35.3 |
| | difluorocyclohexyl)-1H- | |
| | pyrazolo[3,4-d]pyrimidin-4-amine | |
| 262 | 1-(tert-butyl)-3-((4-chloropyridin-2- | 12.0 |
| | yl)oxy)-1H-pyrazolo[3,4- | |
| | d]pyrimidin-4-amine | |
| 192 | 1-cyclopentyl-3-((4- | 19.2 |
| | (trifluoromethyl)pyridin-2-yl)oxy)- | |
| | 1H-pyrazolo[3,4-d]pyrimidin-4- | |
| | amine | |
| 203 | 2-((4-amino-1-(4,4- | 84.8 |
| | difluorocyclohexyl)-1H- | |
| | pyrazolo[3,4-d]pyrimidin-3- | |
| | yl)oxy)isonicotinonitrile | |
| 204 | 2-((4-amino-1-cyclopentyl-1H- | 67.0 |
| | pyrazolo[3,4-d]pyrimidin-3- | |
| | yl)oxy)isonicotinamide | |
| 205 | 2-((4-amino-1-cyclohexyl-1H- | 26.8 |
| | pyrazolo[3,4-d]pyrimidin-3- | |
| | yl)oxy)isonicotinamide | |
| 206 | 2-((4-amino-1-(4,4- | 96.8 |
| | difluorocyclohexyl)-1H- | |
| | pyrazolo[3,4-d]pyrimidin-3- | |
| | yl)oxy)isonicotinamide | |
| 207 | 2-((4-amino-1-((cis)-4- | 23.7 |
| | (trifluoromethyl)cyclohexyl)-1H- | |
| | pyrazolo[3,4-d]pyrimidin-3- | |
| | yl)oxy)isonicotinonitrile | |
| 208 | 2-((4-amino-1-((cis)-4- | 90.1 |
| | (trifluoromethyl)cyclohexyl)-1H- | |
| | pyrazolo[3,4-d]pyrimidin-3- | |
| | yl)oxy)isonicotinamide | |
| 209 | 2-((4-amino-1-((trans)-4- | 26.0 |
| | (trifluoromethyl)cyclohexyl)-1H- | |
| | pyrazolo[3,4-d]pyrimidin-3- | |
| | yl)oxy)isonicotinonitrile | |
| 263 | 2-((4-amino-1-((3,3- | 39.2 |
| | difluoropiperidin-4-yl)methyl)-1H- | |
| | pyrazolo[3,4-d]pyrimidin-3- | |
| | yl)oxy)isonicotinonitrile | |
| 212 | tert-butyl 4-((4-amino-3-((4- | 31.2 |
| | chloropyridin-2-yl)oxy)-1H- | |
| | pyrazolo[3,4-d]pyrimidin-1- | |
| | yl)methyl)-3,3-difluoropiperidine-1- | |
| | carboxylate | |
| 213 | 3-((4-chloropyridin-2-yl)oxy)-1- | 9.7 |
| | ((3,3-difluoropiperidin-4-yl)methyl)- | |
| | 1H-pyrazolo[3,4-d]pyrimidin-4- | |
| | amine | |
| 214 | 3-((4-chloropyridin-2-yl)oxy)-1- | 4.6 |
| | cyclohexyl-1H-pyrazolo[3,4- | |
| | d]pyrimidin-4-amine | |
| 215 | 2-((4-amino-1-cyclohexyl-1H- | 12.3 |
| | pyrazolo[3,4-d]pyrimidin-3- | |
| | yl)oxy)isonicotinonitrile | |
| 216 | 3-((4-chloropyridin-2-yl)oxy)-1- | 15.8 |
| | (tetrahydro-2H-pyran-3-yl)-1H- | |
| | pyrazolo[3,4-d]pyrimidin-4-amine | |
| 219 | 3-((4-methoxypyridin-2-yl)oxy)-1- | 77.4 |
| | (tetrahydrofuran-3-yl)-1H- | |
| | pyrazolo[3,4-d]pyrimidin-4-amine | |
| 220 | 3-((4-methoxypyridin-2-yl)oxy)-1- | 54.4 |
| | (tetrahydro-2H-pyran-4-yl)-1H- | |
| | pyrazolo[3,4-d]pyrimidin-4-amine | |
| 221 | 2-((4-amino-1-(cyclopropylmethyl)- | 24.1 |
| | 1H-pyrazolo[3,4-d]pyrimidin-3- | |
| | yl)oxy)isonicotinonitrile | |
| 222 | 3-((4-chloropyridin-2-yl)oxy)-1- | 24.6 |
| | (piperidin-3-yl)-1H-pyrazolo[3,4- | |
| | d]pyrimidin-4-amine | |
| 223 | 1-(cyclopropylmethyl)-3-((4- | 25.1 |
| | methoxypyridin-2-yl)oxy)-1H- | |
| | pyrazolo[3,4-d]pyrimidin-4-amine | |
| 224 | tert-butyl 3-(4-amino-3-((4- | 43.7 |
| | chloropyridin-2-yl)oxy)-1H- | |
| | pyrazolo[3,4-d]pyrimidin-1- | |
| | yl)piperidine-1-carboxylate | |
| 225 | 3-((4-chloropyridin-2-yl)oxy)-1- | 9.0 |
| | (cyclopropylmethyl)-1H- | |
| | pyrazolo[3,4-d]pyrimidin-4-amine | |
| 264 | 2-((4-amino-1-(tert-butyl)-1H- | 13.9 |
| | pyrazolo[3,4-d]pyrimidin-3- | |
| | yl)oxy)pyridin-4-ol | |
| 265 | 3-(4-amino-3-(3-chlorophenoxy)-1H- | 47.9 |
| | pyrazolo[3,4-d]pyrimidin-1-yl)-2,2- | |
| | dimethylpropan-1-ol | |
| 227 | 1-(4-(dimethylamino)cyclohexyl)-3- | 28.5 |
| | ((4-(trifluoromethyl)pyridin-2- | |
| | yl)oxy)-1H-pyrazolo[3,4- | |
| | d]pyrimidin-4-amine | |
| 228 | methyl 4-(4-amino-3-((4- | 25.4 |
| | (trifluoromethyl)pyridin-2-yl)oxy)- | |
| | 1H-pyrazolo[3,4-d]pyrimidin-1- | |
| | yl)cyclohexanecarboxylate | |
| 266 | 1-(trans-4- | 7.2 |
| | (methylamino)cyclohexyl)-3-((4- | |
| | (trifluoromethyl)pyridin-2-yl)oxy)- | |
| | 1H-pyrazolo[3,4-d]pyrimidin-4- | |
| | amine | |
| 267 | 1-(cis-4-(methylamino)cyclohexyl)- | 6.3 |
| | 3-((4-(trifluoromethyl)pyridin-2- | |
| | yl)oxy)-1H-pyrazolo[3,4- | |
| | d]pyrimidin-4-amine | |
| 268 | 1-(4-amino-3-((4- | 11.7 |
| | (trifluoromethyl)pyridin-2-yl)oxy)- | |
| | 1H-pyrazolo[3,4-d]pyrimidin-1- | |
| | yl)but-3-en-2-ol | |
| 269 | 5-((4-amino-3-((4- | 28.6 |
| | (trifluoromethyl)pyridin-2-yl)oxy)- | |
| | 1H-pyrazolo[3,4-d]pyrimidin-1- | |
| | yl)methyl)-1,2-oxaborolan-2-ol | |
| 270 | (3-((4-amino-3-((4- | 28.7 |
| | (trifluoromethyl)pyridin-2-yl)oxy)- | |
| | 1H-pyrazolo[3,4-d]pyrimidin-1- | |
| | yl)methyl)cyclobutyl)boronic acid | |
| 271 | 1-(3,3-difluoro-4-piperidyl)-3-[[4- | 19.8 |
| | (trifluoromethyl)-2- | |
| | pyridyl]oxy]pyrazolo[3,4- | |
| | d]pyrimidin-4-amine | |
| 272 | 1-(1-(trifluoromethyl)cyclobutyl)-3- | 20.7 |
| | ((4-(trifluoromethyl)pyridin-2- | |
| | yl)oxy)-1H-pyrazolo[3,4- | |
| | d]pyrimidin-4-amine | |
| 273 | 3-[[4-amino-3-[[4-(trifluoromethyl)- | 35.8 |
| | 2-pyridyl]oxy]pyrazolo[3,4- | |
| | d]pyrimidin-1- | |
| | yl]methyl]cyclobutanol | |
| 274 | 2-[4-amino-3-[[4-(trifluoromethyl)-2- | 35.0 |
| | pyridyl]oxy]pyrazolo[3,4- | |
| | d]pyrimidin-1-yl]-4,4-difluoro- | |
| | cyclohexanol | |
| 275 | [2-(4-amino-1-cyclopropyl- | 16.1 |
| | pyrazolo[3,4-d]pyrimidin-3-yl)oxy-4- | |
| | pyridyl]acetate | |
| 276 | 1-(2-fluorocyclohexen-1-yl)-3-[[4- | 18.6 |
| | (trifluoromethyl)-2- | |
| | pyridyl]oxy]pyrazolo[3,4- | |
| | d]pyrimidin-4-amine and 1-(2- | |
| | fluorocyclohex-2-en-1-yl)-3-((4- | |
| | (trifluoromethyl)pyridin-2-yl)oxy)- | |
| | 1H-pyrazolo[3,4-d]pyrimidin-4- | |
| | amine | |
| 277 | [2-[4-amino-1-(4,4- | 21.5 |
| | difluorocyclohexyl)pyrazolo[3,4- | |
| | d]pyrimidin-3-yl]oxy-4-pyridyl] | |
| | acetate | |
| 278 | 2-((4-amino-1-((1S,2S)-2- | 18.9 |
| | hydroxycyclohexyl)-1H- | |
| | pyrazolo[3,4-d]pyrimidin-3- | |
| | yl)oxy)pyridin-4-ol | |
| 279 | 1-(2,2-difluorocyclohexyl)-3-[[4- | 35.7 |
| | (trifluoromethyl)-2- | |
| | pyridyl]oxy]pyrazolo[3,4- | |
| | d]pyrimidin-4-amine | |
| 280 | 1-(2,2,2-trifluoroethyl)-3-((4- | 38.6 |
| | (trifluoromethyl)pyridin-2-yl)oxy)- | |
| | 1H-pyrazolo[3,4-d]pyrimidin-4- | |
| | amine | |
| 281 | (1R,2R)-2-(4-amino-3-((4- | 26.2 |
| | methoxypyridin-2-yl)oxy)-1H- | |
| | pyrazolo[3,4-d]pyrimidin-1- | |
| | yl)cyclohexan-1-ol | |
| 282 | (1R,2S)-2-(4-amino-3-((4- | 41.3 |
| | methoxypyridin-2-yl)oxy)-1H- | |
| | pyrazolo[3,4-d]pyrimidin-1- | |
| | yl)cyclohexanol | |
| 283 | trans-2-((4-amino-1-(2- | 147.2 |
| | hydroxycyclohexyl)-1H- | |
| | pyrazolo[3,4-d]pyrimidin-3- | |
| | yl)oxy)pyridin-4-ol (single | |
| | enantiomer) | |
| 284 | cis-2-((4-amino-1-(2- | 39.2 |
| | hydroxycyclohexyl)-1H- | |
| | pyrazolo[3,4-d]pyrimidin-3- | |
| | yl)oxy)pyridin-4-ol (single | |
| | enantiomer) | |
| 285 | trans-2-(4-amino-3-((4- | 1403.5 |
| | methoxypyridin-2-yl)oxy)-1H- | |
| | pyrazolo[3,4-d]pyrimidin-1- | |
| | yl)cyclohexan-1-ol (single | |
| | enantiomer) | |
| 286 | cis-2-(4-amino-3-((4- | 144.3 |
| | methoxypyridin-2-yl)oxy)-1H- | |
| | pyrazolo[3,4-d]pyrimidin-1- | |
| | yl)cyclohexan-1-ol (single | |
| | enantiomer) | |
| 287 | ethyl 1-[4-amino-3-[[4- | 728.3 |
| | (trifluoromethyl)-2- | |
| | pyridyl]oxy]pyrazolo[3,4- | |
| | d]pyrimidin-1- | |
| | yl]cyclobutanecarboxylate | |
| 288 | 1-[4-amino-3-[[4-(trifluoromethyl)-2- | 11880.0 |
| | pyridyl]oxy]pyrazolo[3,4- | |
| | d]pyrimidin-1- | |
| | yl]cyclobutanecarboxylic acid | |
| 289 | 2-[4-amino-3-[[4-(trifluoromethyl)-2- | 98.5 |
| | pyridyl]oxy]pyrazolo[3,4- | |
| | d]pyrimidin-1-yl]cyclohexanone | |
| 290 | 2-[4-amino-1-(3- | 8.7 |
| | fluorocyclobutyl)pyrazolo[3,4- | |
| | d]pyrimidin-3-yl]oxypyridin-4-ol | |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound having the structure of formula (I) or a pharmaceutically acceptable salt thereof: 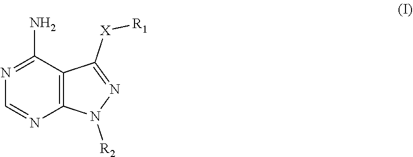
wherein:
X is R⁶ or O;
R¹ is phenyl or 5-10 membered heteroaryl;
R² is C₅₋₇ cycloalkyl, 4-7 membered heterocyclyl, C₁₋₆ alkyl, heteroaralkyl, carbocyclylalkyl, heterocyclylalkyl, C₂₋₆ alkenyl, C₄₋₆ cycloalkenyl, or H; and
R⁶ is C₁₋₆ alkylene.

2. The compound of claim 1, wherein X is R⁶.

3. The compound of claim 1, wherein X is O.

4. The compound of any one of claims 1-3, wherein R¹ is unsubstituted.

5. The compound of any one of claims 1-3, wherein R¹ is substituted with one or mote R⁵, and wherein each R⁵ is independently selected from alkyl, C₁₋₃ haloalkyl, halo, hydroxyl, alkoxy, cyano, acyloxy, and amide.

6. The compound of claim 5, wherein each R⁵ is independently selected from C₁₋₃ alkyl, C₁₋₃ alkoxy, trifluoromethyl, cyano, and halo.

7. The compound of claim 6, wherein each R⁵ is independently selected from methyl, trifluoromethyl, chloro, and fluoro.

8. The compound of any one of claims 5-7, wherein no R⁵ is fluoro.

9. The compound of claim 5, wherein at least one R⁵ is fluoro.

10. The compound of any one of claims 1-9, wherein R¹ is phenyl, pyridyl, or indolyl.

11. The compound of any one of claims 1-10, wherein R¹ is indolyl.

12. The compound of any one of claims 1-10, wherein R¹ is phenyl, substituted at the meta-position with R⁵.

13. The compound of claim 12, wherein R¹ is 3-chlorophenyl, 3-cyanophenyl, or 3-methylphenyl.

14. The compound of claim 10, wherein R¹ is pyridin-2-yl substituted at the 4-position with R⁵.

15. The compound of claim 14, wherein R⁵ is trifluoromethyl, cyano, chloro, methoxy, amide, or hydroxyl.

16. The compound of any one of the preceding claims, wherein R² is C₅₋₇ cycloalkyl.

17. The compound of any one of claims 1-15, wherein R² is 4-7 membered heterocyclyl.

18. The compound of claim 17, wherein R² is tetrahydropyranyl, piperidinyl, pyrrolidinyl, tetrahydrofuranyl, or azabicyclo[2.2.1]heptanyl.

19. The compound of any one of claims 1-15, wherein R² is C₁₋₆ alkyl.

20. The compound of claim 19, wherein R² is tert-butyl, neopentyl, methyl, or ethyl.

21. The compound of claim 19, wherein R² is tert-butyl or neopentyl.

22. The compound of claim 21, wherein the compound is: 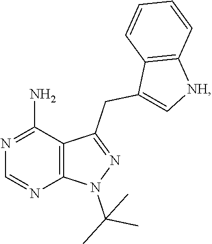 [SMILES: CC(C)(C)n1nc(Cc2c[nH]c3ccccc23)c2c(N)ncnc21]
or a pharmaceutically acceptable salt thereof.

23. The compound of any one of claims 1-15, wherein R² is heteroaralkyl.

24. The compound of claim 23, wherein R² is pyrrolylmethyl.

25. The compound of any one of claims 1-15, wherein R² is carbocyclylalkyl.

26. The compound of claim 25, wherein R² is cyclobutylmethyl or cyclopropylmethyl.

27. The compound of claim 25, wherein R² is cyclopropylmethyl.

28. The compound of any one of claims 1-15, wherein R² is heterocyclylalkyl.

29. The compound of claim 28, wherein R² is 1,2-oxaborolanylmethyl or piperidinylmethyl.

30. The compound of claim 28, wherein R² is piperidinylmethyl.

31. The compound of any one of claims 1-15, wherein R² is C₁₋₆ alkenyl.

32. The compound of claim 31, wherein R² is butenyl.

33. The compound of any one of claims 1-15, wherein R² is C₁₋₆ cycloalkenyl.

34. The compound of claim 33, wherein R² is cyclohexenyl.

35. The compound of any one of the preceding claims, wherein R² is substituted by one or more R⁷ selected from fluoro, hydroxyl, hydroxymethyl, butyloxycarbonylamine, butyloxycarbonyl, amino, trifluoromethyl, methoxycarbonyl, dimethylamine, methoxy, methyl, methylamino, boronic acid, ethoxycarbonyl, carboxy, or oxo.

36. The compound of any one of claims 1-16, wherein R² is selected from dimethylcyclohexyl, cyclohexanoyl, aminocyclopentyl, methylcyclohexyl, dimethylaminocyclohexyl, methoxycyclohexyl, trifluoromethylcyclohexyl, methoxycarbonylcyclohexyl, hydroxycyclohexyl, hydroxymethylcyclohexyl, difluorocyclohexyl, fluorocyclohexyl, hydroxycyclopentyl, (butyloxycarbonyl)aminocyclopentyl, methylaminocyclohexyl, difluorohydroxycyclohexyl, oxocyclohexyl, and aminocyclohexyl.

37. The compound of any one of claims 1-16, wherein R² is unsubstituted cyclopentyl.

38. The compound of any one of claims 1-16, wherein R² is cyclopentyl or cyclohexyl, and is substituted by one or more R⁷ selected from haloalkyl, ester, and carbamate.

39. The compound of any one of the preceding claims, wherein R⁶ is methylene.

40. The compound of any one of the preceding claims, having the structure of formula (Ia) or a pharmaceutically acceptable salt thereof: 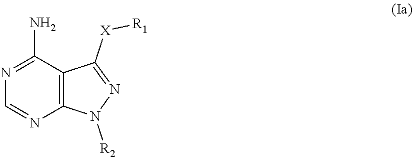
wherein:
X is R⁶ or O;
R¹ is phenyl or 6-membered heteroaryl optionally substituted with one or more R⁵ independently selected from C₁₋₃ alkyl, C₁₋₃ haloalkyl, cyano, acyloxy, hydroxyl, alkoxy, or halo;
R² is C₅₋₇ cycloalkyl, 4-7 membered heterocyclyl, C₁₋₆ alkyl, heteroaralkyl, carbocyclylalkyl, heterocyclylalkyl, C₂₋₆ alkenyl, C₂₋₆ cycloalkenyl, or H; and
R⁶ is C₁₋₃ alkylene.

41. The compound of claim 40, wherein R¹ is pyridinyl optionally substituted with one or more R⁵.

42. The compound of claim 41, wherein the compound is: 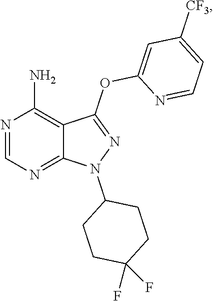 [SMILES: Nc1ncnc2c1c(Oc1cc(C(F)(F)F)ccn1)nn2C1CCC(F)(F)CC1]
or a pharmaceutically acceptable salt thereof.

43. The compound of any one of claims 1-39, having the structure of formula (Ib) or a pharmaceutically acceptable salt thereof: 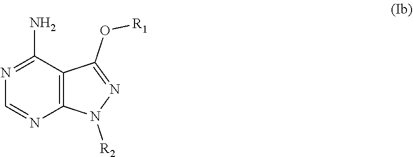
wherein:
R¹ is pyridinyl substituted with one or more R⁵;
R² is C₅₋₇ cycloalkyl, 4-7 membered heterocyclyl, C₁₋₆ alkyl, heteroaralkyl, carbocyclylalkyl, C₂₋₆ alkenyl, C₄₋₆ cycloalkenyl, or heterocyclylalkyl.

44. The compound of claim 43, wherein R¹ is pyridinyl substituted with one or more R⁵ independently selected from trifluoromethyl, cyano, chloro, hydroxyl, methoxy, acetoxy, and amide.

45. The compound of claim 43 or claim 44, wherein R² is C₅₋₇ cycloalkyl.

46. The compound of claim 43 or claim 44, wherein R² is carbocyclylalkyl.

47. The compound of claim 46, wherein R² is cyclobutylmethyl or cyclopropylmethyl.

48. The compound of claim 46, wherein R² is cyclopropylmethyl.

49. The compound of claim 43 or claim 44, wherein R² is heterocyclylalkyl.

50. The compound of claim 49, wherein R² is 1,2-oxaborolanylmethyl or piperidinylmethyl.

51. The compound of claim 49, wherein R² is piperidinylmethyl.

52. The compound of claim 43 or claim 44, wherein R² is C₁₋₆ alkyl.

53. The compound of claim 52, wherein R² is tert-butyl, neopentyl, methyl, or ethyl.

54. The compound of claim 52, wherein R² is tert-butyl or neopentyl.

55. The compound of claim 43 or claim 44, wherein R² is C₁₋₆ alkenyl.

56. The compound of claim 55, wherein R² is butenyl.

57. The compound of claim 43 or claim 44, wherein R² is cycloalkenyl.

58. The compound of claim 57, wherein R² is cyclohexenyl.

59. The compound of claim 43 or claim 44, wherein R² is 4-7 membered heterocyclyl.

60. The compound of claim 59, wherein R² is azabicyclo[2.2.1]heptanyl, tetrahydropyranyl, tetrahydrofuranyl, piperidinyl, or pyrrolidinyl.

61. The compound of claim 43 or 44, wherein R² is cyclohexyl, cyclopentyl, piperidinylmethyl, pyrrolidinylmethyl, cyclopropylmethyl, tert-butyl, neopentyl, tetrahydropyranyl, pyrrolidinyl, piperidinyl, ethyl, methyl, cyclohexenyl, butenyl, 1,2-oxaborolanylmethyl, cyclobutyl methyl, or azabicyclo[2.2.1]heptanyl, substituted by one or more R⁷ selected from hydroxyl, fluoro, hydroxymethyl, butyloxycarbonylamino, amino, trifluoromethyl, methoxycarbonyl, dimethylamino, butyloxycarbonyl, methoxy, methyl, methylamino, boronic acid, and oxo.

62. The compound of claim 43, wherein the compound is: 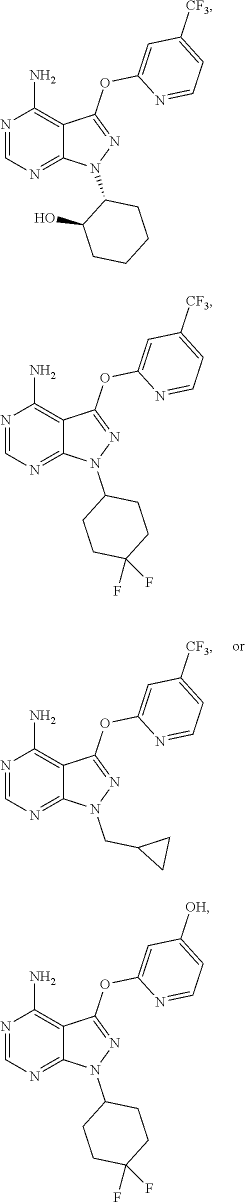 [SMILES: Nc1ncnc2c1c(Oc1cc(C(F)(F)F)ccn1)nn2[C@@H]1CCCC[C@H]1O ; Nc1ncnc2c1c(Oc1cc(C(F)(F)F)ccn1)nn2C1CCC(F)(F)CC1 ; Nc1ncnc2c1c(Oc1cc(C(F)(F)F)ccn1)nn2CC1CC1 ; Nc1ncnc2c1c(Oc1cc(O)ccn1)nn2C1CCC(F)(F)CC1]
or a pharmaceutically acceptable salt thereof.

63. The compound of any one of the preceding claims, wherein the compound has a greater than 10-fold selectivity for a protozoan CDPK1 versus human SRC kinase.

64. The compound of any one of the preceding claims, wherein the compound has a greater than 30-fold selectivity for a protozoan CDPK1 versus human SRC kinase.

65. The compound of any one of the preceding claims, wherein the compound has a greater than 100-fold selectivity for a protozoan CDPK1 versus human SRC kinase.

66. The compound of any one of claims 63-65, wherein the protozoan is an Apicomplexan protozoan.

67. The compound of claim 66, wherein the protozoan is T. gondii, P. falciparum, C. hominis, or C. parvum.

68. The compound of claim 66, wherein the protozoan is T. gondii.

69. A pharmaceutical composition comprising a compound of any one of the preceding claims and a pharmaceutically acceptable carrier.

70. A method of treating an infection, comprising administering a compound or composition of any one of claims 1-69.

71. The method of claim 70, wherein the infection is caused by a protozoan.

72. The method of claim 71, wherein the protozoan is an Apicomplexan protozoan.

73. The method of claim 72, wherein the protozoan is T. gondii, P. falciparum, C. hominis, or C. parvum.

74. The method of claim 73, wherein the protozoan is T. gondii.

75. A compound or composition of any one of claims 1-69, for use in the treatment of an infection.

76. The compound of claim 75, wherein the infection is caused by a protozoan.

77. The compound of claim 76, wherein the protozoan is an Apicomplexan protozoan.

78. The compound of claim 77, wherein the protozoan is T. gondii, P. falciparum, C. hominis, or C. parvum.

79. The compound of claim 78, wherein the protozoan is T. gondii.
